(19) United States
(12) Patent Application Publication
(10) Pub. No.: US 2012/0283310 A1
(43) Pub. Date: Nov. 8, 2012

(54) MicroRNA Signatures Associated with Human Chronic Lymphocytic Leukemia (CLL) and Uses Thereof

(76) Inventors: Carlo M. Croce, Columbus, OH (US); George A. Calin, Pearland, TX (US)

(21) Appl. No.: 13/536,837

(22) Filed: Jun. 28, 2012

Related U.S. Application Data
(62) Division of application No. 12/919,904, filed on Nov. 12, 2010.
(60) Provisional application No. 61/067,406, filed on Feb. 28, 2008.

(51) Int. Cl. A61K 31/7088; A61P 35/02; C12N 5/09

(52) U.S. Cl. 514/44 A; 435/375

(57) ABSTRACT

Methods and compositions for the diagnosis, prognosis and/or treatment of leukemia associated diseases are disclosed.

### CROSS-REFERENCE TO RELATED APPLICATIONS

This application is a divisional application of Ser. No. 12/919,904 national stage entry on Nov. 12, 2010, from PCT Application No. PCT/US2009/035463, filed Feb. 27, 2009, which claims benefit to U.S. Provisional Application No. 61/067,406 filed Feb. 28, 2008; the entire disclosure of each aforementioned application is expressly incorporated herein by reference for all purposes.

### STATEMENT REGARDING FEDERALLY SPONSORED RESEARCH

This invention was made with government support under the NCI Grant Number(s) CA76259 and CA81533. The government has certain rights in this invention.

### REFERENCE TO SEQUENCE LISTING SUBMITTED VIA EFS-WEB

This application is being filed electronically via the USPTO EFS-WEB server, as authorized and set forth in MPEP§1730 II.B.2(a)(A), and this electronic filing includes an electronically submitted sequence (SEQ ID) listing. The entire content of this sequence listing is herein incorporated by reference for all purposes. The sequence listing is identified on the electronically filed .txt file as follows: 604_{—}54189_SEQ_ID_Txt_OSU-2008-077(2).TXT, created on Jun. 27, 2012 and is 1,147 bytes in size.

### TECHNICAL FIELD AND INDUSTRIAL APPLICABILITY OF THE INVENTION

This invention relates generally to the field of molecular biology. Certain aspects of the invention include application in diagnostics, therapeutics, and prognostics of leukemia related disorders.

### BACKGROUND OF THE INVENTION

There is no admission that the background art disclosed in this section legally constitutes prior art.

MicroRNAs (miRNAs) are short noncoding RNAs of ≈19-24 nt, that regulate gene expression by imperfect base-pairing with complementary sequences located mainly, but not exclusively, in the 3′ UTRs of target mRNAs. MiRNAs represent one of the major regulatory family of genes in eukaryotic cells by inducing translational repression and transcript degradation (1-4). Different algorithms such as TargetScan (5), PicTar (6), and Diana microT (7) have been developed to identify miRNA targets, but only few of these predictions have been experimentally validated, supporting the rationale for a combination of bioinformatics and biological strategies to this aim. Two independent studies predicted that 20-30% of human genes could be controlled by miRNAs (8, 9). Deviations from normal miRNA expression patterns play roles in human diseases, including cancer (for reviews see refs. 10-15).

The miR-15α/16-1 cluster resides at chromosome 13q14.3, a genomic region frequently deleted in B cell chronic lymphocytic leukemias (CLLs), and the two members of the cluster are cotranscribed and down-regulated in the majority of CLL patients (16).

CLL is a disease with a frequent association in families (10-20% of patients have at least one first-degree relative with CLL) (17). Previously, we identified germ-line or somatic mutations in several miRNAs (including miR-16-1) in ≈15% of CLL patients, with the majority of the patients having a known personal or family history of CLL or other hematopoietic and solid tumors (18). We used these findings, together with the identification of an abnormal miR-15α/16-1 locus in the NZB strain of mice that naturally develop CLL (19), to now show herein that this cluster also plays a role in familial CLL.

Among the targets of miR-15α and miR-16, we identified the antiapoptotic protein Bc12, which is overexpressed in the malignant, mostly nondividing B cells of CLL (20), and in many solid and hematologic malignancies (21). Restoration of miR-15-a/16-1 induces apoptosis in MEG-01, a cell line derived from acute megakaryocytic leukemia (22).

In spite of considerable research into therapies to treat these diseases, they remain difficult to diagnose and treat effectively, and the mortality observed in patients indicates that improvements are needed in the diagnosis, treatment and prevention of the disease.

### SUMMARY OF THE INVENTION

In a first aspect, there is provided herein a signature of genes whose silencing characterizes the miR-15a/16-1-induced phenotype in chronic lymphocytic leukemias (CLL).

In another aspect, there is provided herein use of one or more of miRs in the miR-15a/16-1 cluster for deregulating genes in one or more of a leukemic cell model and in primary chronic lymphocytic leukemias (CLLs).

In another aspect, there is provided herein a method for developing therapeutic approaches for CLLs using the signature described herein.

In another aspect, there is provided herein use of miR-15a and miR-16-1 cluster as tumor-suppressor in chronic lymphocytic leukemias (CLL).

In another aspect, there is provided herein a method for inhibiting the growth of tumor engraftments of leukemic cells comprising exposing such cells to one or more of miRs in the miR-15a/116-1 cluster wherein a tumor suppressor function is exerted on such cells.

In another aspect, there is provided herein a method for exerting an antileukemic effect in a subject in need thereof, comprising directly silencing IGSF4 by administering one or more of the miRs in the miR-15a/116-1 cluster, or functional variants thereof, to the subject.

In another aspect, there is provided herein a signature of genes in common between CLLs and MEG-01 transfected with miR-15a/116-1, comprising one or more of the genes listed in FIG. 15—Table 11.

In another aspect, there is provided herein use of one or more of miRs in the miR-15a/16-1 cluster and miR-29s in the treatment of CLL.

In another aspect, there is provided herein use of one or more of miRs in the miR-15a/16-1 cluster and miR-29s in the treatment of CLL, including targeting both MCL1 and c-JUN transcripts, wherein the impact of the miR-15a/16-1 cluster on the survival of B-CLL cells is increased.

In another aspect, there is provided herein a method for reducing expression of one or more of PDCD4, RAB21, IGSF4, SCAP2 and/or proteomics identified proteins (Bc12, Wt1), comprising transfecting cells in need thereof with one or more miRs in the miR-15a/16-1 cluster.

In another aspect, there is provided herein use of miR-15a/16-1 cluster to directly target IGSF4.

In another aspect, there is provided herein a method of inhibiting the growth of cells, comprising contacting a cell expressing IGSF4 with one or more miRs in the miR-15a/16-1 cluster, or functional variants thereof, under conditions such that the expression of IGSFS in the cell is inhibited.

In certain embodiments, the cell is a cancer cell.

In certain embodiments, the cell is a chronic lymphocytic leukemia cell.

In certain embodiments, the cell is in an organism.

In certain embodiments, the organism is an animal.

In certain embodiments, the organism has been diagnosed with cancer.

In another aspect, there is provided herein a method of inhibiting the formation of a selected miRNA known to inhibit translation of one or more identified proteins, comprising administering one or more miRs selected from the miR-15a16-1 cluster to a subject in need thereof.

In another aspect, there is provided herein a CLL signature comprising one or more miR 15a/16-1 down-regulated genes listed in FIG. 7—Table 3.

In another aspect, there is provided herein a CLL signature comprising one or more miR 15a/16-1 down-regulated genes listed in FIG. 8—Table 4.

In another aspect, there is provided herein a CLL signature comprising one or more miR 15a/16-1 down-regulated genes listed in FIG. 11—Table 7.

In another aspect, there is provided herein a CLL signature comprising one or more miR 15a/16-1-regulated genes listed in FIG. 12—Table 8:

In another aspect, there is provided herein a CLL signature comprising one or more miR 15a/16-1-regulated genes listed in FIG. 13—Table 9.

In another aspect, there is provided herein a CLL signature comprising one or more miR 15a/16-1 down-regulated genes listed in FIG. 14—Table 10.

In another aspect, there is provided herein a CLL signature comprising one or more miR 15a/16-1 down-regulated genes listed in FIG. 15—Table 11.

In another aspect, there is provided herein a CLL signature comprising one or more miR 15a/16-1 down-regulated genes listed in FIG. 16—Table 12.

In another aspect, there is provided herein a method for determining diagnosing whether a subject has or will develop chronic lymphocytic leukemia (CLL) comprising examining a sample from the subject and determining whether there is a positive correlation of expression of miRs selected from the miR15a/16-1 cluster.

In another aspect, there is provided herein a method of using a signature described herein in one or more of the diagnosis of, the treatment of, or the determination of the prognosis of a subject who has or may develop chronic lymphocytic leukemia (CLL).

In another aspect, there is provided herein a method for predicting an outcome of a patient suffering from chronic lymphocytic leukemia (CLL), comprising: determining a distinct signature of miRNA expression compared with normal cells, wherein the signature comprises one or more of the miRNAs signatures described herein.

In another aspect, there is provided herein a method of: i) diagnosing whether a subject has, or is at risk for developing chronic lymphocytic leukemia (CLL), ii) determining a prognosis of such subject, and/or iii) treating such subject, comprising: measuring the level of at least one biomarker in a test sample from the subject, wherein the biomarker is selected from one or more of the CLL signatures described herein, and, wherein an alteration in the level of the biomarker in the test sample, relative to the level of a corresponding biomarker in a control sample, is indicative of the subject either having, or being at risk for developing, CLL.

In certain embodiments, the level of the at least one biomarker in the test sample is less than the level of the corresponding biomarker in the control sample.

In certain embodiments, the level of the at least one biomarker in the test sample is greater than the level of the corresponding biomarker in the control sample.

In another aspect, there is provided herein a method for influencing transcript abundance and/or protein expression of target mRNAs in chronic lymphocytic leukemia (CLL), comprising deregulating one or more microRNAs in a subject in need thereof.

In certain embodiments, the method further comprises inhibiting the protein expression of cancer-related genes.

In another aspect, there is provided herein use of a large-scale gene expression profiling of both microRNAs and protein-encoding RNAs to identify alterations in microRNA function that occur in human chronic lymphocytic leukemia (CLL).

In another aspect, there is provided herein a method of determining the prognosis of a subject with chronic lymphocytic leukemia (CLL), comprising measuring the level of at least one biomarker in a test sample from the subject, wherein: the biomarker is associated with an adverse prognosis in such cancer; and an alteration in the level of the at least one biomarker in the test sample, relative to the level of a corresponding biomarker in a control sample, is indicative of an adverse prognosis.

In another aspect, there is provided herein a method of determining the prognosis of a subject with chronic lymphocytic leukemia (CLL), comprising diagnosing whether a subject has, or is at risk for developing, CLL, comprising: reverse transcribing RNA from a test sample obtained from the subject to provide a set of target oligodeoxynucleotides; hybridizing the target oligodeoxynucleotides to a microarray comprising miRNA-specific probe oligonucleotides to provide a hybridization profile for the test sample; and comparing the test sample hybridization profile to a hybridization profile generated from a control sample, wherein an alteration in the signal of at least one miRNA is indicative of the subject either having, or being at risk for developing, such AML.

In certain embodiments, the signal of at least one miRNA, relative to the signal generated from the control sample, is down-regulated, and/or wherein the signal of at least one miRNA, relative to the signal generated from the control sample, is up-regulated.

In certain embodiments, an alteration in the signal of at least one biomarker selected from the miRs of the miR15a/16-1 cluster, which is indicative of the subject either having, or being at risk for developing, CLL cancer with an adverse prognosis.

In another aspect, there is provided herein a method for regulating protein expression in leukemia cells, comprising modulating the expression of one or more of: miRs of the miR15a/16-1 cluster in the leukemia cells.

In another aspect, there is provided herein a composition for modulating expression of one or more of protein levels in leukemia cells, the composition comprising one or more of: miRs of the miR15a/16-1 cluster, or functional variants thereof.

In another aspect, there is provided herein a composition comprising one or more antisense miRs of the miR15a/16-1 cluster, useful to increase protein levels in leukemia cells in a subject in need thereof.

In another aspect, there is provided herein a method of treating chronic lymphocytic leukemia (CLL) in a subject who has a leukemia in which at least one biomarker is down-regulated or up-regulated in the cancer cells of the subject relative to control cells, comprising: when the at least one biomarker is down-regulated in the cancer cells, administering to the subject an effective amount of at least one isolated biomarker, or an isolated variant or biologically-active fragment thereof, such that proliferation of cancer cells in the subject is inhibited; or, when the at least one biomarker is up-regulated in the cancer cells, administering to the subject an effective amount of at least one compound for inhibiting expression of the at least one biomarker, such that proliferation of cancer cells in the subject is inhibited.

In another aspect, there is provided herein a method of treating leukemia in a subject, comprising: determining the amount of at least one biomarker in leukemia cells, relative to control cells; wherein the biomarker is selected from one or more of the miRs of the miR15a/16-1 cluster, or functional variants thereof, and altering the amount of biomarker expressed in the leukemia cells by: administering to the subject an effective amount of at least one isolated biomarker, if the amount of the biomarker expressed in the cancer cells is less than the amount of the biomarker expressed in control cells; or administering to the subject an effective amount of at least one compound for inhibiting expression of the at least one biomarker, if the amount of the biomarker expressed in the cancer cells is greater than the amount of the biomarker expressed in control cells.

In another aspect, there is provided herein a pharmaceutical composition for treating leukemia, comprising at least one isolated biomarker, wherein the biomarker is selected from one or more of the miRs of the miR15a/16-1 cluster, or functional variants thereof, and a pharmaceutically-acceptable carrier.

In certain embodiments, the pharmaceutical composition comprises at least one miR expression-inhibitor compound and a pharmaceutically-acceptable carrier.

In another aspect, there is provided herein a method of identifying an anti-leukemia agent, comprising providing a test agent to a cell and measuring the level of at least one biomarker associated with decreased expression levels in leukemia cells, wherein the biomarker is selected from one or more of the miRs of the miR15a/16-1 cluster, or functional variants thereof, and wherein an increase in the level of the biomarker in the cell, relative to a control cell, is indicative of the test agent being an anti-leukemia agent.

In another aspect, there is provided herein a method of identifying an anti-leukemia agent, comprising providing a test agent to a cell and measuring the level of at least one biomarker associated with increased expression levels in leukemia cells, wherein a decrease in the level of the biomarker in the cell, relative to a control cell, is indicative of the test agent being an anti-cancer agent, wherein the biomarker is selected from one or more of the miRs of the miR15a/16-1 cluster, or functional variants thereof.

In another aspect, there is provided herein a method of assessing the effectiveness of a therapy to prevent, diagnose and/or treat a chronic lymphocytic leukemia (CLL) associated disease, comprising: subjecting an animal to a therapy whose effectiveness is being assessed, and determining the level of effectiveness of the treatment being tested in treating or preventing the disease, by evaluating at least one biomarker, wherein the biomarker is selected from one or more of the miRs of the miR15a/16-1 cluster, or functional variants thereof.

In certain embodiments, the candidate therapeutic agent comprises one or more of:

pharmaceutical compositions, nutraceutical compositions, and homeopathic compositions.

In certain embodiments, the therapy being assessed is for use in a human subject.

In another aspect, there is provided herein an article of manufacture comprising: at least one capture reagent that binds to a marker for a leukemia associated disease comprising at least one biomarker, wherein the biomarker is selected from one or more of the miRs of the miR15a/16-1 cluster, or functional variants thereof.

In another aspect, there is provided herein a kit for screening for a candidate compound for a therapeutic agent to treat a leukemia associated disease, wherein the kit comprises: one or more reagents of at least one biomarker and a cell expressing at least one biomarker, wherein the biomarker is selected from one or more of the miRs of the miR15a/16-1 cluster, or functional variants thereof.

In certain embodiments, the presence of the biomarker is detected using a reagent comprising an antibody or an antibody fragment which specifically binds with at least one biomarker.

In another aspect, there is provided herein use of an agent that interferes with a chronic lymphocytic leukemia (CLL) associated disease response signaling pathway, for the manufacture of a medicament for treating, preventing, reversing or limiting the severity of the disease complication in an individual, wherein the agent comprises at least one biomarker, wherein the biomarker is selected from one or more of the miRs of the miR15a/16-1 cluster, or functional variants thereof.

In another aspect, there is provided herein a method of treating, preventing, reversing or limiting the severity of a leukemia associated disease complication in an individual in need thereof, comprising: administering to the individual an agent that interferes with at least a leukemia associated disease response cascade, wherein the agent comprises at least one biomarker, wherein the biomarker is selected from one or more of the miRs of the miR15a/16-1 cluster, or functional variants thereof.

In another aspect, there is provided herein use of an agent that interferes with at least a chronic lymphocytic leukemia (CLL) associated disease response cascade, for the manufacture of a medicament for treating, preventing, reversing or limiting the severity of a leukemia-related disease complication in an individual, wherein the agent comprises at least one biomarker, wherein the biomarker is selected from one or more of the miRs of the miR15a/16-1 cluster, or functional variants thereof.

In another aspect, there is provided herein a composition comprising an antisense inhibitor of one or more of miRs of the miR15a/16-1 cluster, or functional variants thereof.

In another aspect, there is provided herein a method of treating chronic lymphocytic leukemia (CLL) in a subject in need thereof, comprising administering to a subject a therapeutically effective amount of the composition.

In certain embodiments, the composition is administered prophylactically.

In certain embodiments, administration of the composition delays the onset of one or more symptoms of CLL.

In certain embodiments, administration of the composition inhibits development of CLL.

In certain embodiments, administration of the composition inhibits CLL.

In another aspect, there is provided herein a method for detecting the presence of leukemia in a biological sample, comprising: exposing the biological sample suspected of containing leukemia to a biomarker therefor; wherein the biomarker is selected from one or more of the miRs of the miR15a/16-1 cluster, or functional variants thereof, and detecting the presence or absence of the marker, if any, in the sample.

In certain embodiments, the biomarker includes a detectable label.

In certain embodiments, the method further comprises comparing the amount of the biomarker in the biological sample from the subject to an amount of the biomarker in a corresponding biological sample from a normal subject.

In certain embodiments, the method further comprises collecting a plurality of biological samples from a subject at different time points and comparing the amount of the marker in each biological sample to determine if the amount of the marker is increasing or decreasing in the subject over time.

In another aspect, there is provided herein a method for treating chronic lymphocytic leukemia (CLL) in a subject, the method comprising: a leukemia receptor agonist.

In certain embodiments, the receptor agonist is an antisense inhibitor of one or more of: the miRs of the miR15a/16-1 cluster, or functional variants thereof.

In another aspect, there is provided herein a use, to manufacture a drug for the treatment of acute myeloid leukemia, comprised of a nucleic acid molecule chosen from among the miRs of the miR15a/16-1 cluster, or functional variants thereof, a sequence derived therefrom, a complementary sequence from such miR and a sequence derived from such a complementary sequence.

In certain embodiments, the drug comprises a nucleic acid molecule presenting a sequence chosen from among one or more of the miRs of the miR15a/16-1 cluster, or functional variants thereof, a sequence derived from such miRs, the complementary sequence of such miRs, and a sequence derived from such a complementary sequence.

In another aspect, there is provided herein an in vitro method to identify effective therapeutic agents or combinations of therapeutic agents to induce the differentiation of chronic lymphocytic leukemia (CLL) cells, the method comprising the stages of: culturing of cells derived from CLL cells, adding at least one compound to the culture medium of the cell line, analyzing the evolution of the level of expression of at least one miR between stages (i) and (ii), and identifying compounds or combinations of compounds inducing a change in the level of expression of the miR between stages (i) and (ii).

In certain embodiments, stage (iii) includes the analysis of the level of expression of at least one miR.

In certain embodiments, stage (iv) includes the identification of the compounds or combinations of compounds modulating the level of expression of at least one miR.

In certain embodiments, stage (iv) includes the identification of compounds or combinations of compounds reducing the level of expression of at least one miR.

In certain embodiments, the compound is a therapeutic agent for the treatment of cancer.

Various objects and advantages of this invention will become apparent to those skilled in the art from the following detailed description of the preferred embodiment, when read in light of the accompanying drawings.

### BRIEF DESCRIPTION OF THE DRAWINGS

FIGS. 1A-1C: MiR15a/16-1 cluster inhibits the growth of MEG-01 tumor engraftments in nude mice.

FIG. 1A: Growth curve of engrafted tumors in nude mice injected with MEG-01 cells pretransfected with pRS-E or pRS15/16 or mock transfected.

FIG. 1B: Comparison of tumor engraftment sizes of mock-, pRS-E-, and pRS15/16-transfected MEG-01 cells 28 days after injection in nude mice.

FIG. 1C: Tumor weights ±SD in nude mice.

FIG. 2: Validation of some of the targets of miR-15a/16-1 identified by microarray or proteomics in MEG-01.

FIG. 2A: qRT-PCR validation of PDCD4, RAB21, IGSF4, SCAP2 (down-regulated in the microarray), BCL2, and WT1 (down-regulated in proteomics). IFG1, ACE, and ERBB2 are negative controls. The results were normalized to pRS-E-transfected cells. Samples were normalized with β-tubulin.

FIG. 2B: Luciferase assay of IGSF4 in MEG-01 cells, showing that the miR-15a/16-1 cluster directly targets this gene.

FIG. 3: Gene expression profile of MEG-01 cells transfected with miR-15α/16-1. Cluster of samples according to the expression of 5,659 probes differentially expressed between MEGO1 transfected with empty vector and with miR-15/16 expressing vector. Dark shading indicates an expression value higher than average value across all samples, medium shading indicates an expression value lower.

FIG. 4: Venn diagrams matching predicted and experimentally (microarray) deregulated targets of miR-15a16-1 in MEG-01. Results of the match between targets predicted by TargetScan, MiRanda, and PicTar, and experimentally down-regulated transcripts. The number outside the Venn diagram (4,769) indicates the number of transcripts, which are down-regulated in the microarray but are not predicted to be a target by any of the considered algorithms

FIG. 5—Table 1: Cluster distribution of ARE-mRNAs deregulated in MEG-01 cells after miR-15α/16-1 cluster transfection.

FIG. 6—Table 2: Most significant GO categories after miR-15α/16-1 cluster transfection in MEG-01 cells.

FIG. 7—Table 3: Examples of proteins down-regulated by the miR-15α/16-1 cluster identified by proteomics in MEG-01 cells.

FIG. 8—Table 4: Examples of the CLL signature of miR-15a/16-1 down-regulated genes by microarray.

FIG. 9—Table 5: Deregulated transcripts after transfection of MEG-01 cells with miR-15a/16-1.

FIG. 10—Table 6: Down-regulated transcripts after transfection of MEG-01 cells with miR-15a16-1, and predicted targets by TargetScan, PicTar, and MiRanda.

FIG. 11—Table 7: ARE-mRNAs among the transcripts which are up-/down-regulated after transfection of MEG-01 cells with miR-15a16-1. In bold are upregulated genes; in standard font are down-regulated genes “(DEAD (Asp-Glu-Ala-Asp) disclosed as SEQ ID NO:3).”

FIG. 12—Table 8: Gene Ontology of down-regulated transcripts after transfection of MEG-01 cells with miR-15a16-1, with respect to empty vector.

FIG. 13—Table 9: Proteins down-regulated by the miR-15a/16-1 cluster identified by proteomics in MEG-01 cells.

FIG. 14—Table 10: Comparison between 8 CLLs with high miR-15a16-1 levels and 8 CLLs with low miR-15a/16-1 levels. 678 transcripts result significantly differentially expressed “(DEAD (Asp-Glu-Ala-Asp) disclosed as SEQ ID NO: 3; DEAH (Asp-Glu-Ala-His) disclosed as SEQ ID NO:4).”

FIG. 15—Table 11: The CLL signature of miR-15α/16-1 down-regulated genes.

FIG. 16—Table 12: Gene Ontology of transcripts that are down-regulated after transfection of MEG-01 cells with miR-15a/16-1 (with respect to empty vector) and are down-regulated in CLL patients with high expression of miR-15a16-1.

### DETAILED DESCRIPTION OF THE PREFERRED EMBODIMENT

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Described herein is a role for miR-15a and miR-16-1 as tumor-suppressor genes (TSGs) in CLLs and perhaps in other malignancies in which these genes are lost or down-regulated.

Also disclosed herein is the mechanism of action of miR-15a and miR-16-1 as tumor suppressors in leukemias. We analyzed the effects of miR-15a and miR-16-1 on transcriptome and proteome in MEG-01 leukemic cells. This approach allowed us to validate a number of target genes, whose expression was also investigated in cases of CLL.

The present invention is further explained in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference.

### EXAMPLE I

In Vivo Effects of miR-15α/miR-16-1 Transfection into MEG-01 Leukemic Cells

We reported that miR-15a16-1 cluster induces apoptosis of MEG-01 cells by activating the intrinsic apoptosis pathway as identified by activation of the APAF-1-caspase9-PARP pathway (22).

To further investigate the effect of these miRNAs, we tested their tumor-suppression function in vivo. Ten million viable MEG-01 cells, transfected in vitro with pRS15/16, pRS-E, or mock transfected, were inoculated s.c. in the flanks of immunocompromised “nude” mice (5 per group).

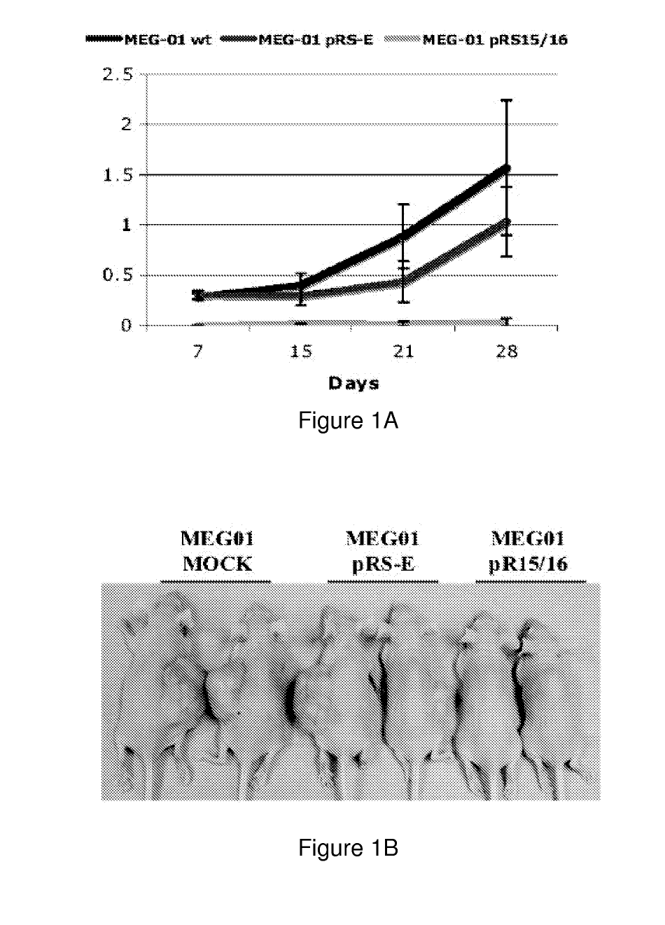

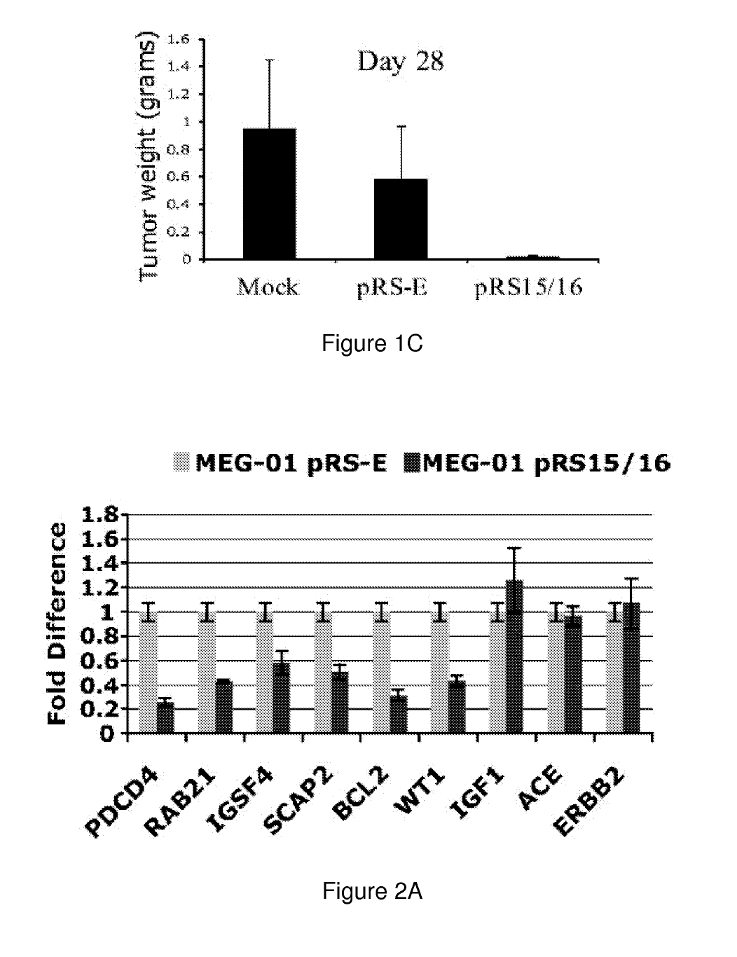

As shown in FIG. 1A, the miR-15a16-1 cluster inhibits the growth of MEG-01 tumor engraftments. After 28 days, tumor growth was completely suppressed in three of five (60%) mice inoculated with miR-15α/16-1-transfected MEG-01 (FIG. 1B).

At day 28, the average tumor weights for the untreated and empty vector-treated mice were 0.95±0.5 g and 0.58±0.39 g, respectively; in mice inoculated with miR-15α/16-1-treated cells, the average was 0.020±0.01 g (P <0.003) (FIG. 1C).

Thus, the results of these experiments demonstrate the tumor-suppressor function of miR-15α/16-1 cluster in MEG-01 leukemia cells.

Transcriptional Effects of Exogenous Expression of miR-15a and mirR-16-1.

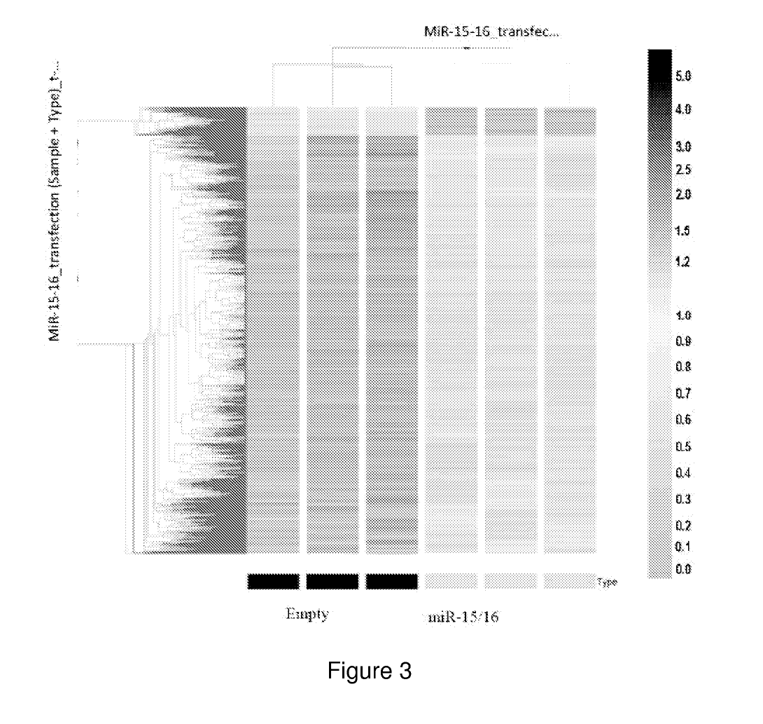

To characterize the molecular basis of miR-15α/16-1 tumor suppression in leukemias, we first investigated the effect of miRNAs on genome-wide transcription of protein-coding genes. We transiently transfected the pRS15/16 vector into MEG-01 cells. This vector contains a genomic region encoding for both miRNAs as described (22). Transfection with the empty vector (pRS-E) was used as control. The success of transfection was assessed by measuring the expression levels of miR-15α, and miR-16-1 by quantitative (q)RT-PCR as described in ref. 18 (data not shown). Genome-wide transcriptome was investigated by using Affymetrix microarray. The microarray analysis clearly shows a different pattern of gene expression among pRS15/16—and pRS-E-transfected cells (FIG. 3).

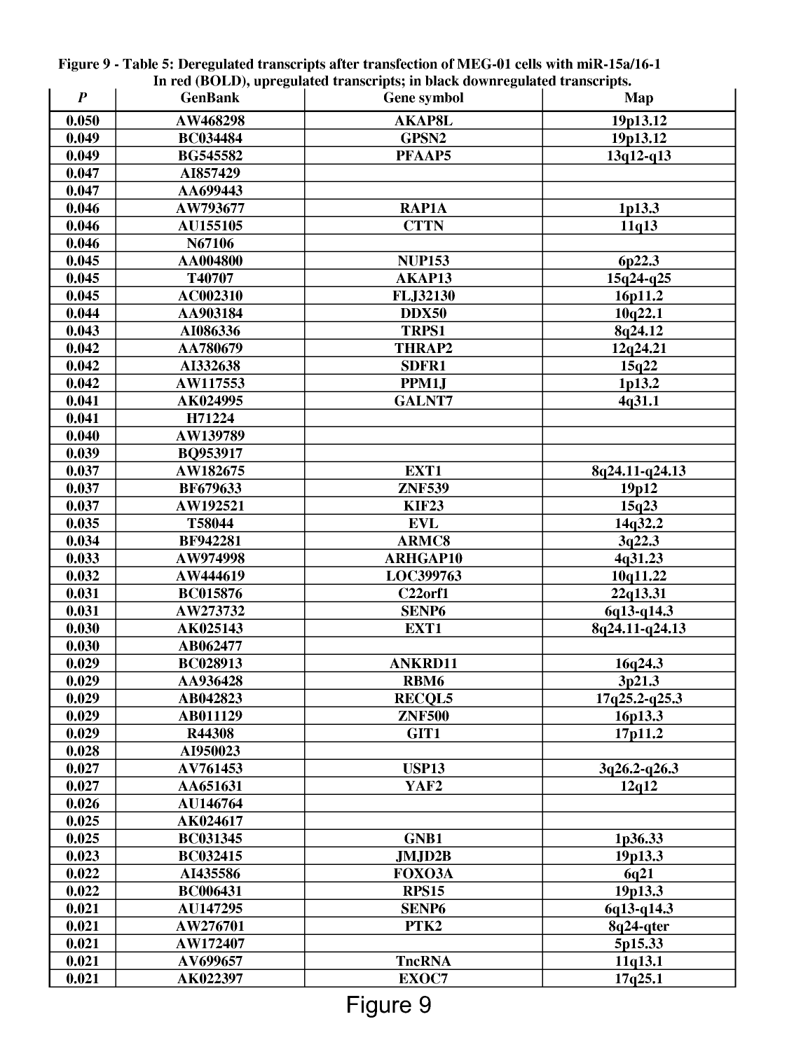

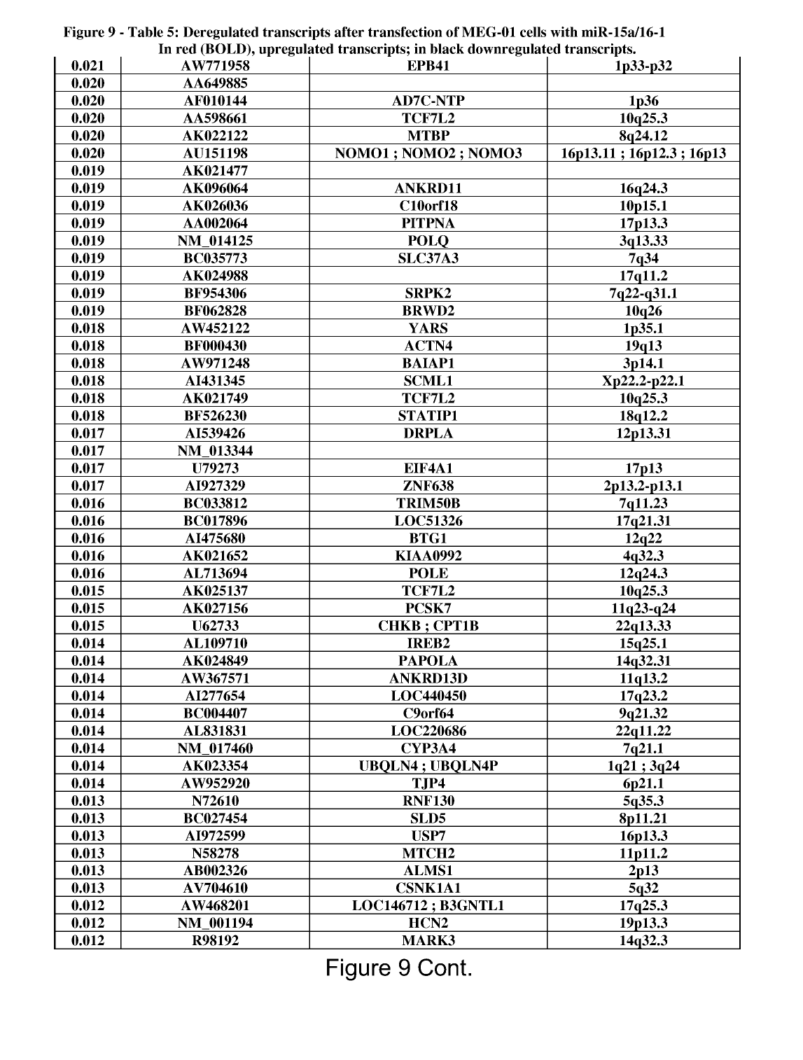

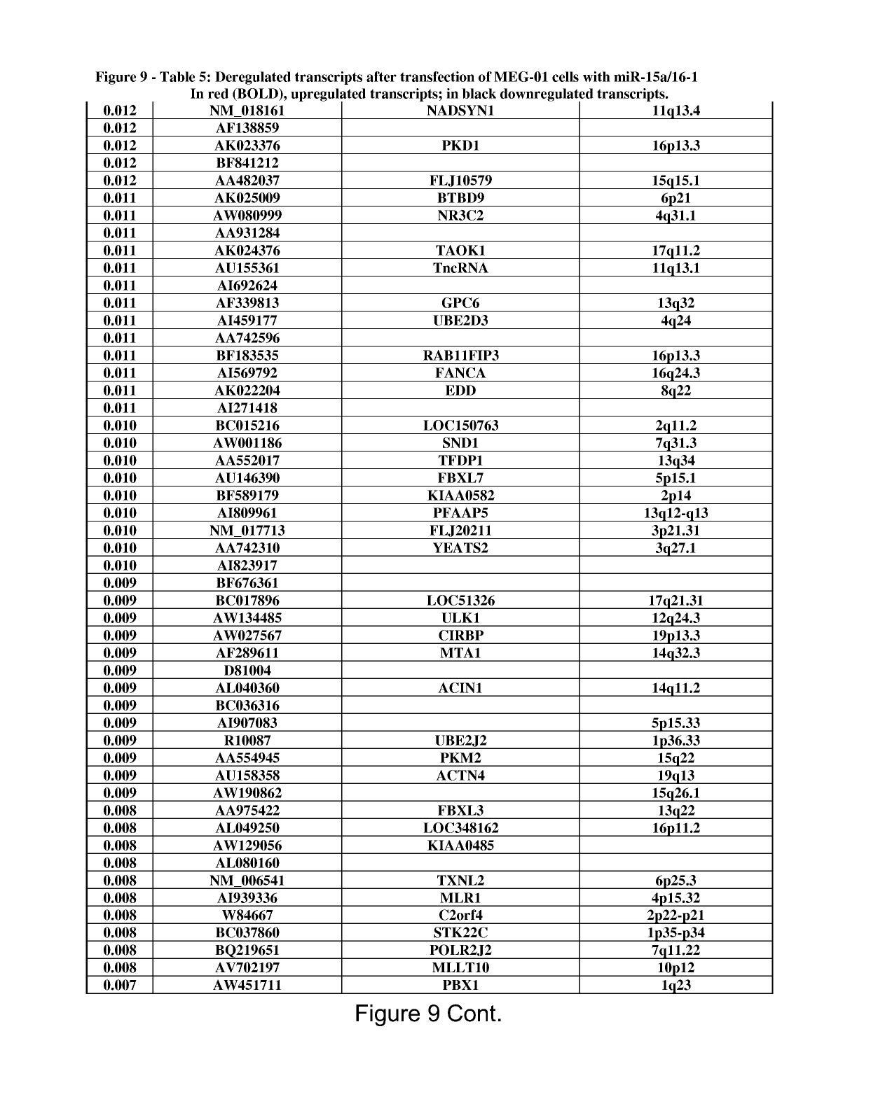

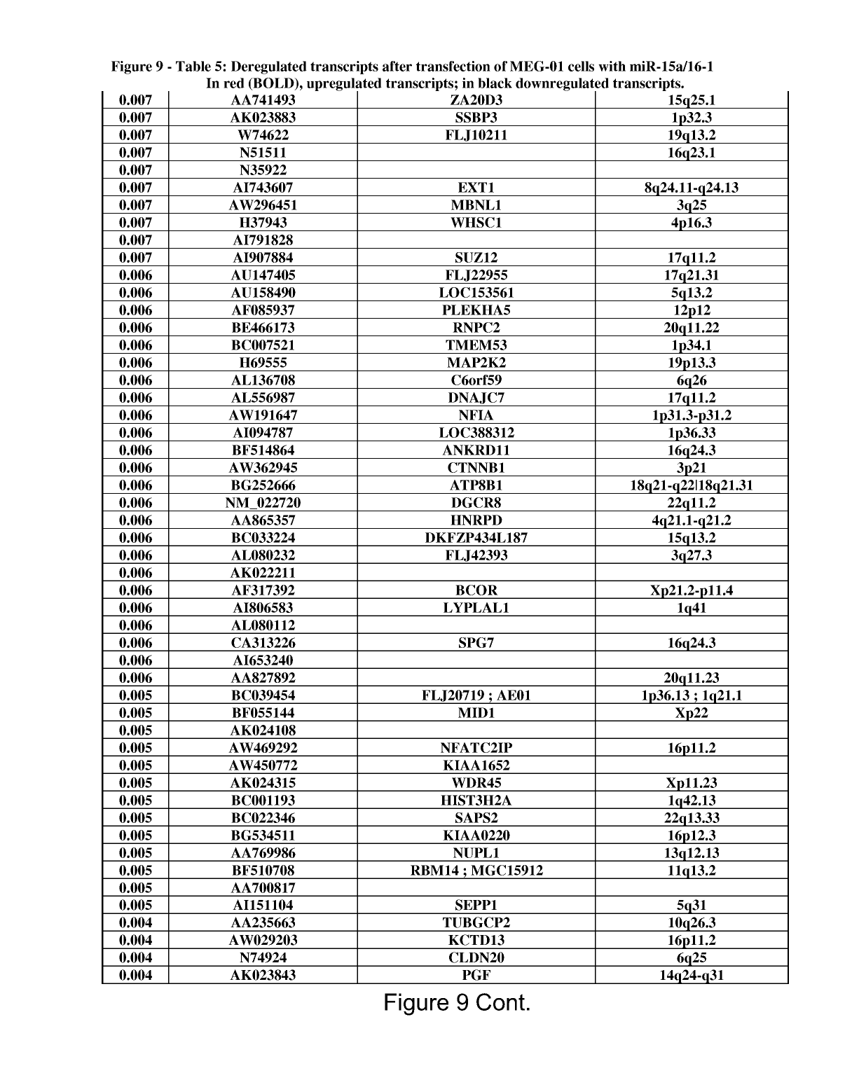

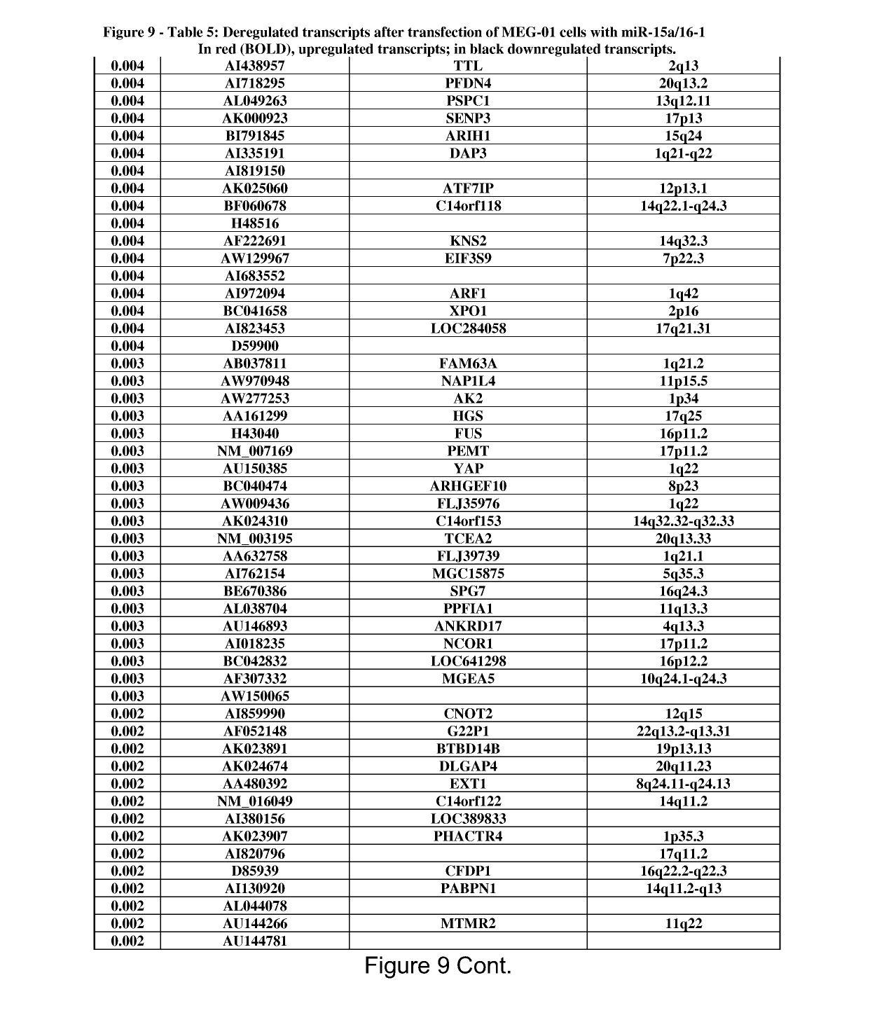

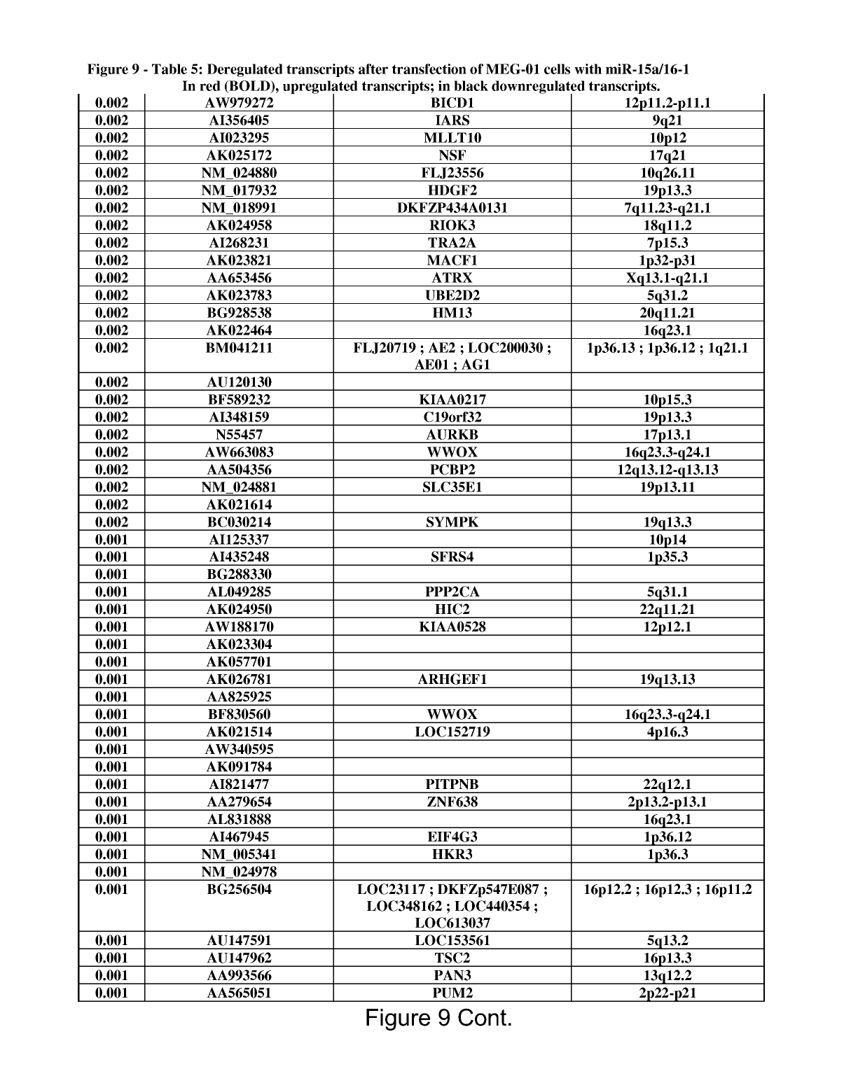

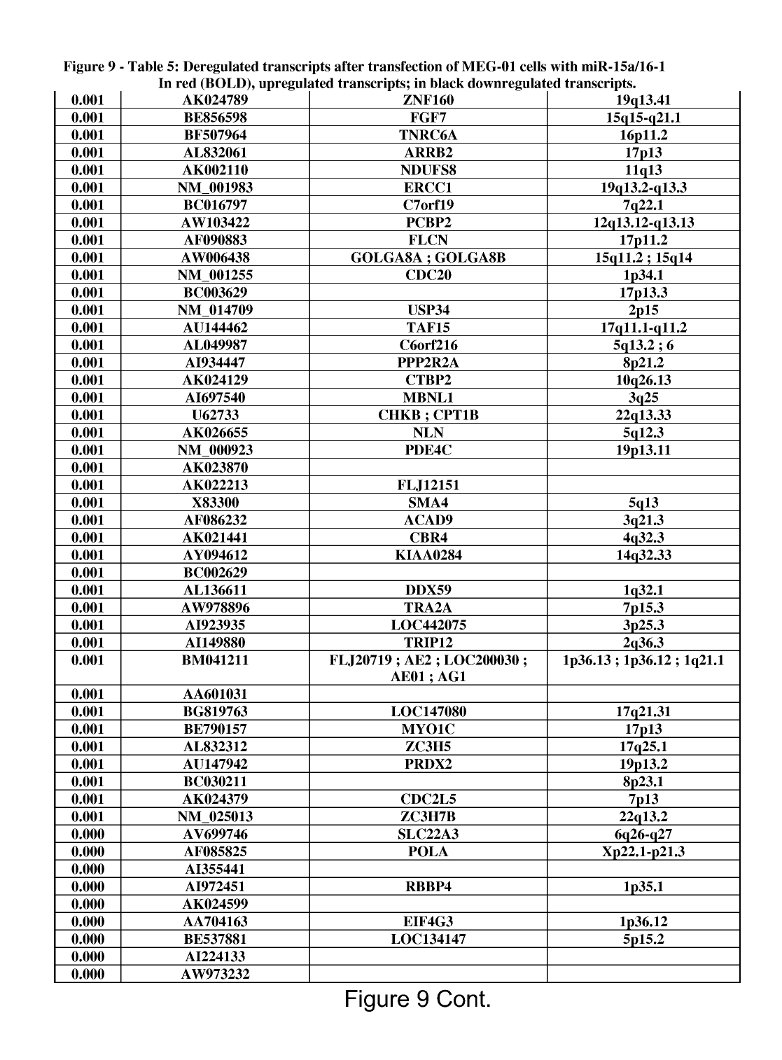

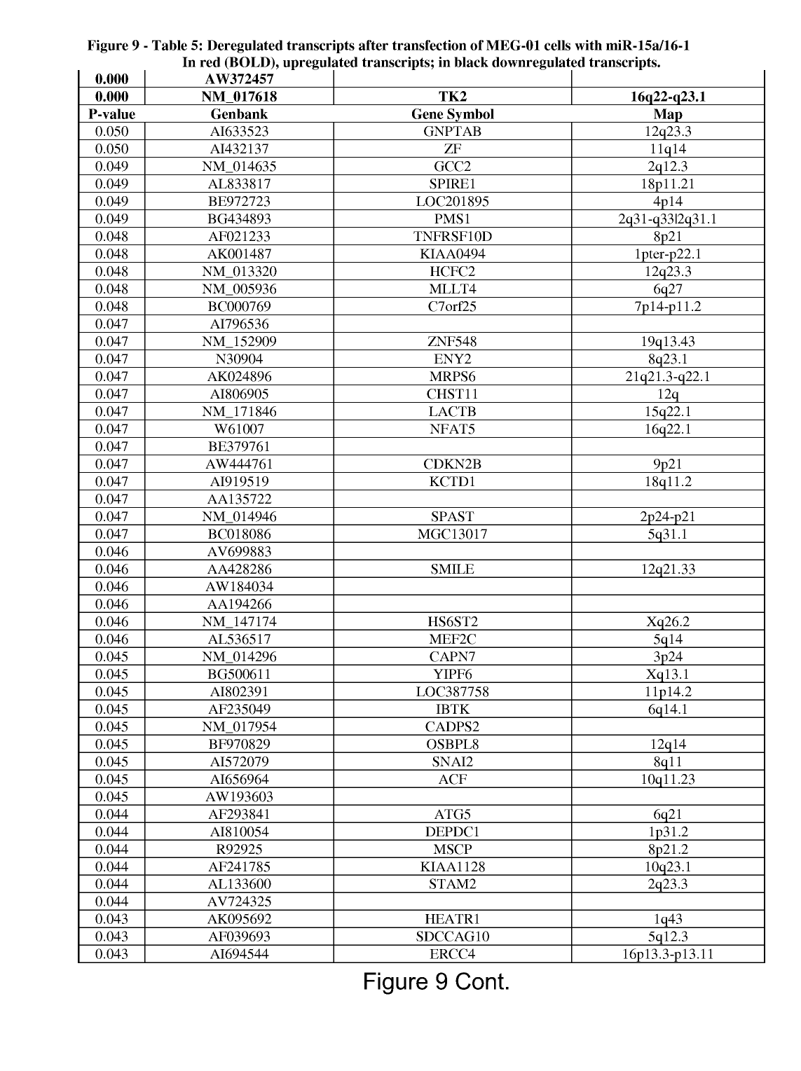

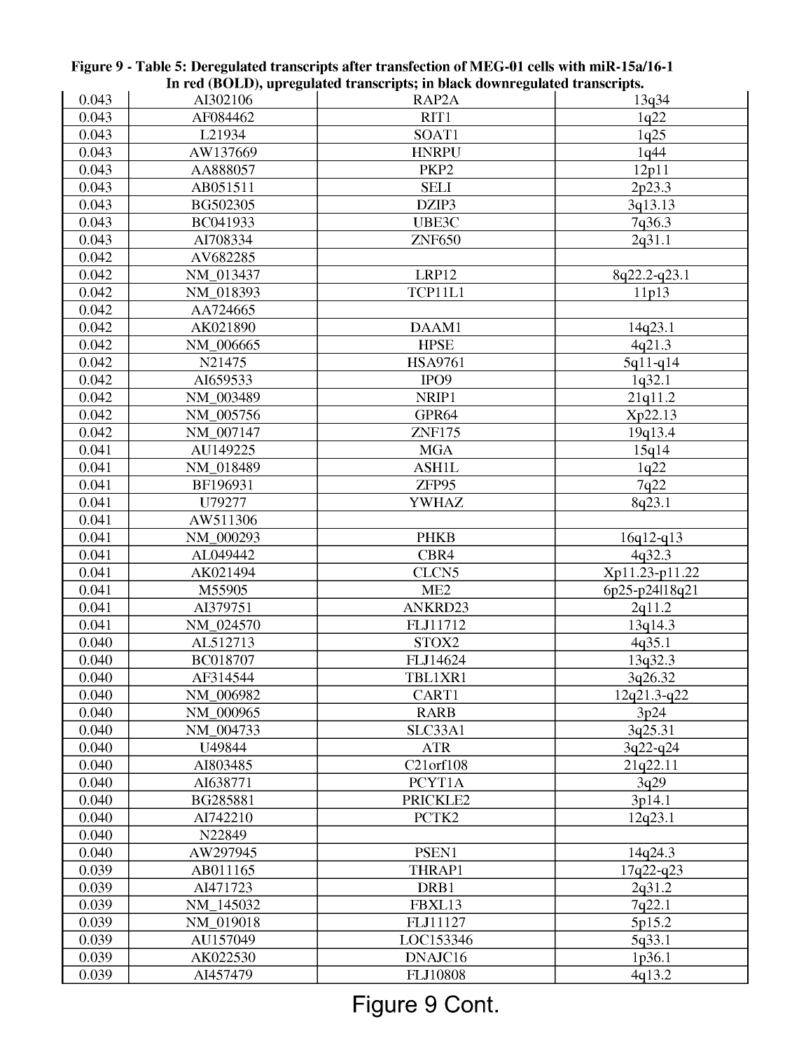

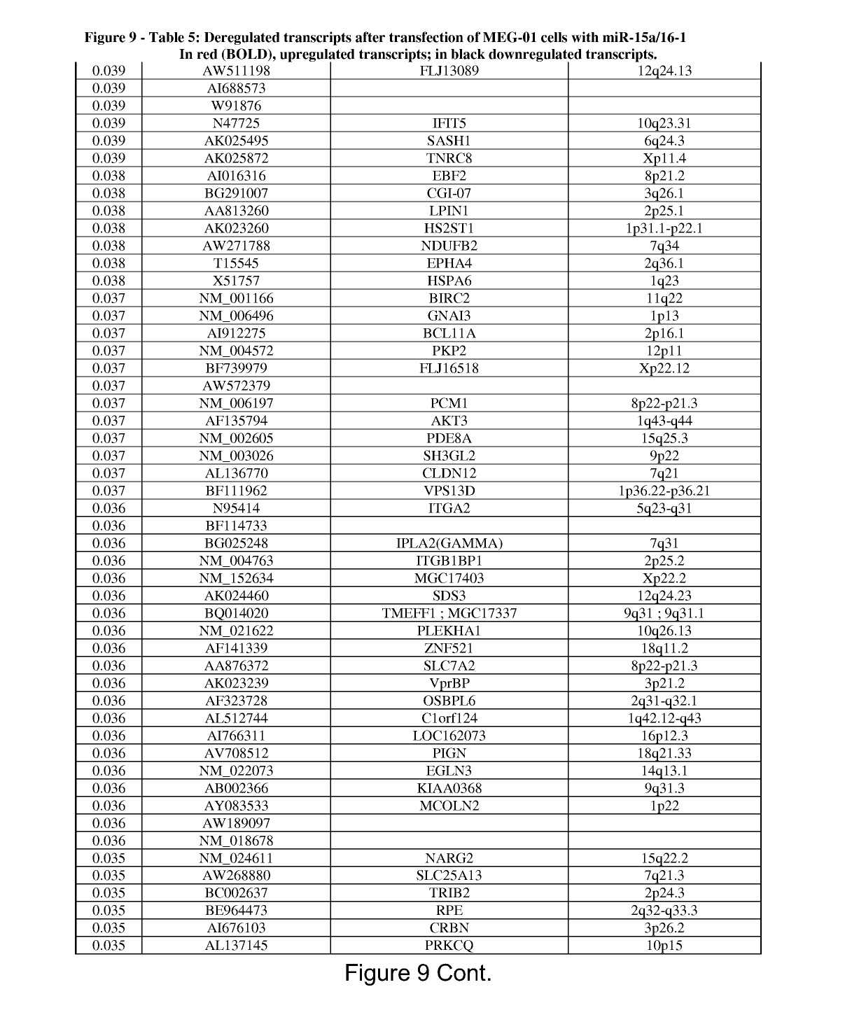

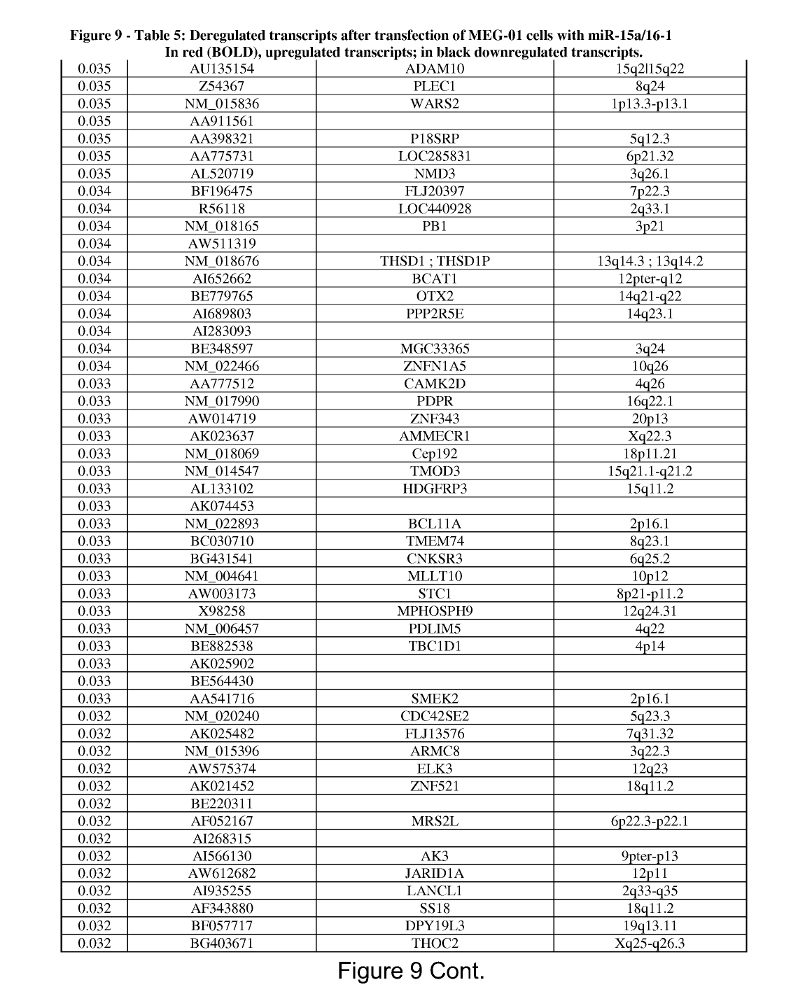

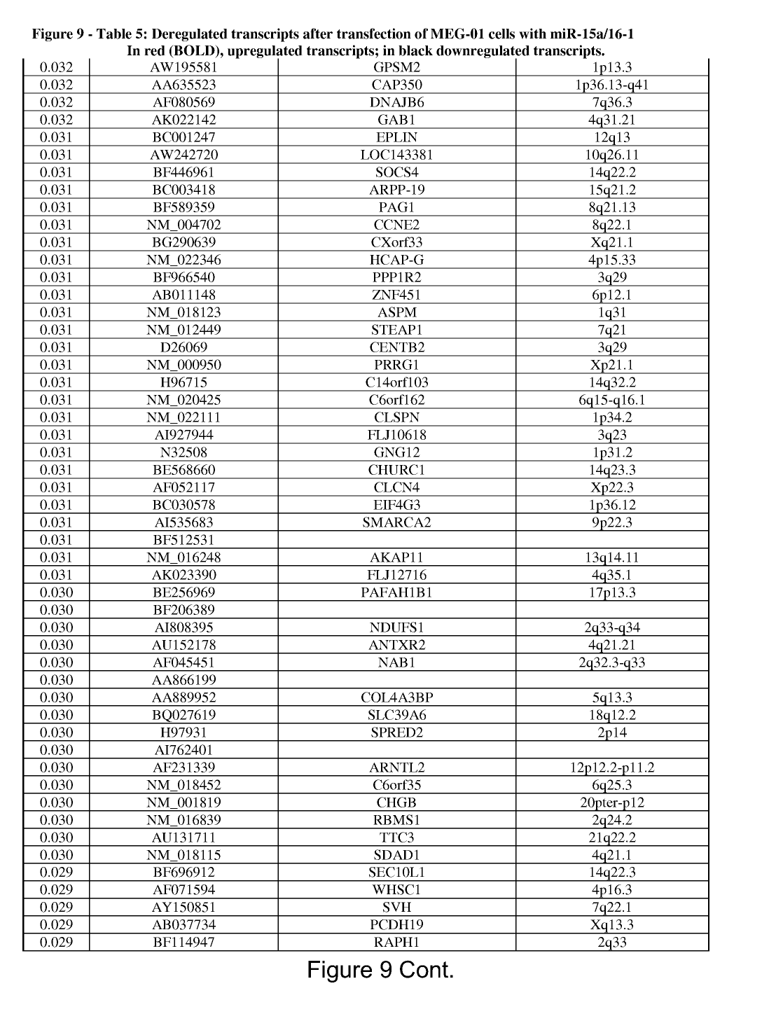

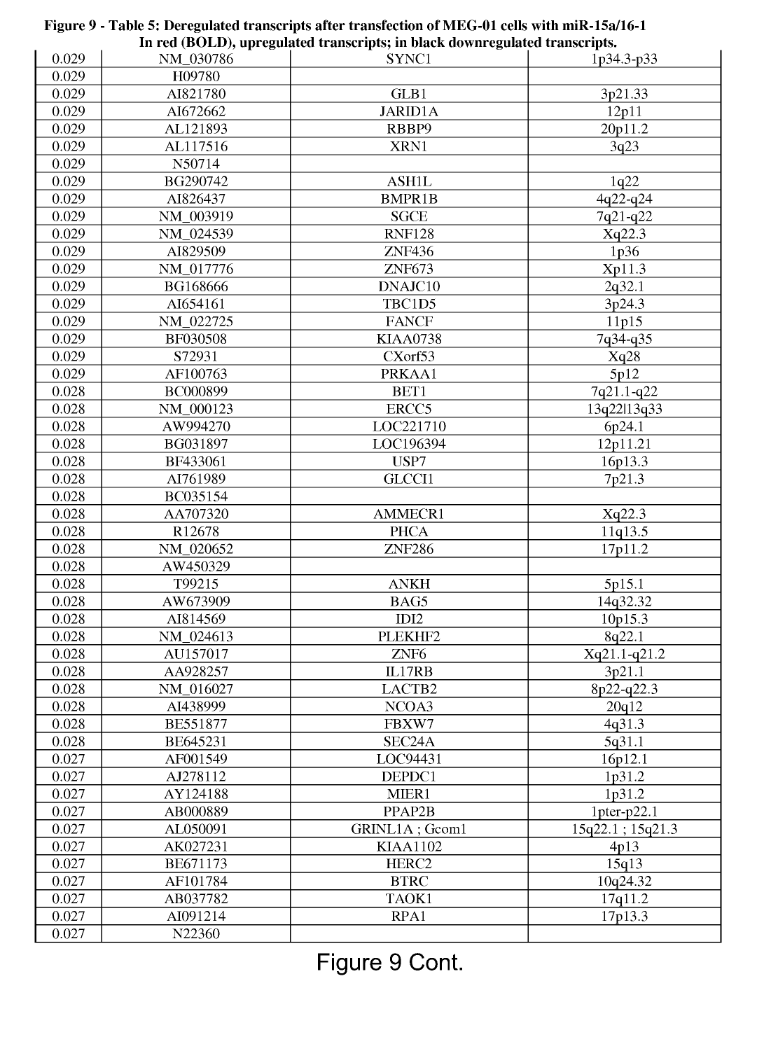

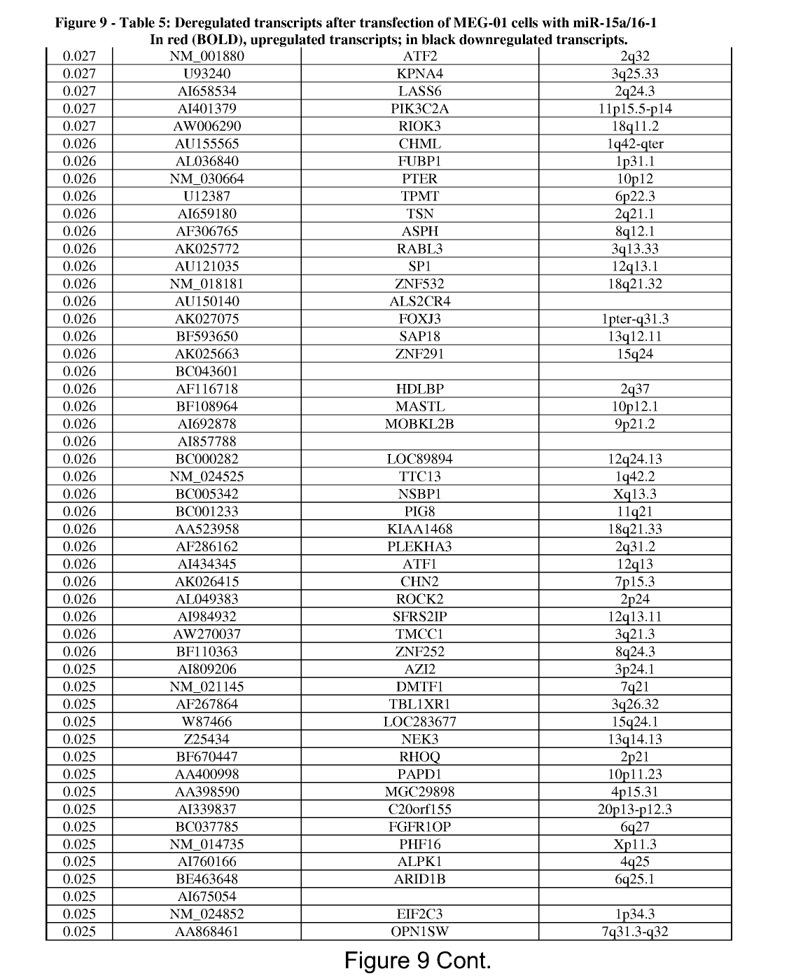

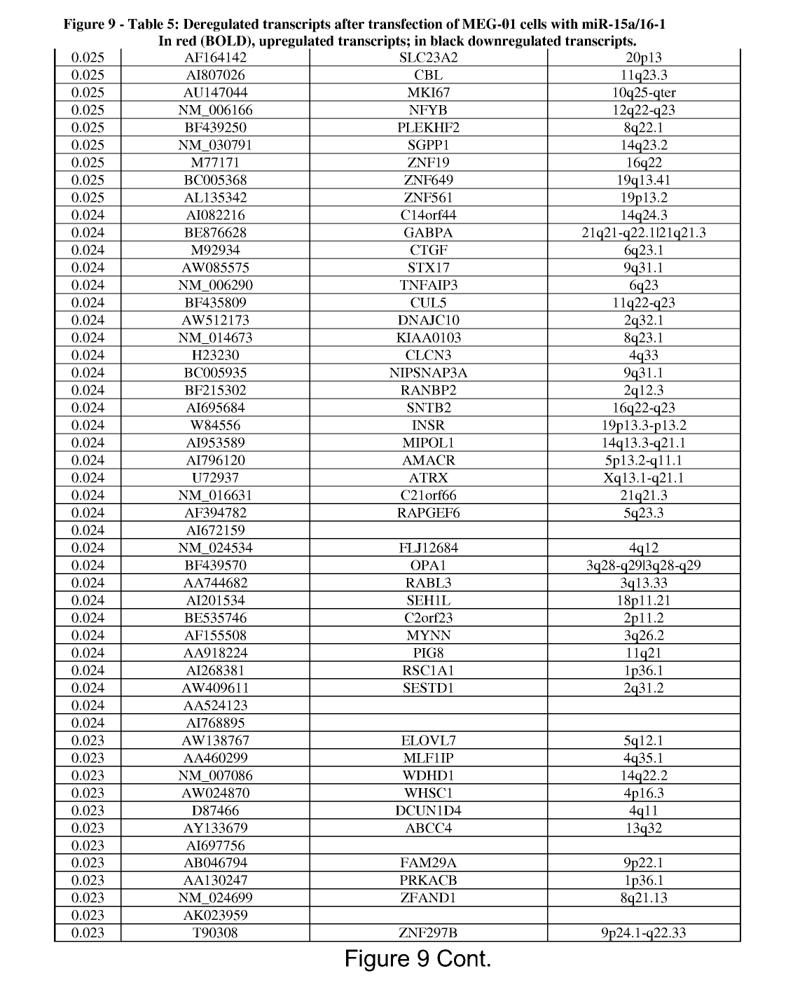

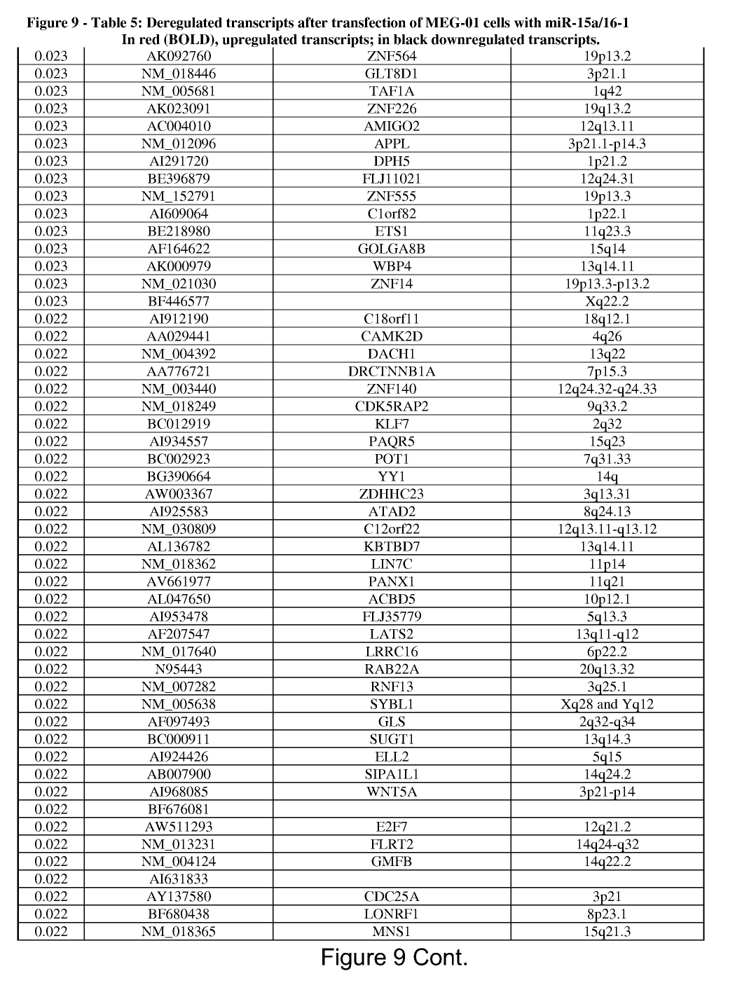

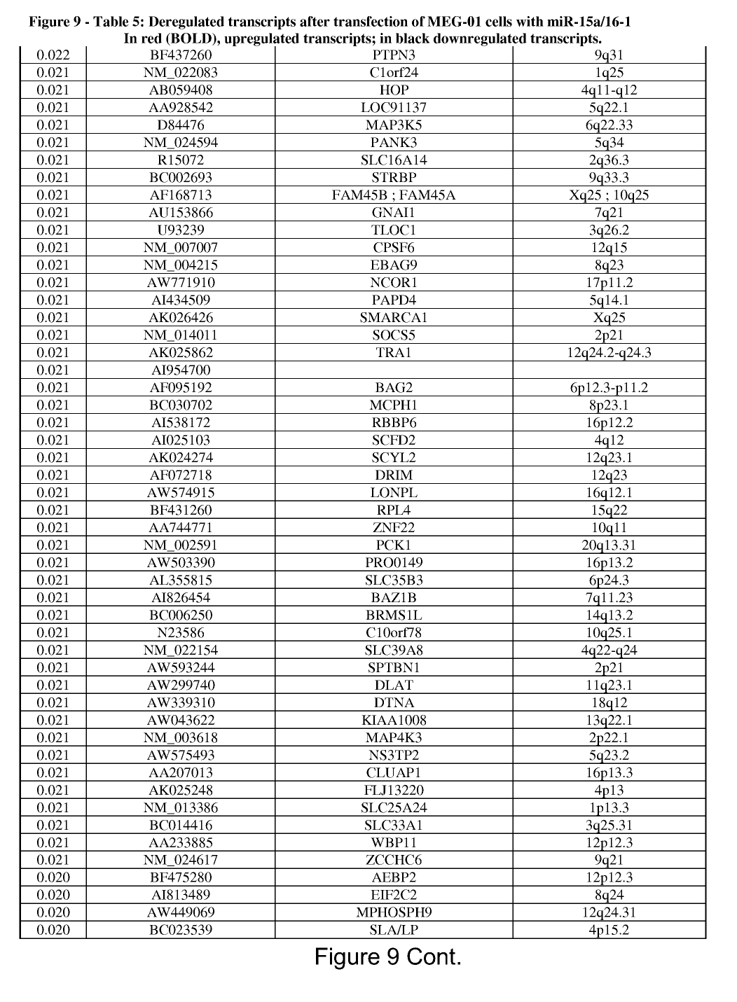

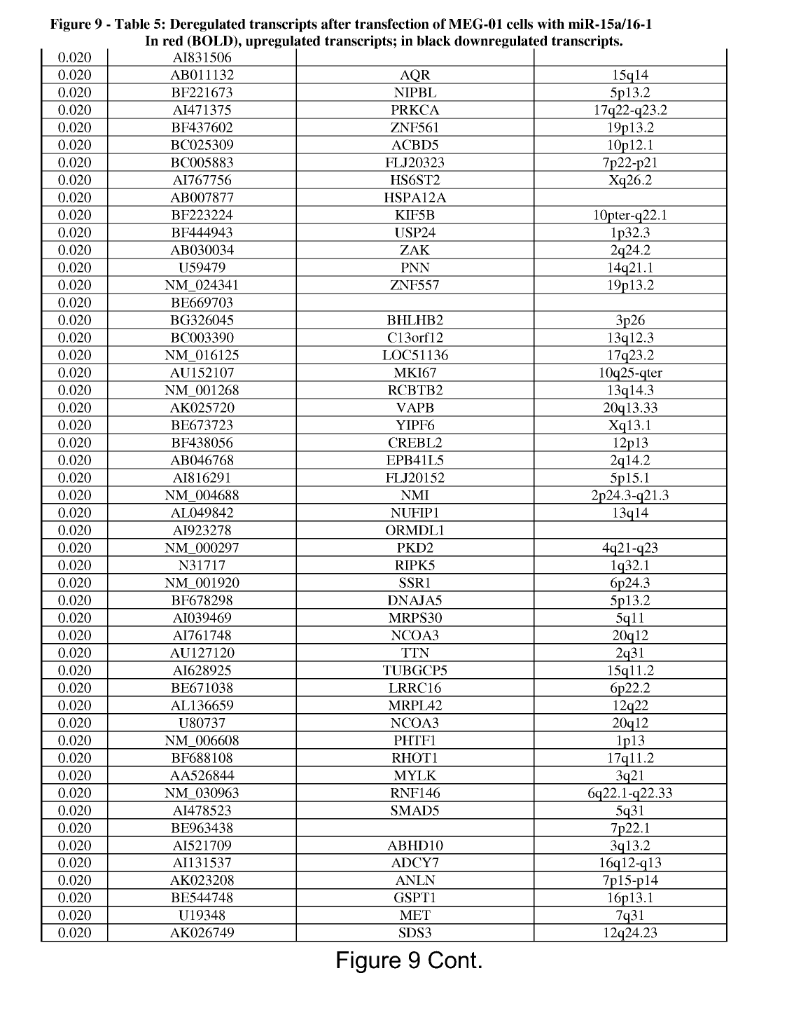

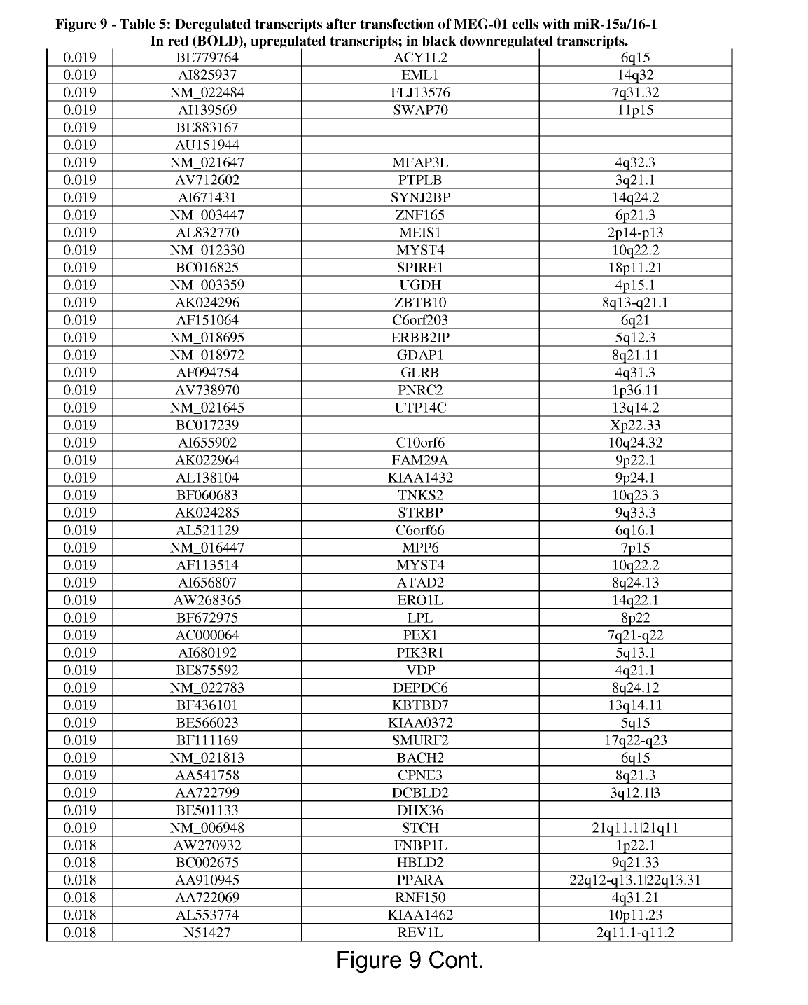

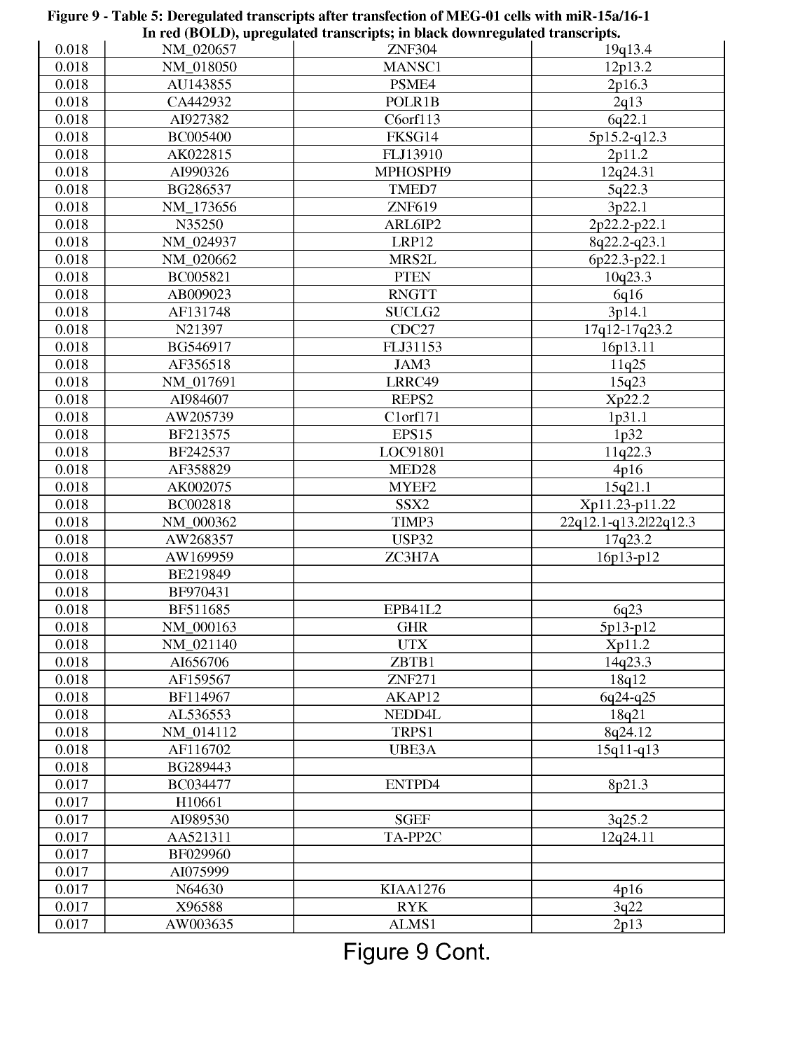

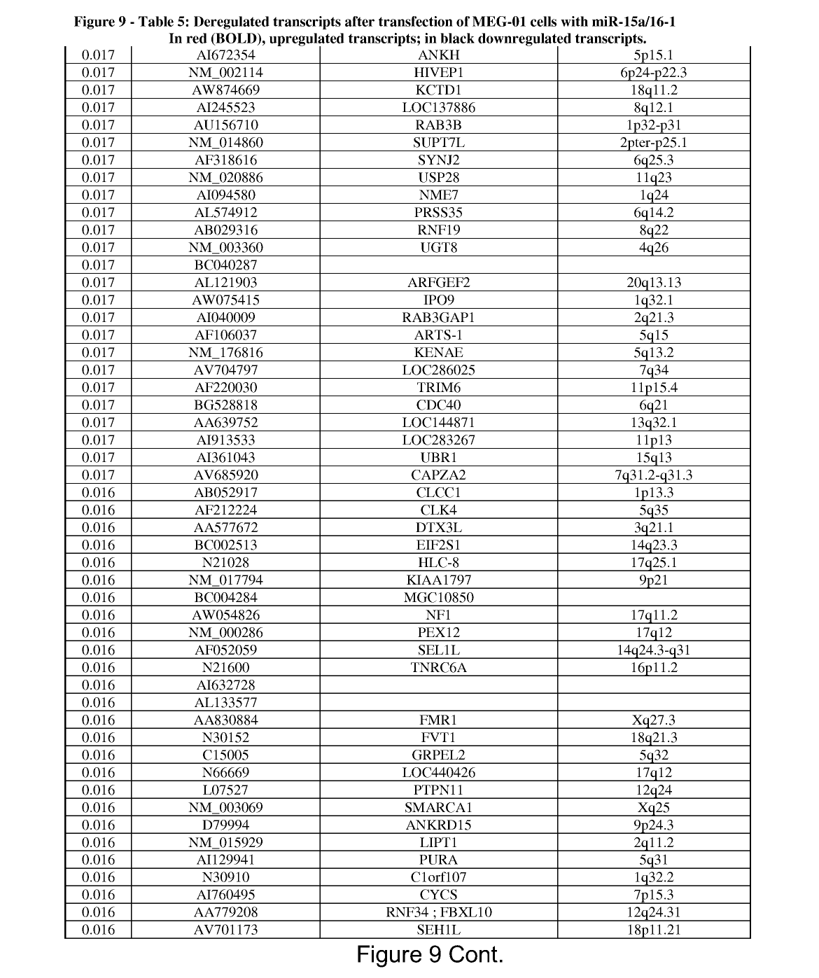

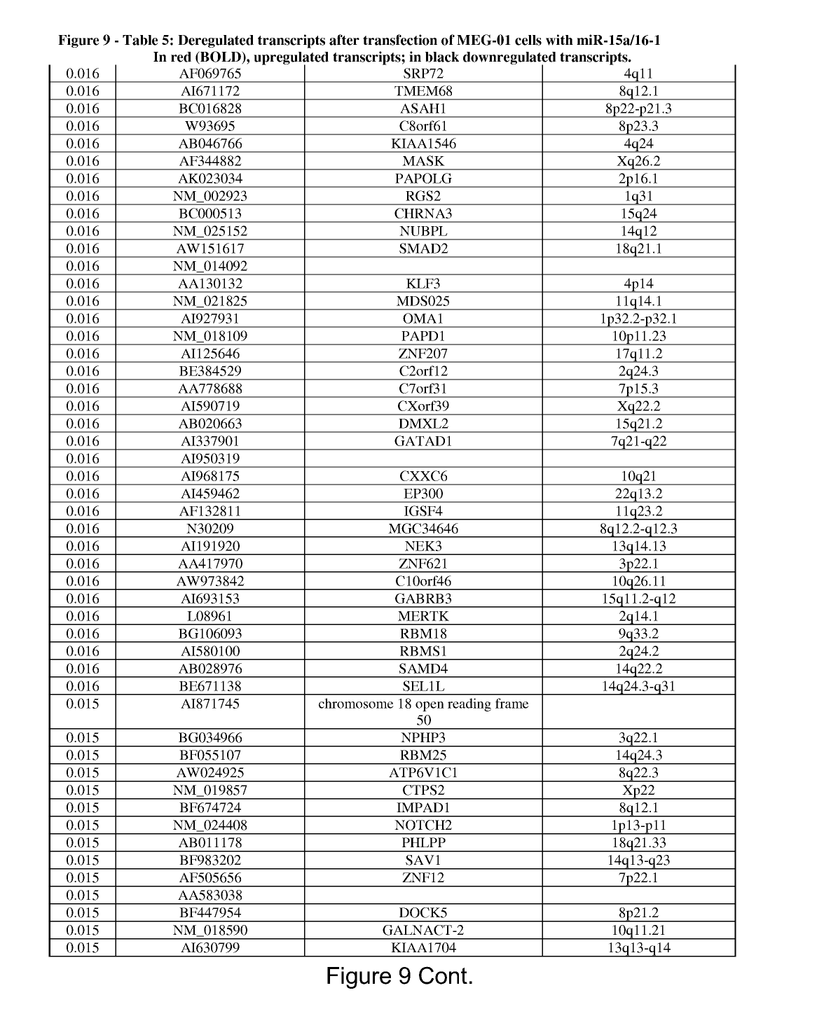

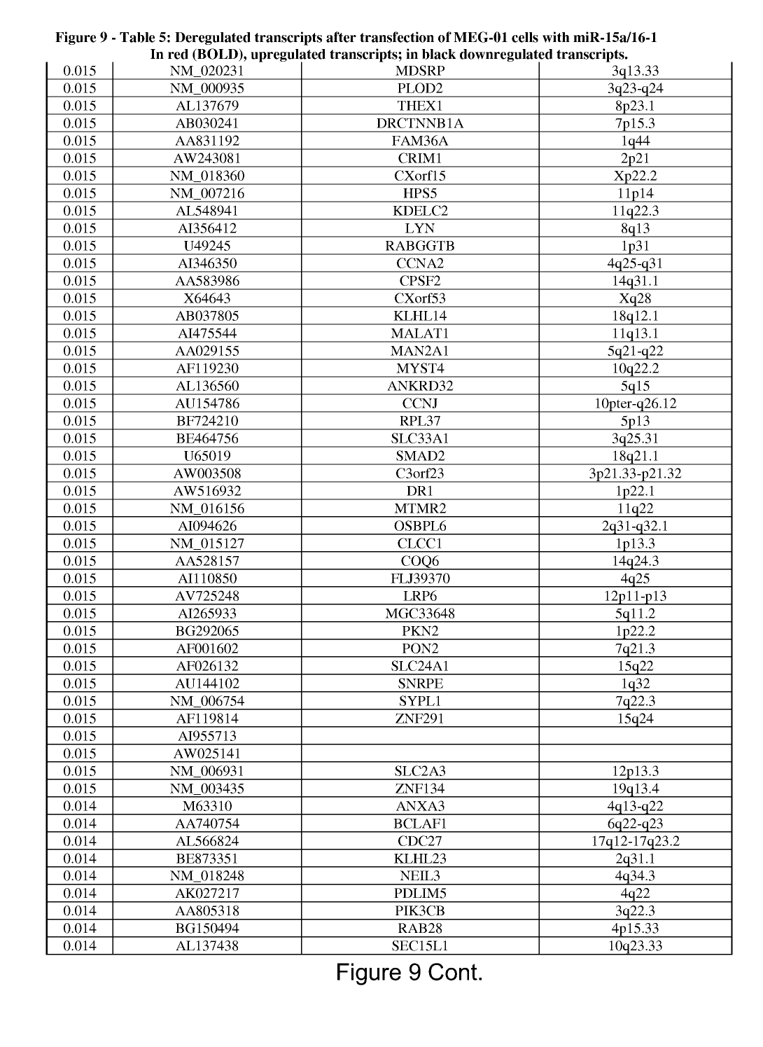

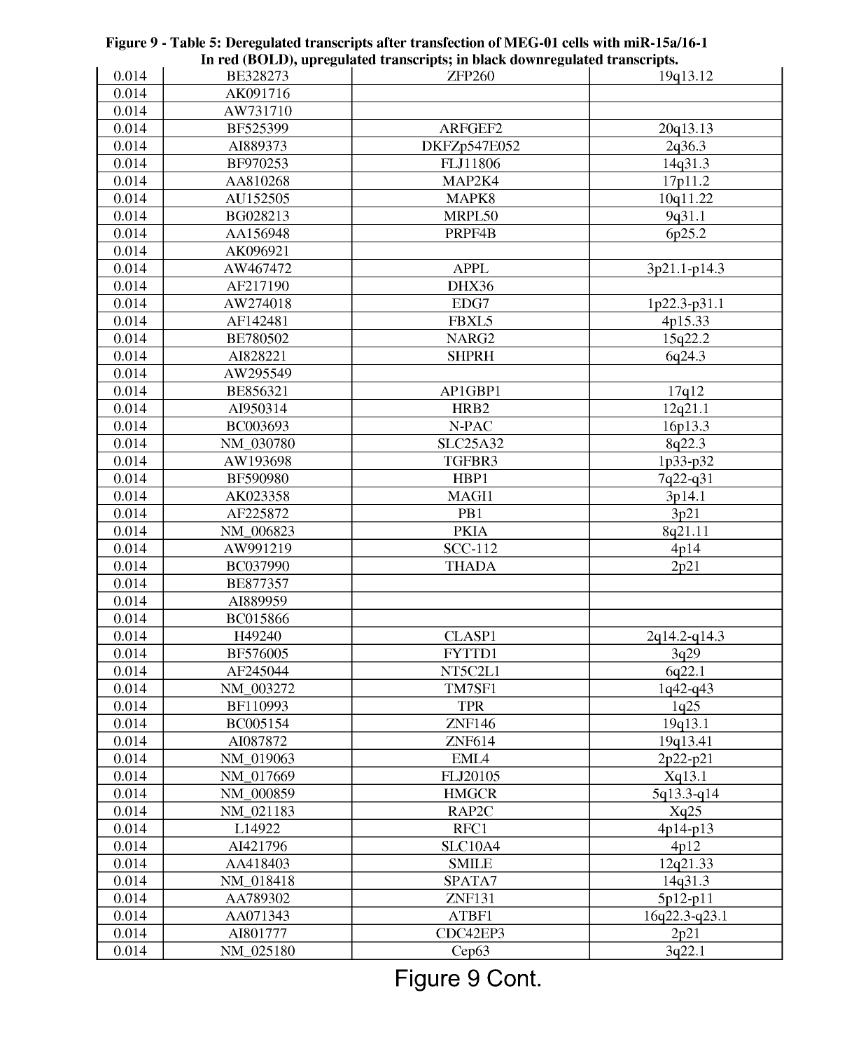

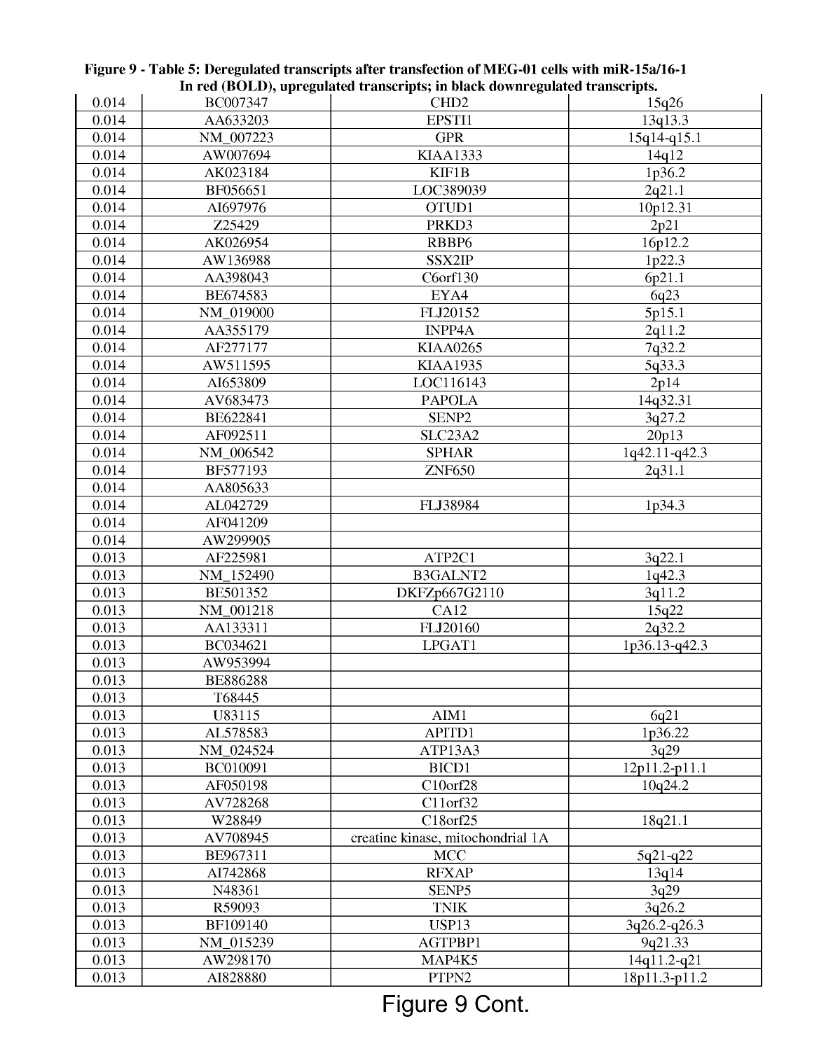

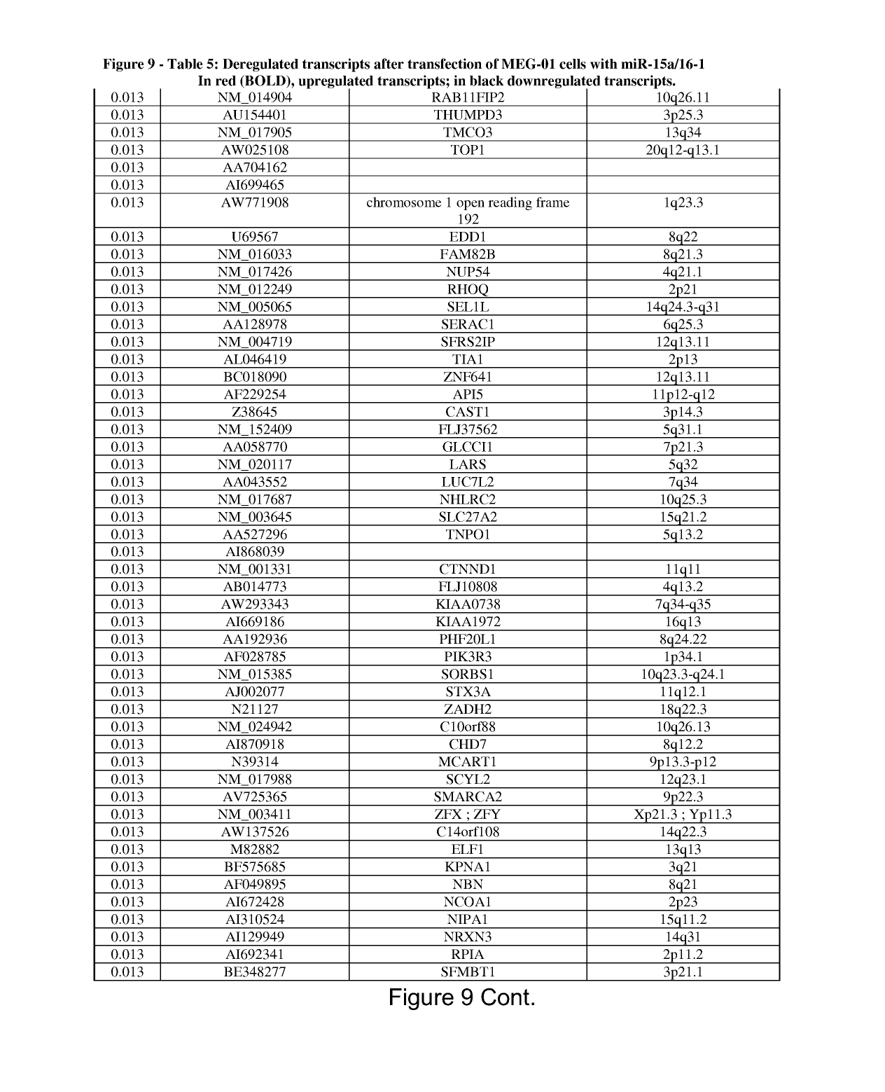

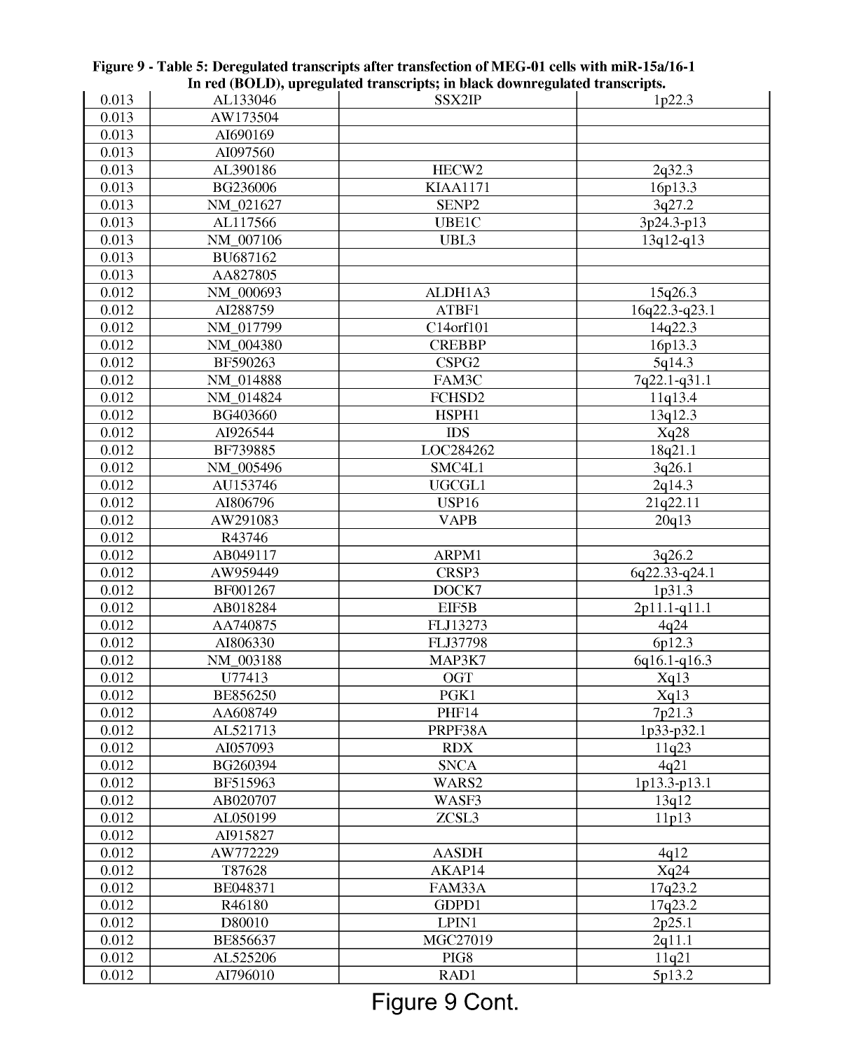

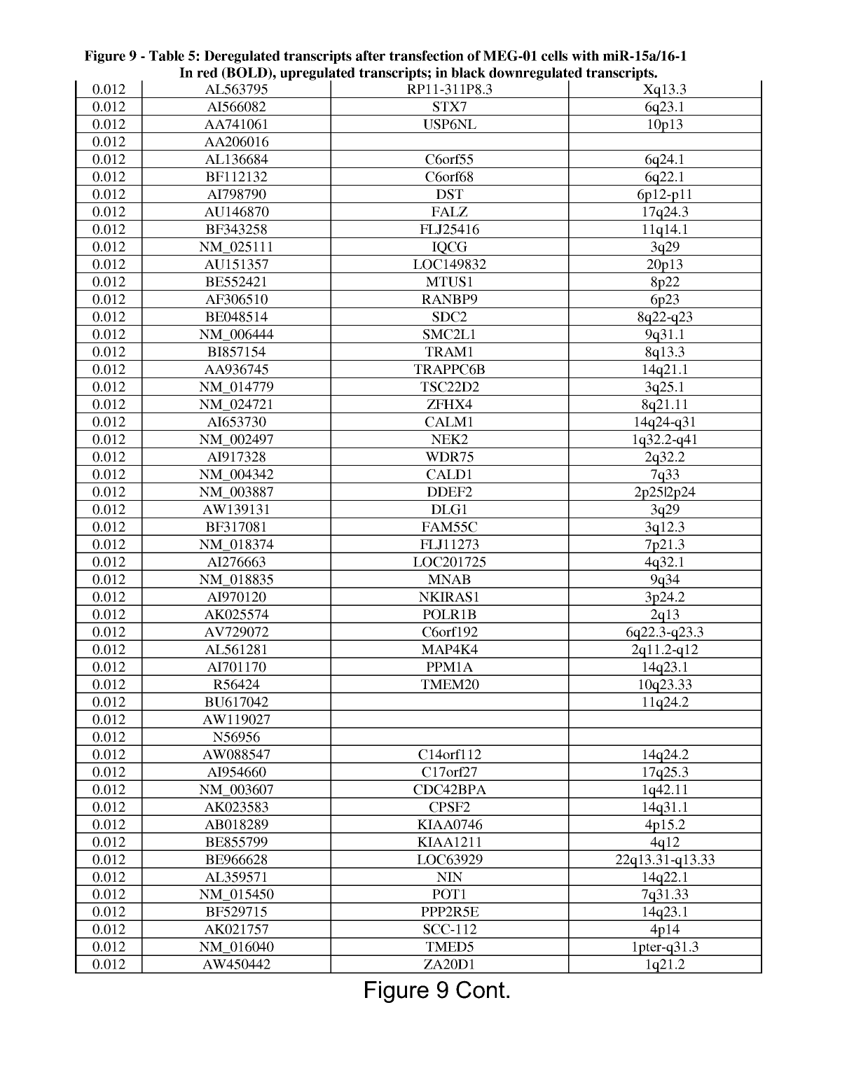

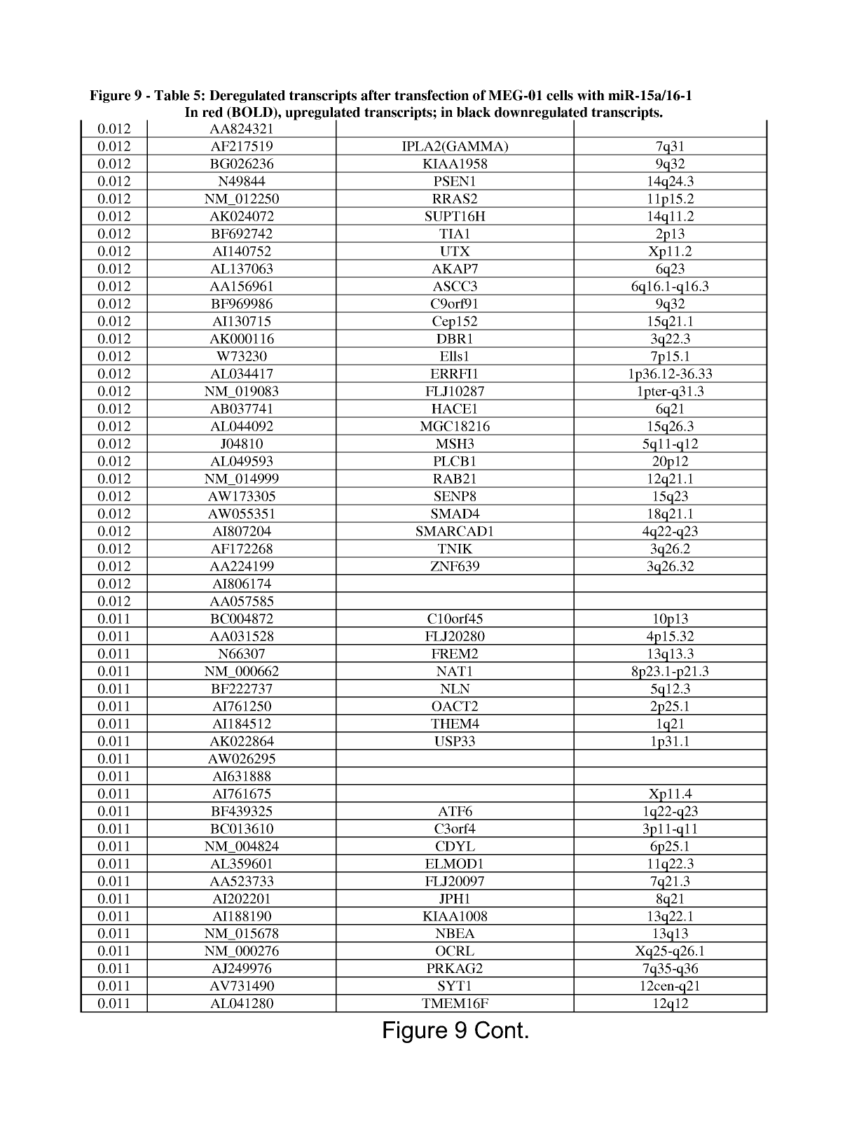

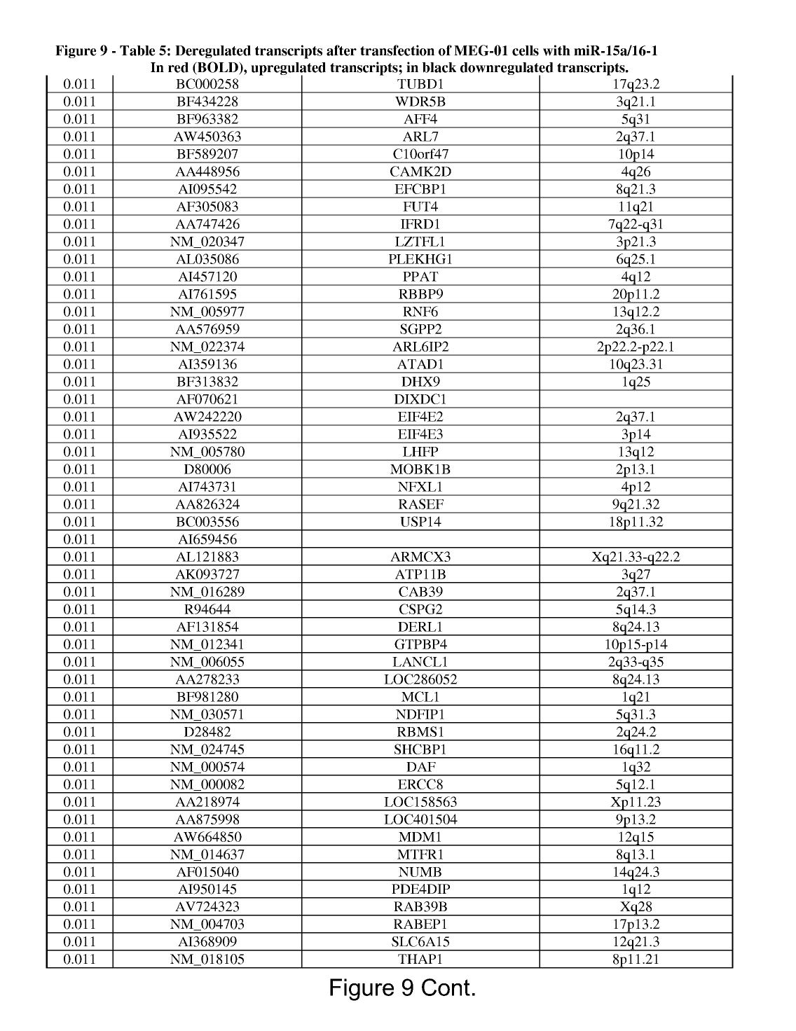

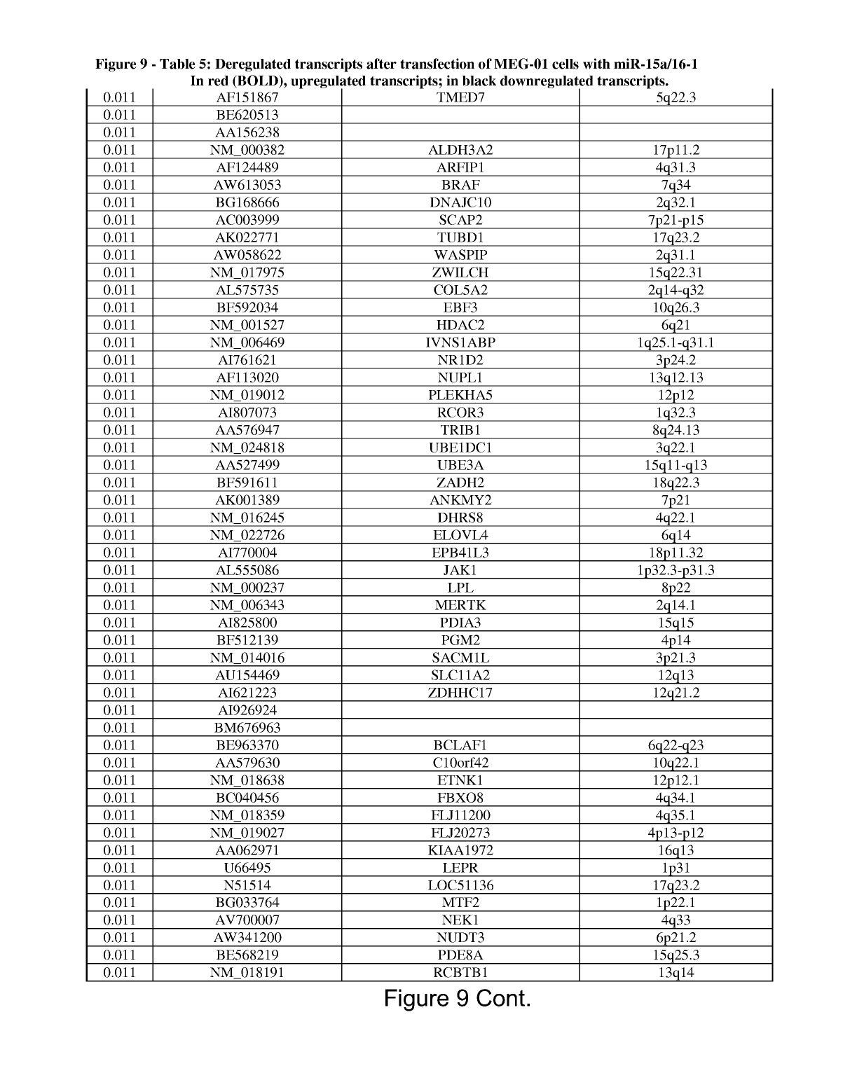

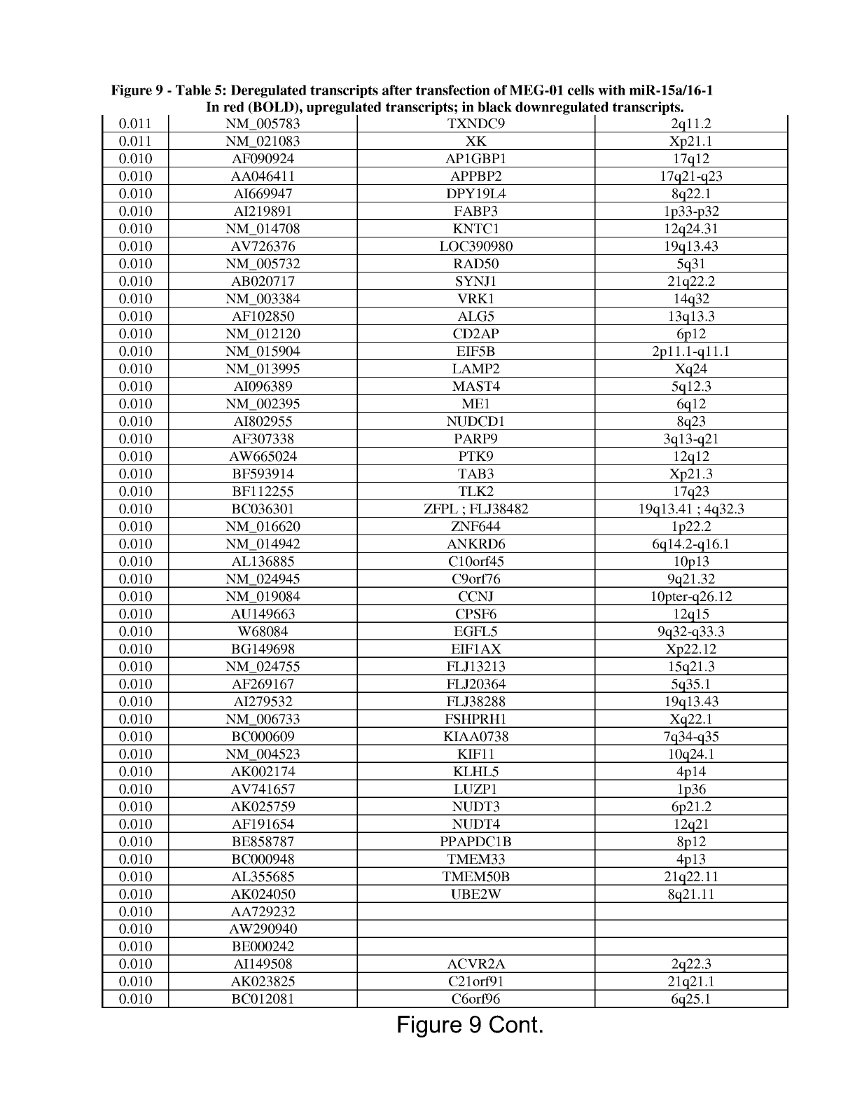

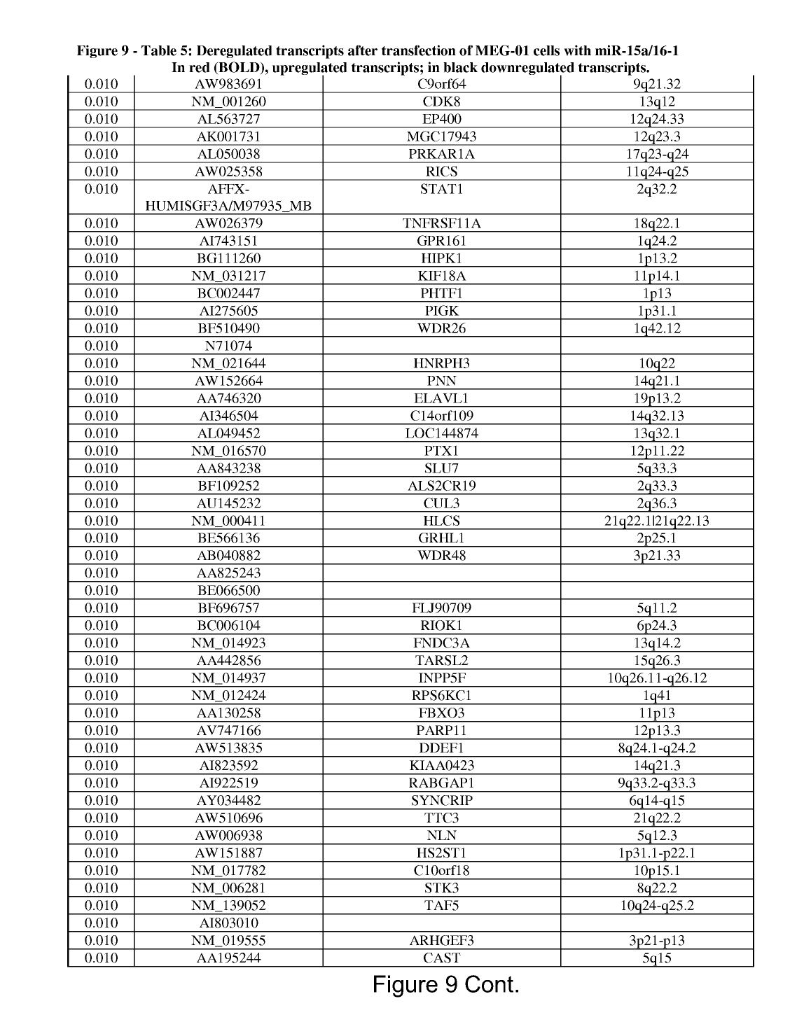

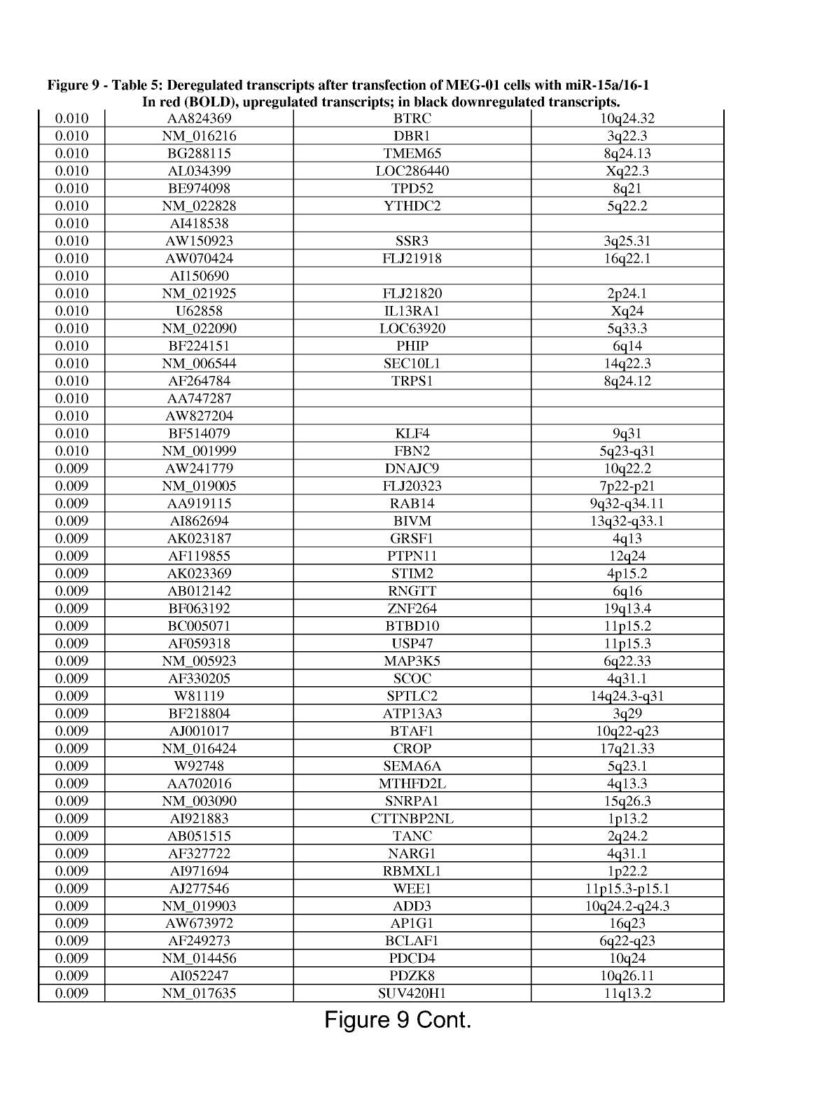

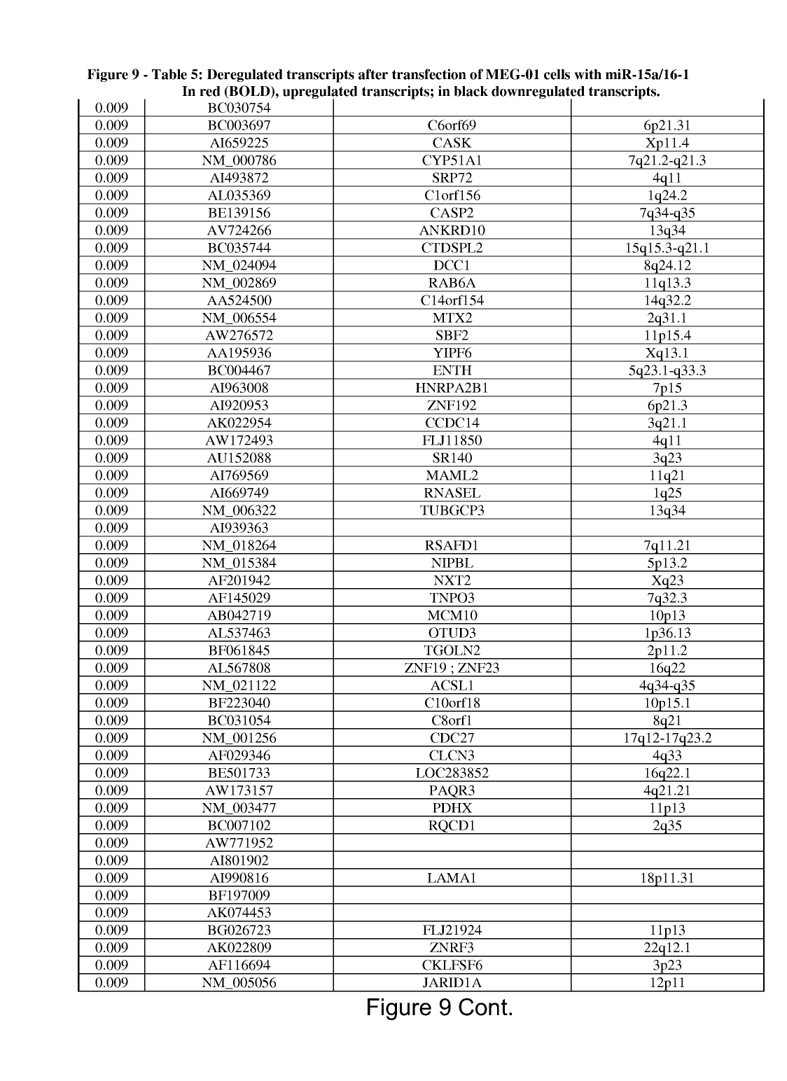

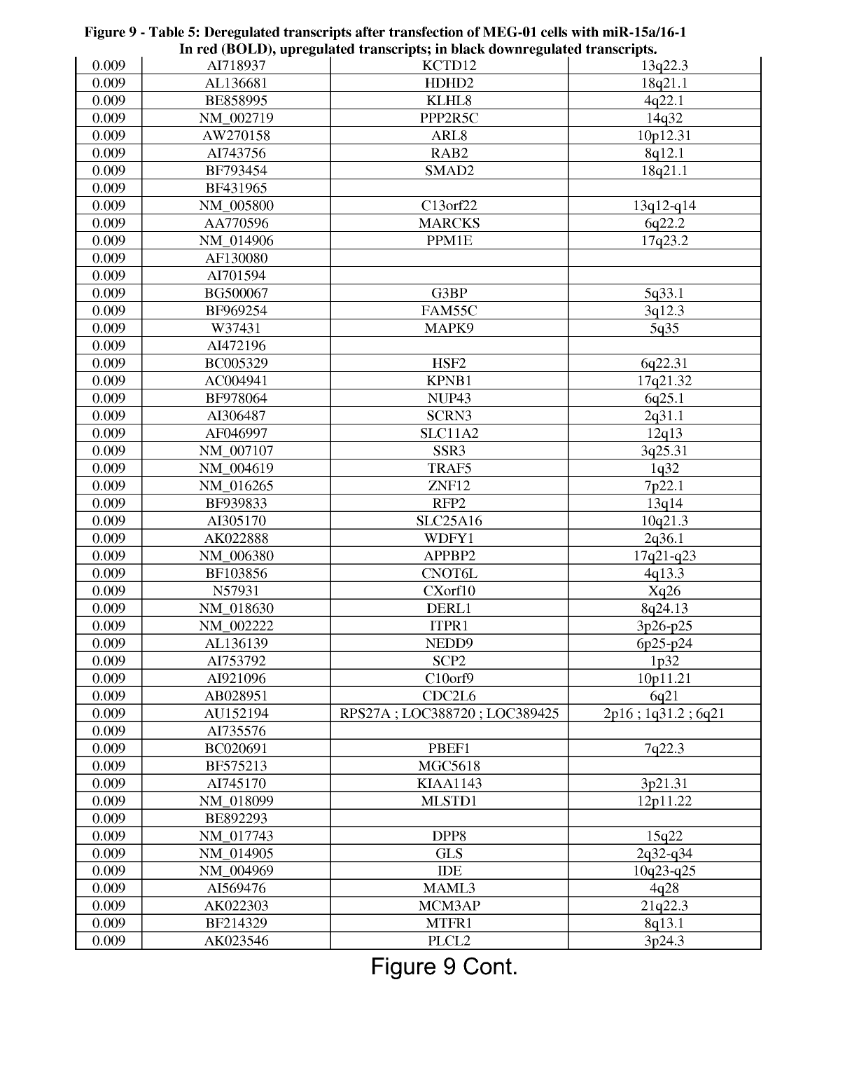

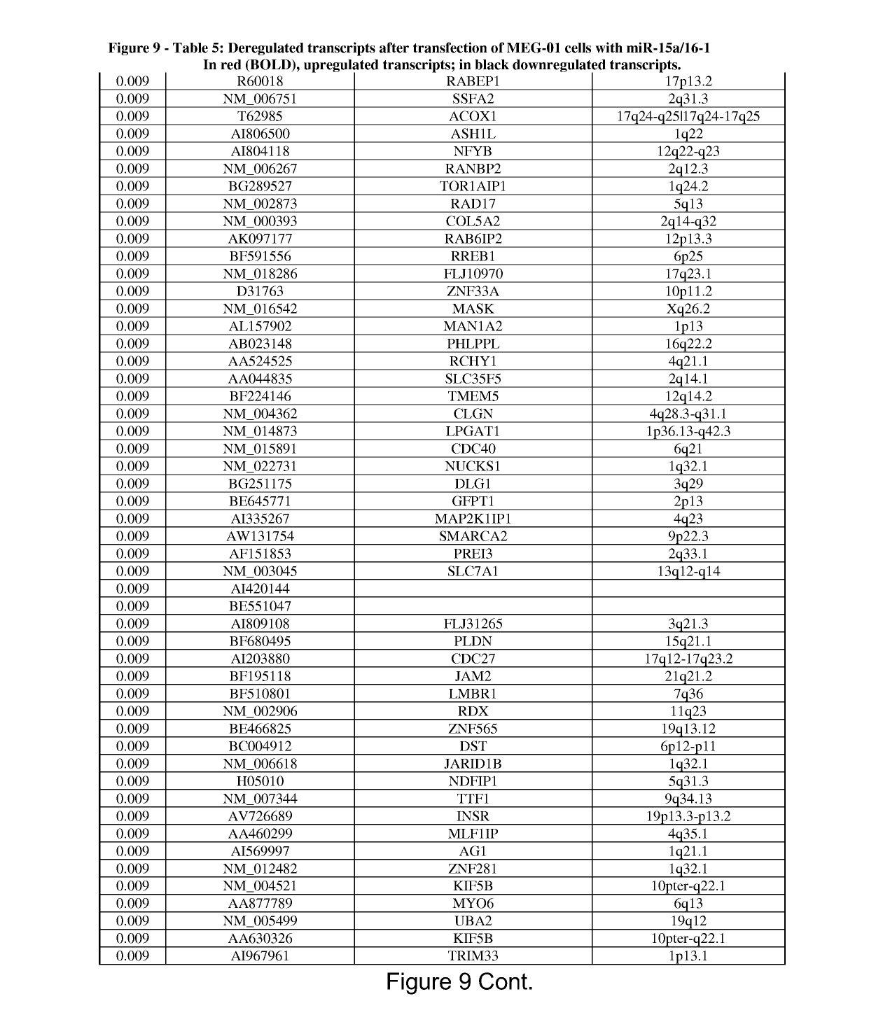

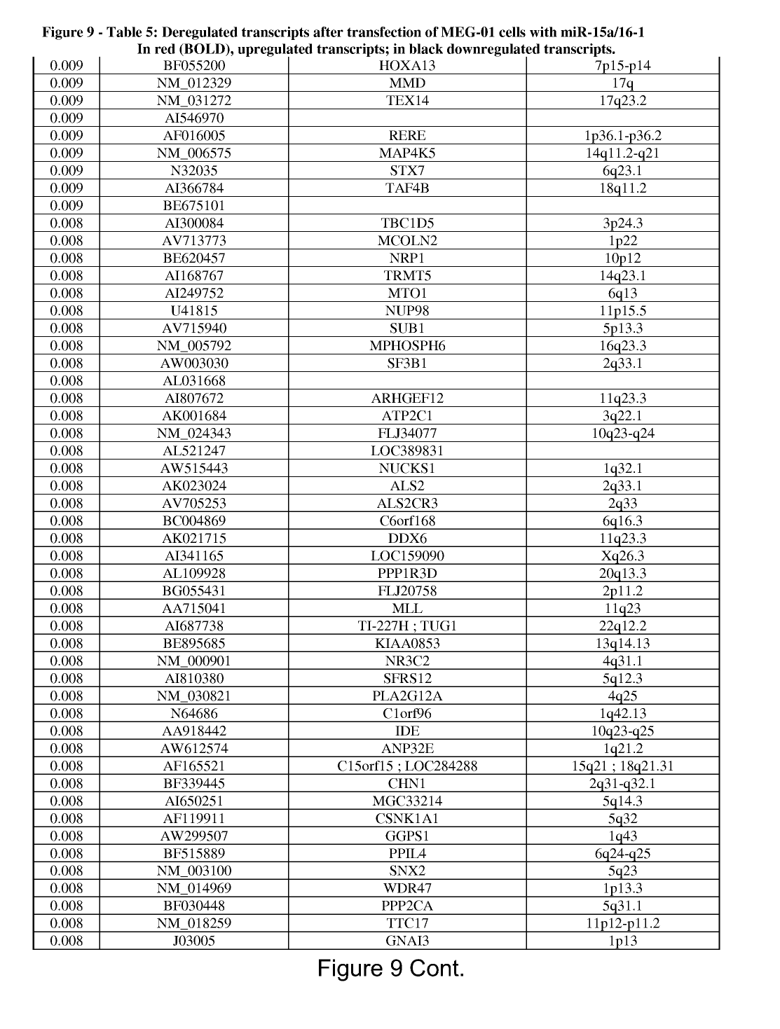

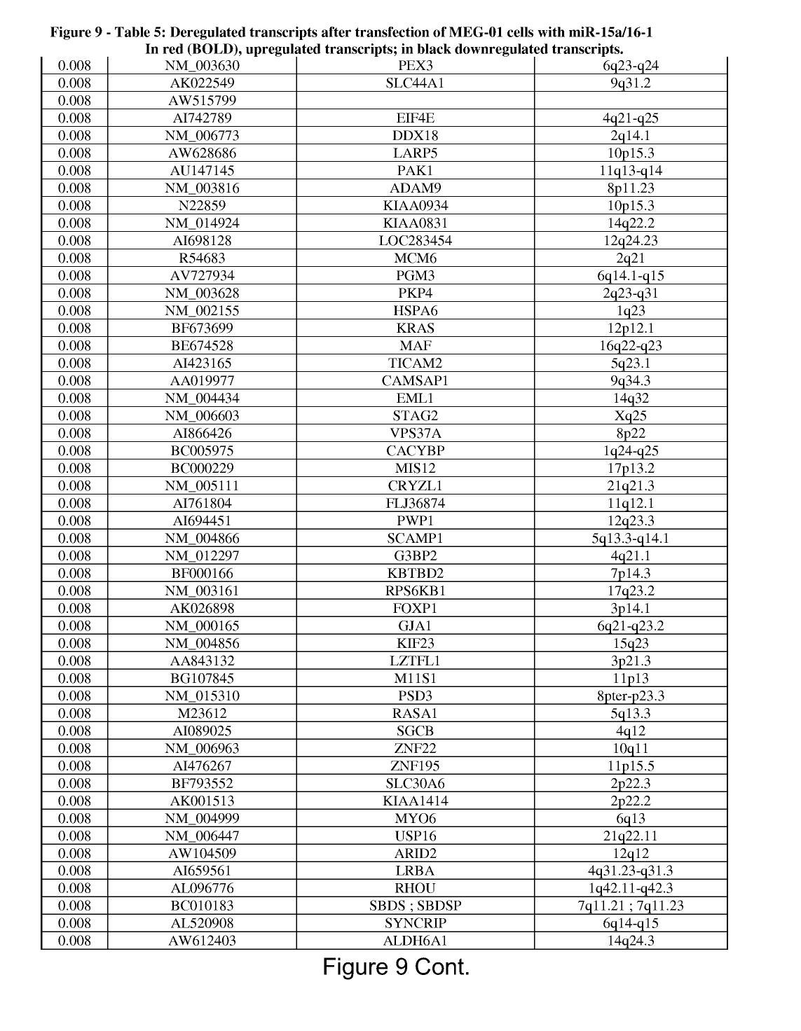

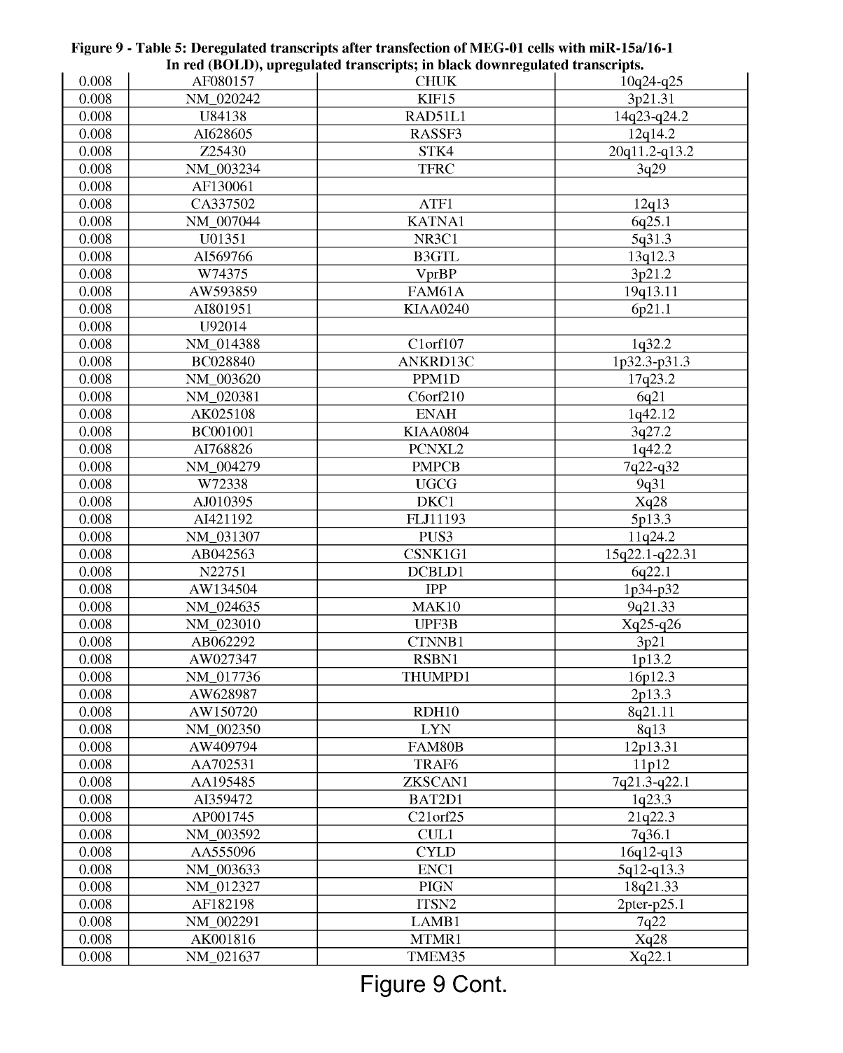

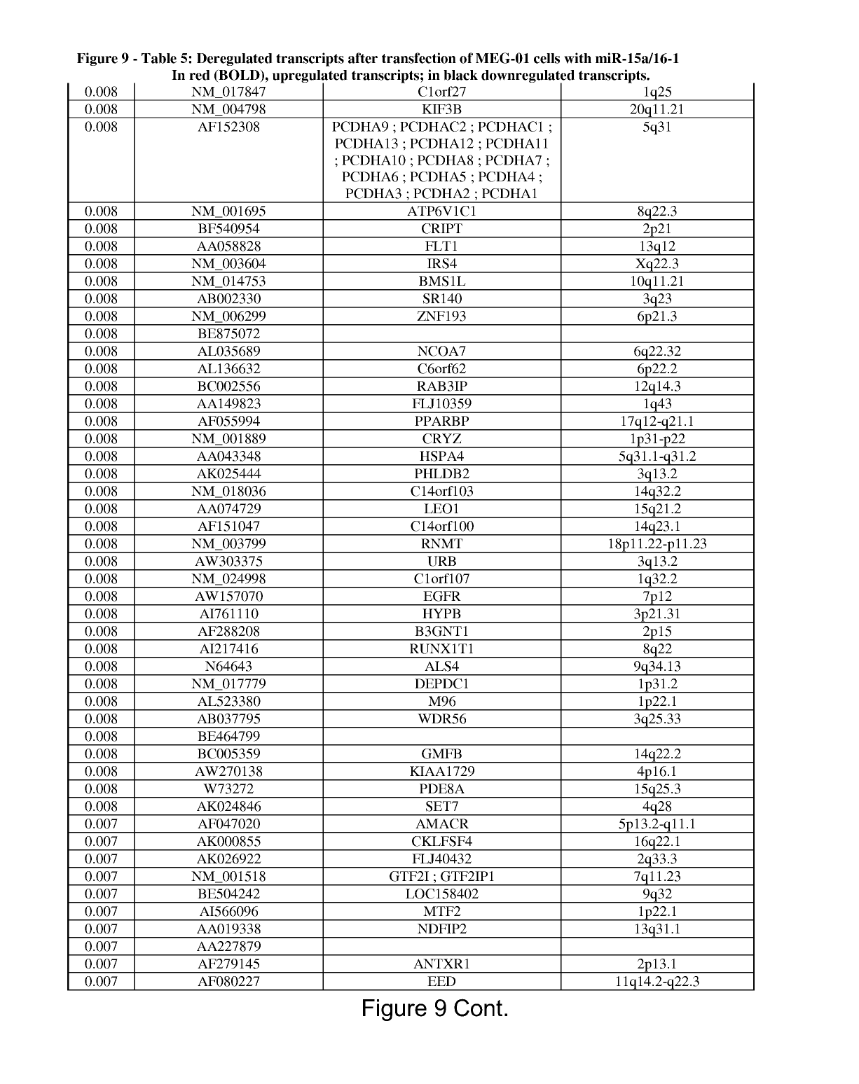

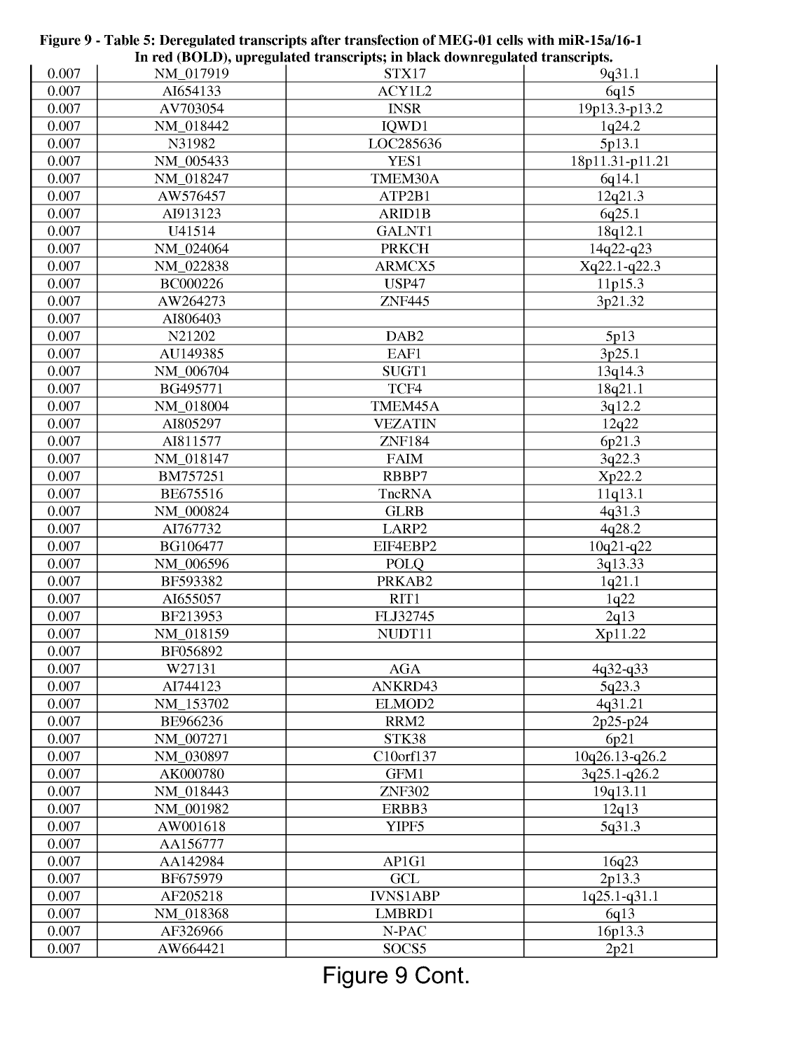

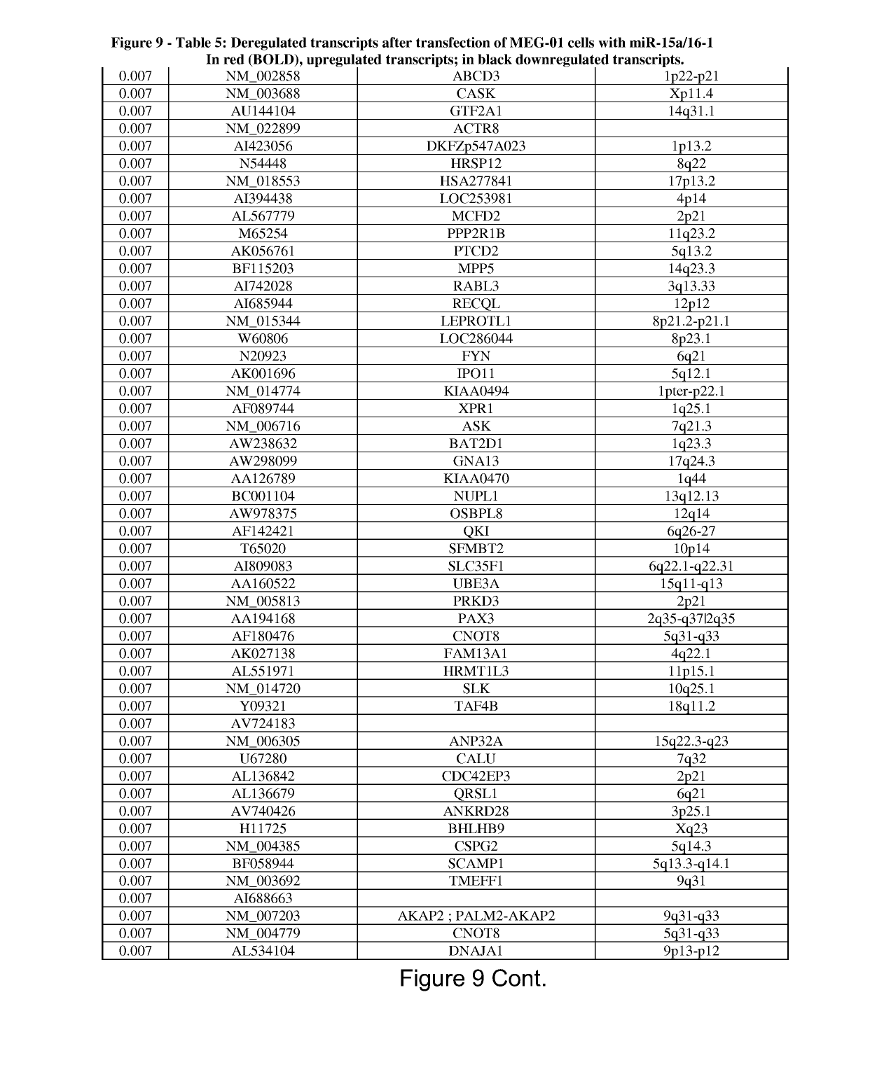

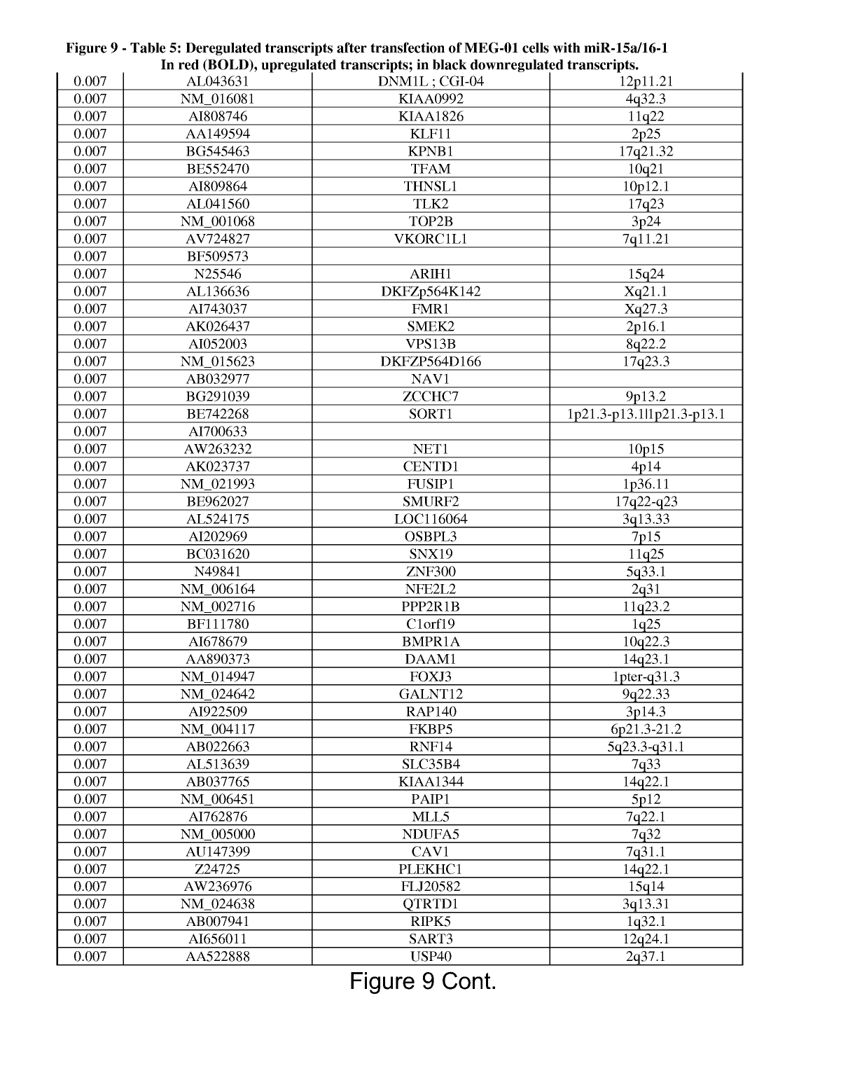

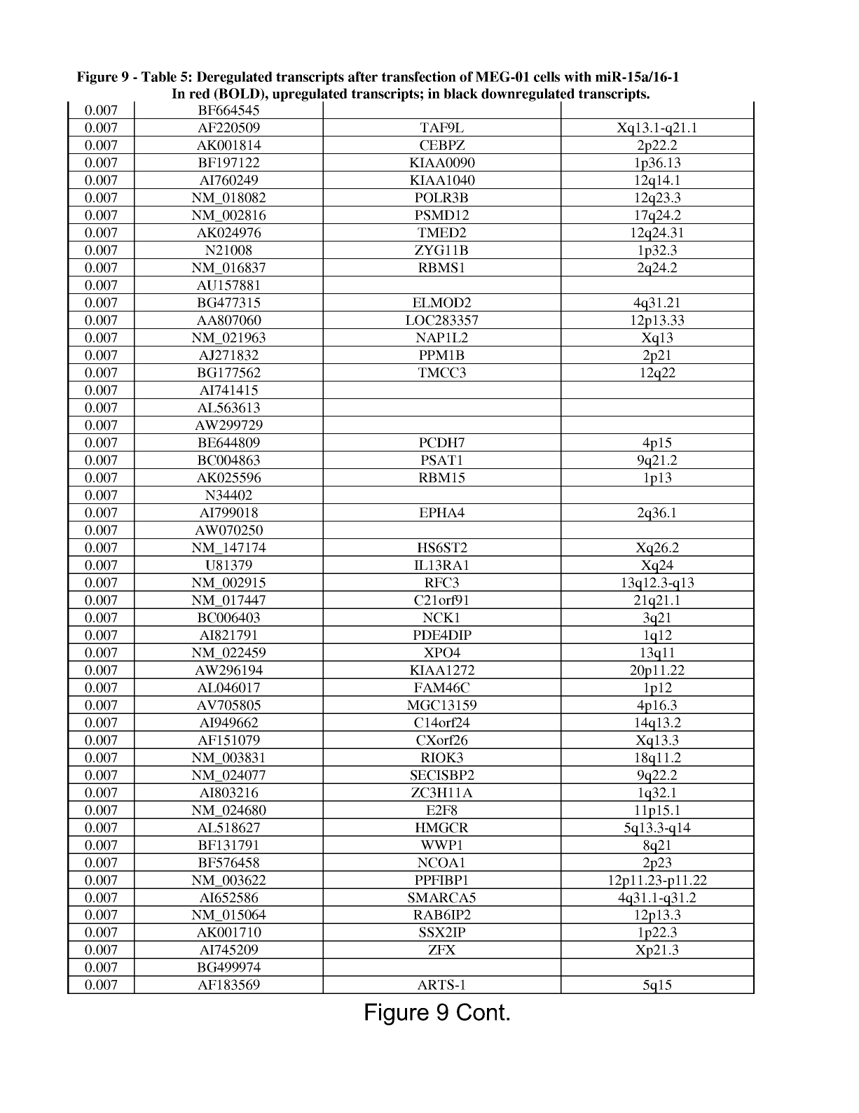

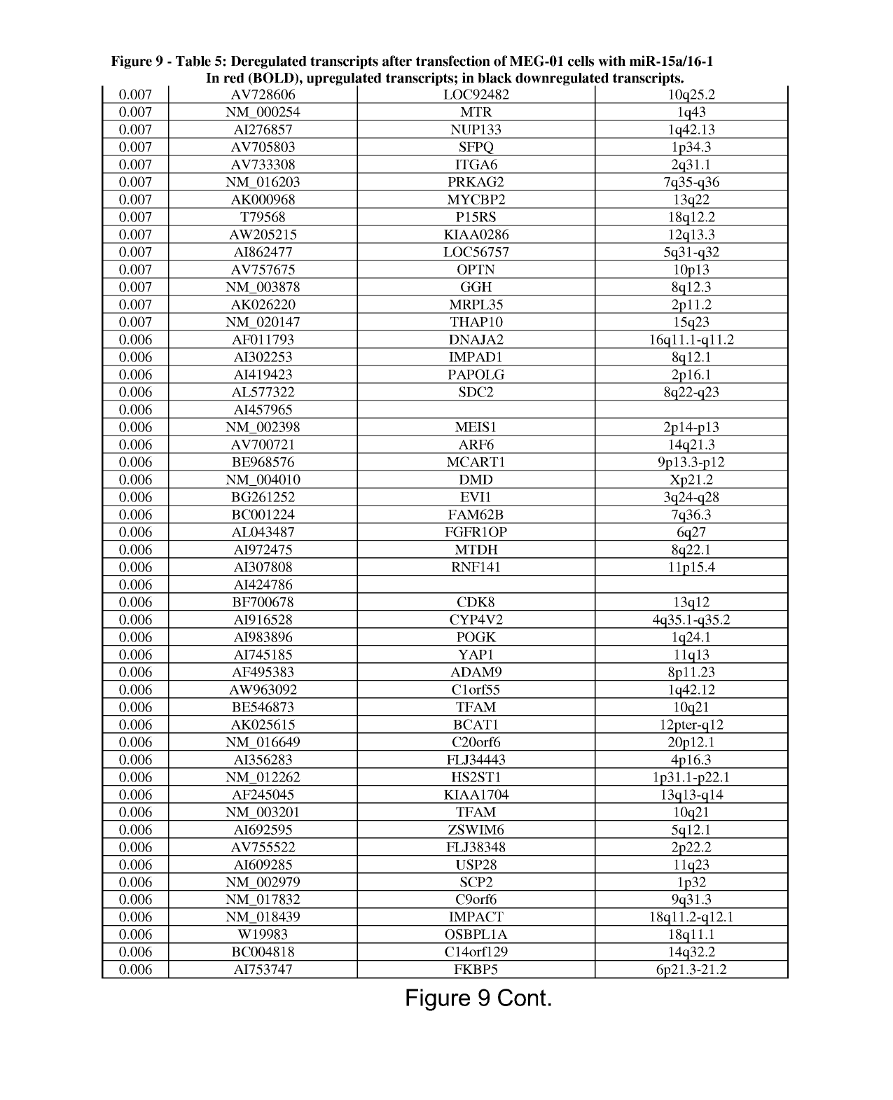

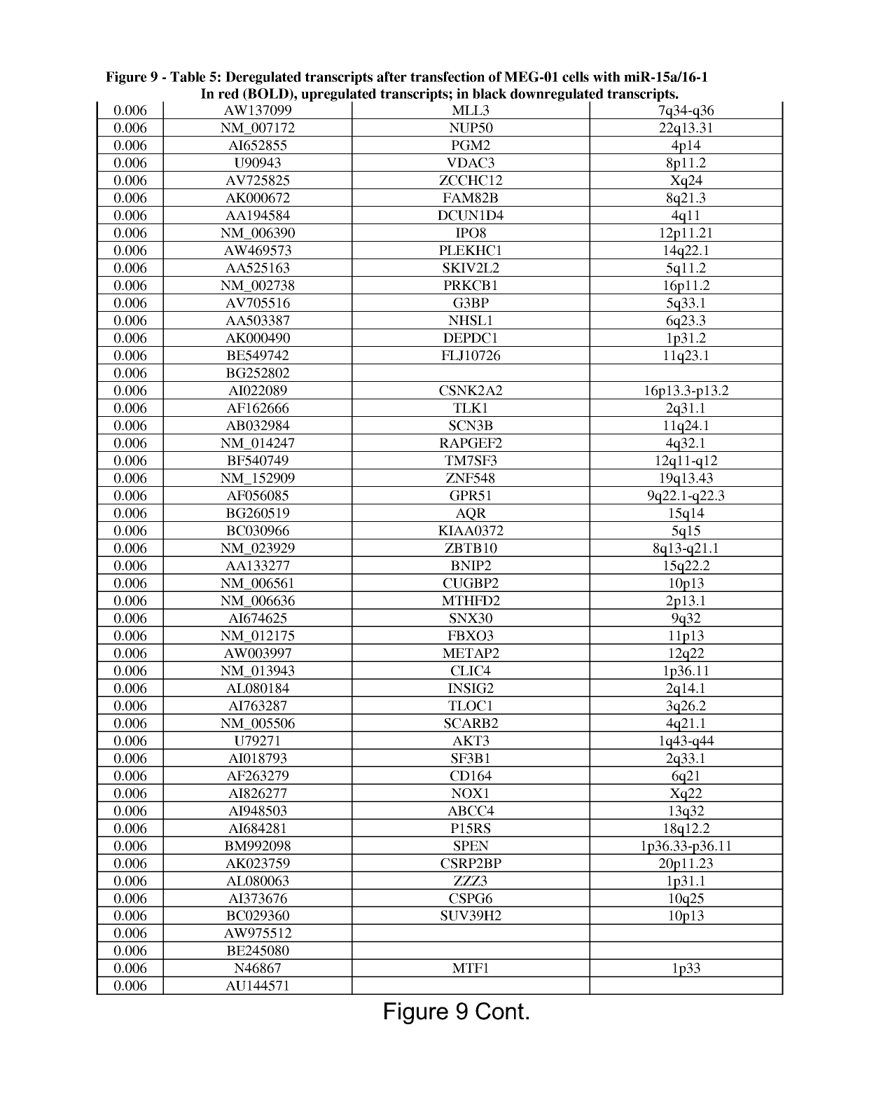

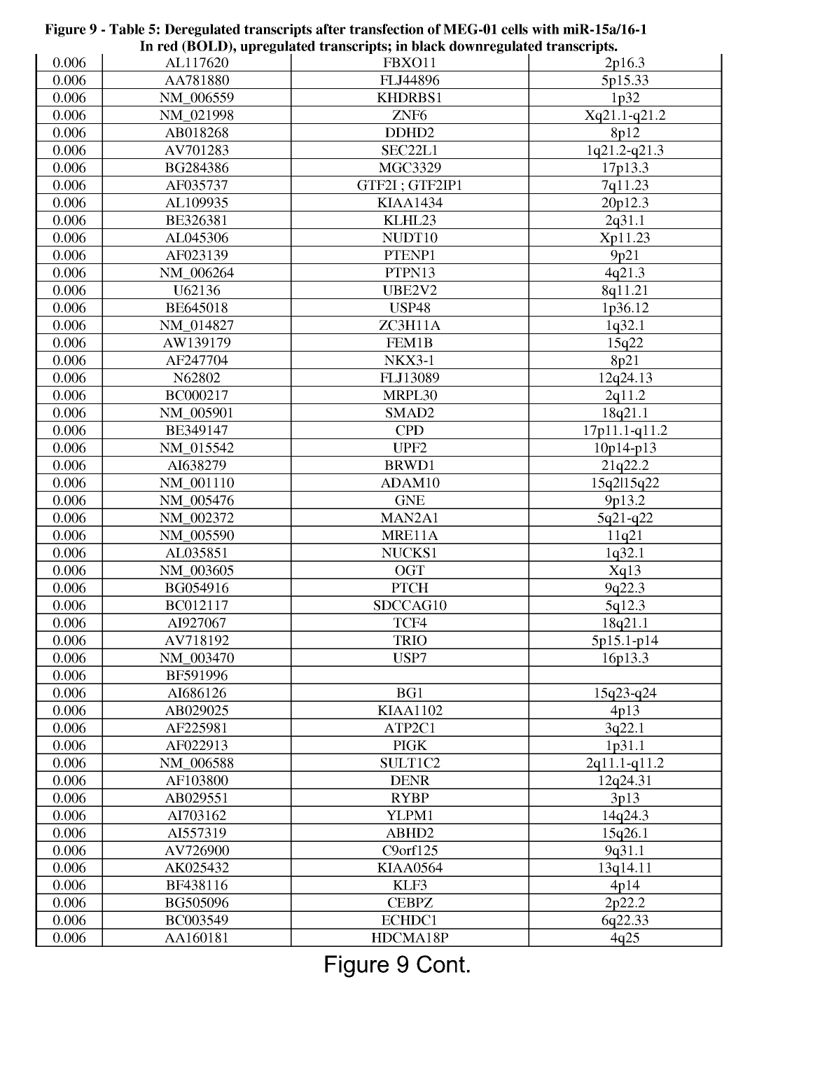

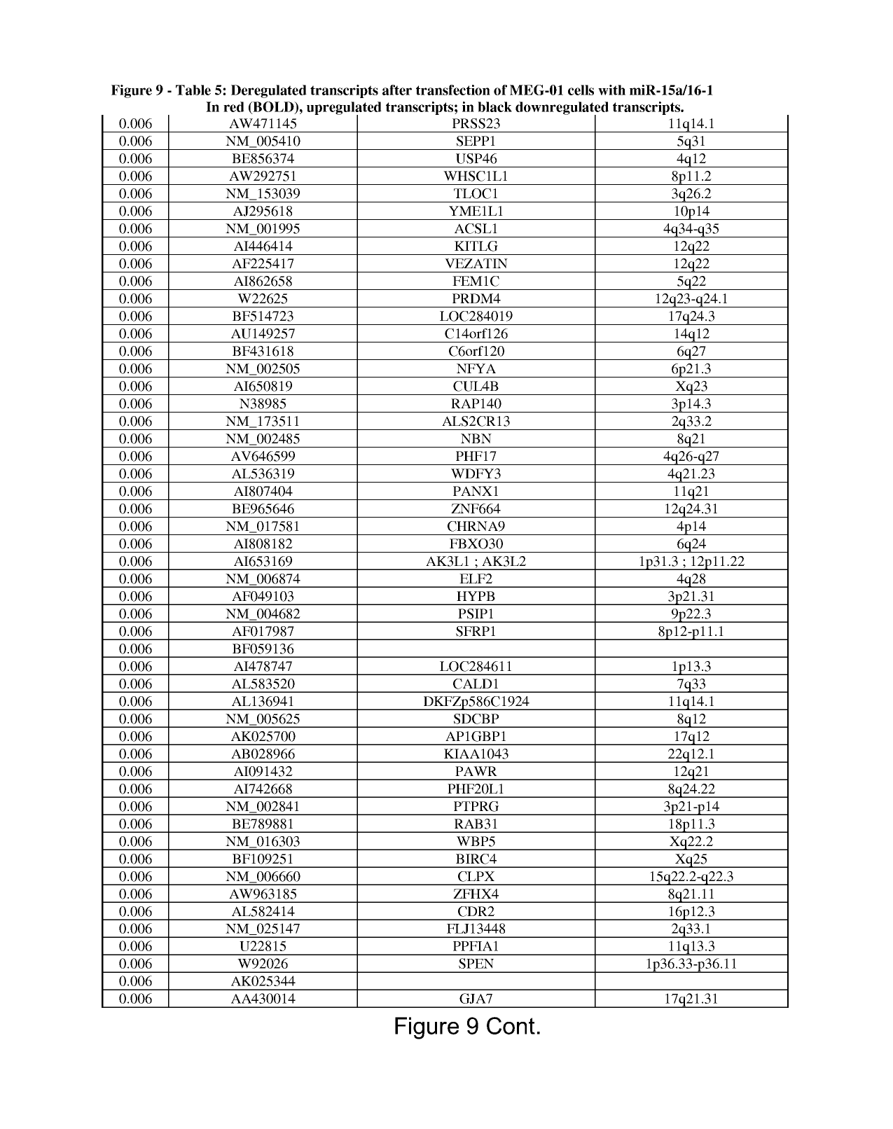

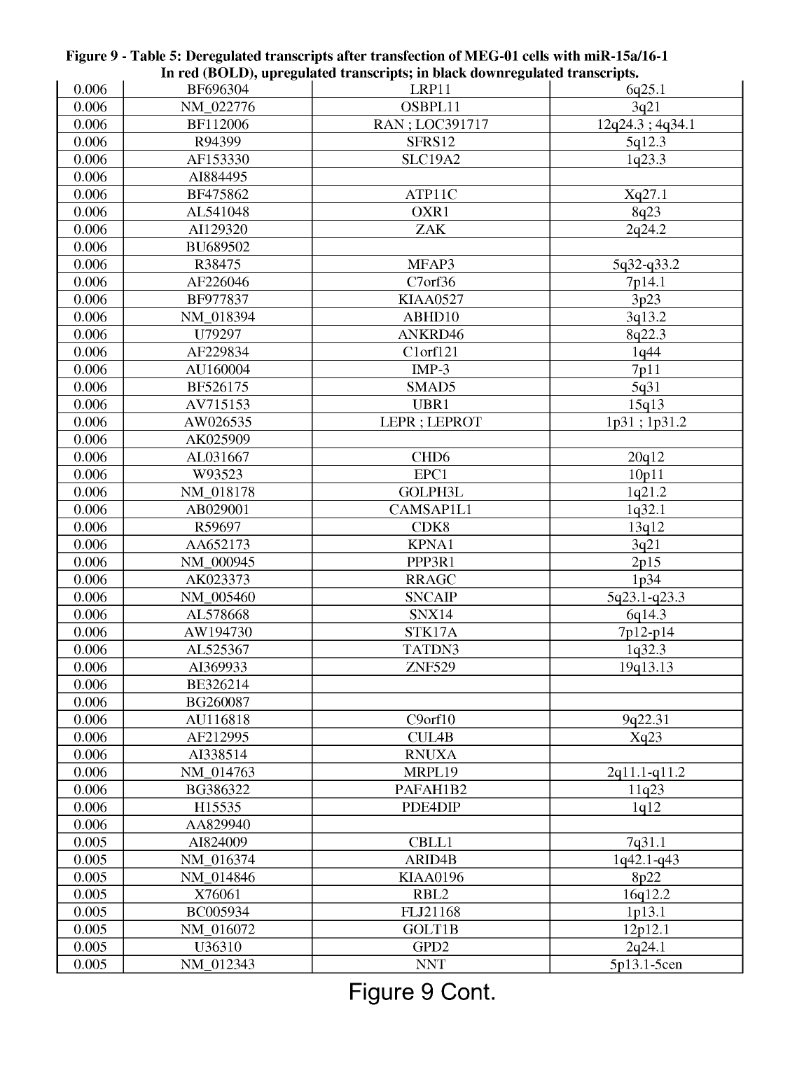

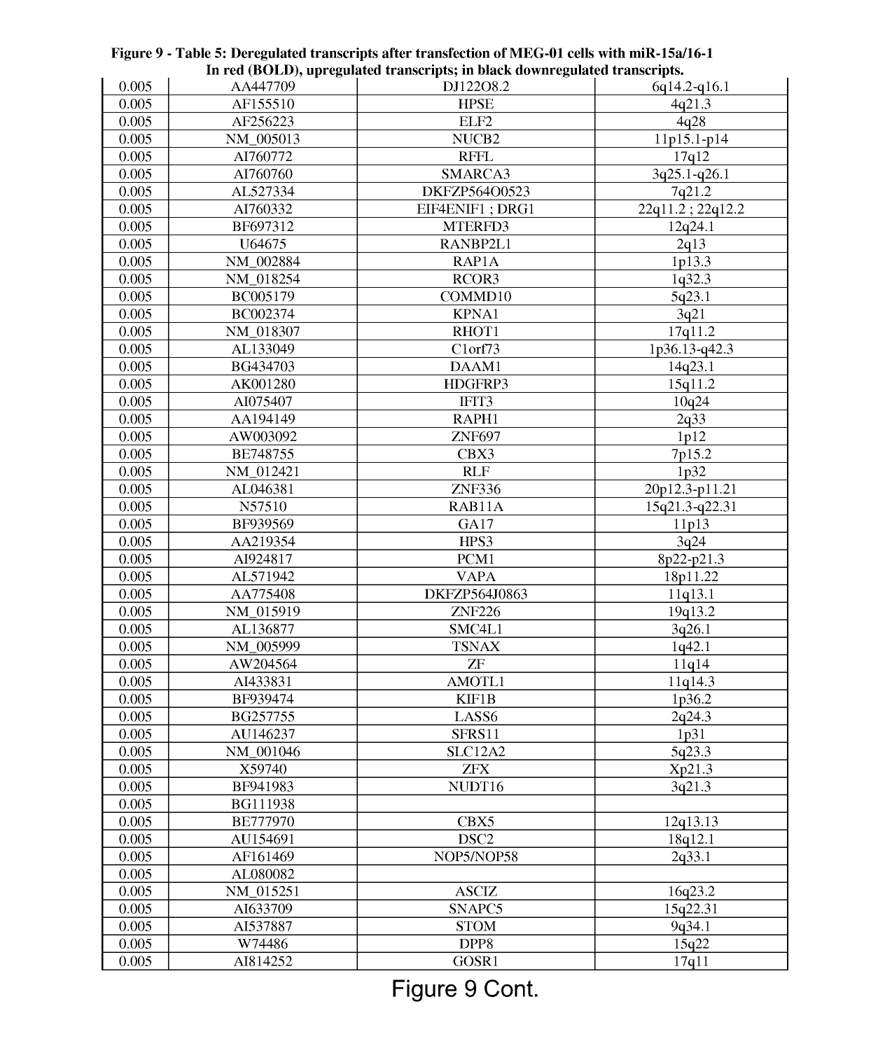

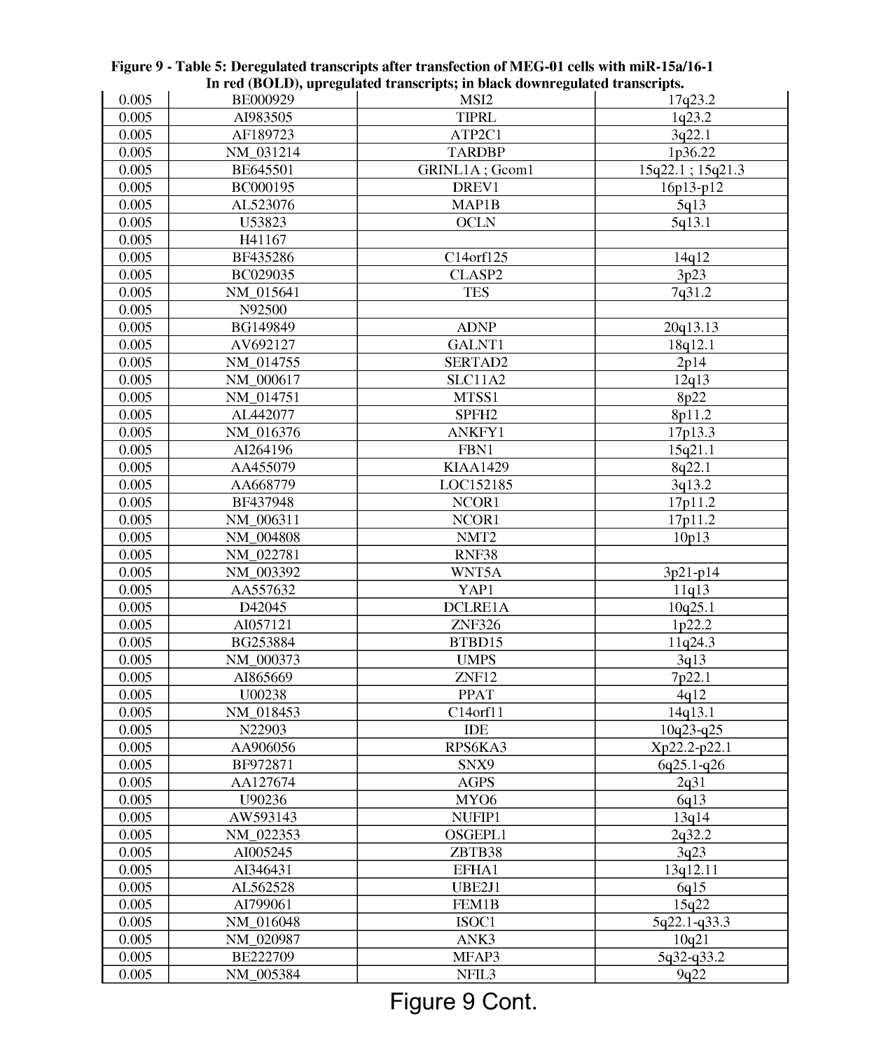

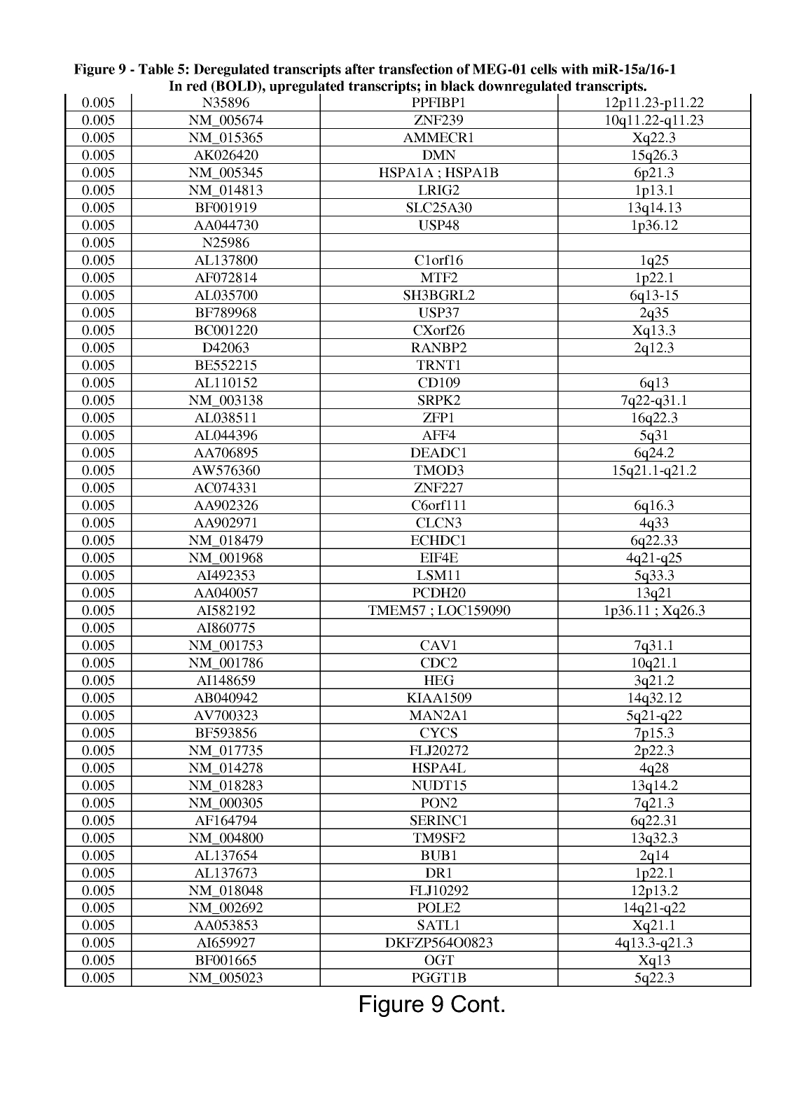

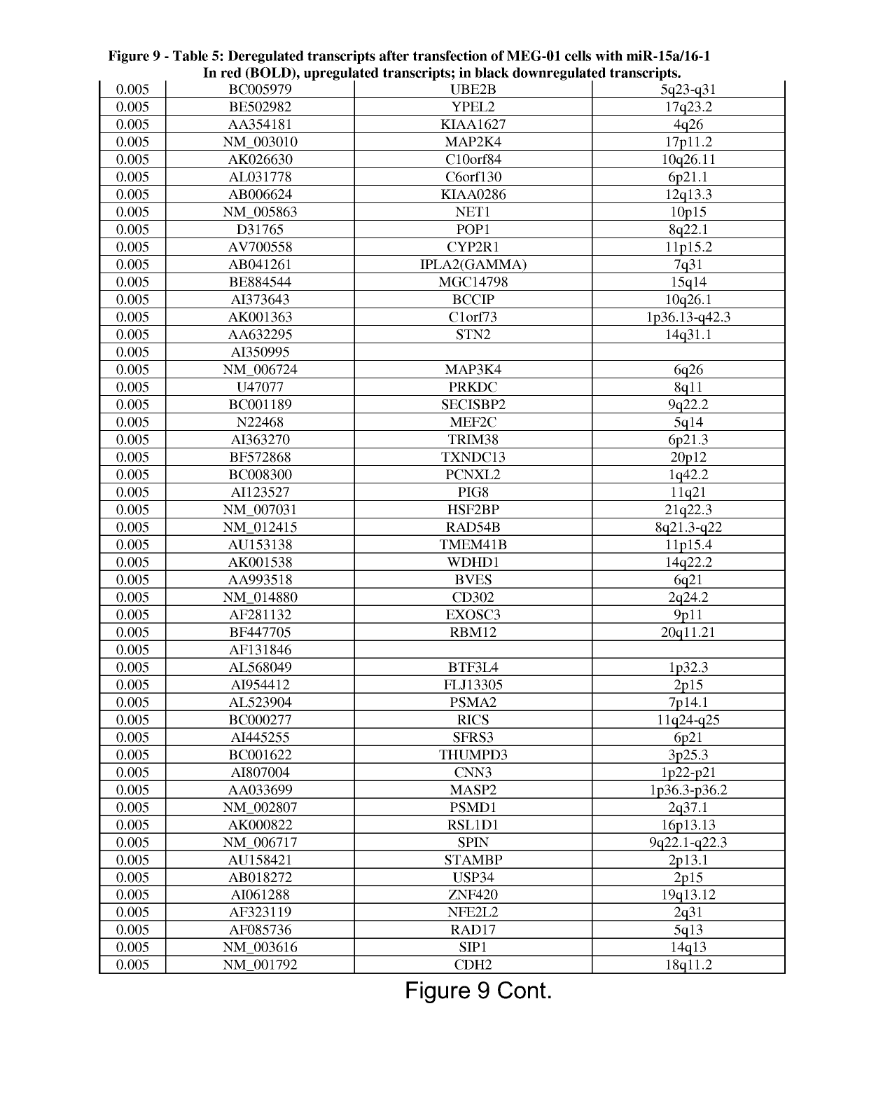

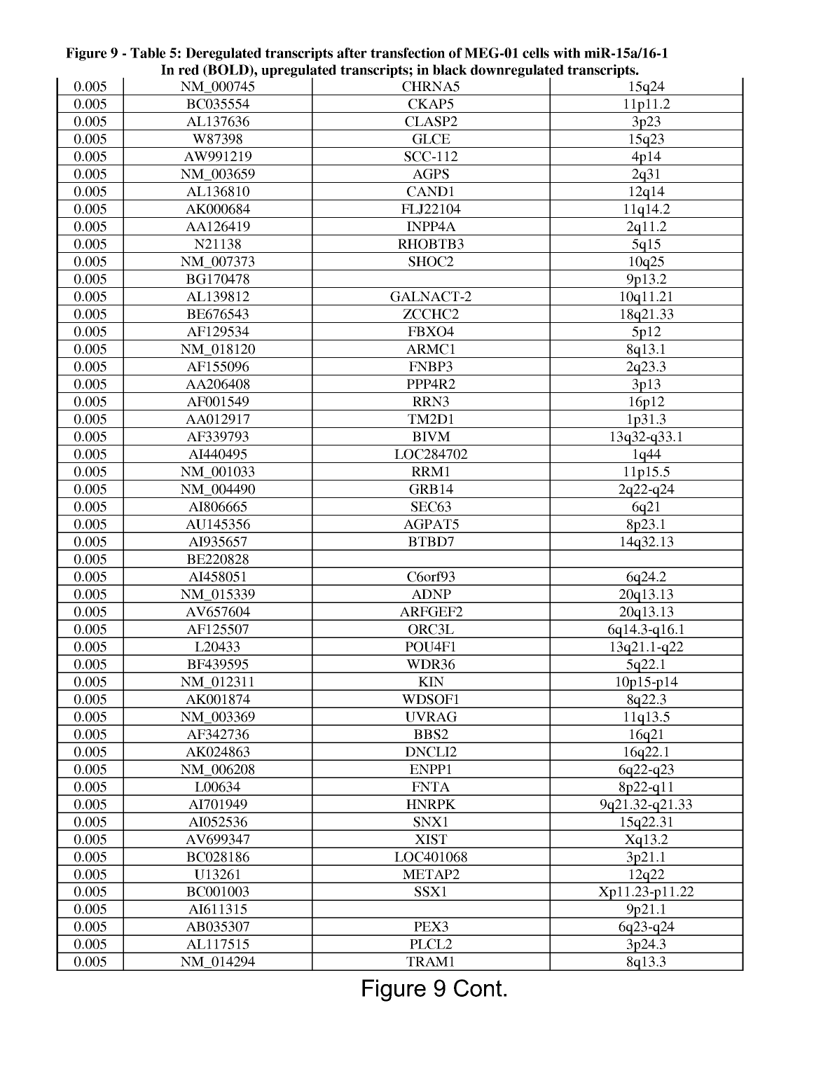

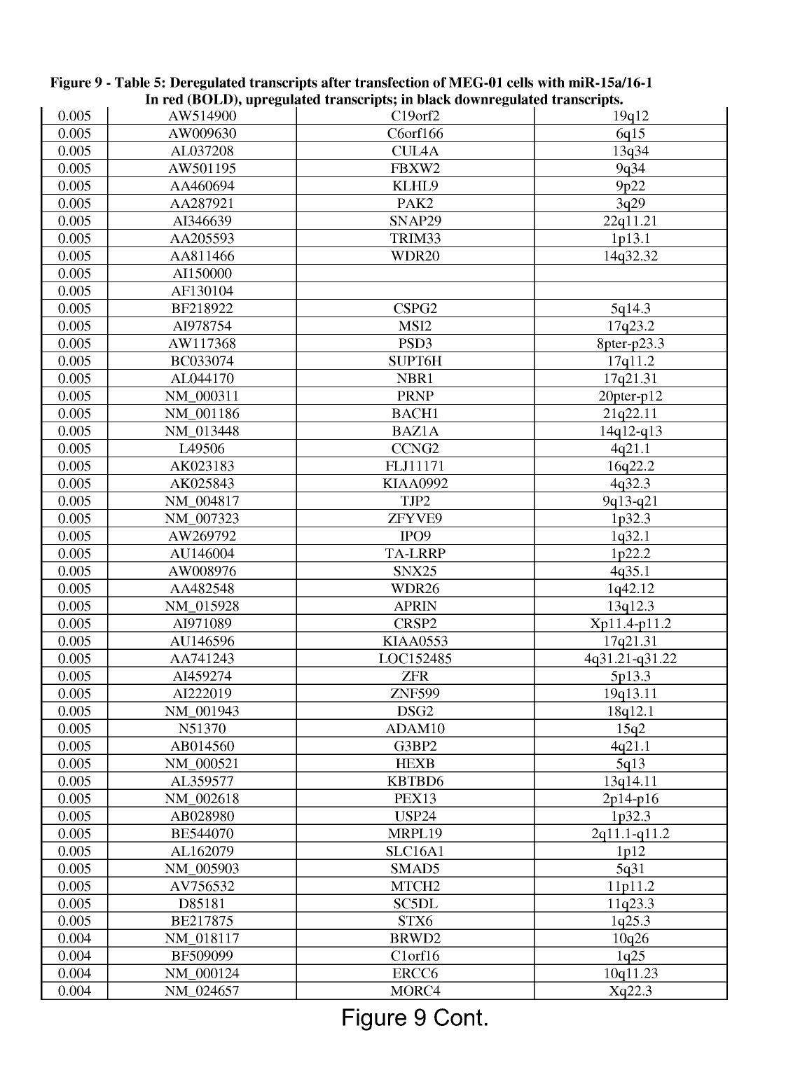

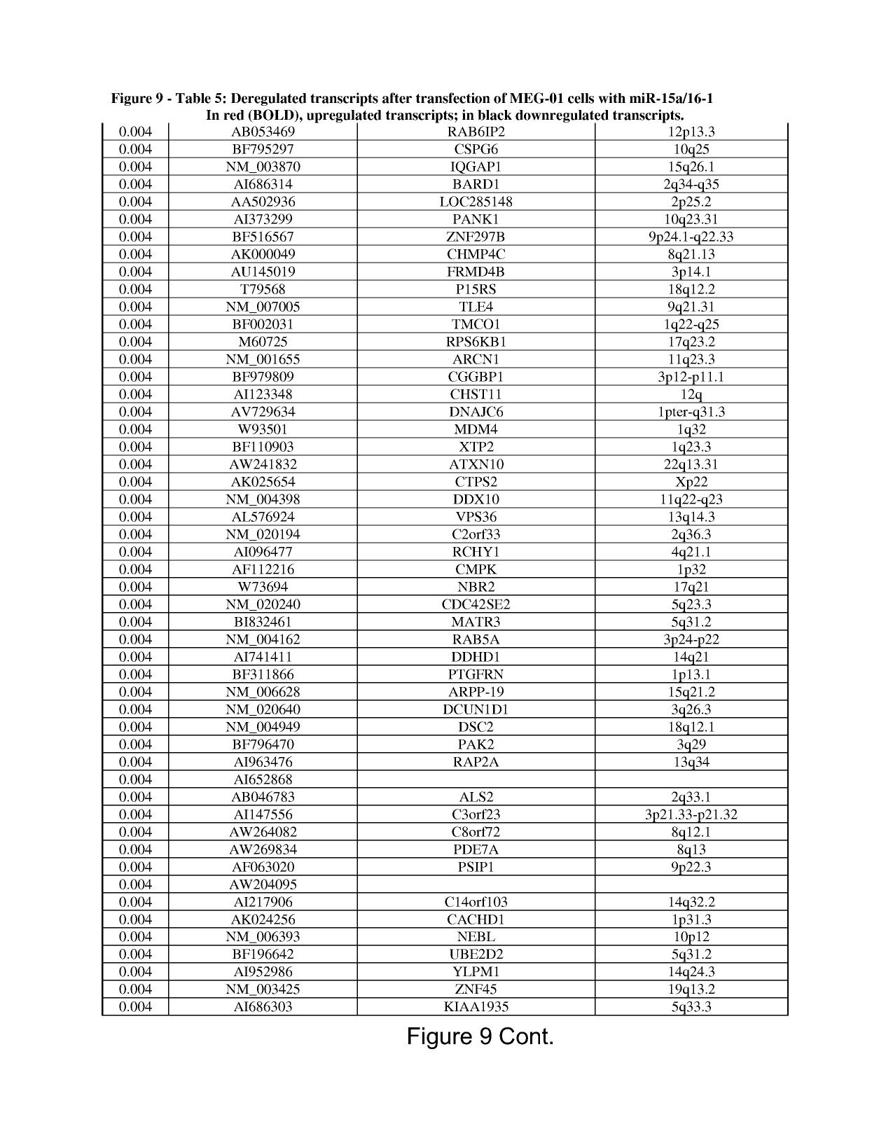

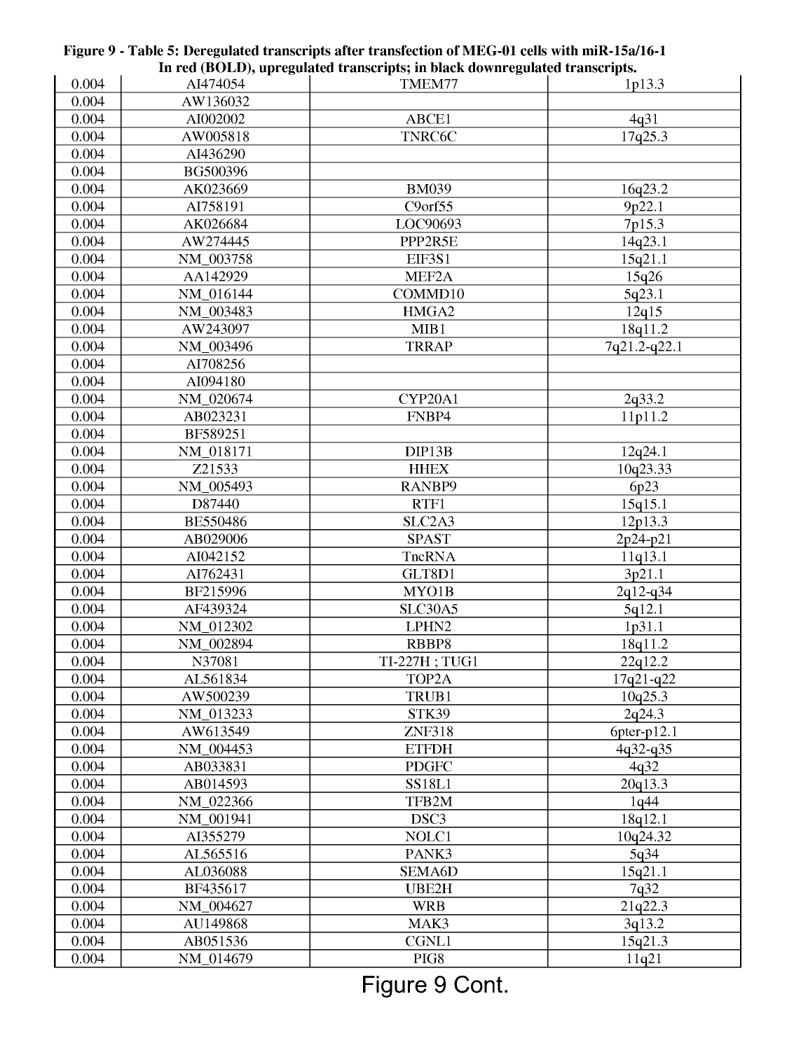

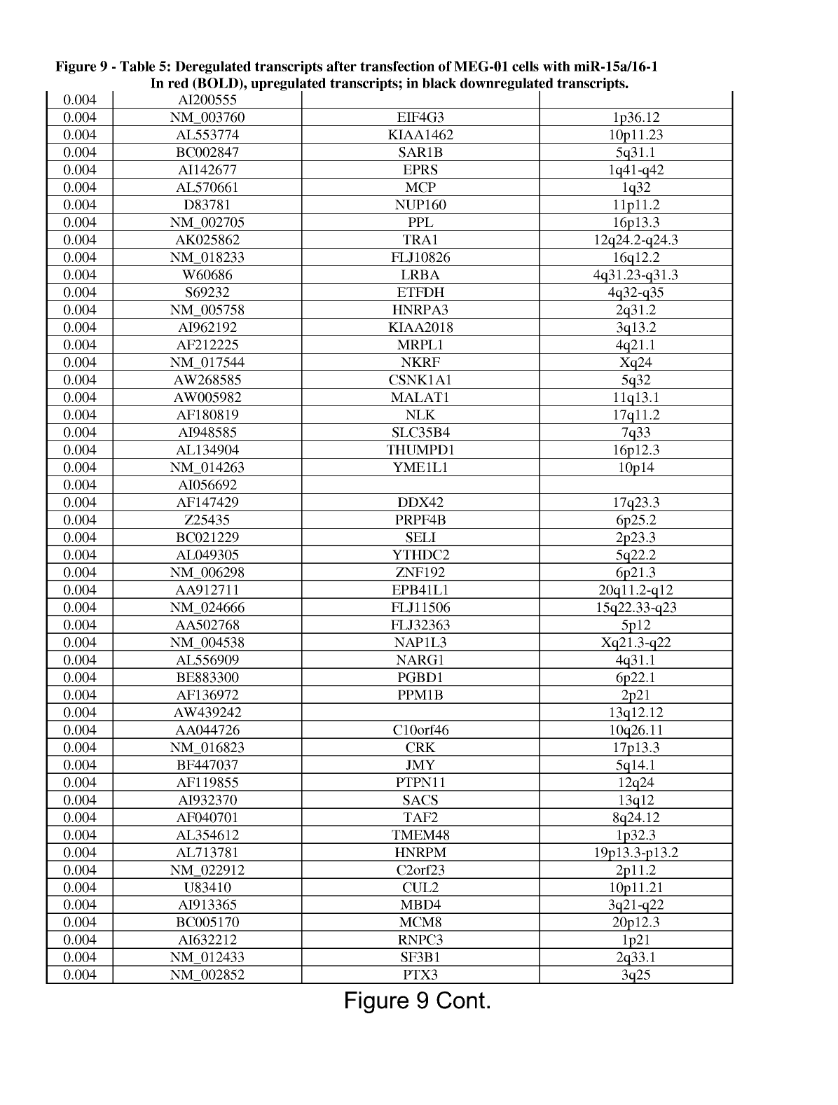

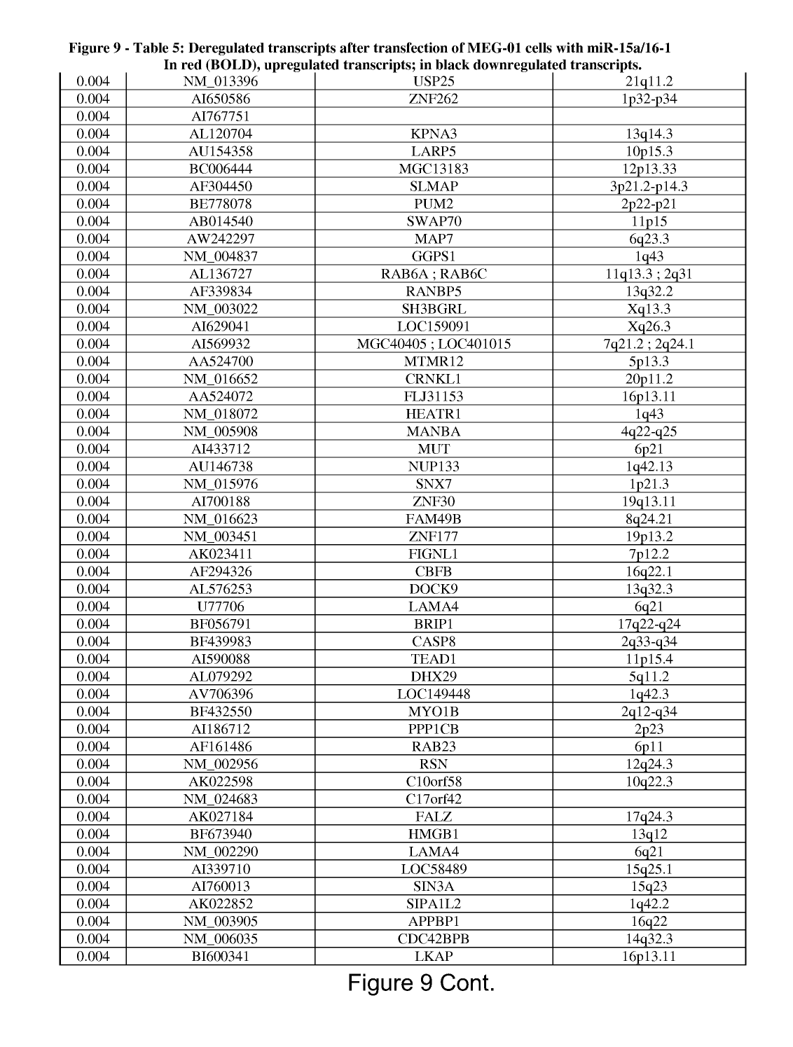

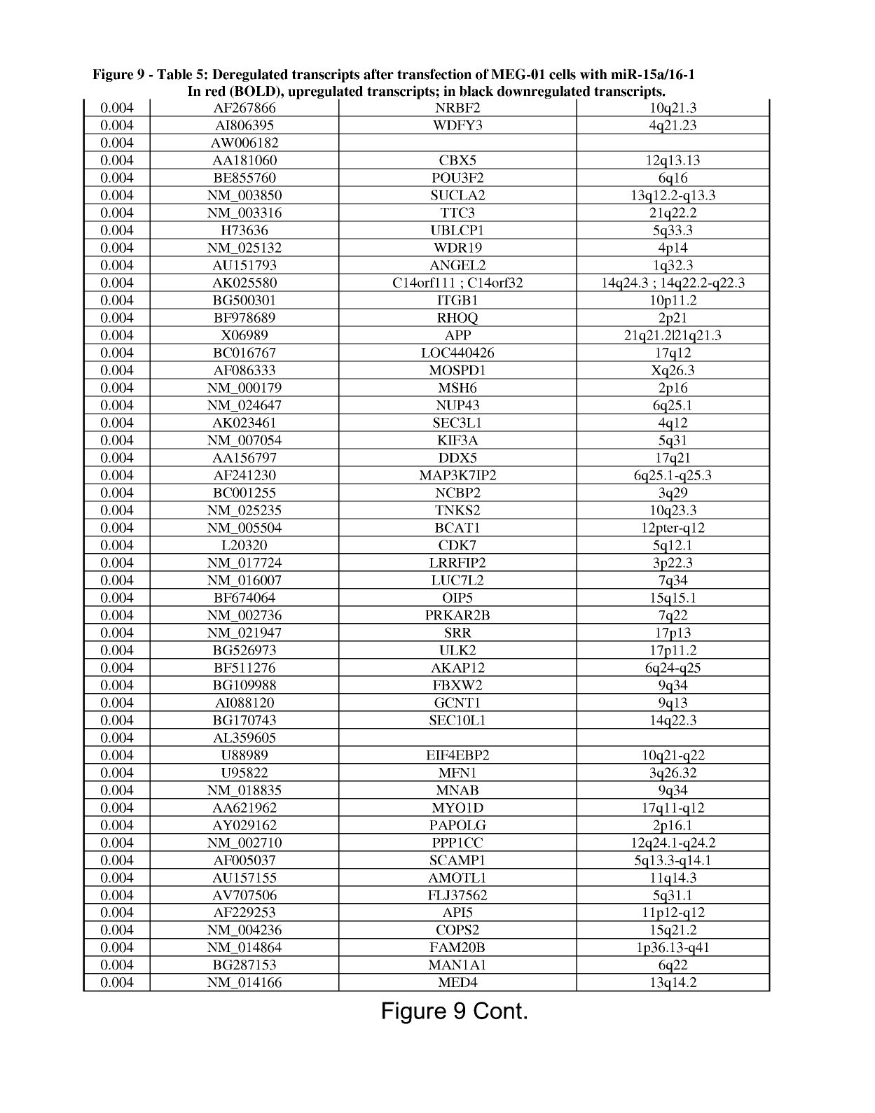

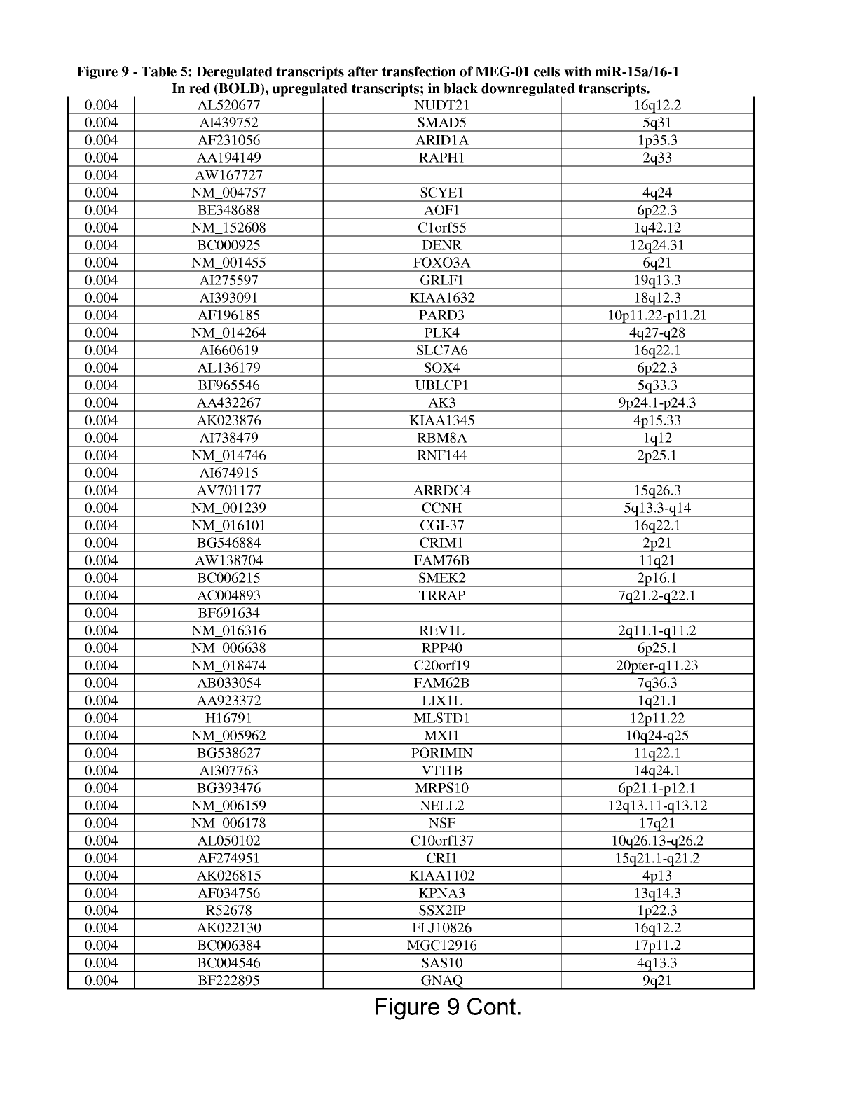

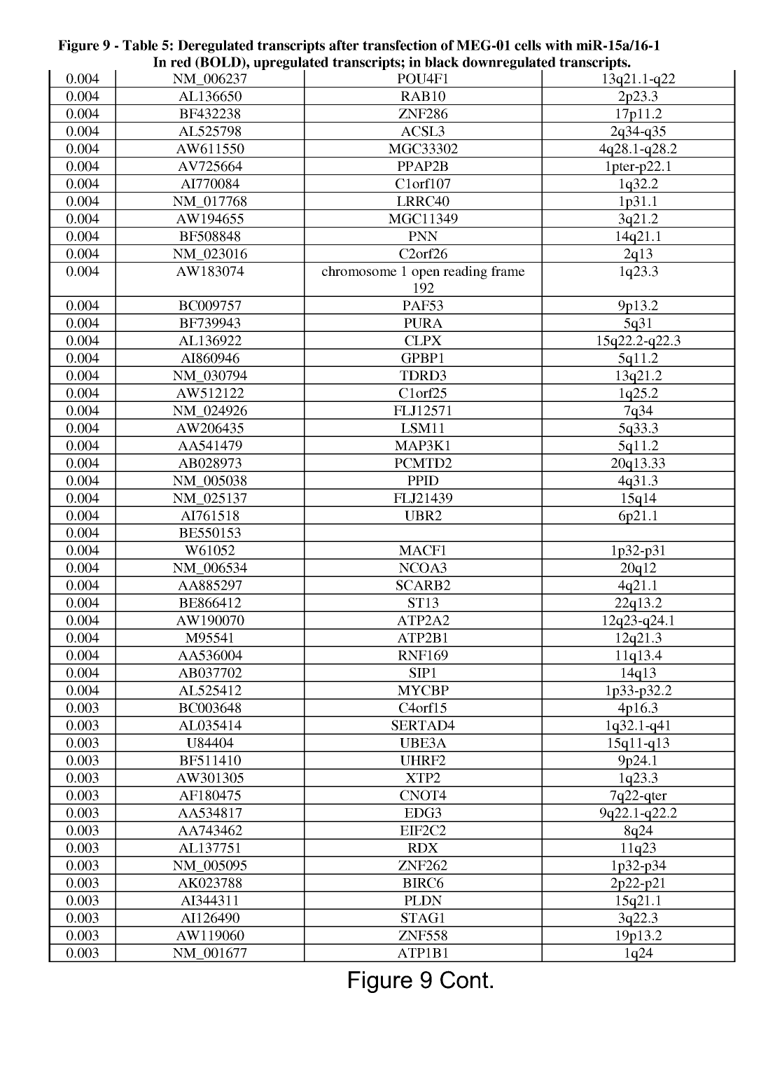

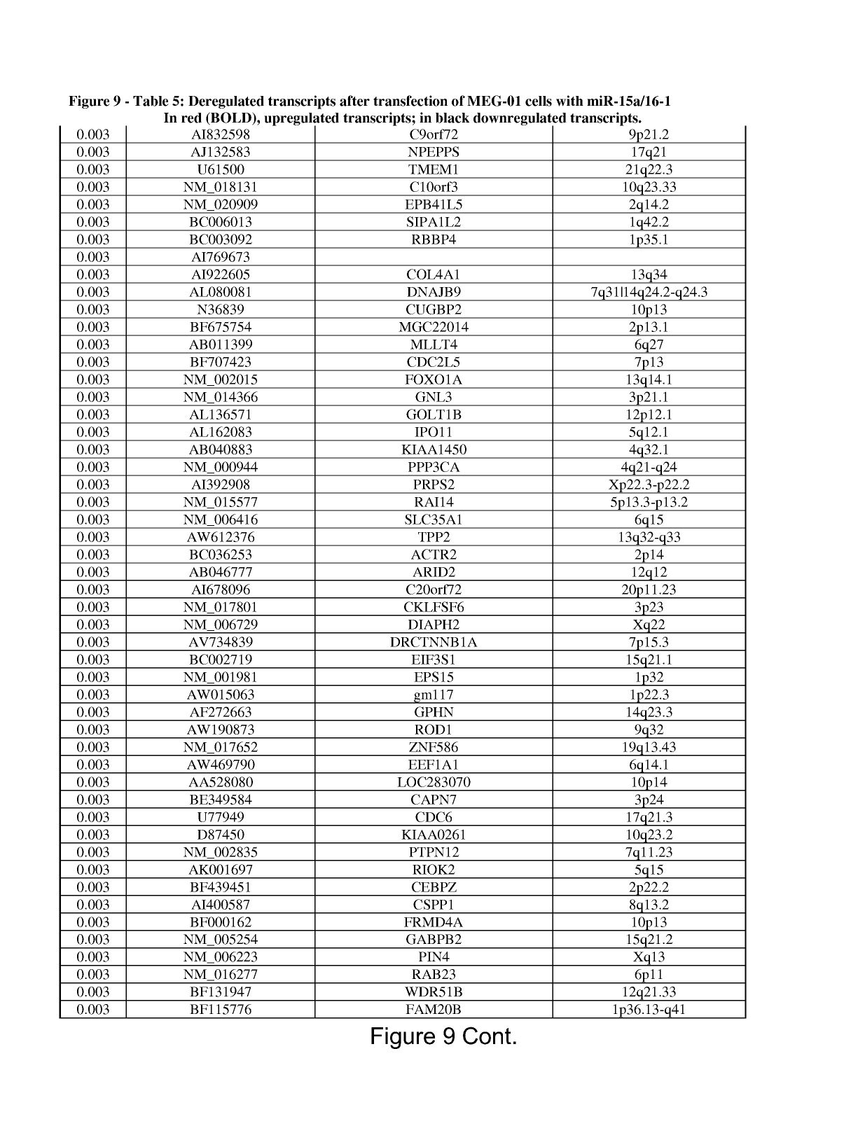

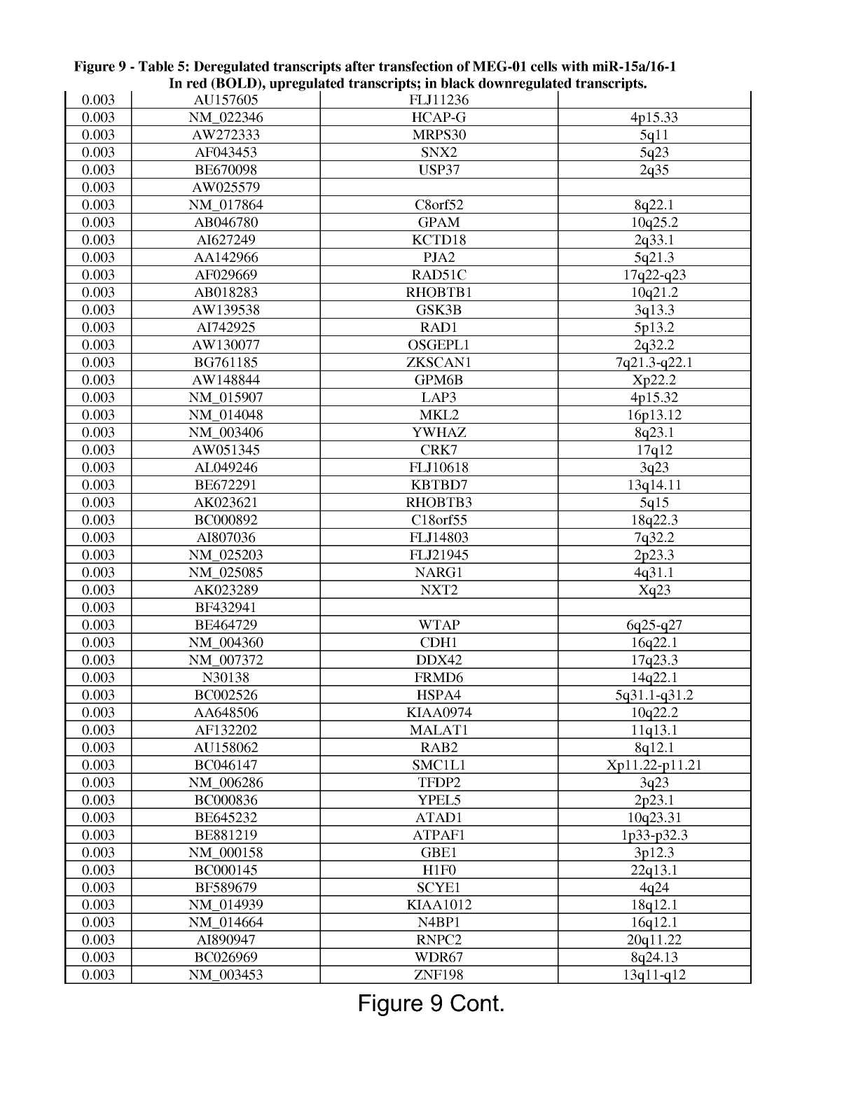

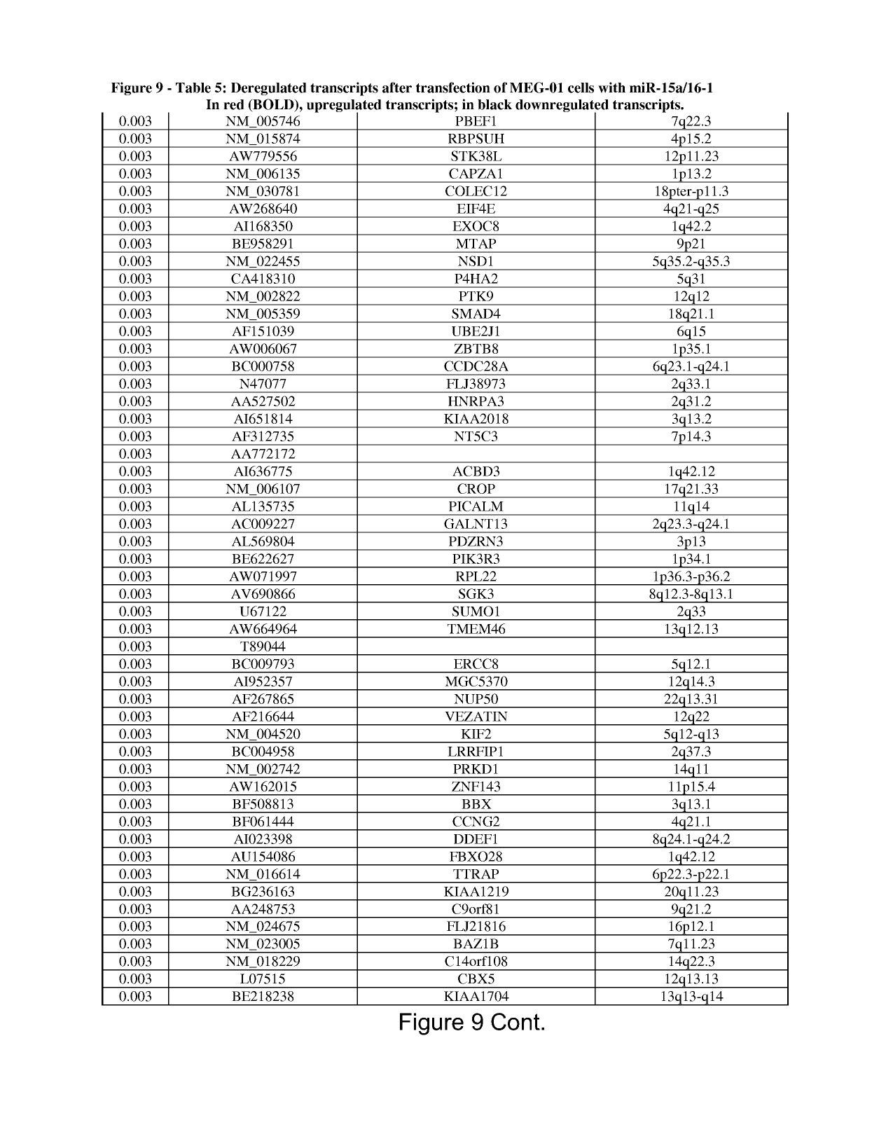

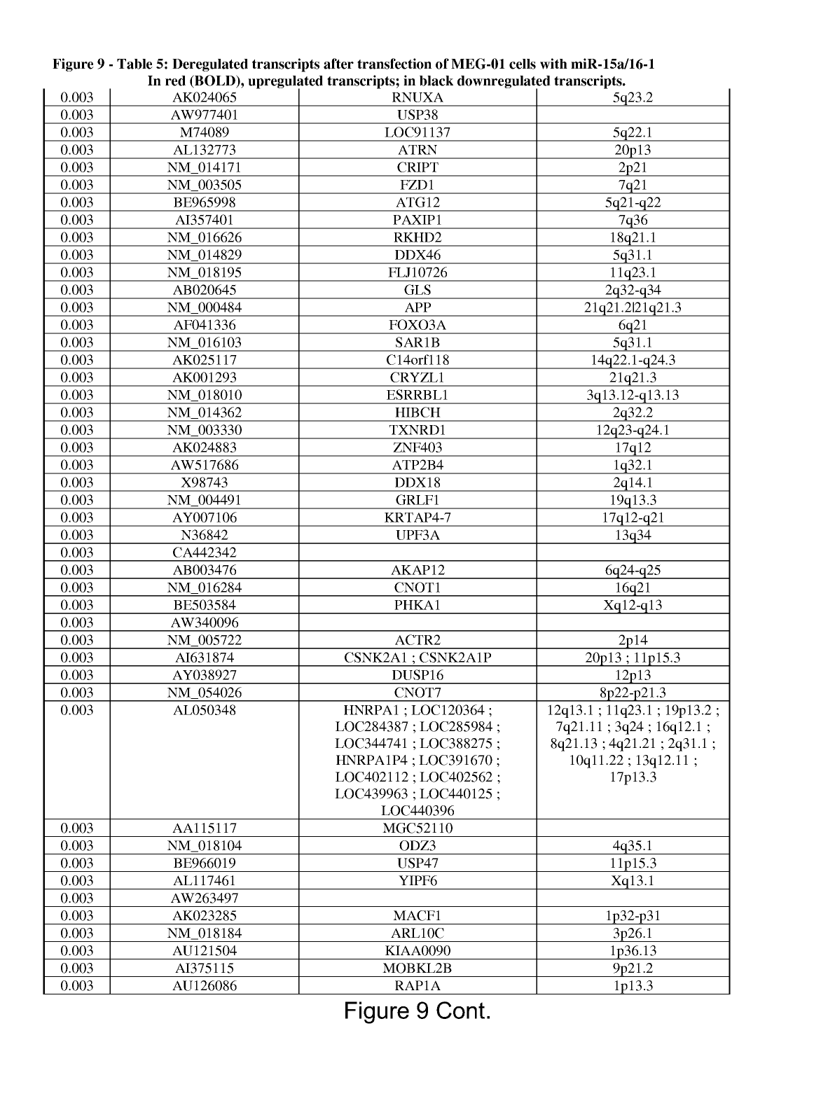

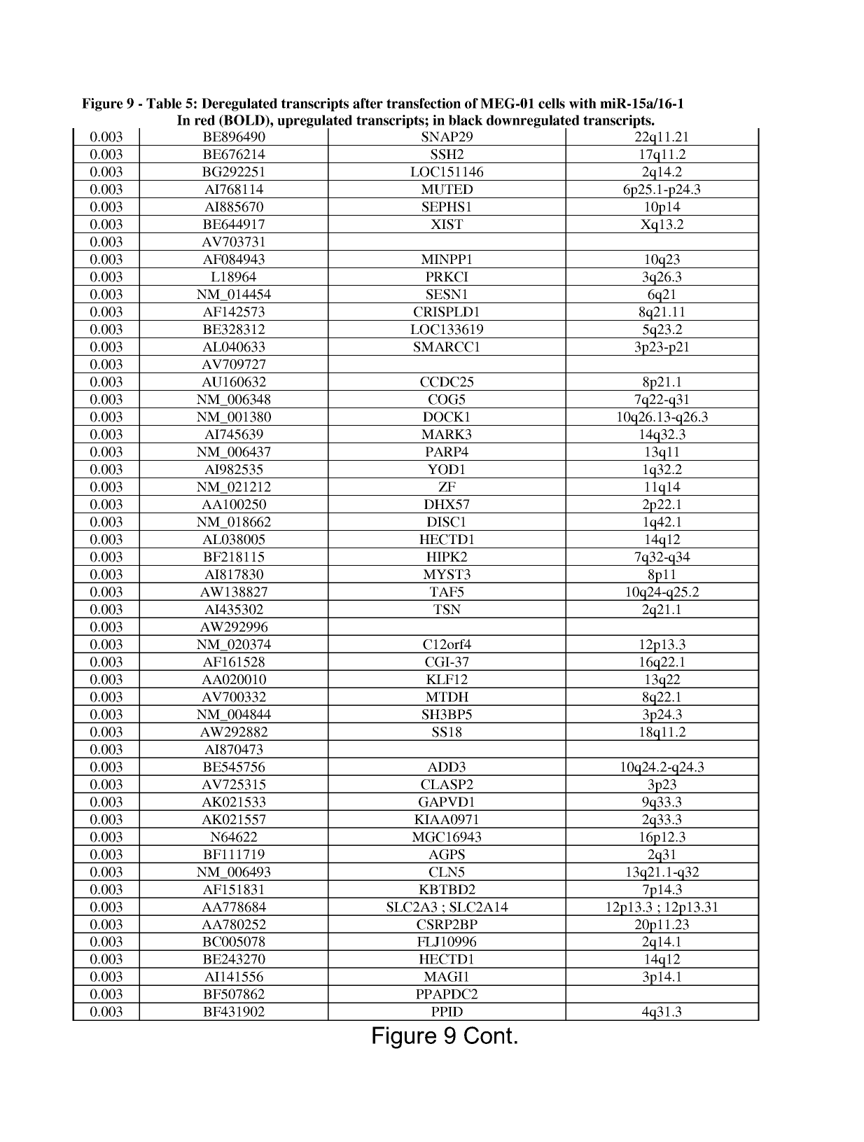

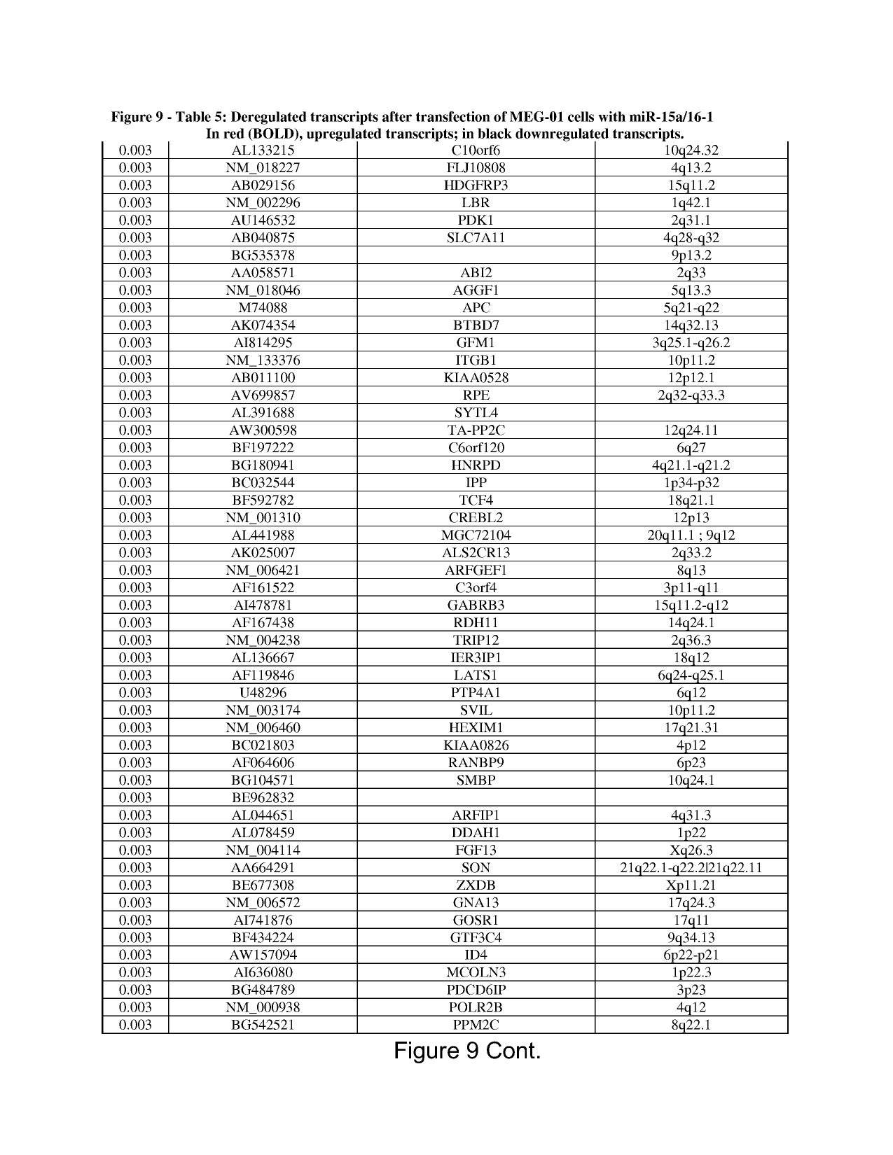

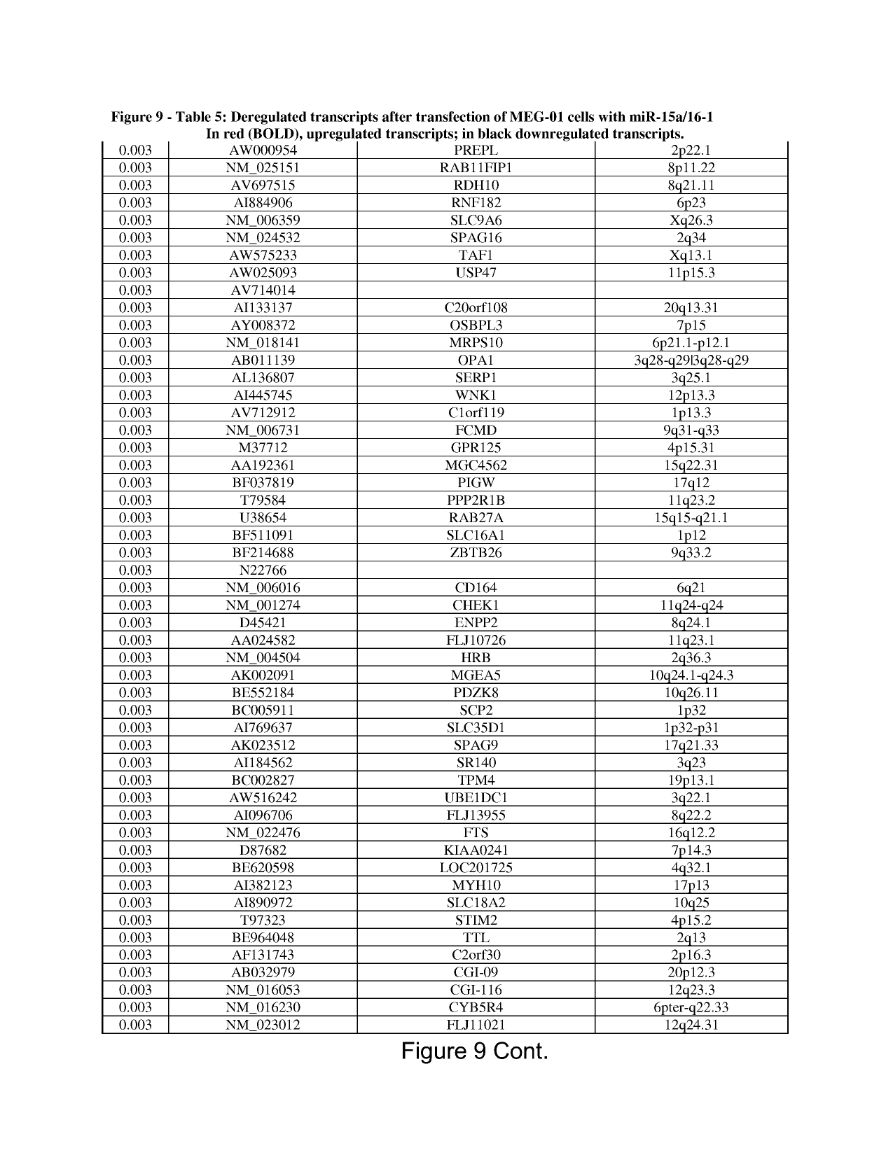

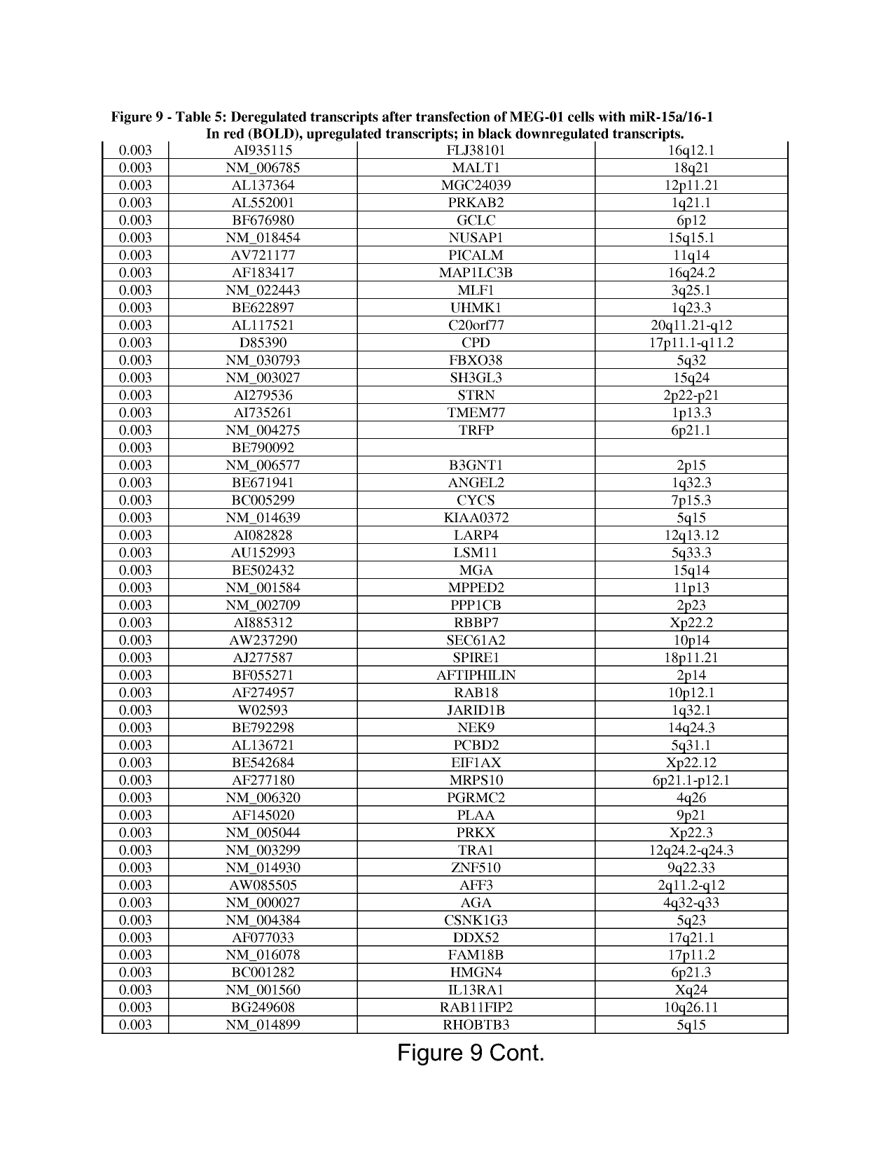

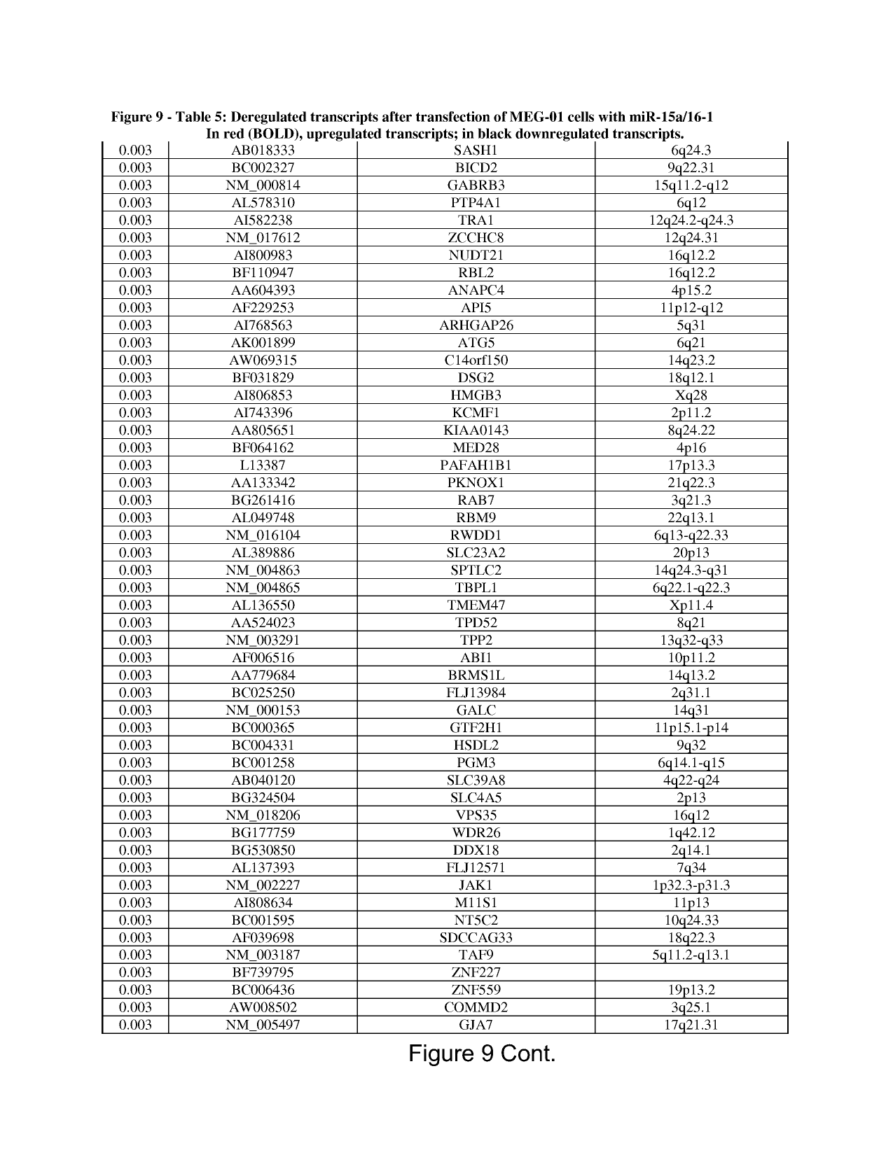

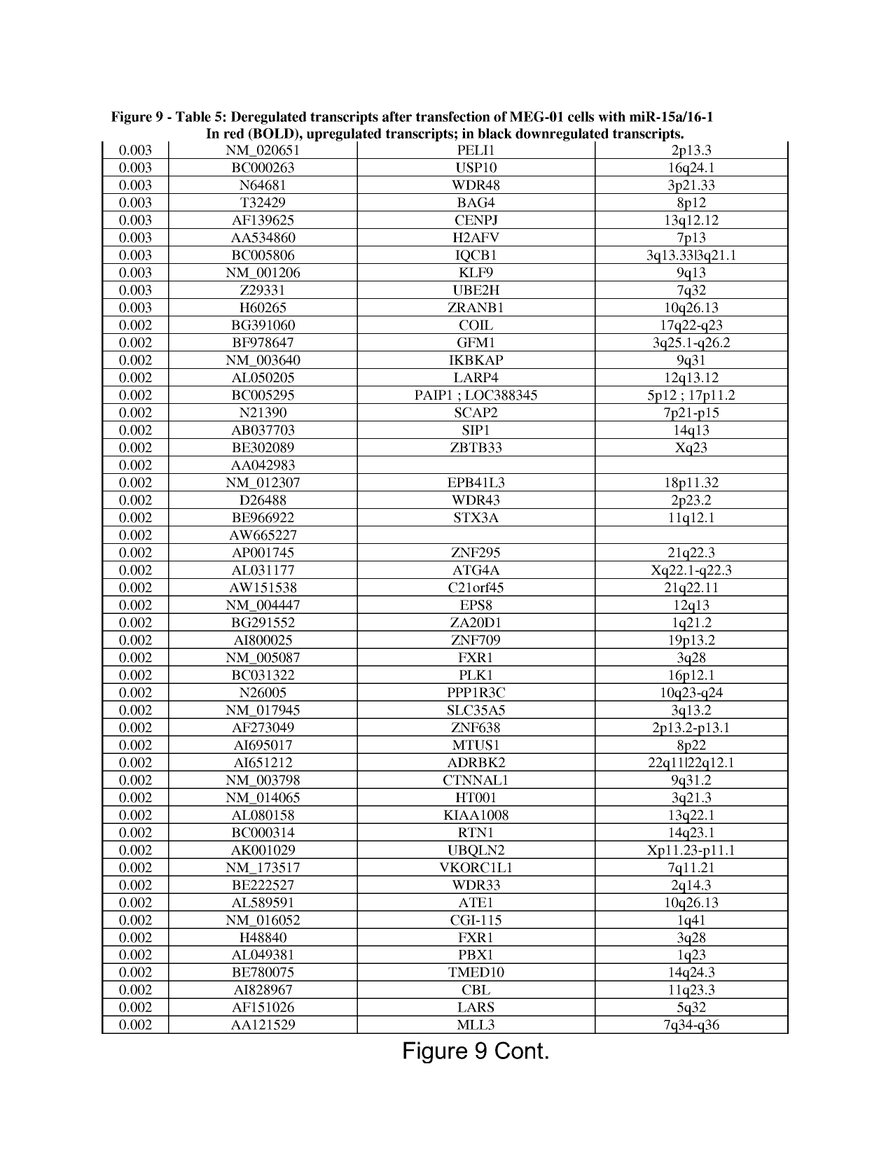

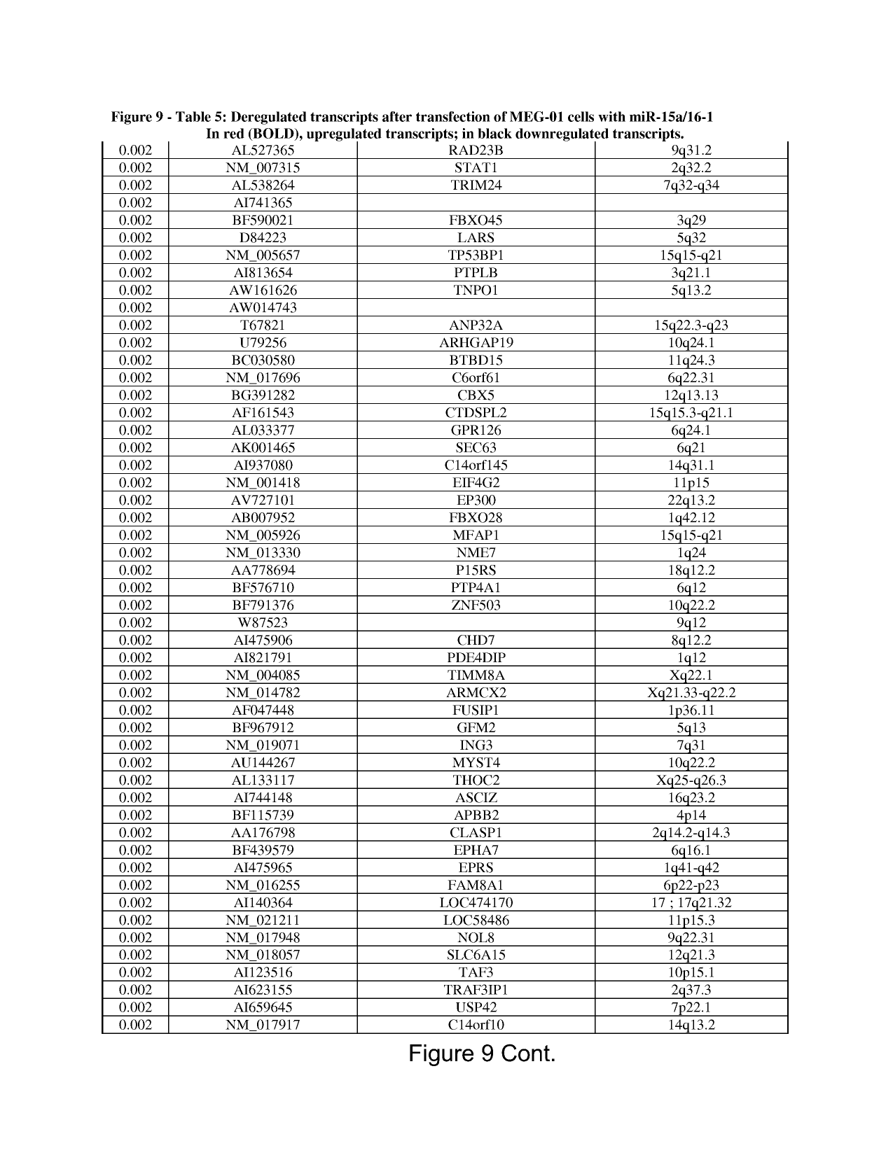

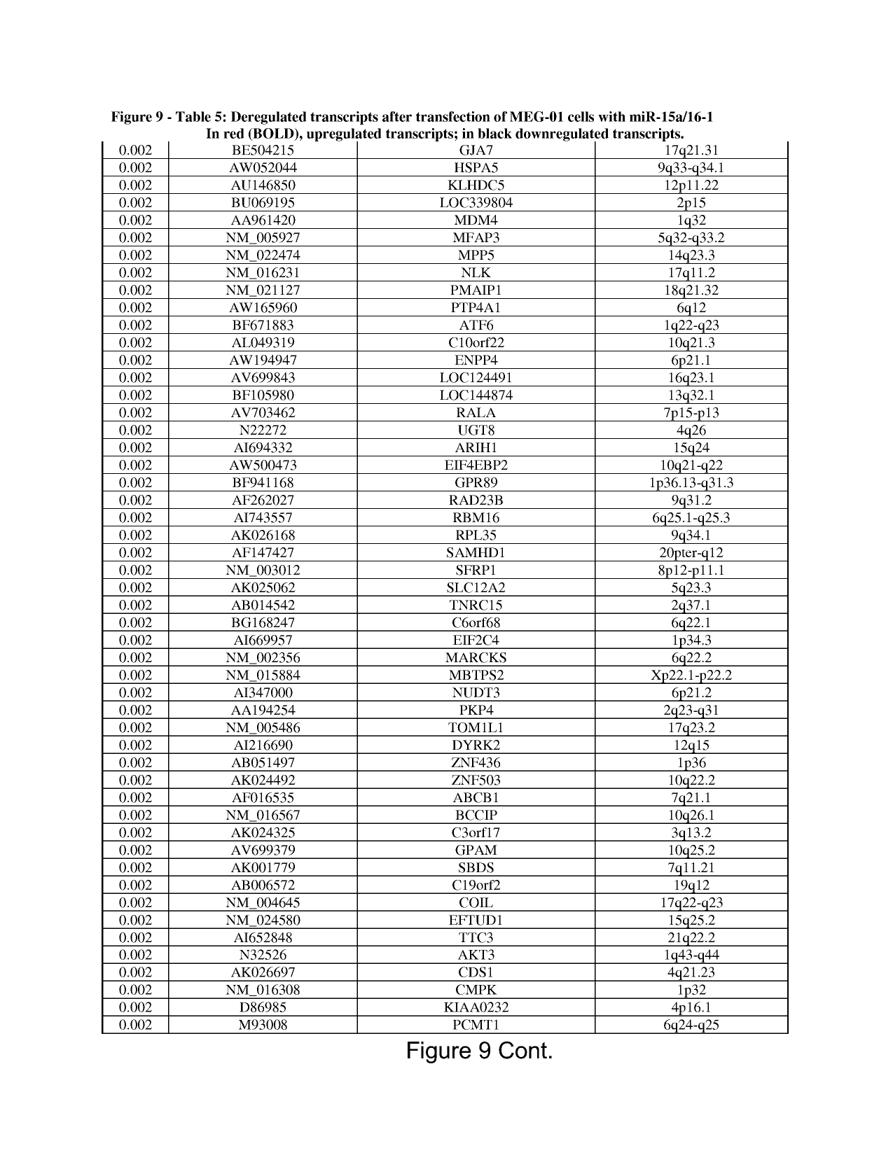

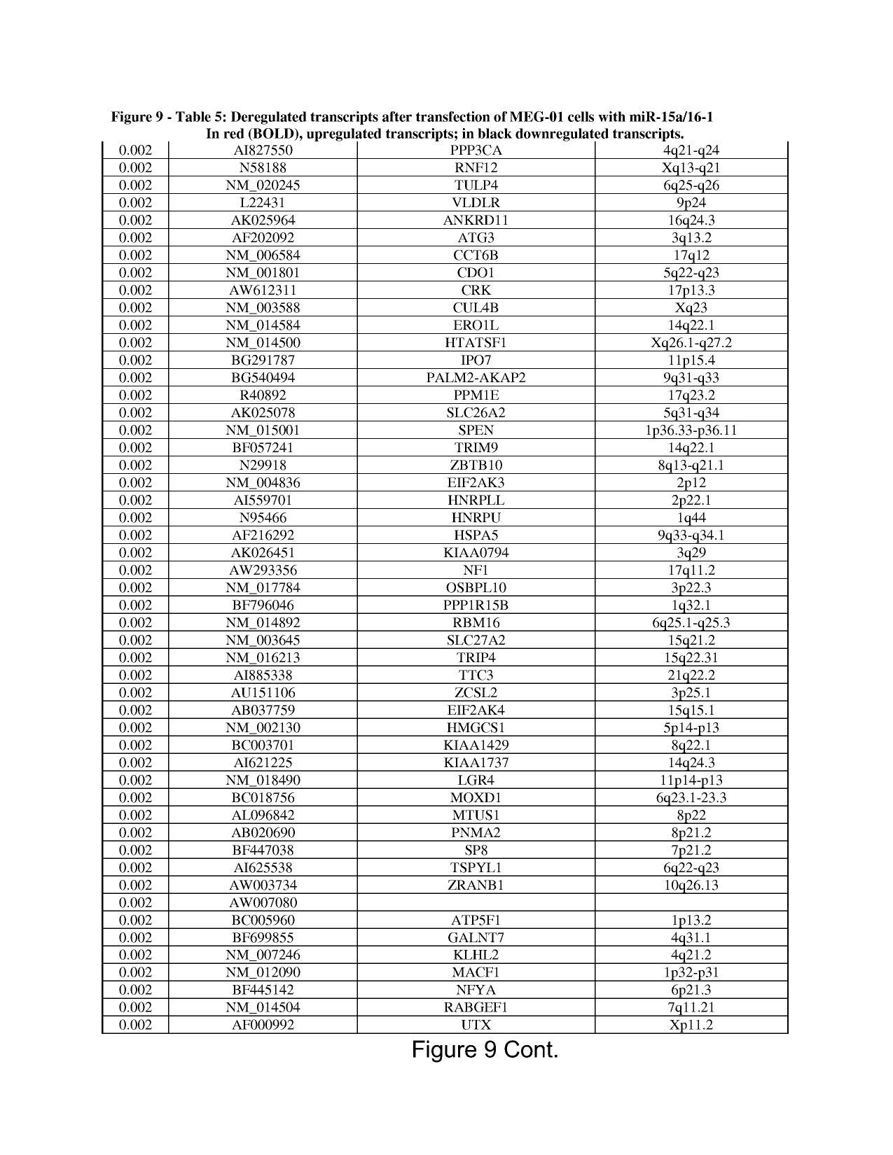

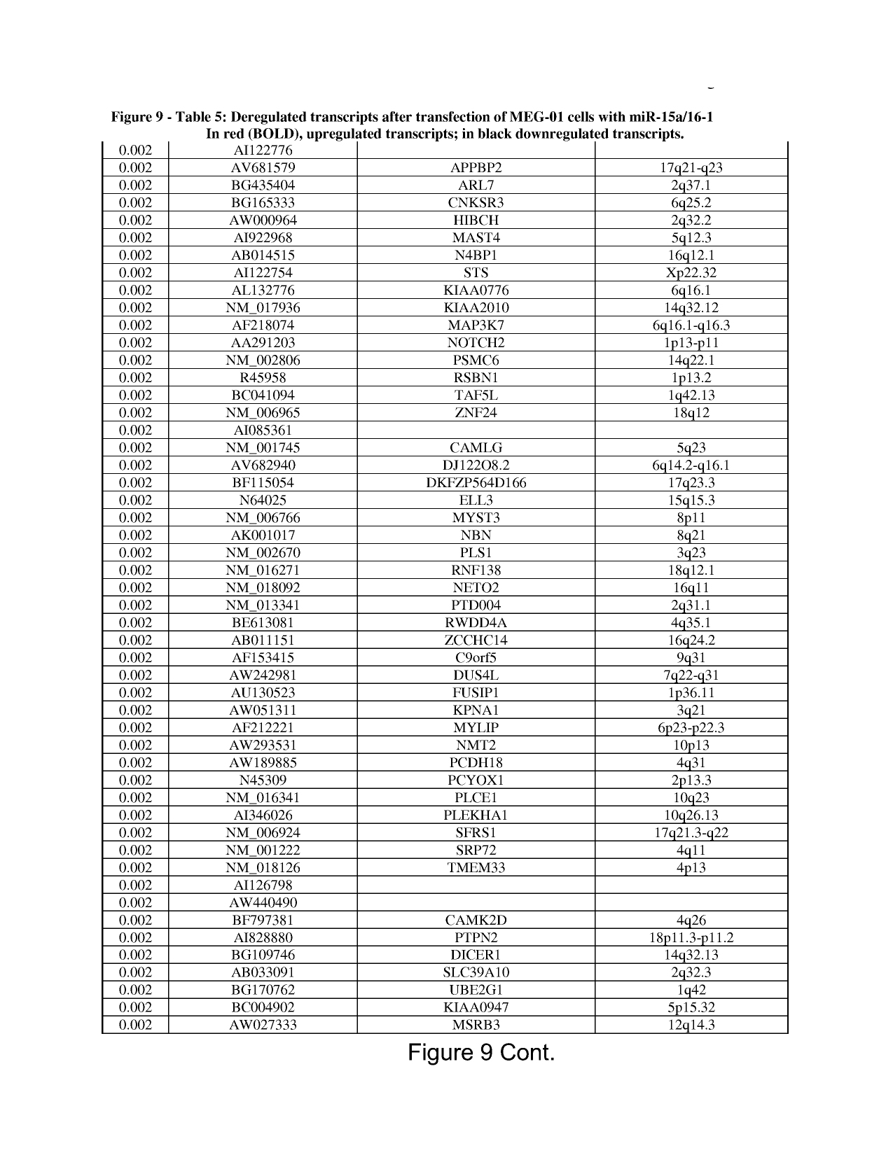

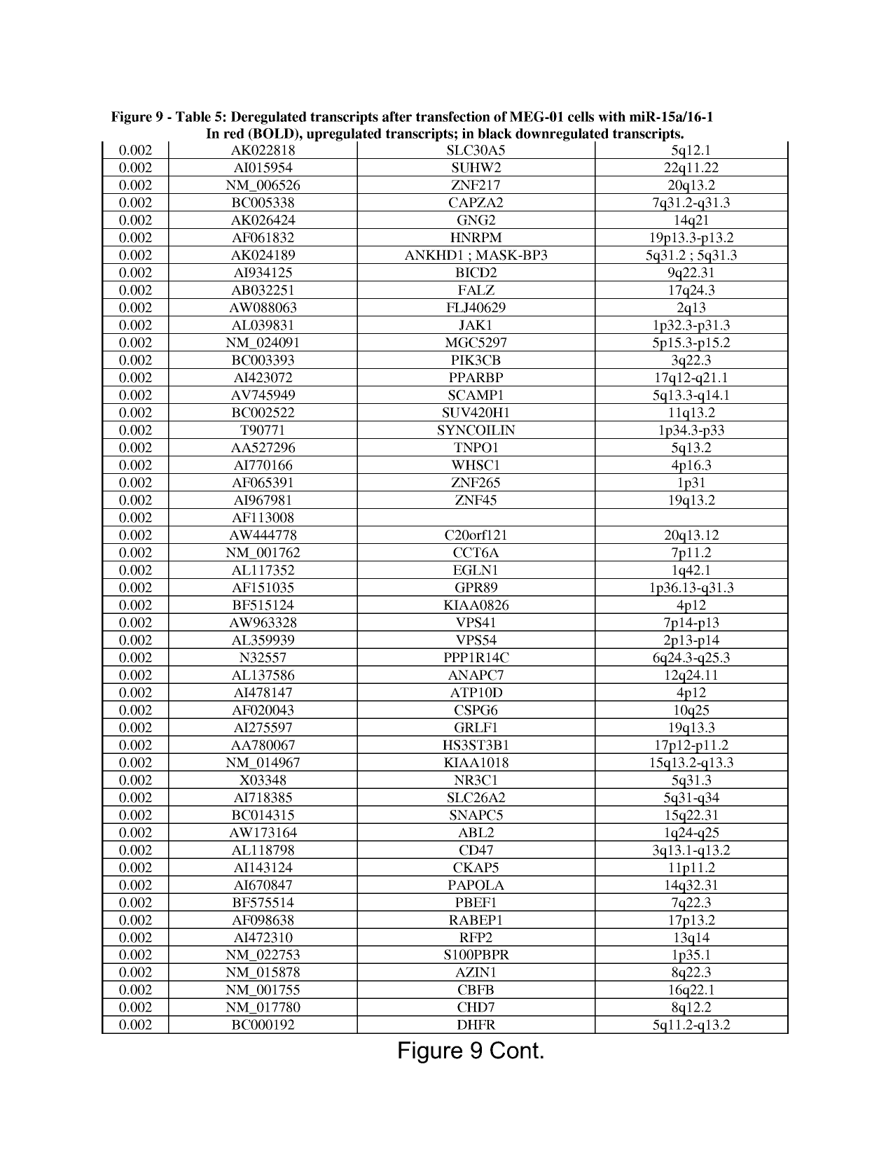

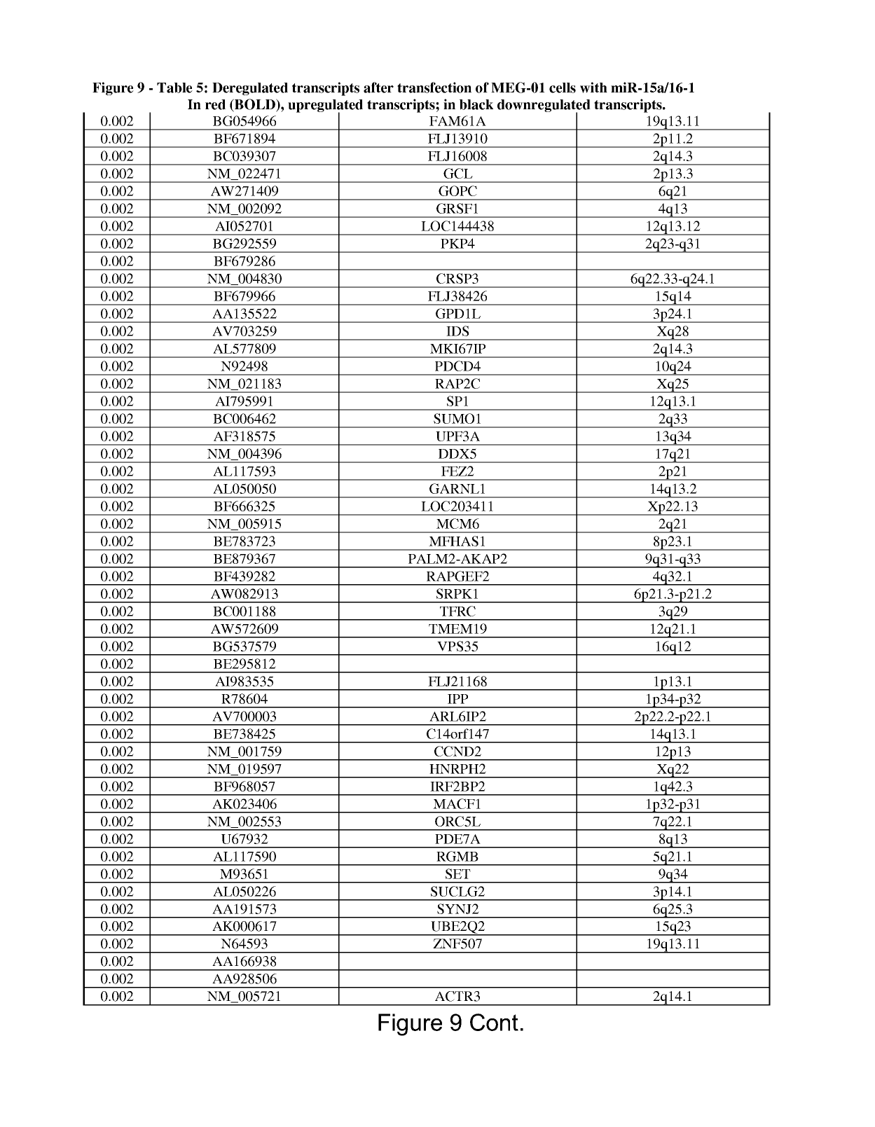

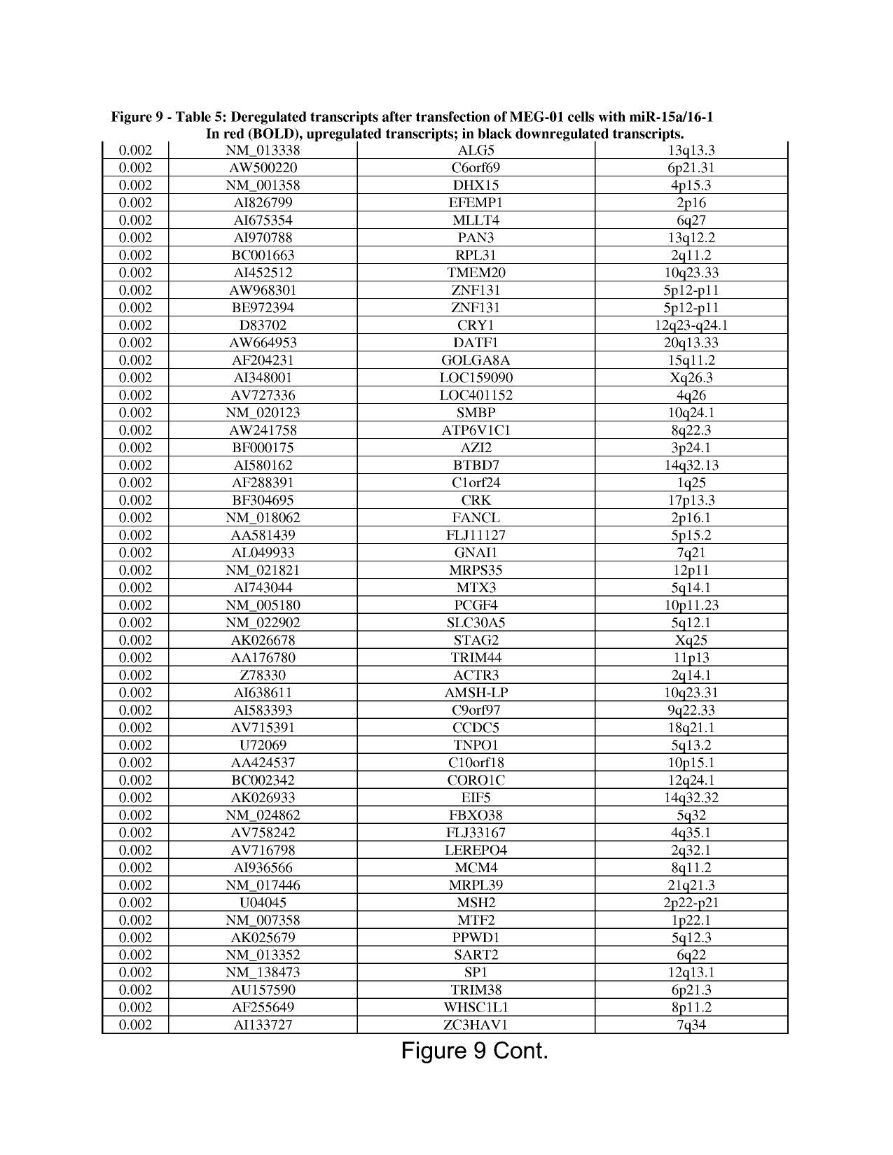

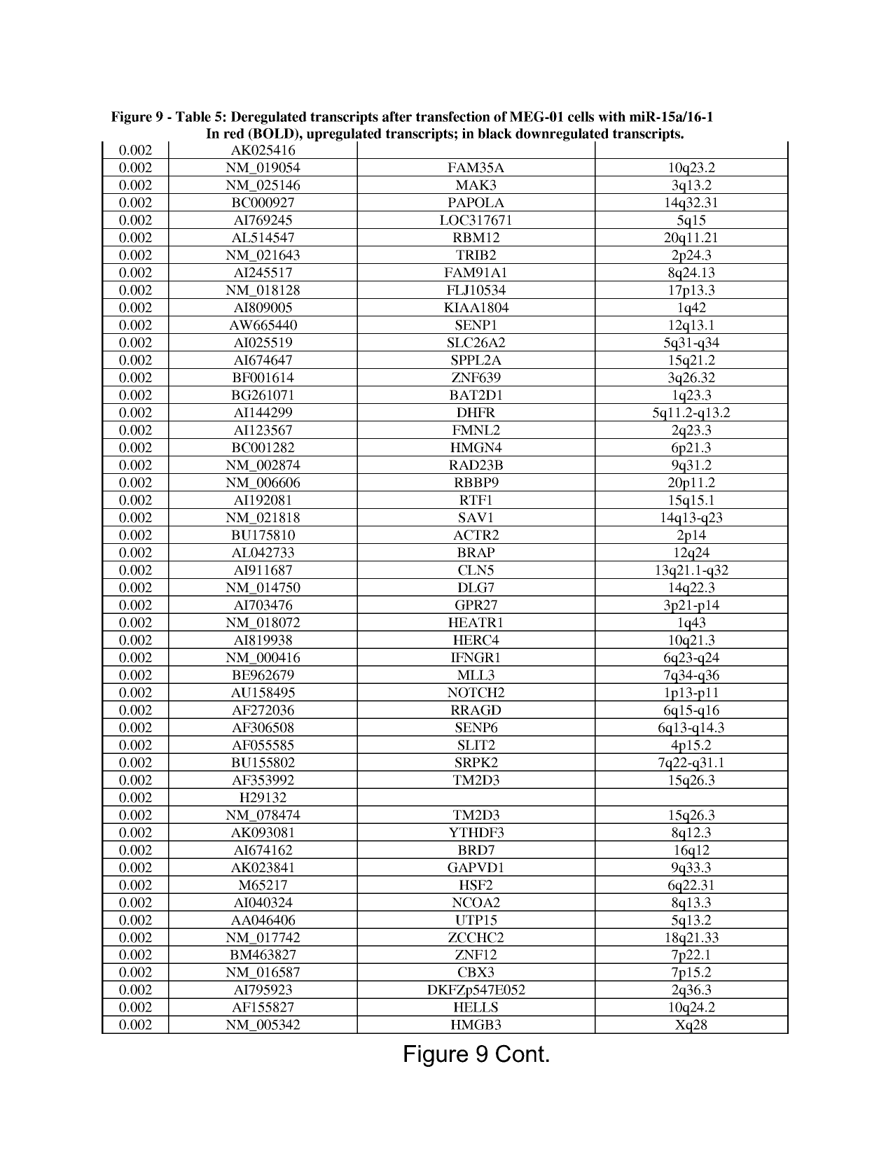

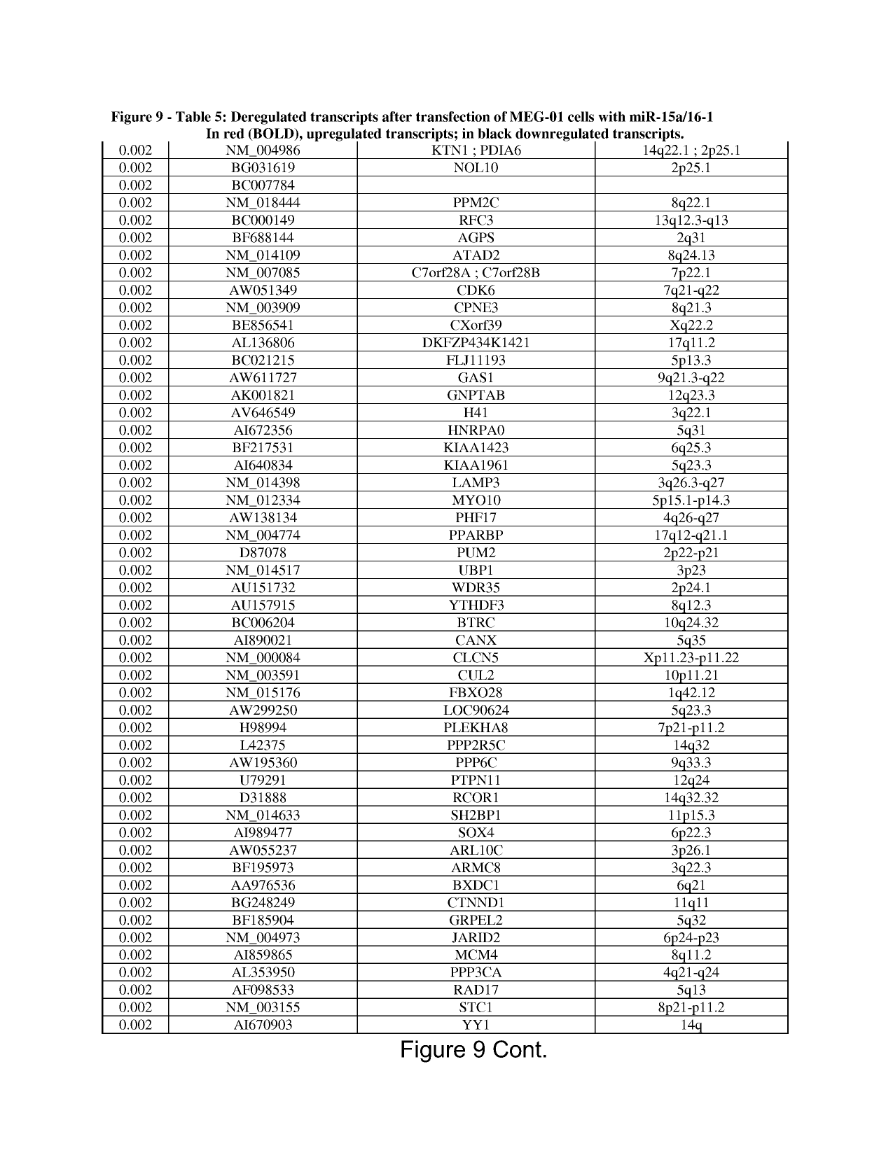

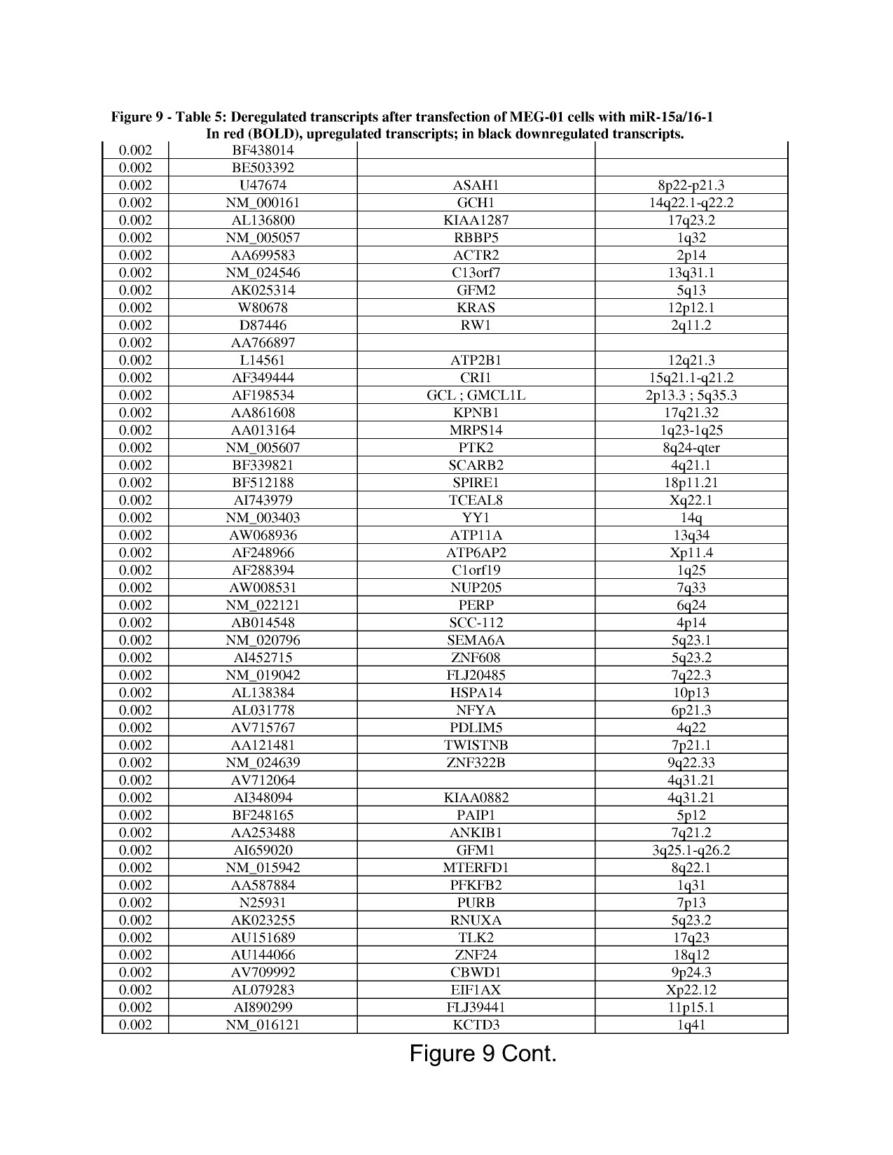

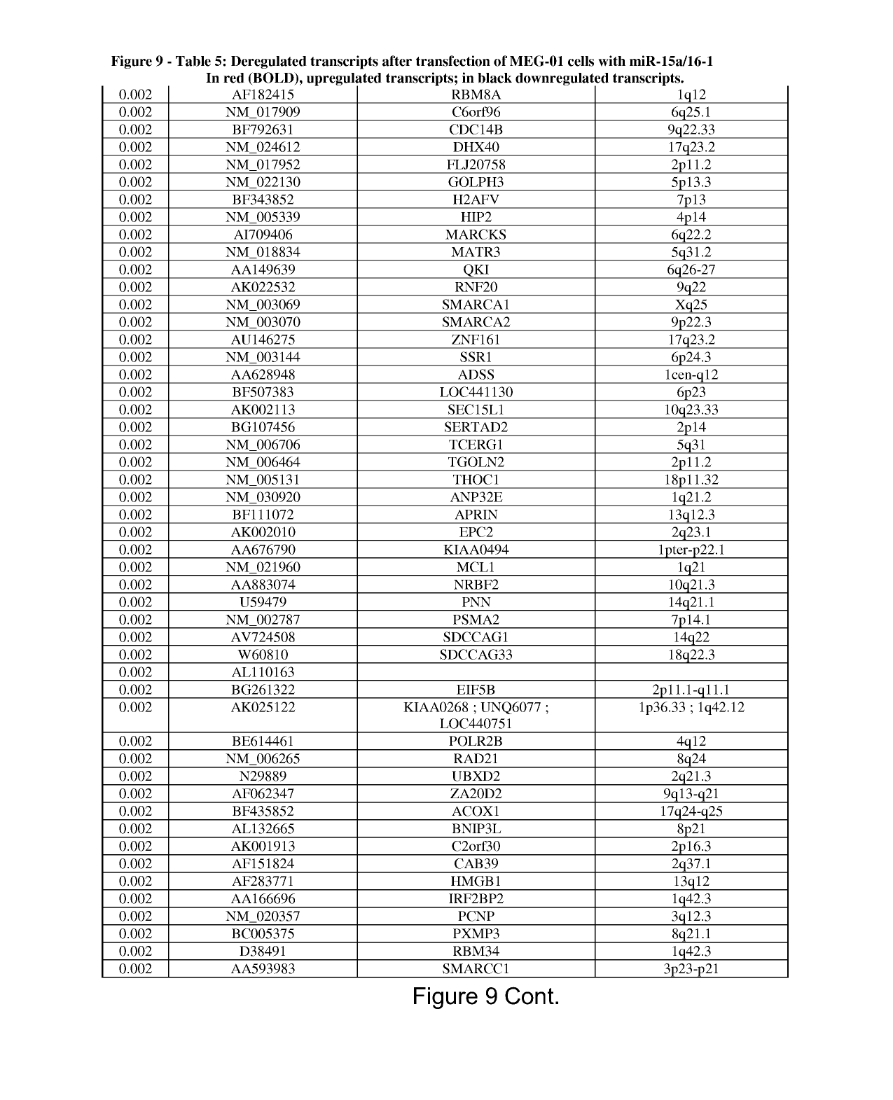

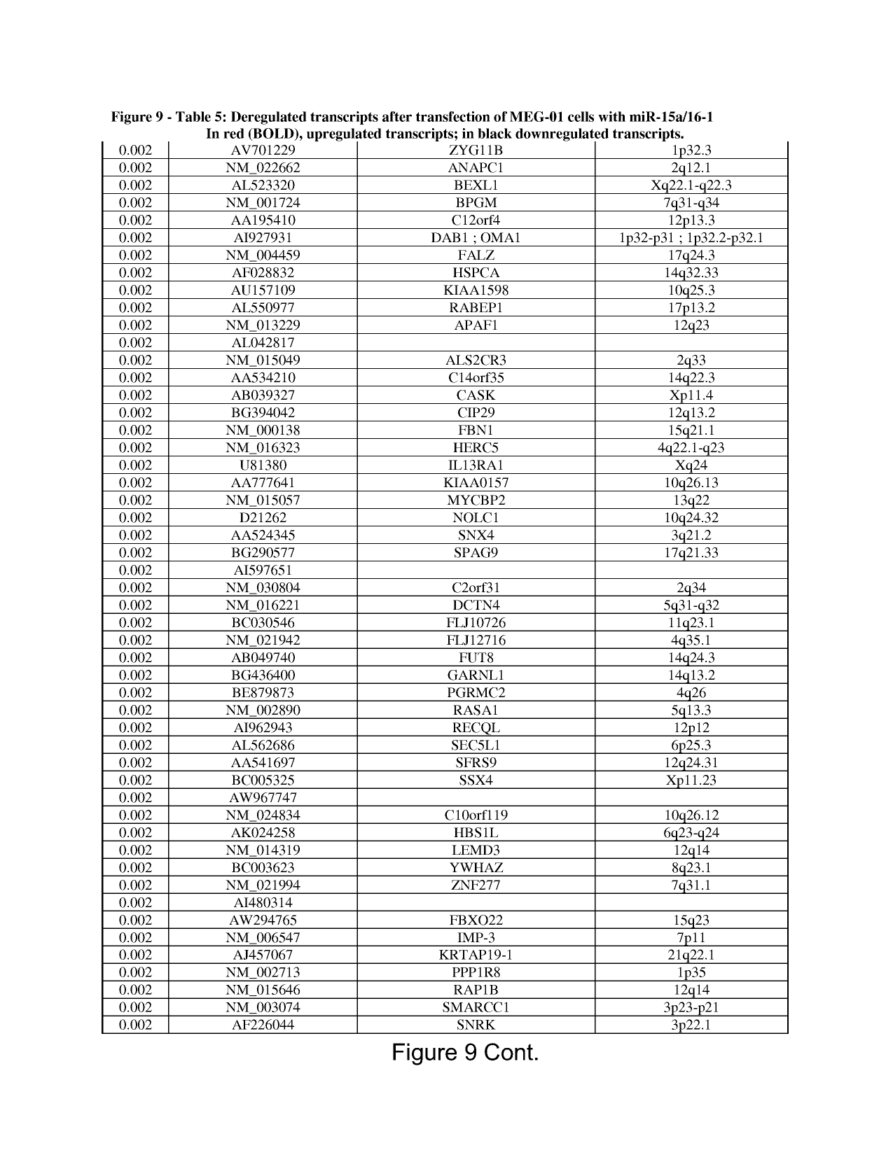

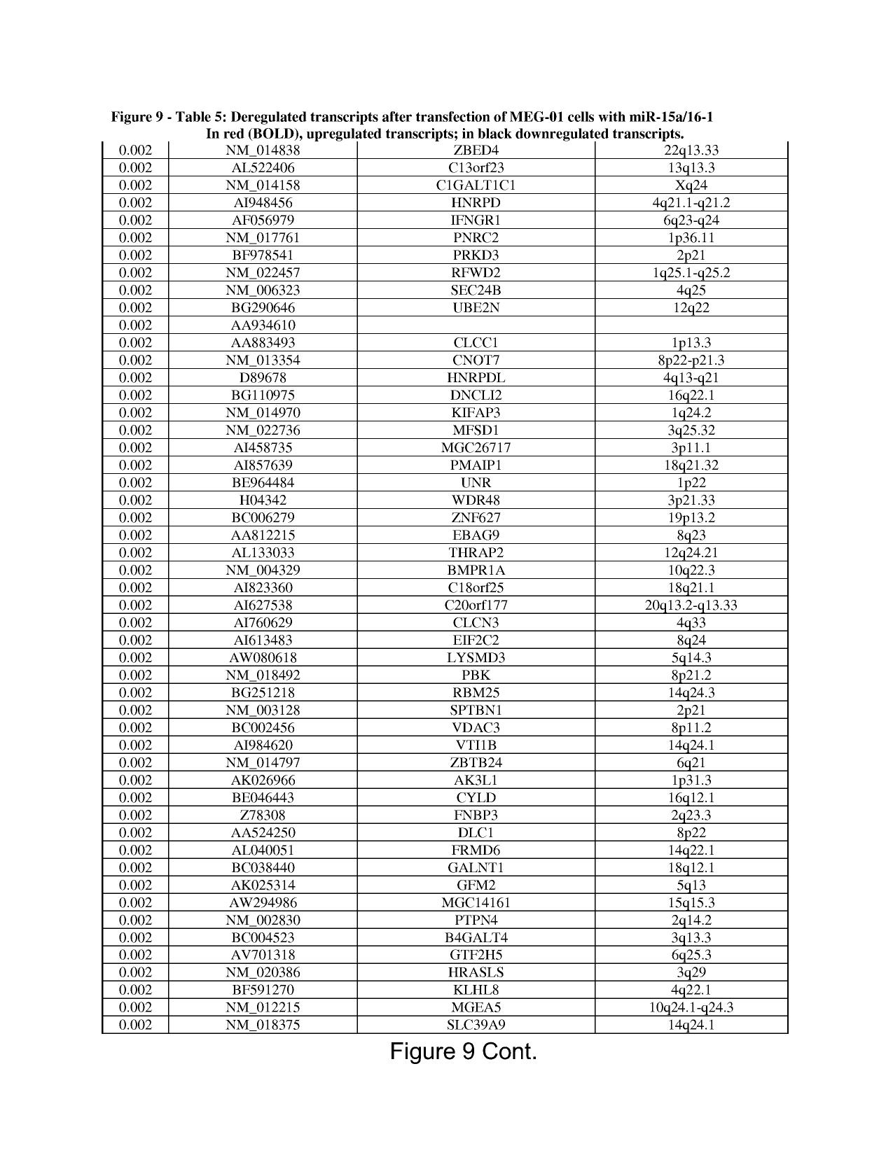

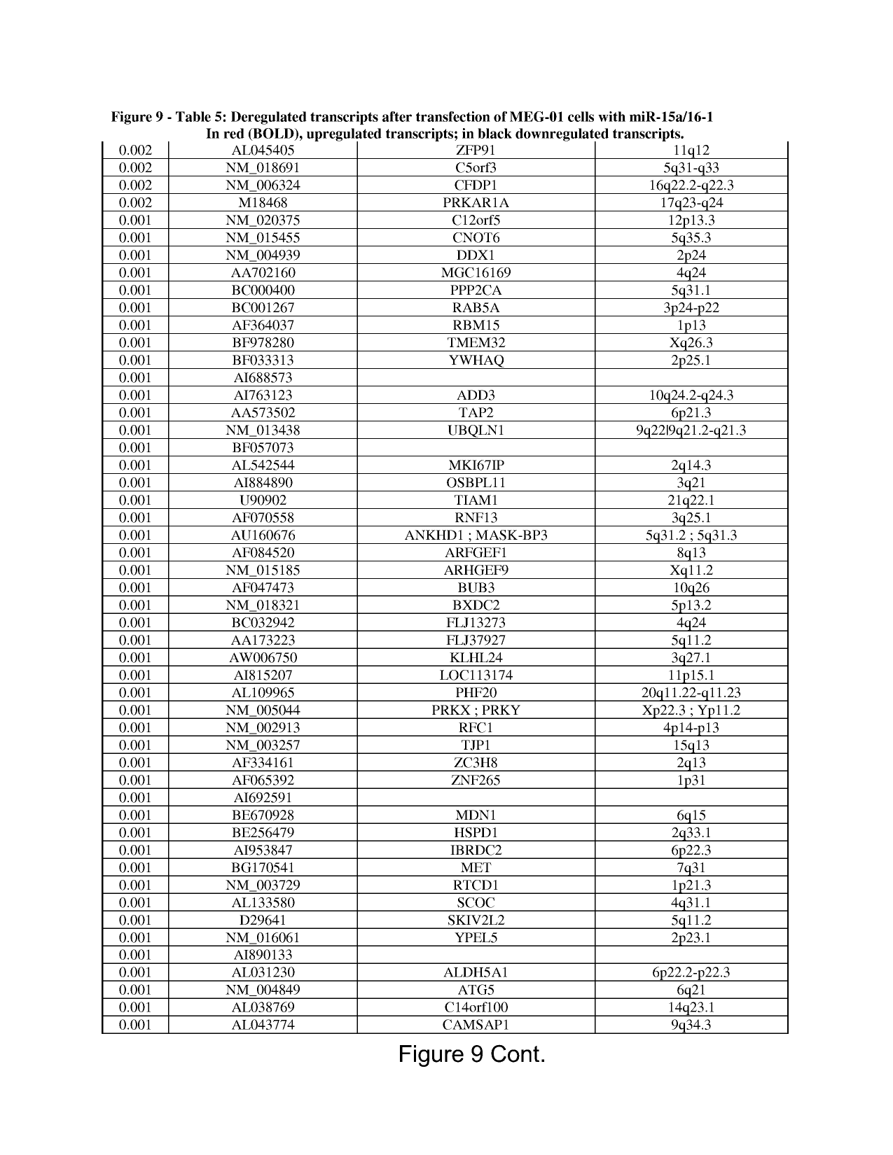

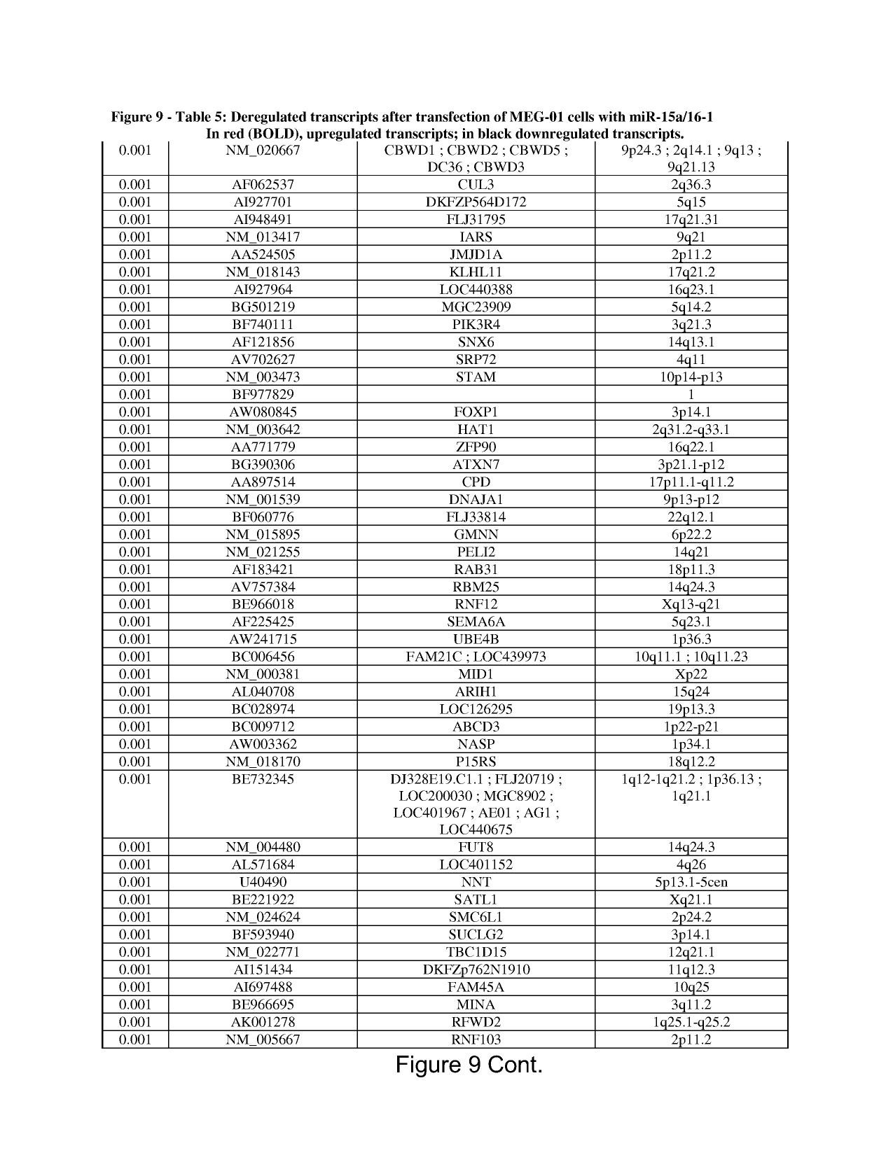

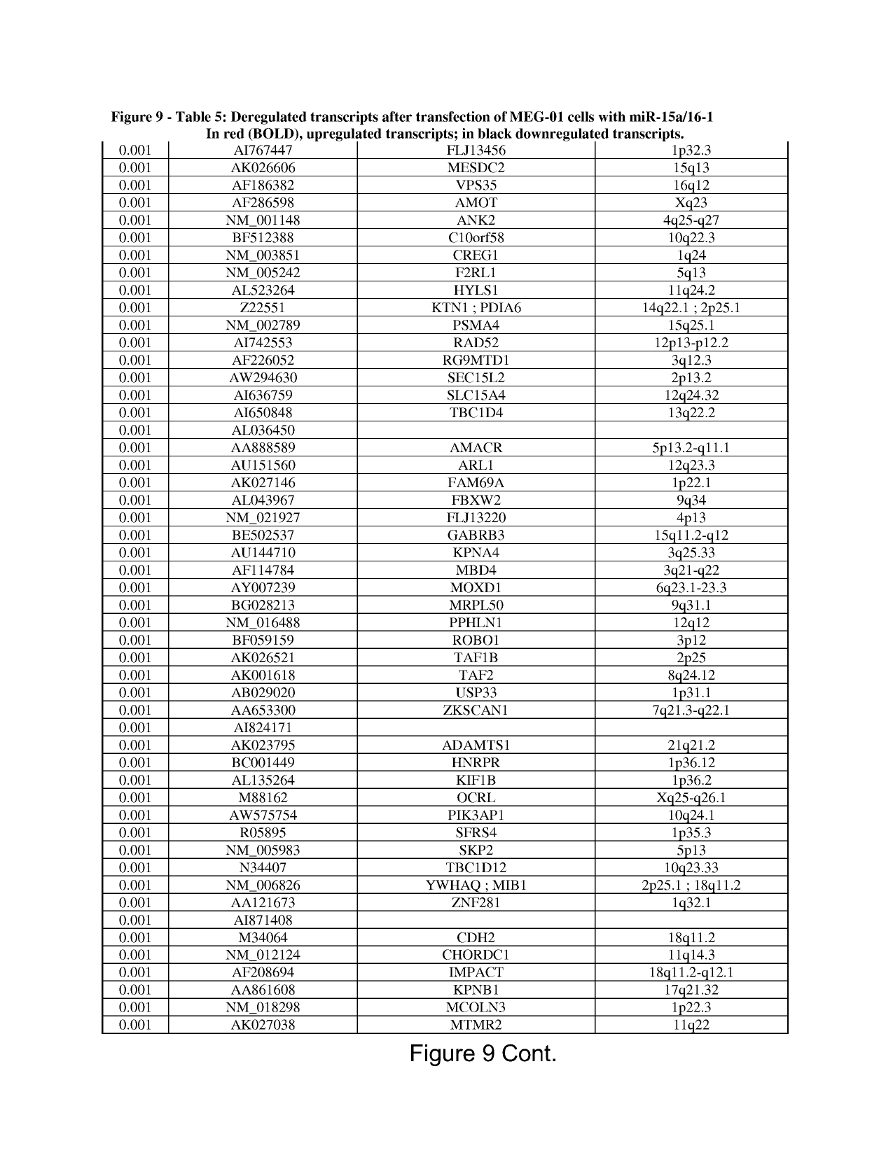

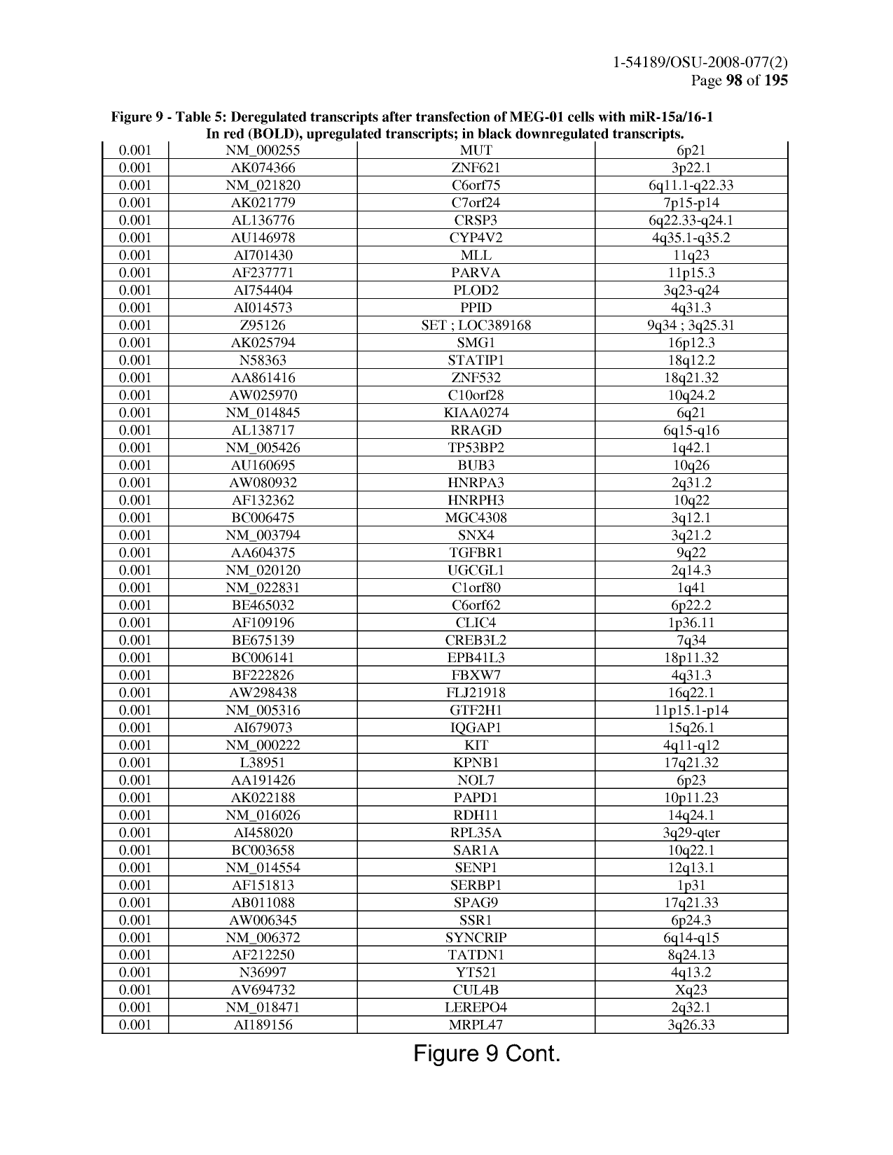

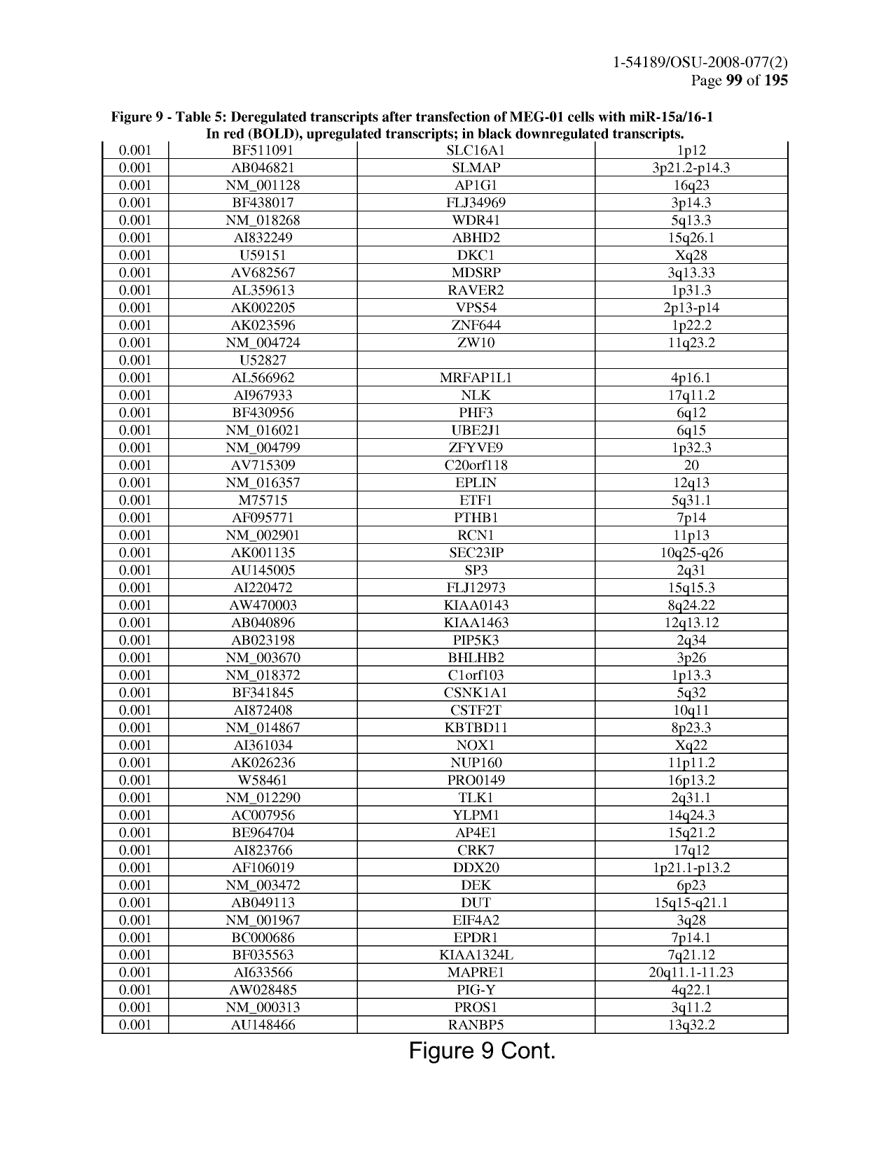

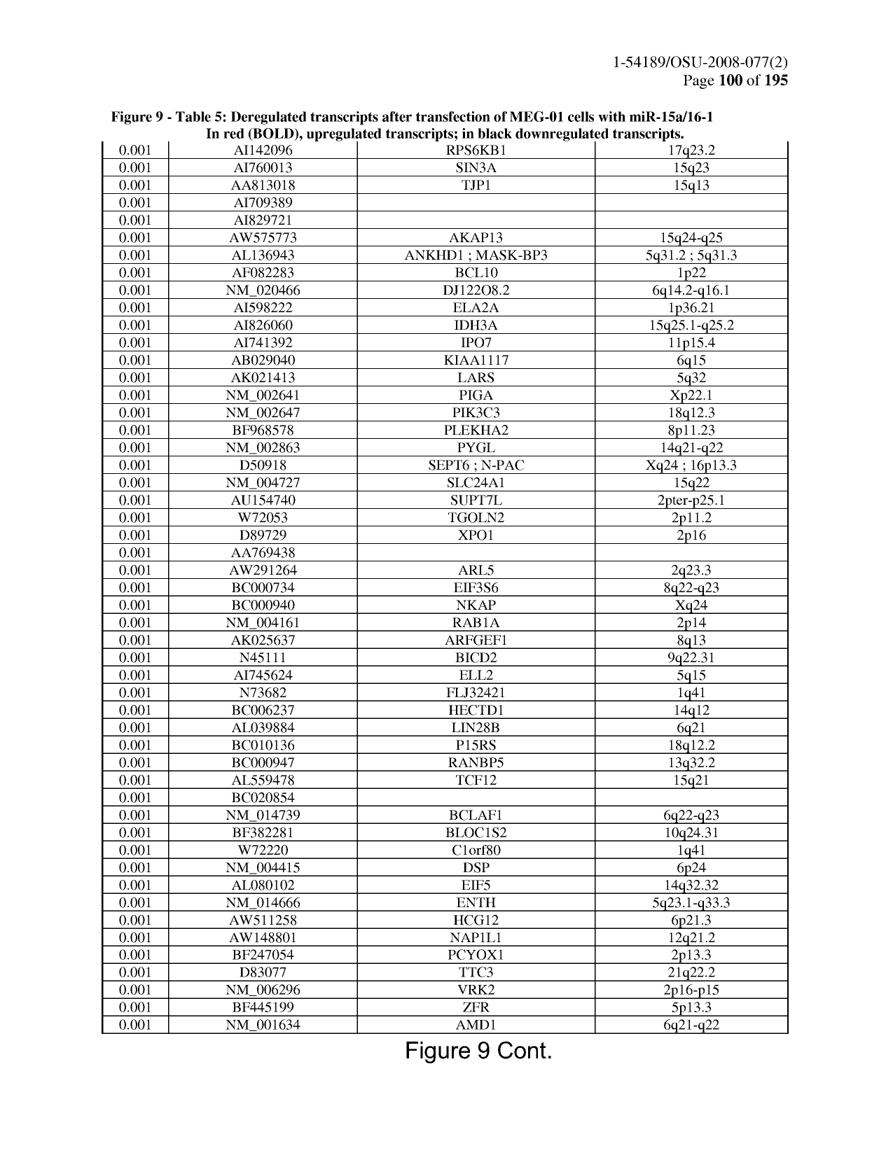

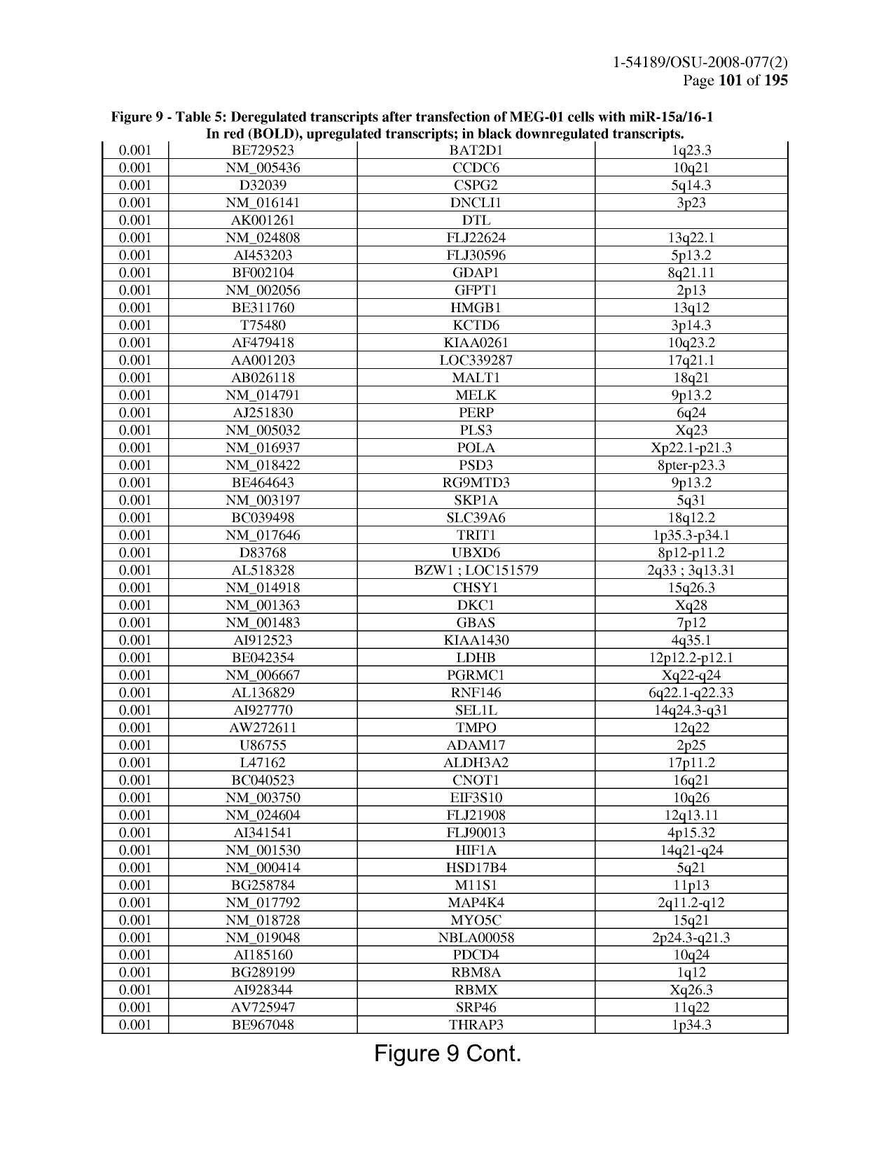

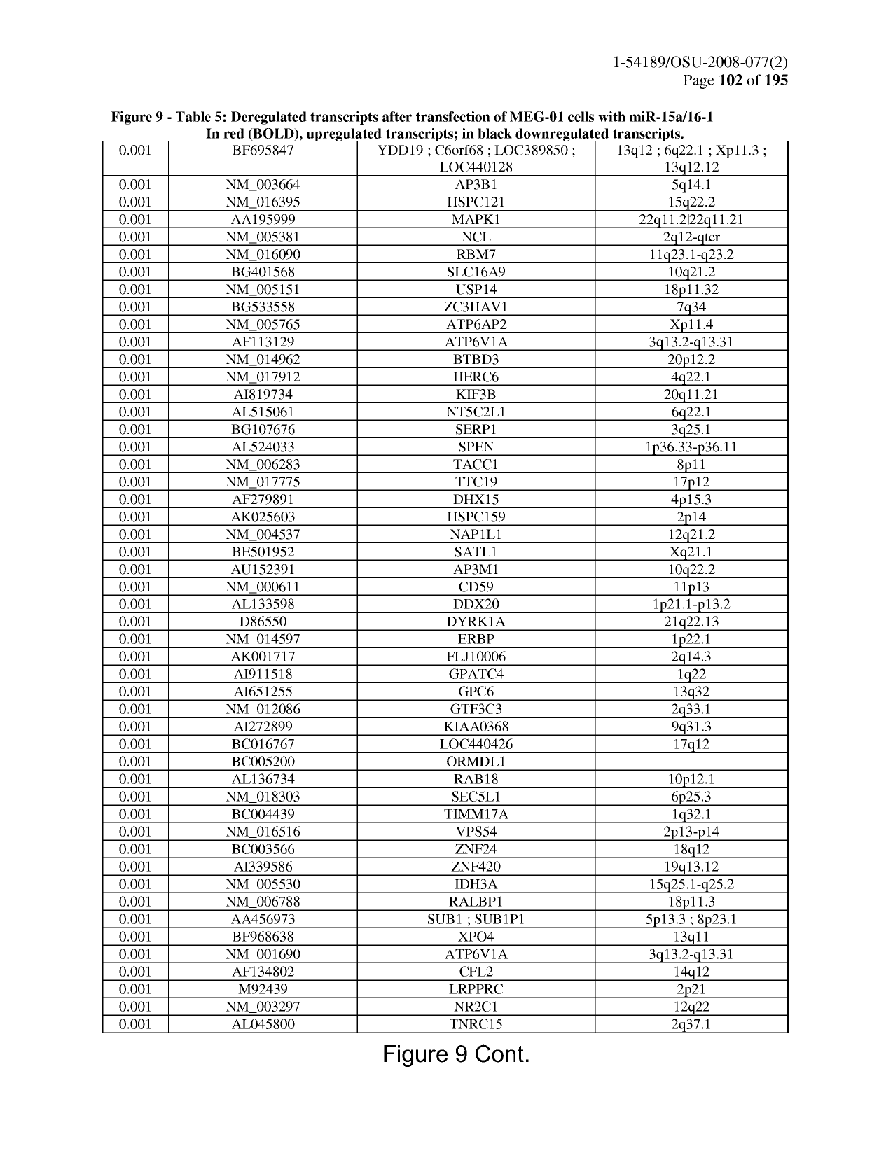

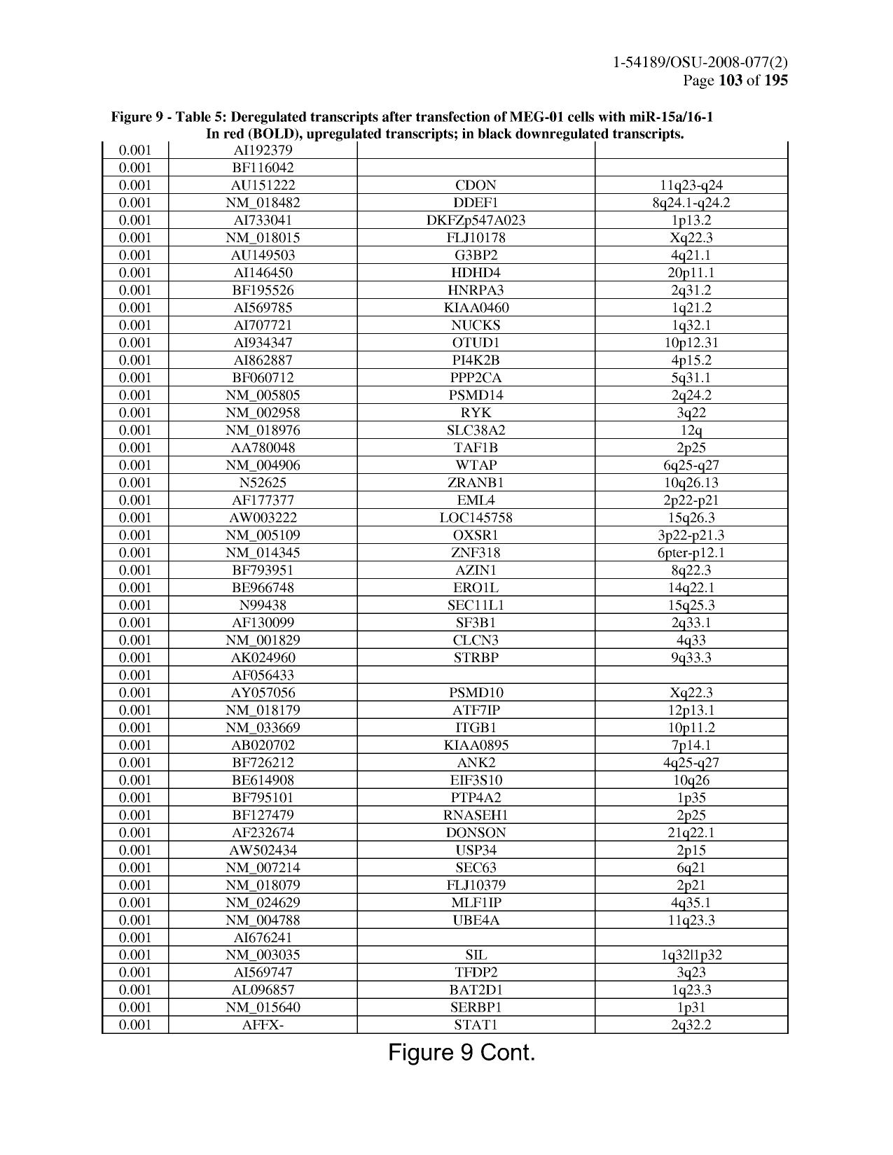

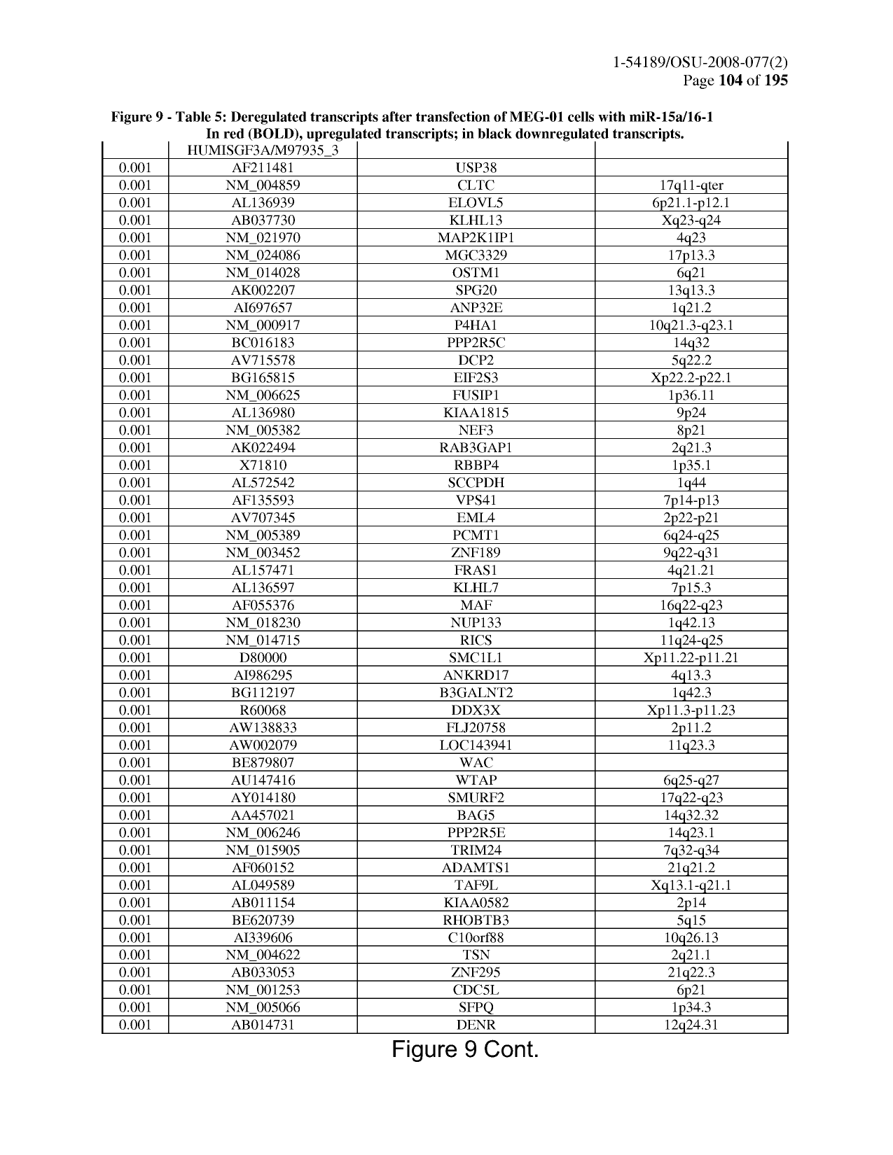

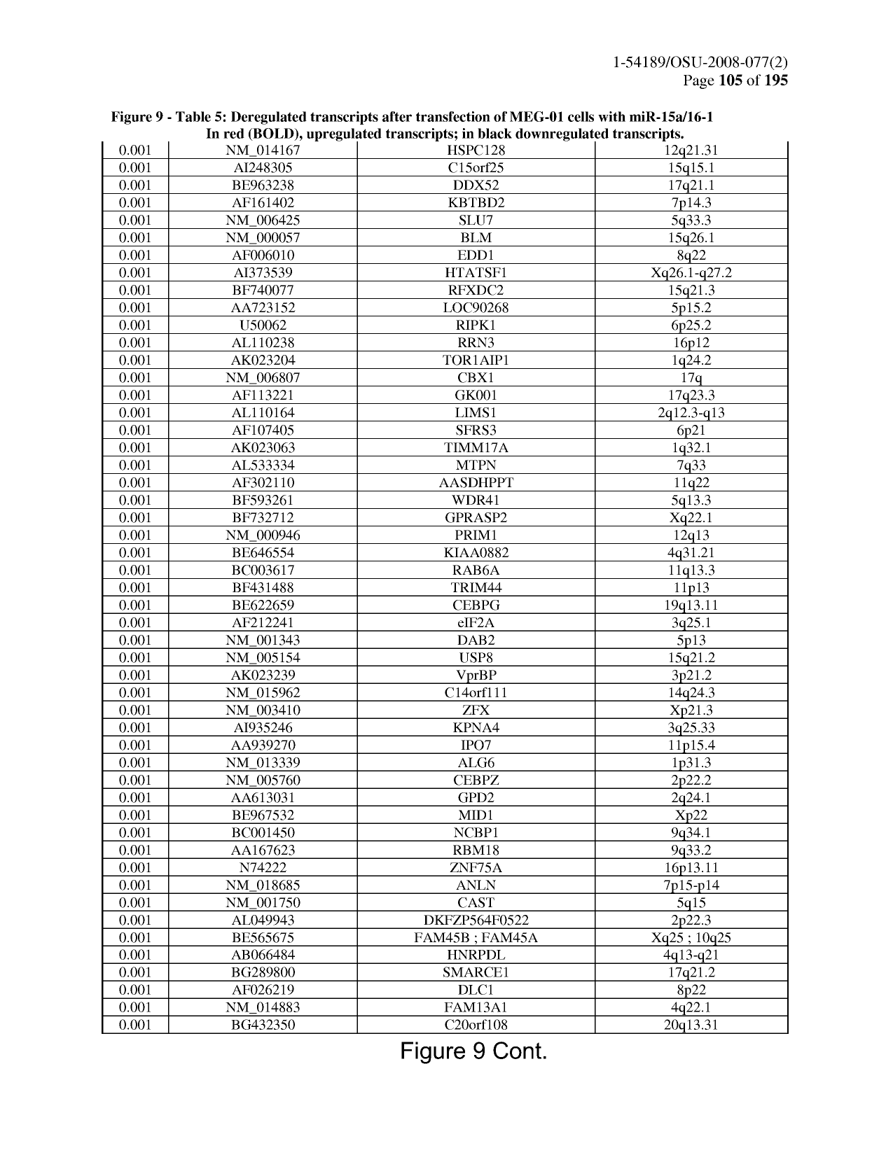

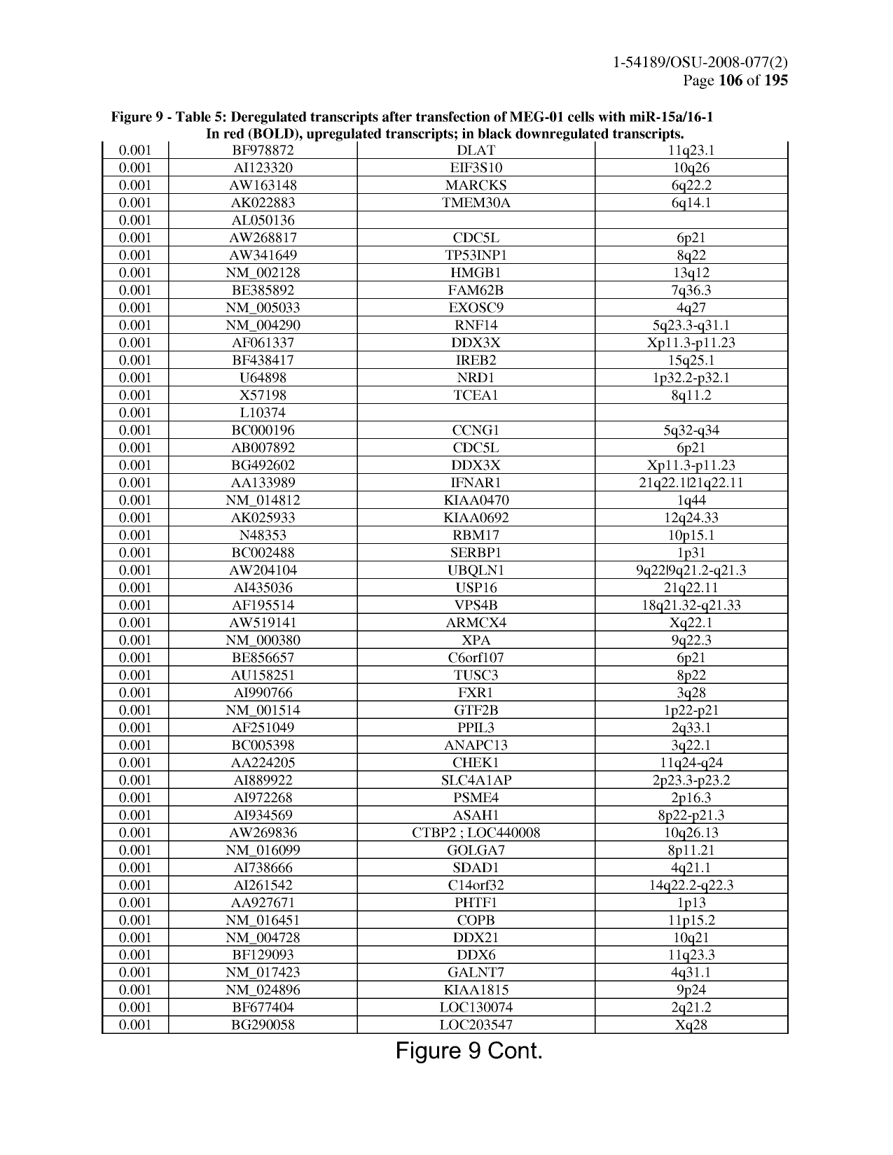

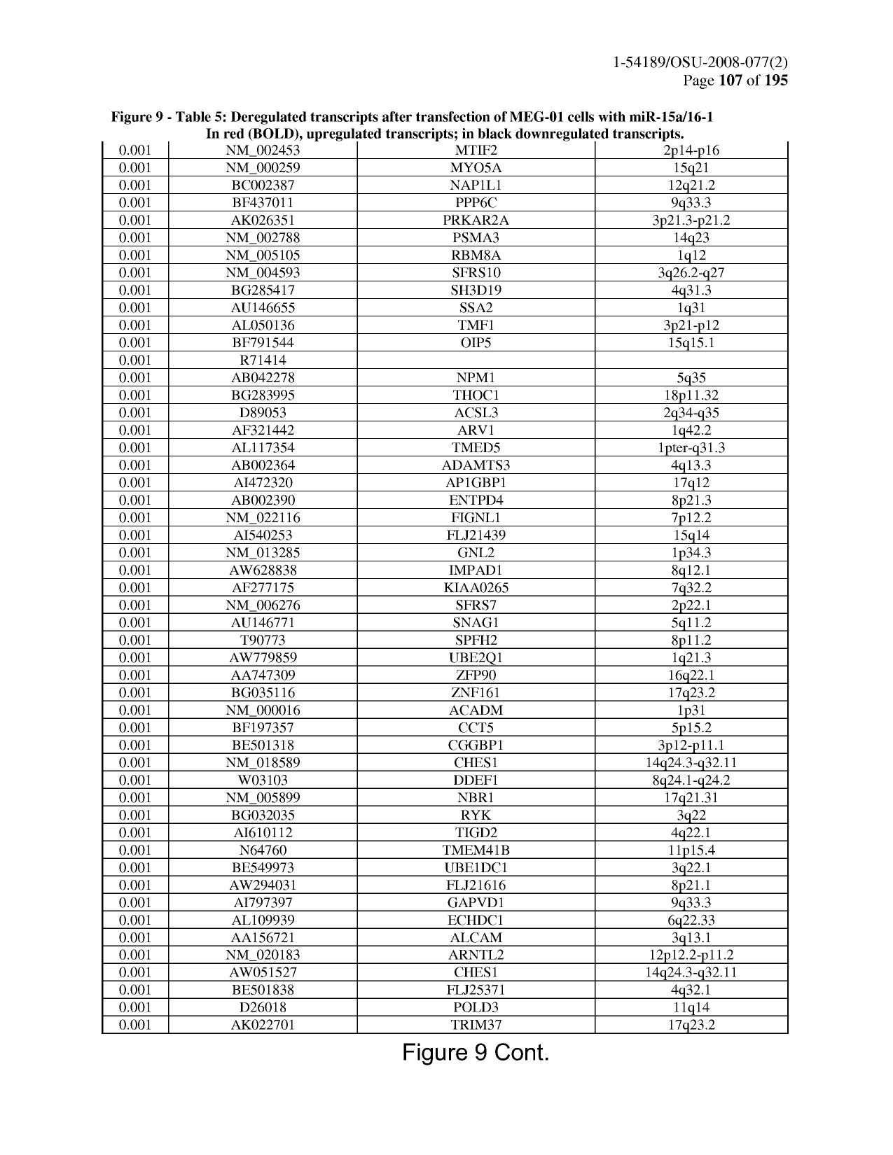

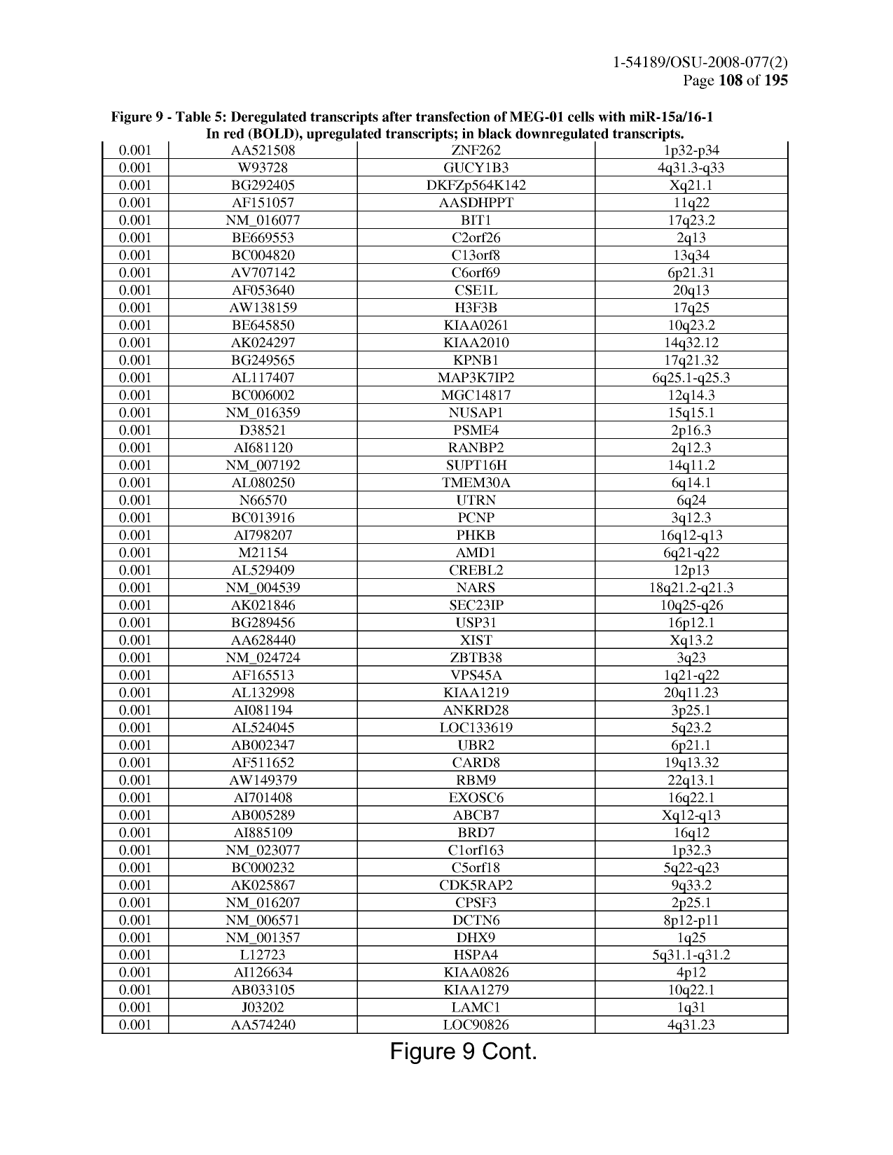

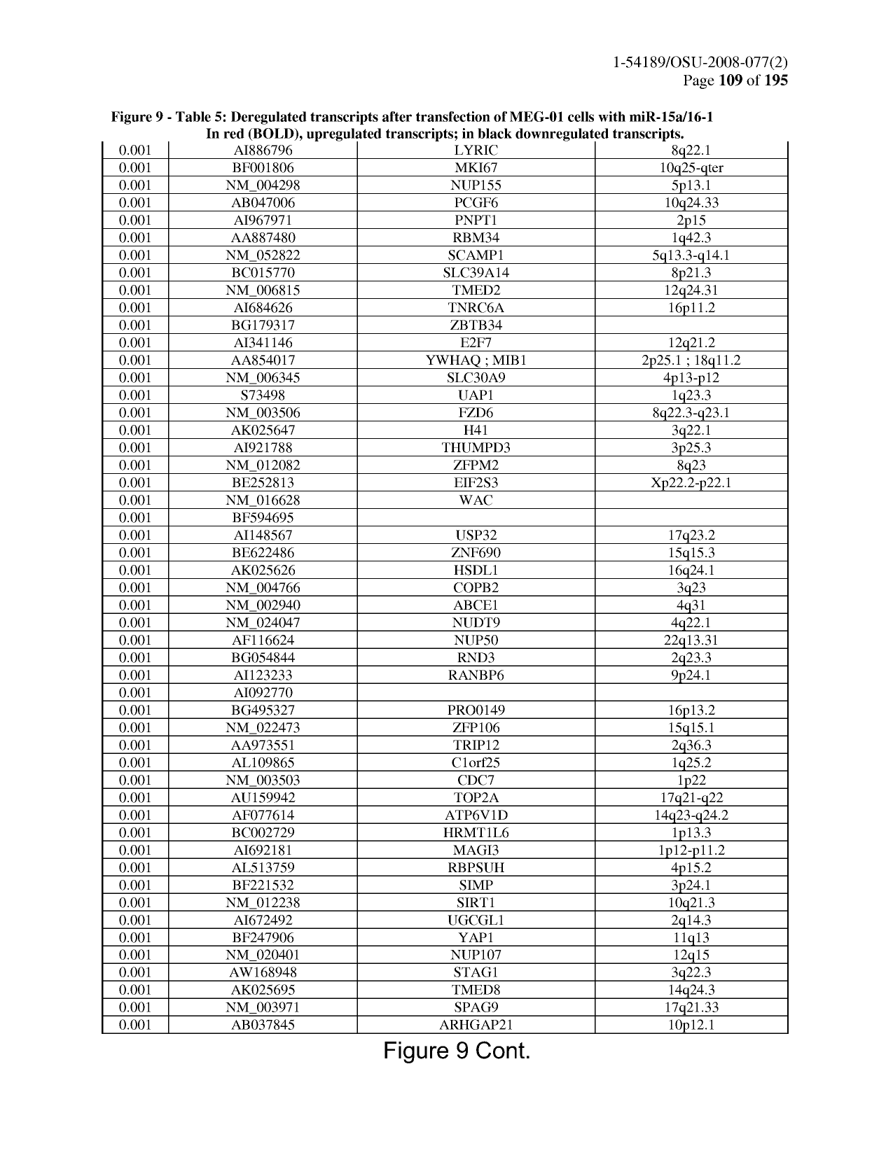

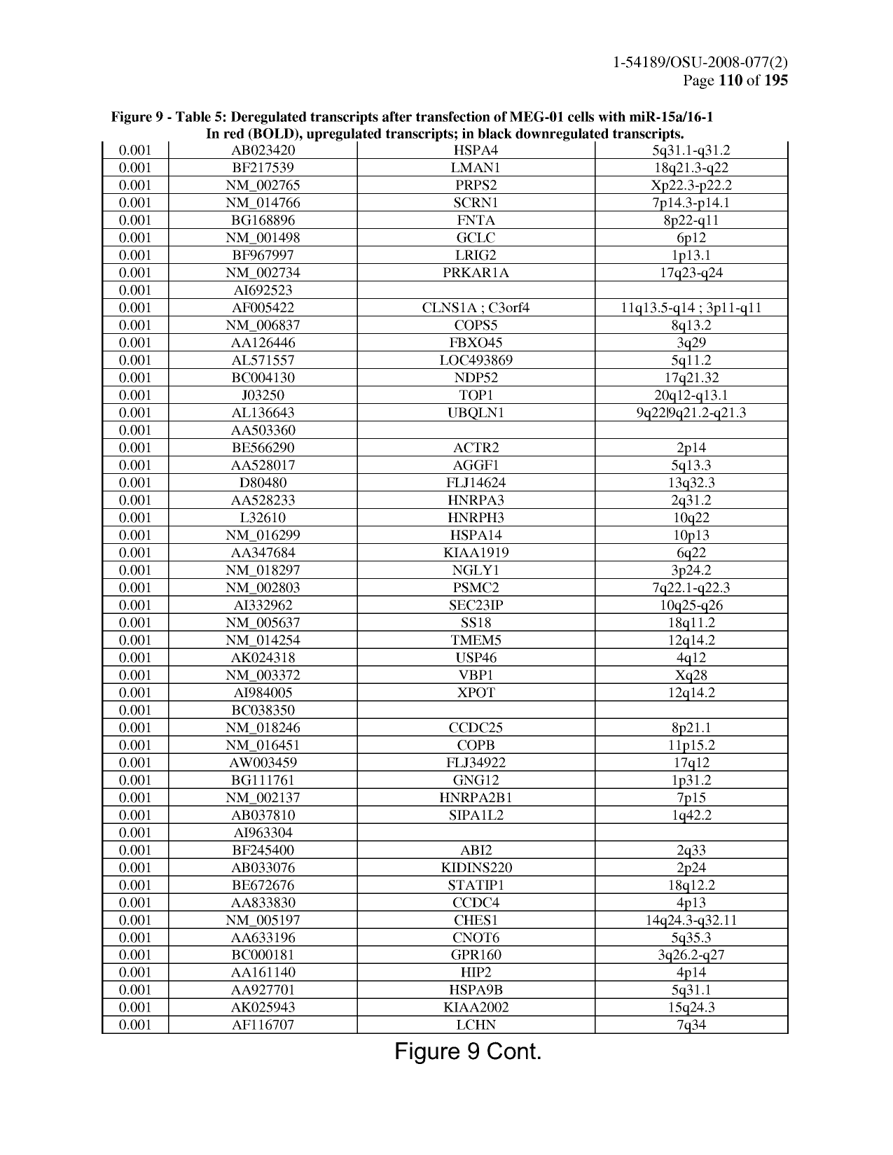

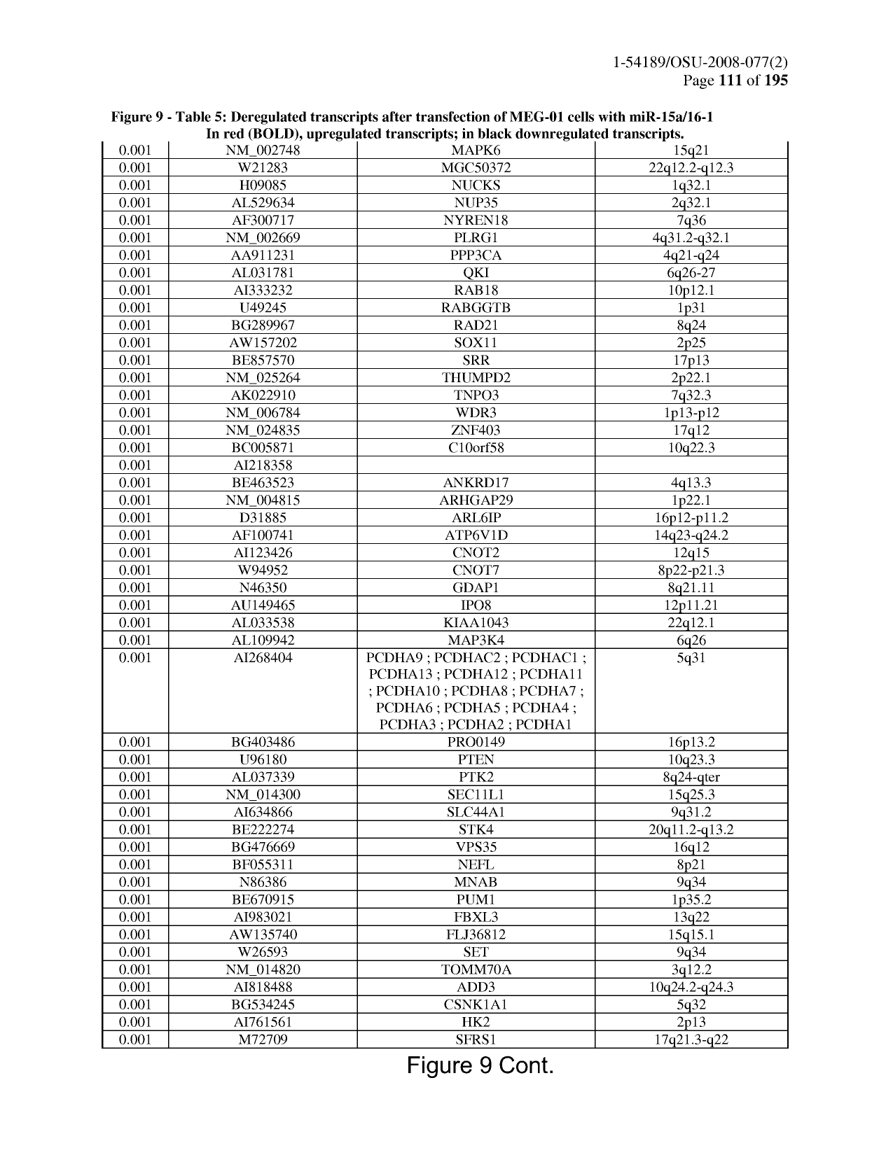

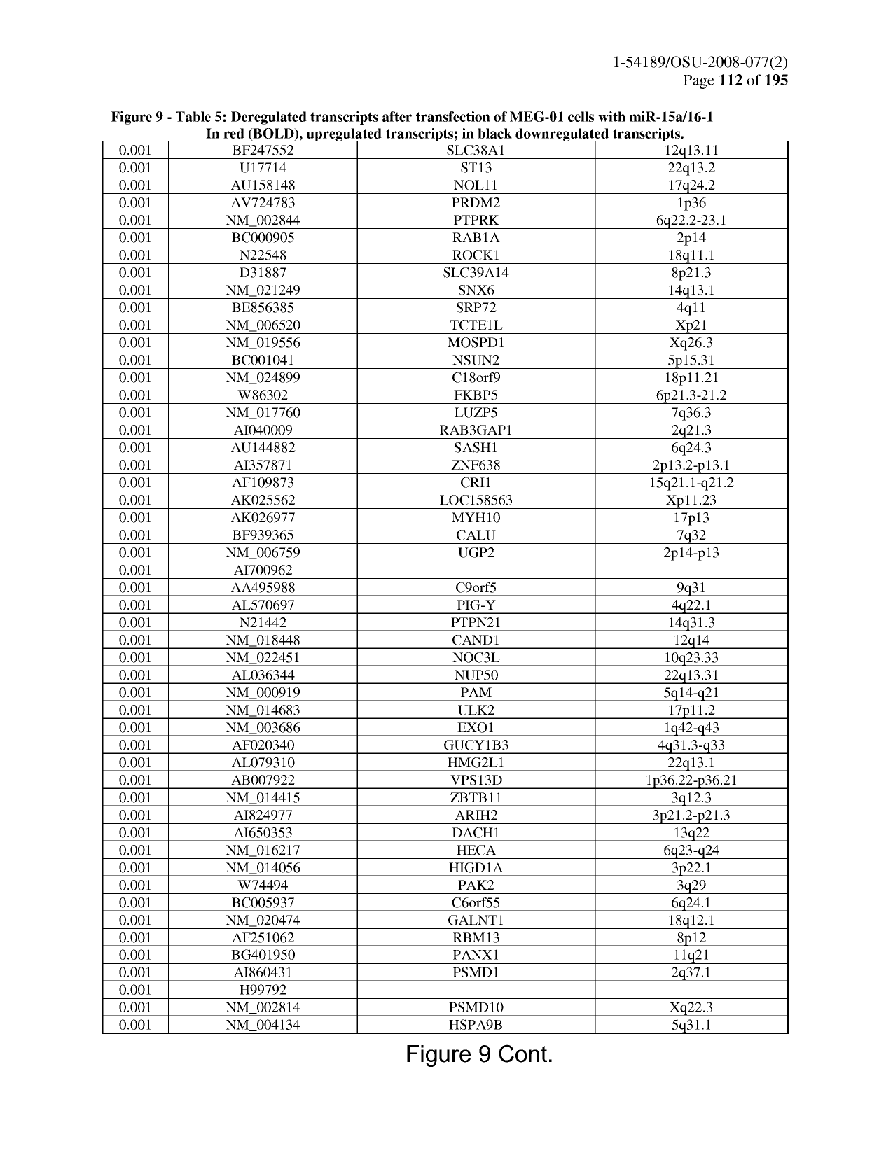

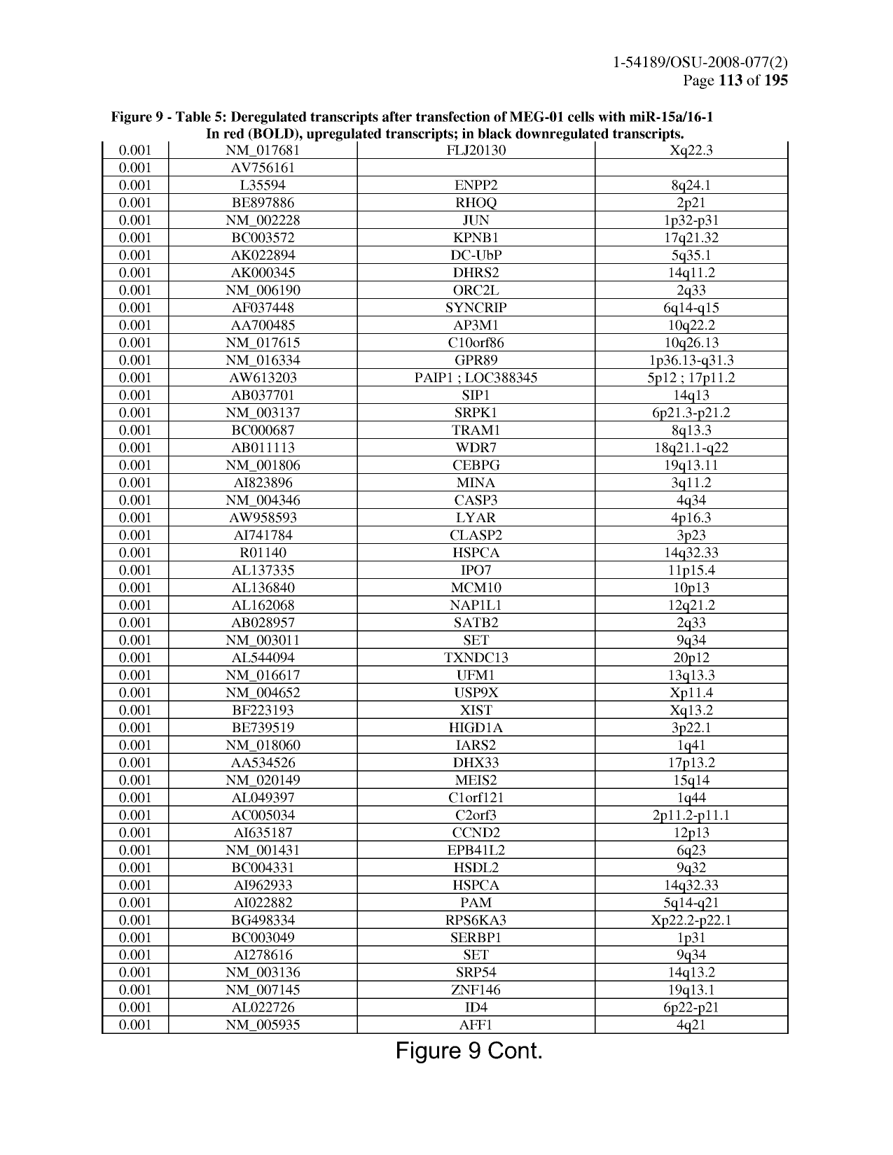

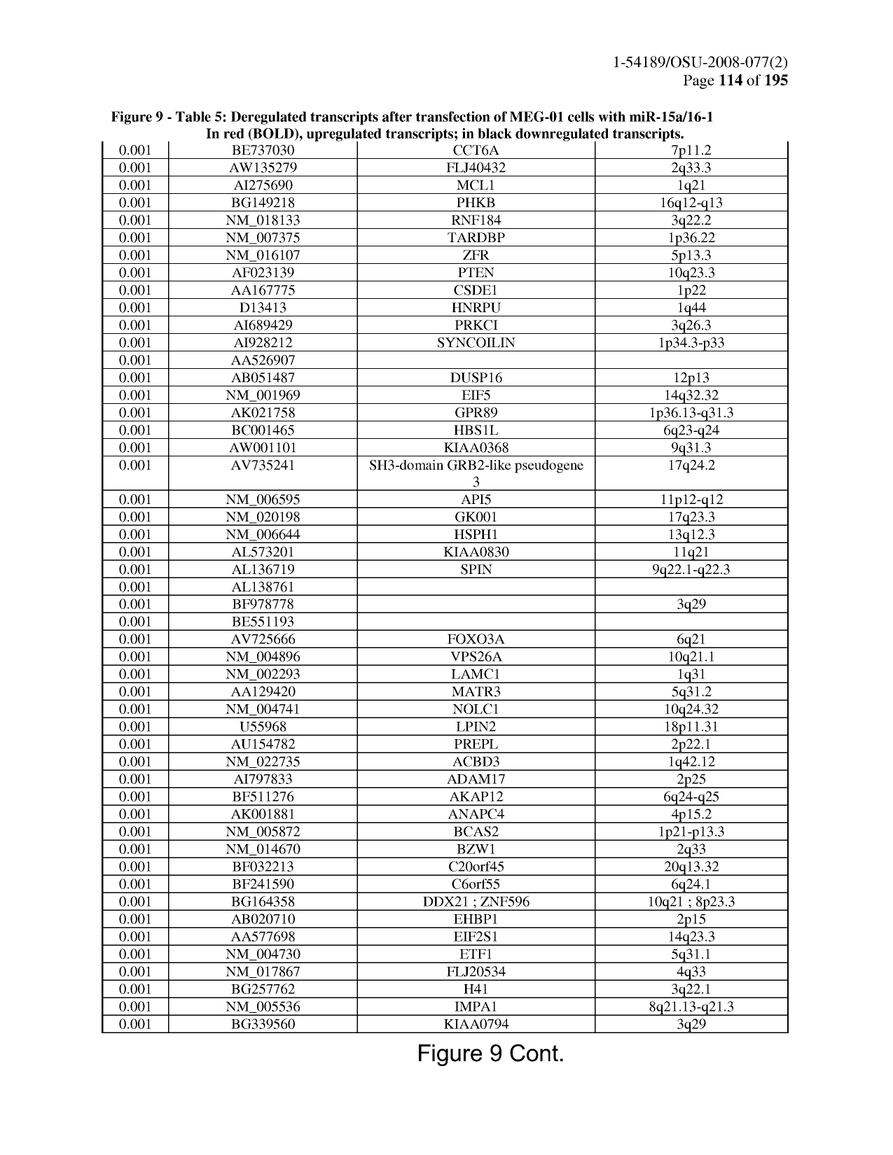

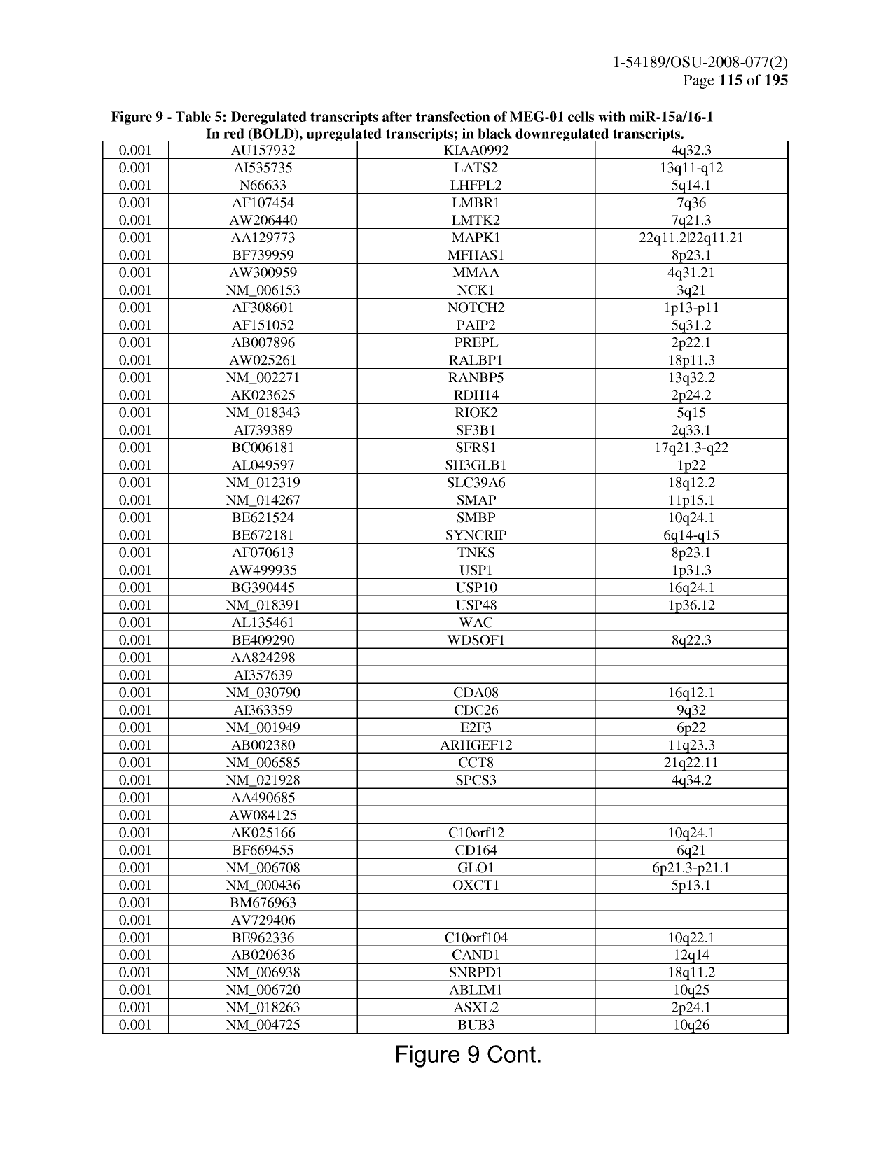

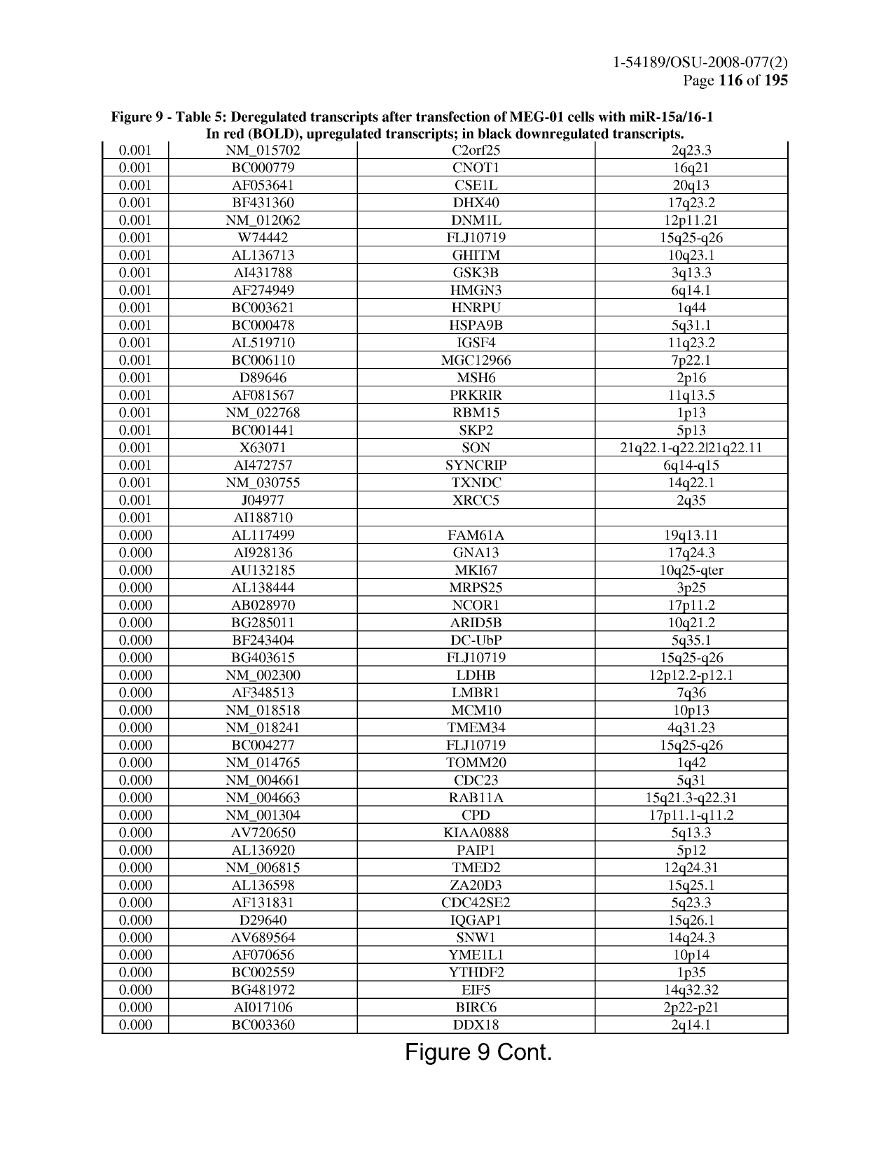

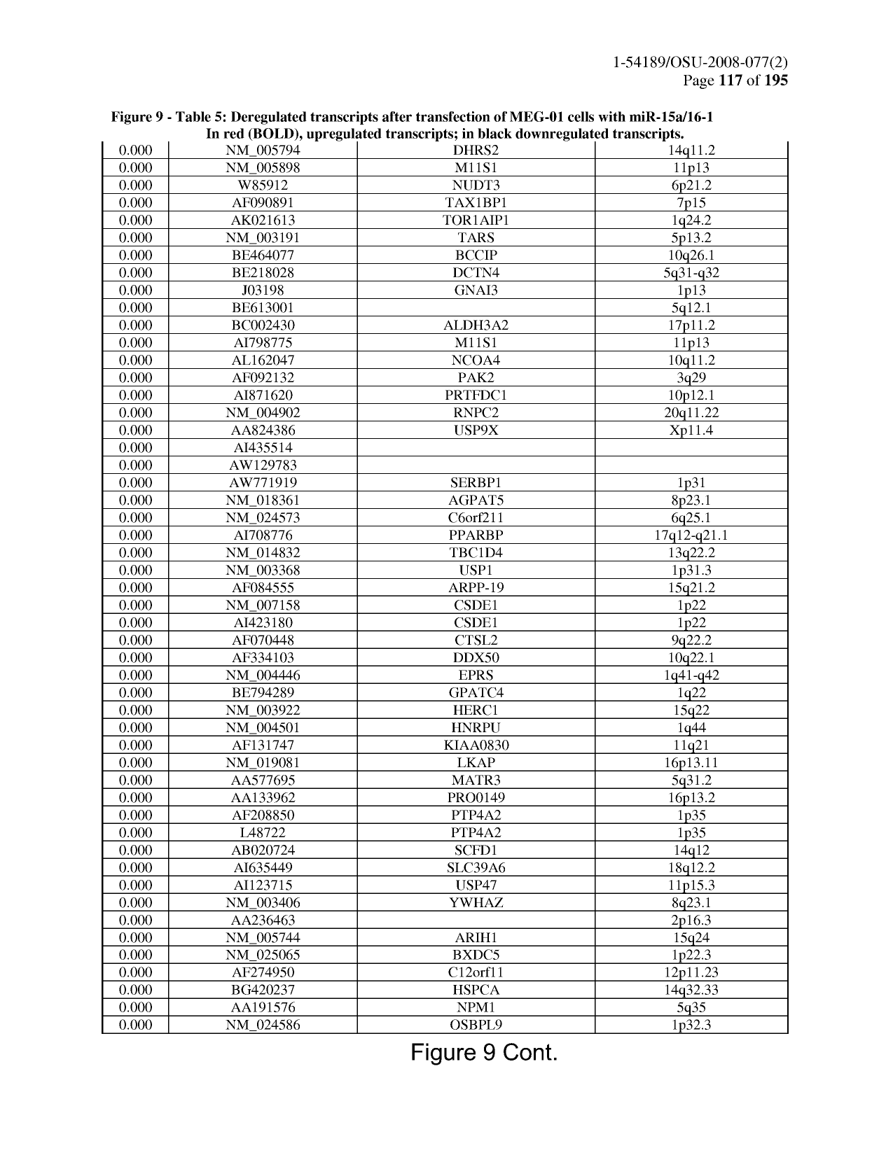

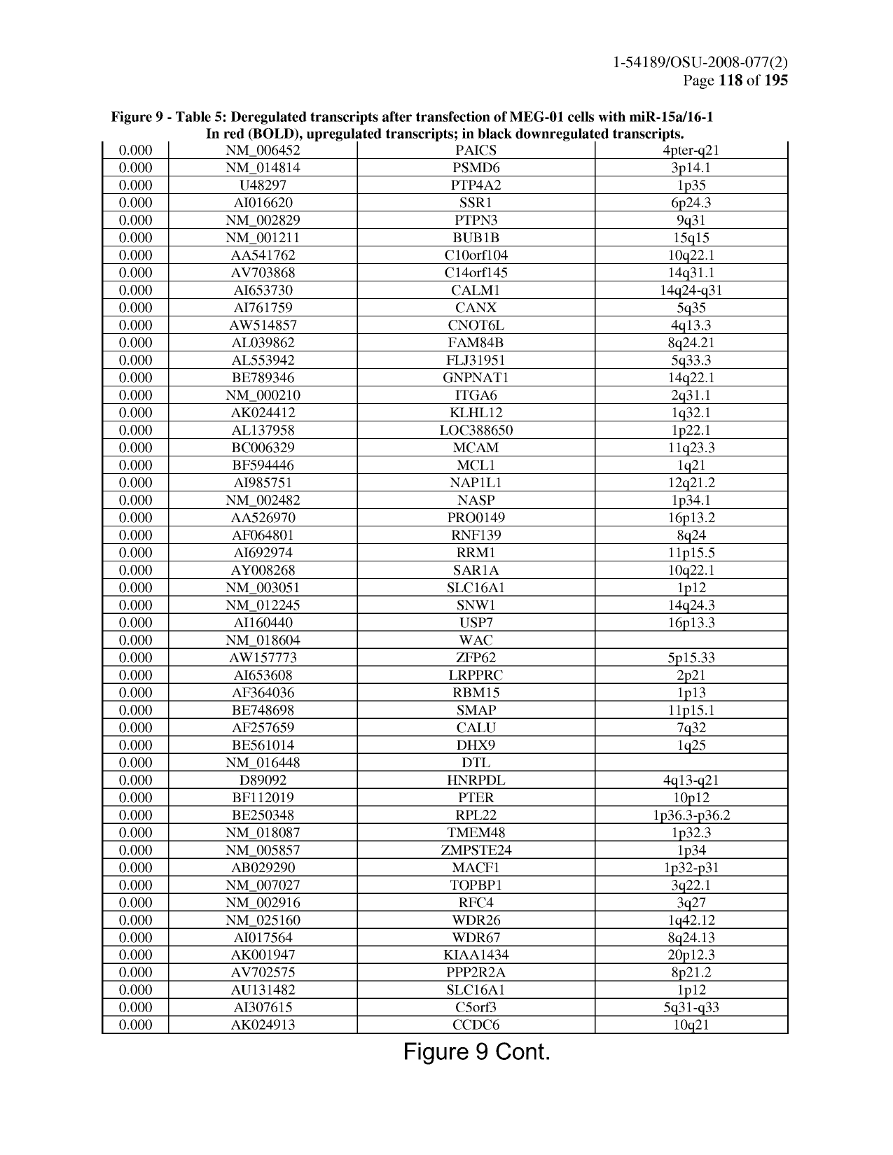

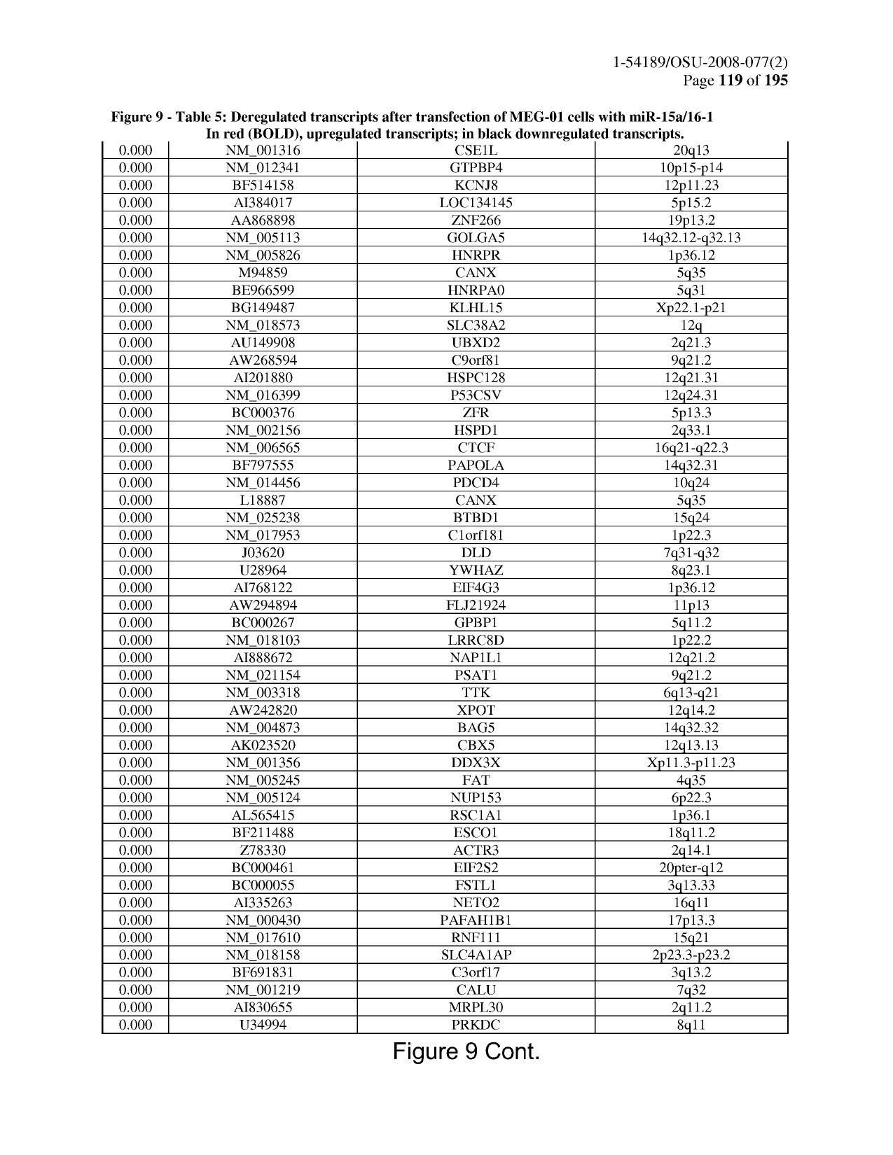

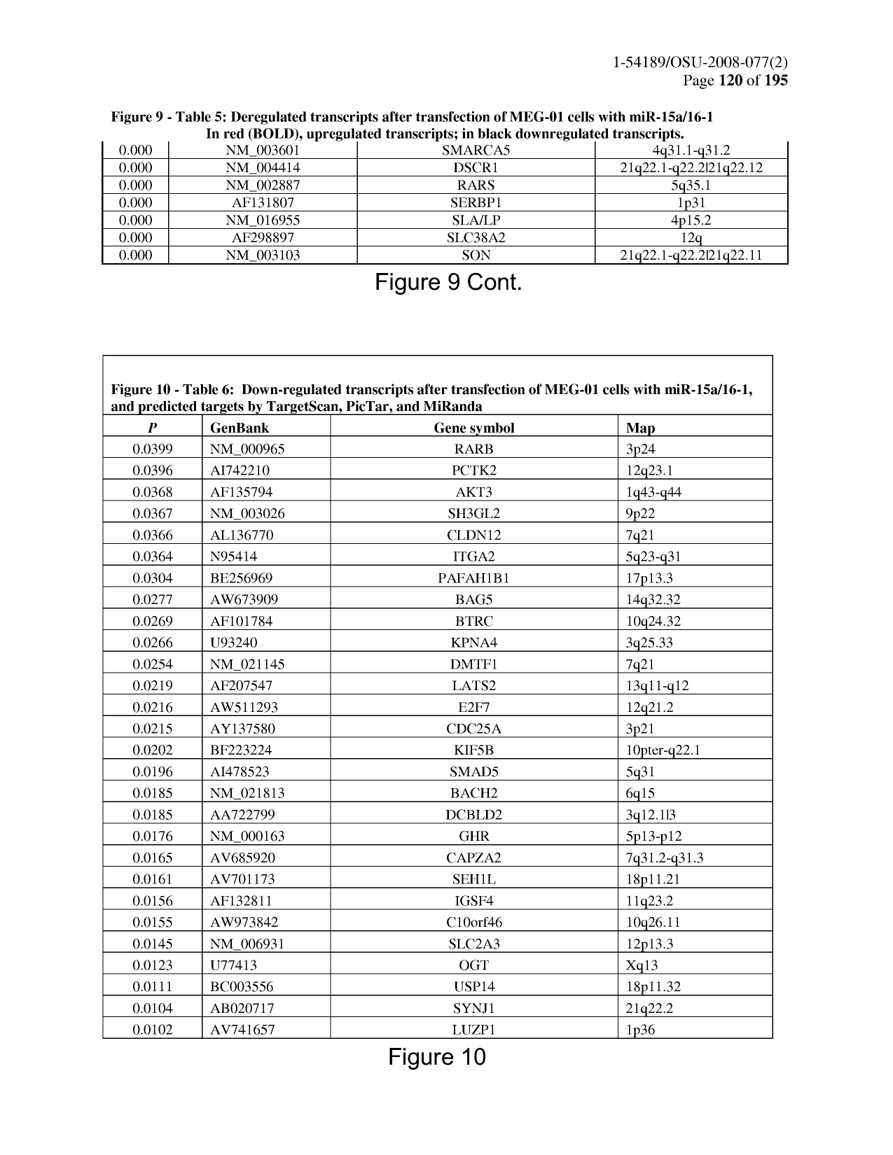

After transfection with miR-15α/16-1 cluster, 355 probes (265 genes) were significantly up-regulated and 5,304 probes (3,307 genes) down-regulated (FIG. 9—Table S5).

The cluster analysis, performed with the differentially expressed genes, shows a clearly distinct gene expression profile between pRS15/16- and pRS-E-transfected cells (FIG. 3).

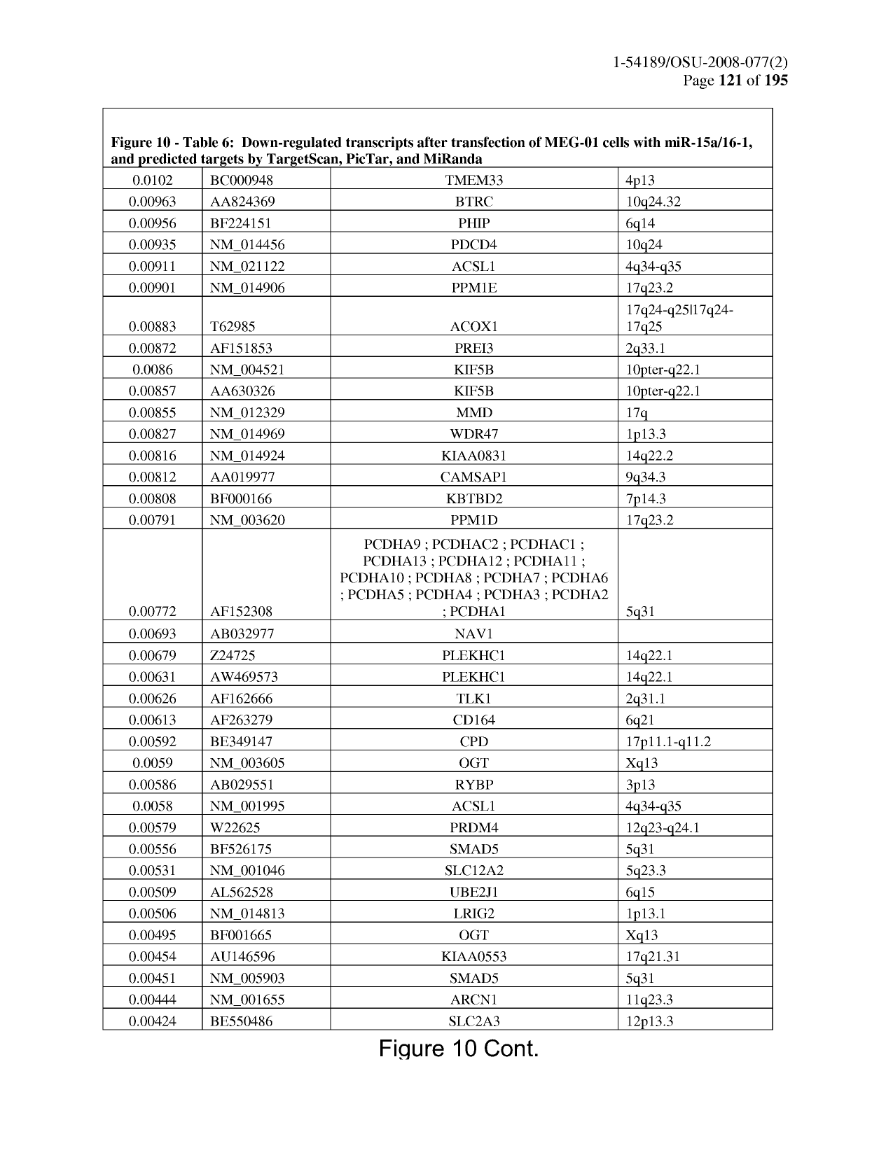

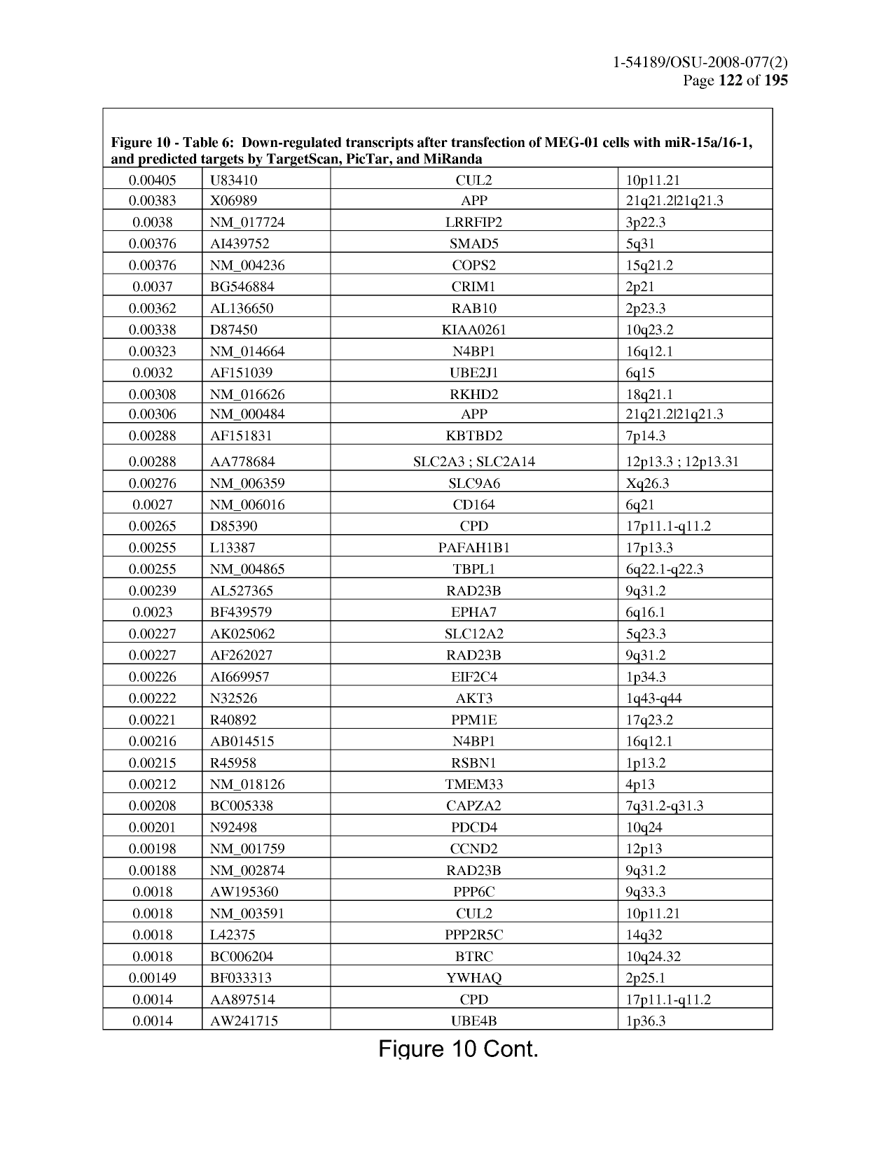

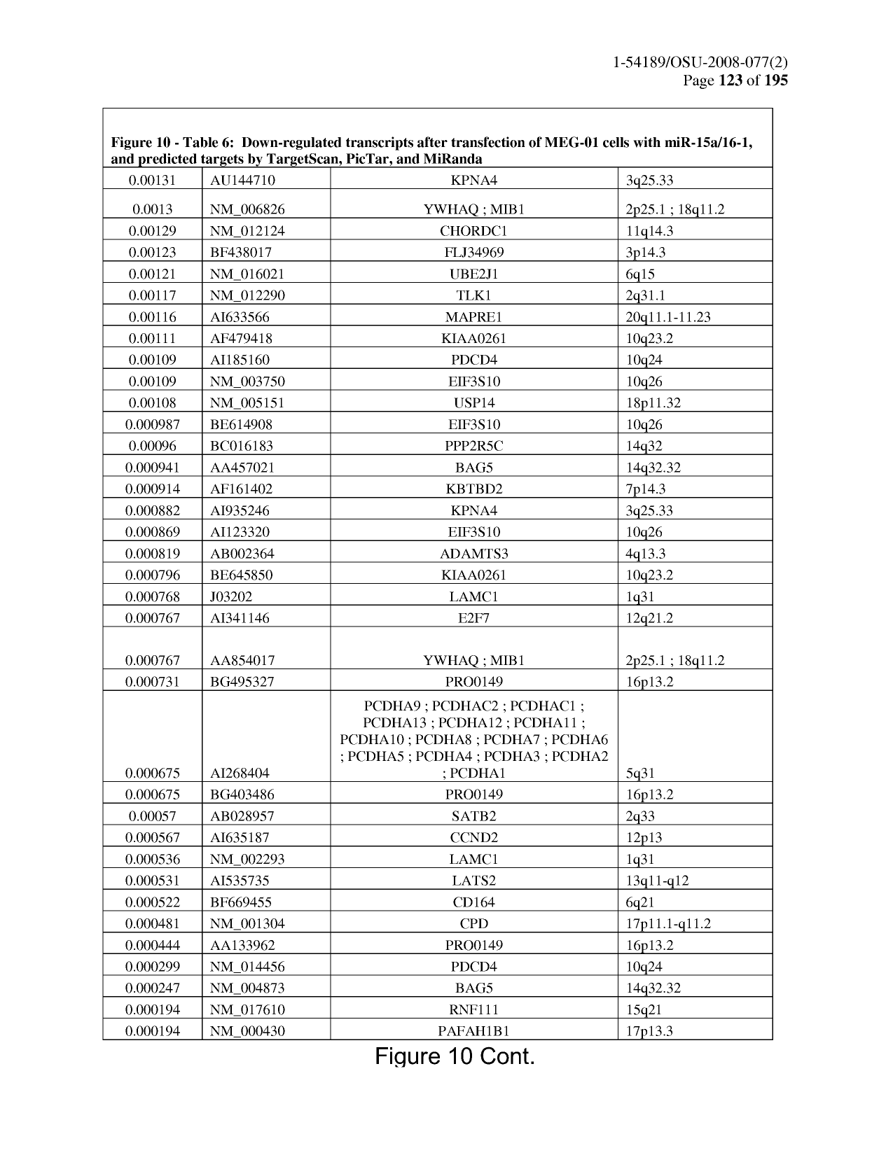

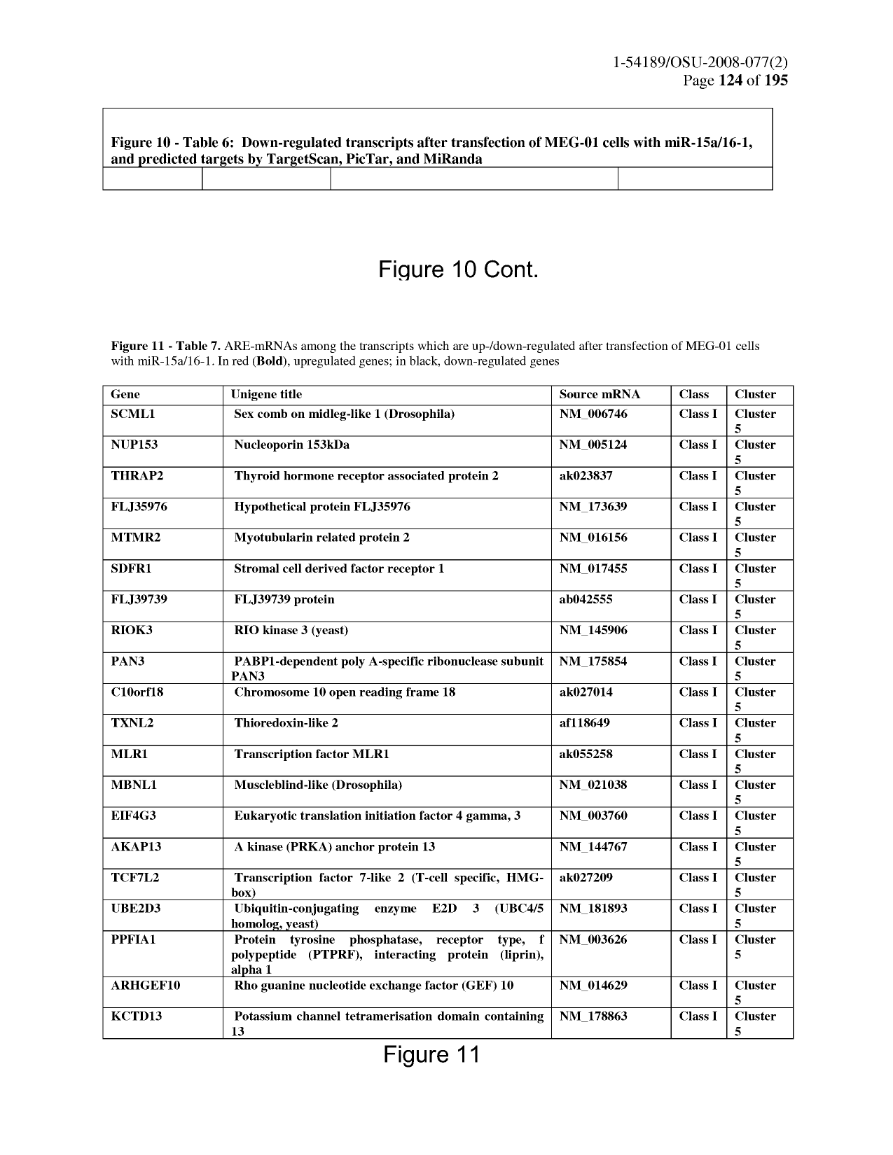

Among the down-regulated probes, 140 (85 genes) are predicted as targets of miR-15/16 by three of the most used software algorithms (TargetScan, PicTar, and MiRanda), that are built on different prediction criteria and, therefore, used in combination, give the highest probability of target identification. If we consider only one prediction program, we found that 370, 332, and 312 transcripts, respectively, are predicted to be direct targets of these miRNAs (FIG. 3, FIG. 10—Table 6).

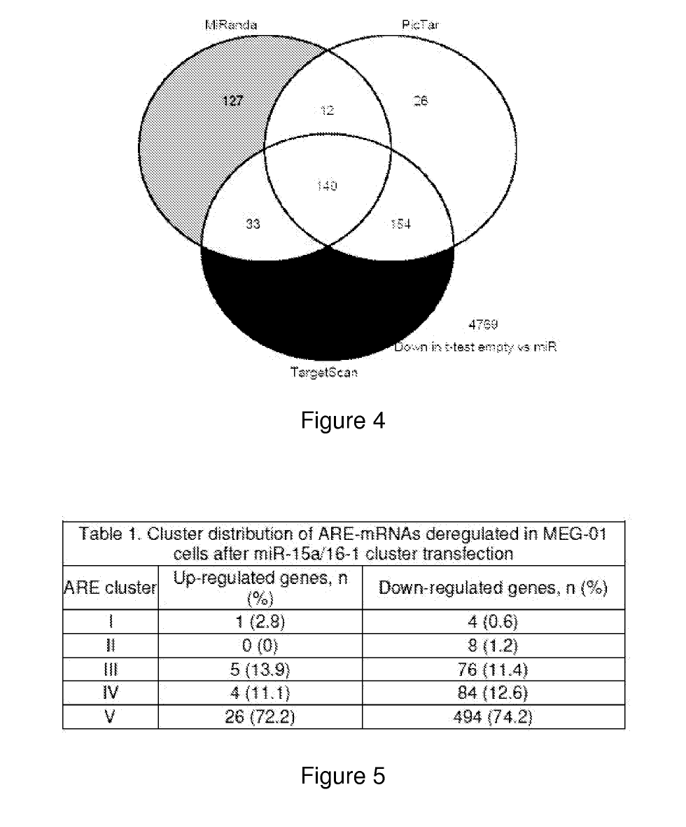

Among the up-regulated genes, there are no commonly predicted targets. Therefore, the miR-15α/16-1 cluster seems to regulate, directly or indirectly, ≈14% (265 genes up- and 3,307 down-regulated) of the 25,000 total predicted genes in the human genome (23) (FIG. 4).

AU-Rich Elements (AREs) Are More Frequently Found Among miR-15α/miR-16-1 Down-Regulated Genes, in MEG-01

Because for miR-16-1 both a direct interaction in the “seed” region of the target mRNAs (22) and an ARE-mediated mRNA instability (24) have been reported, we investigated the frequency of ARE-containing mRNAs among the miR-15α/16-1-deregulated transcripts.

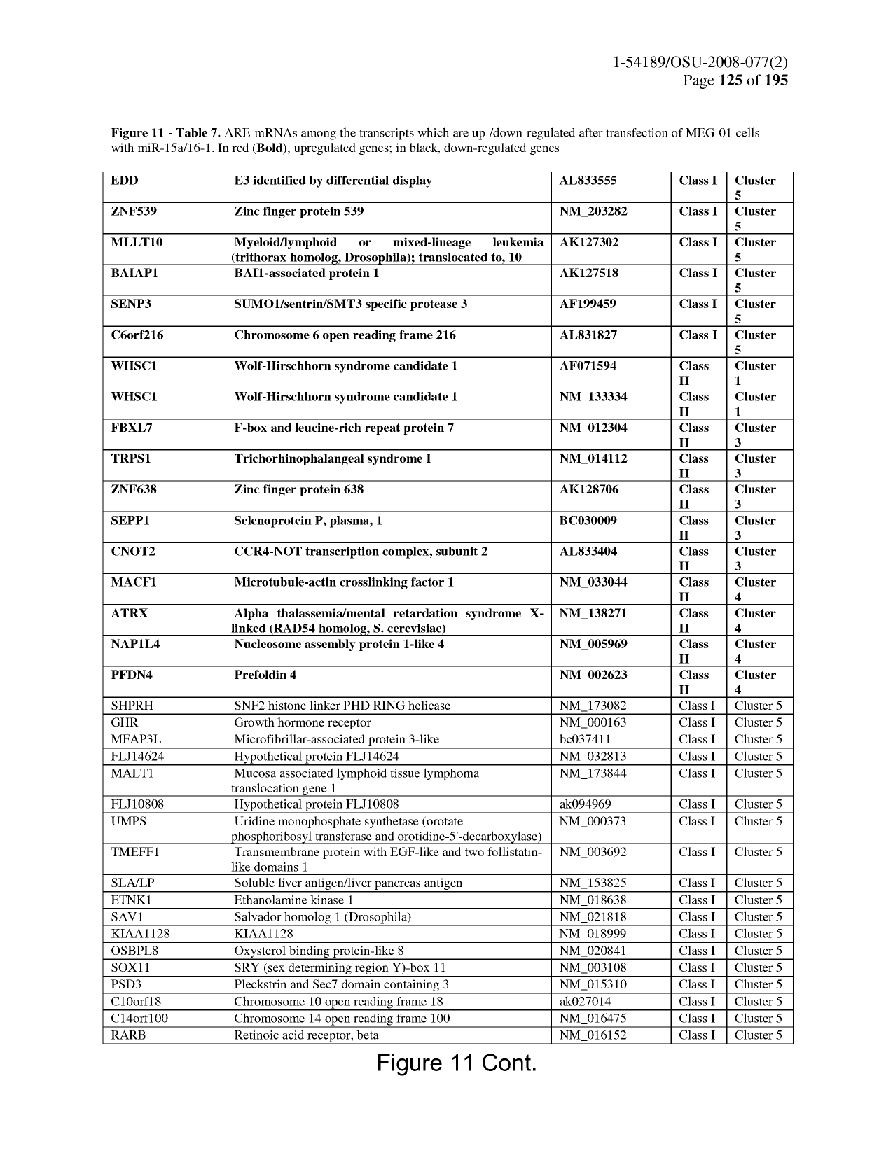

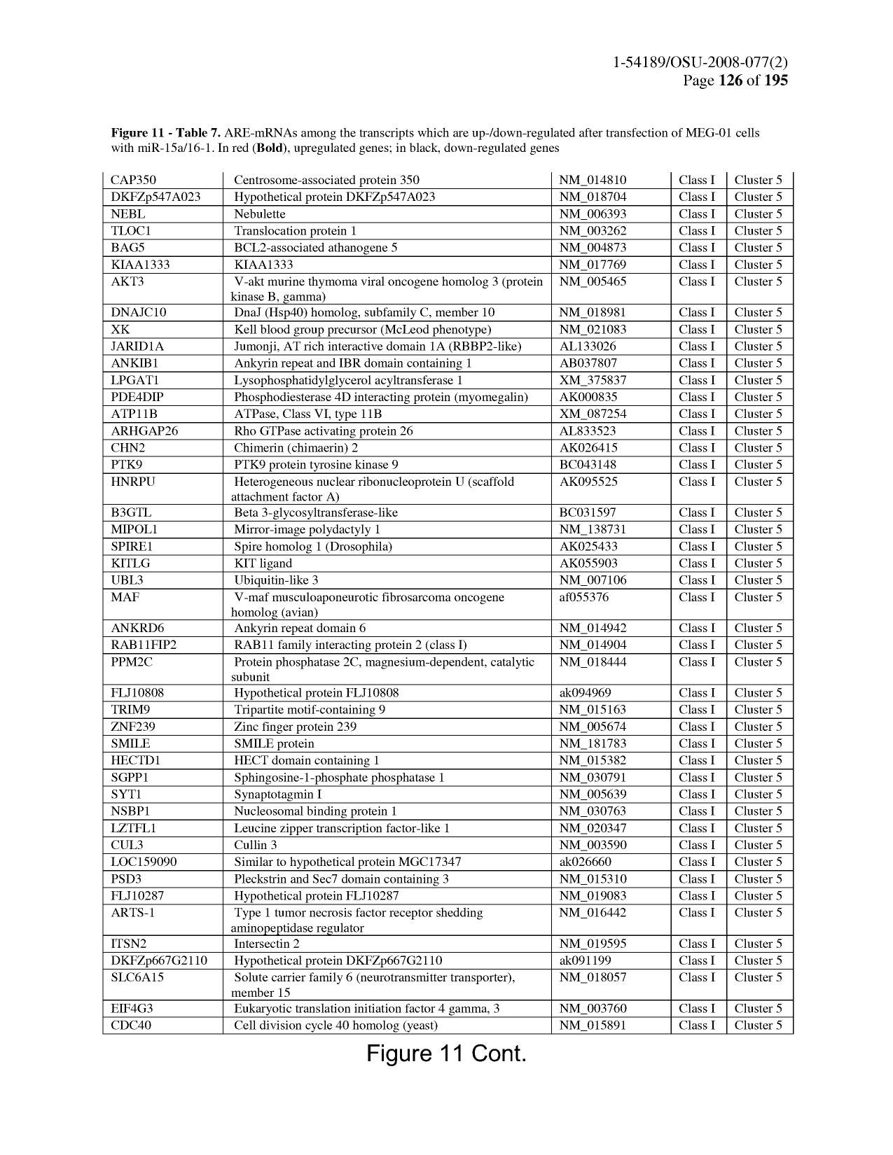

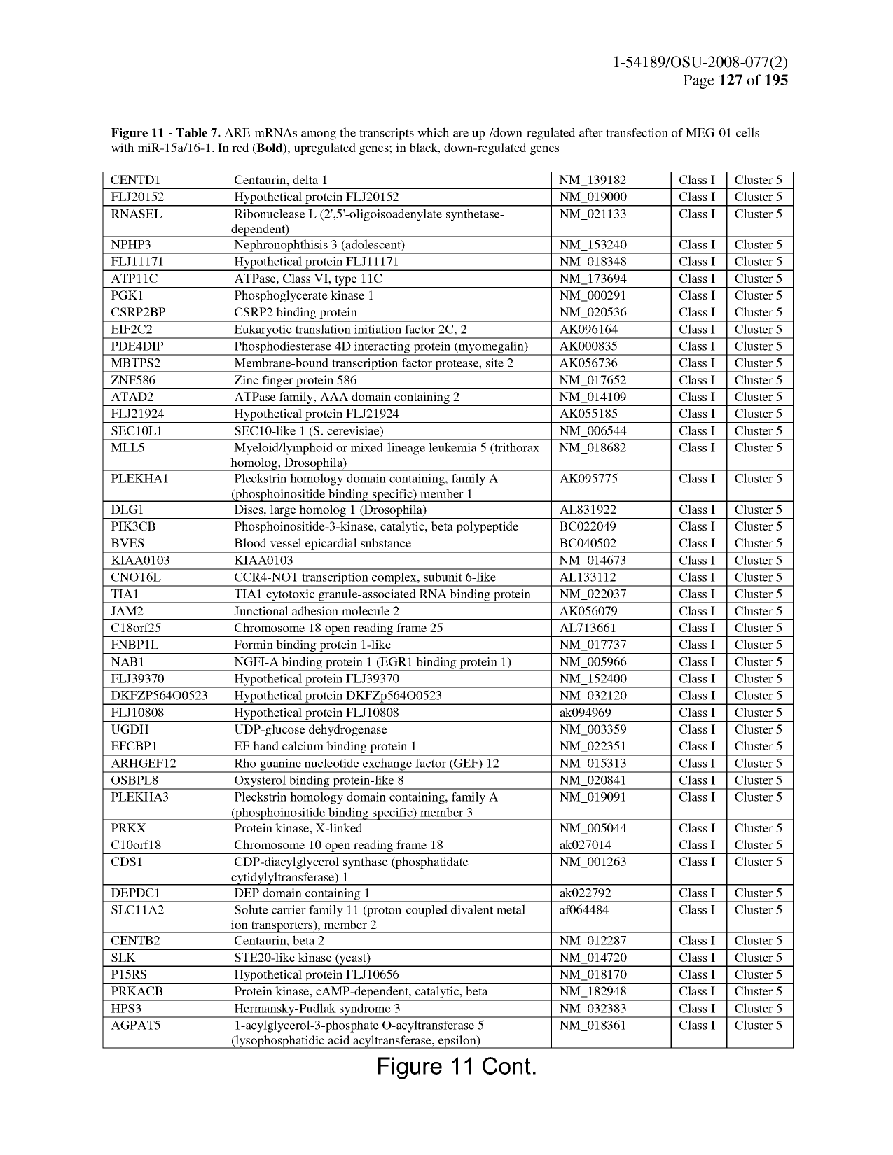

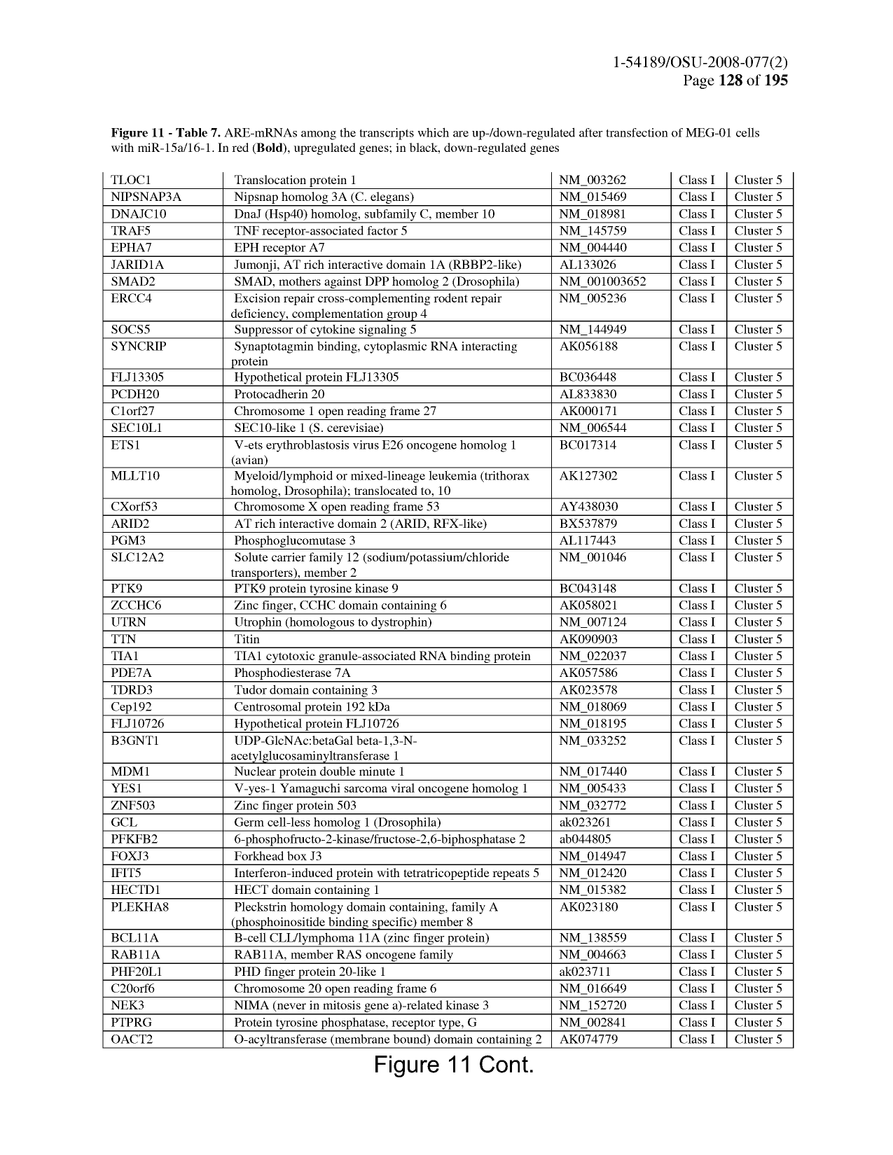

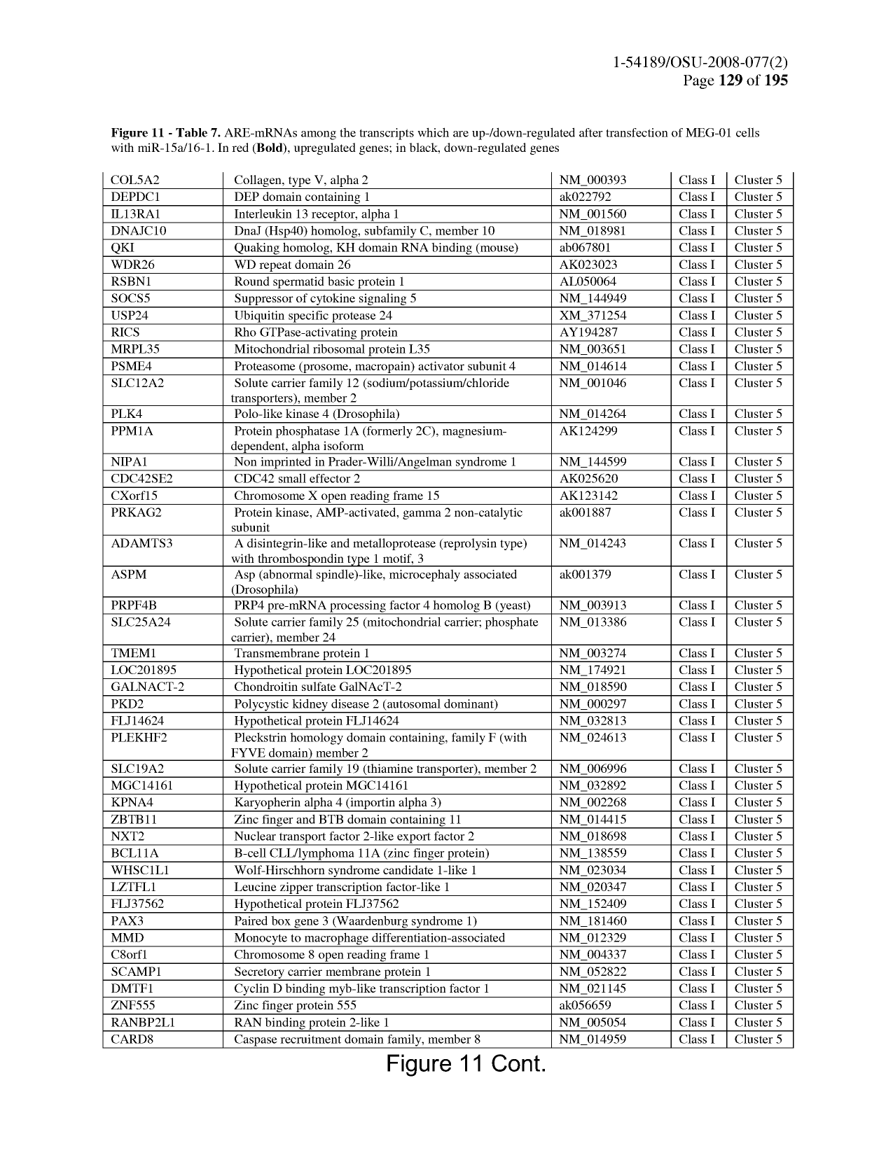

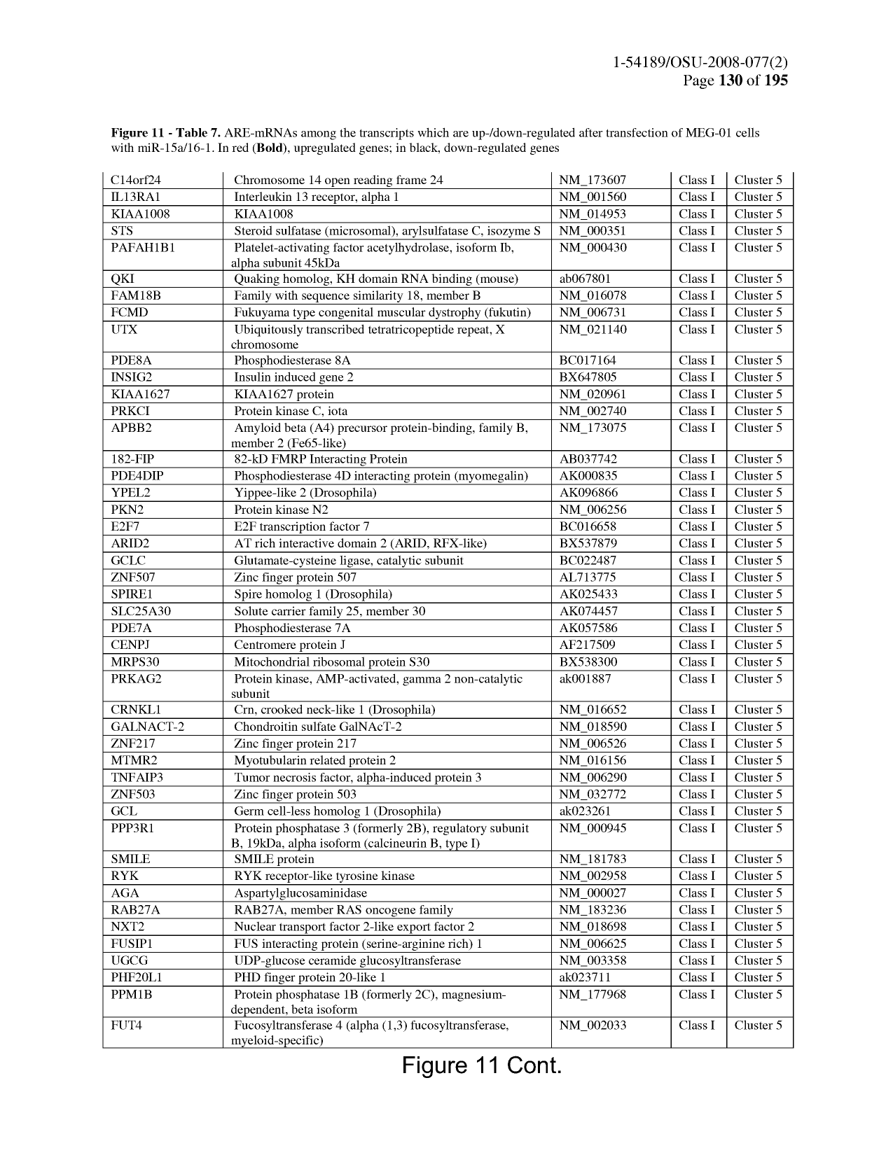

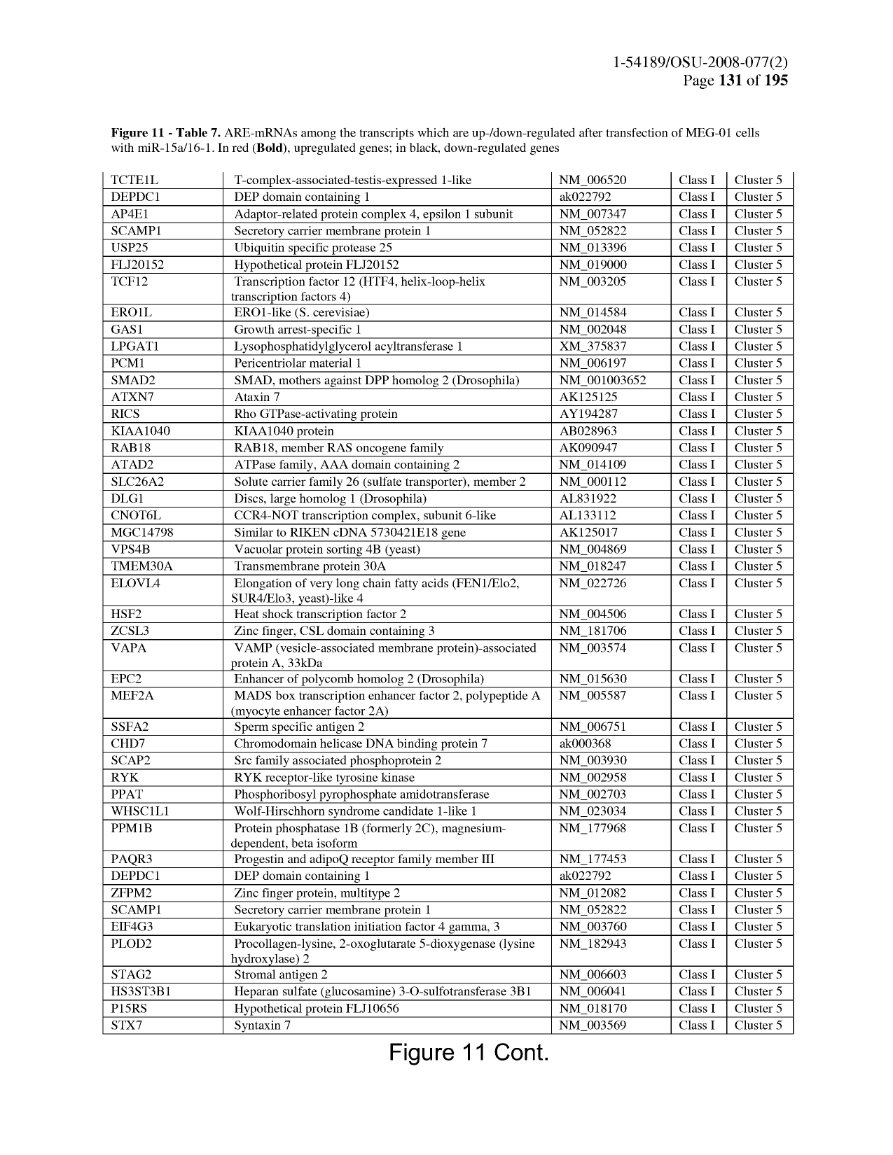

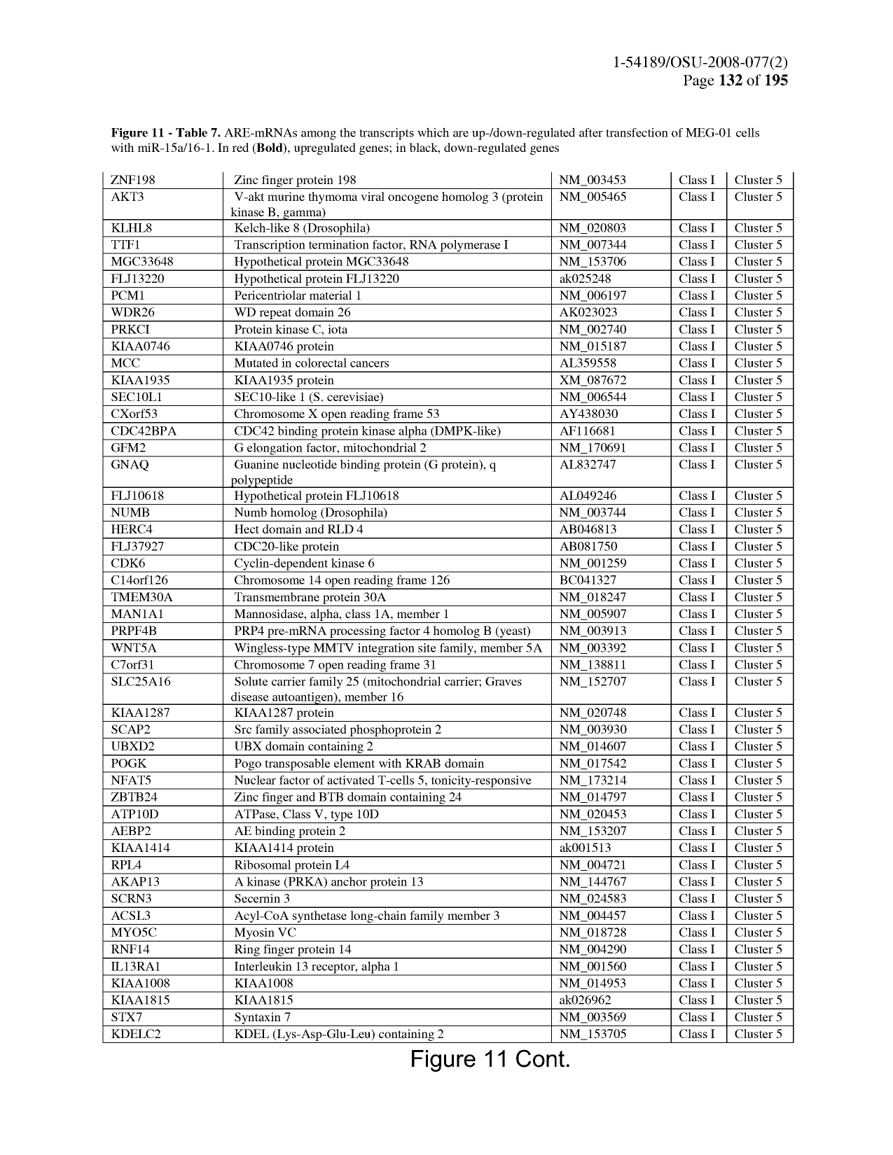

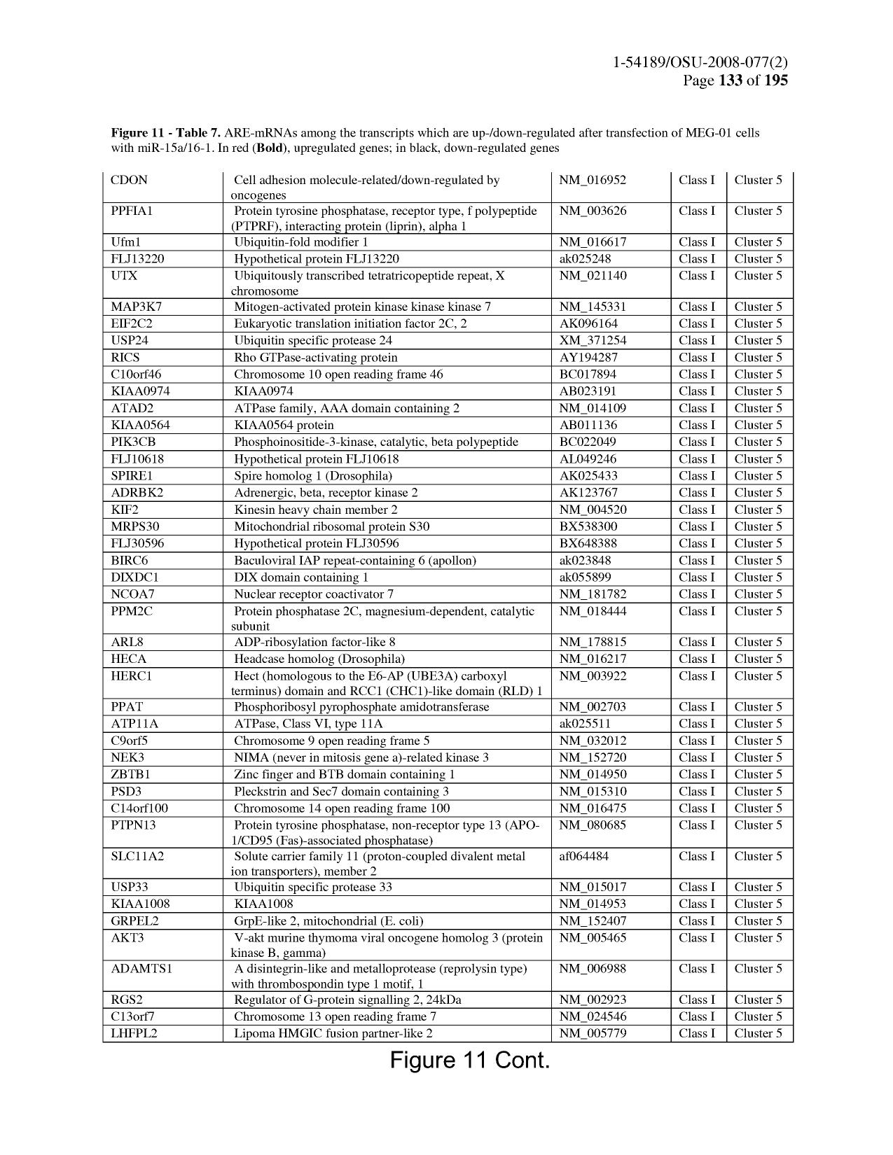

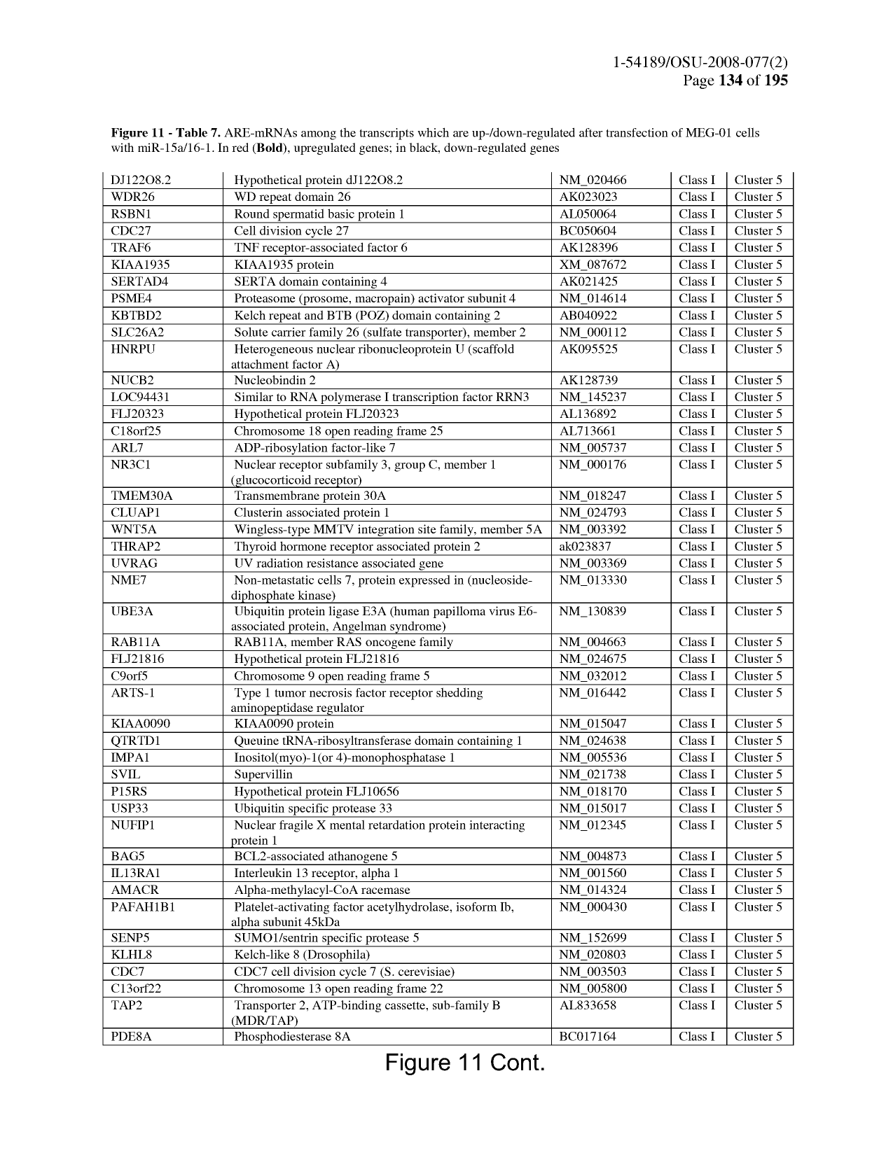

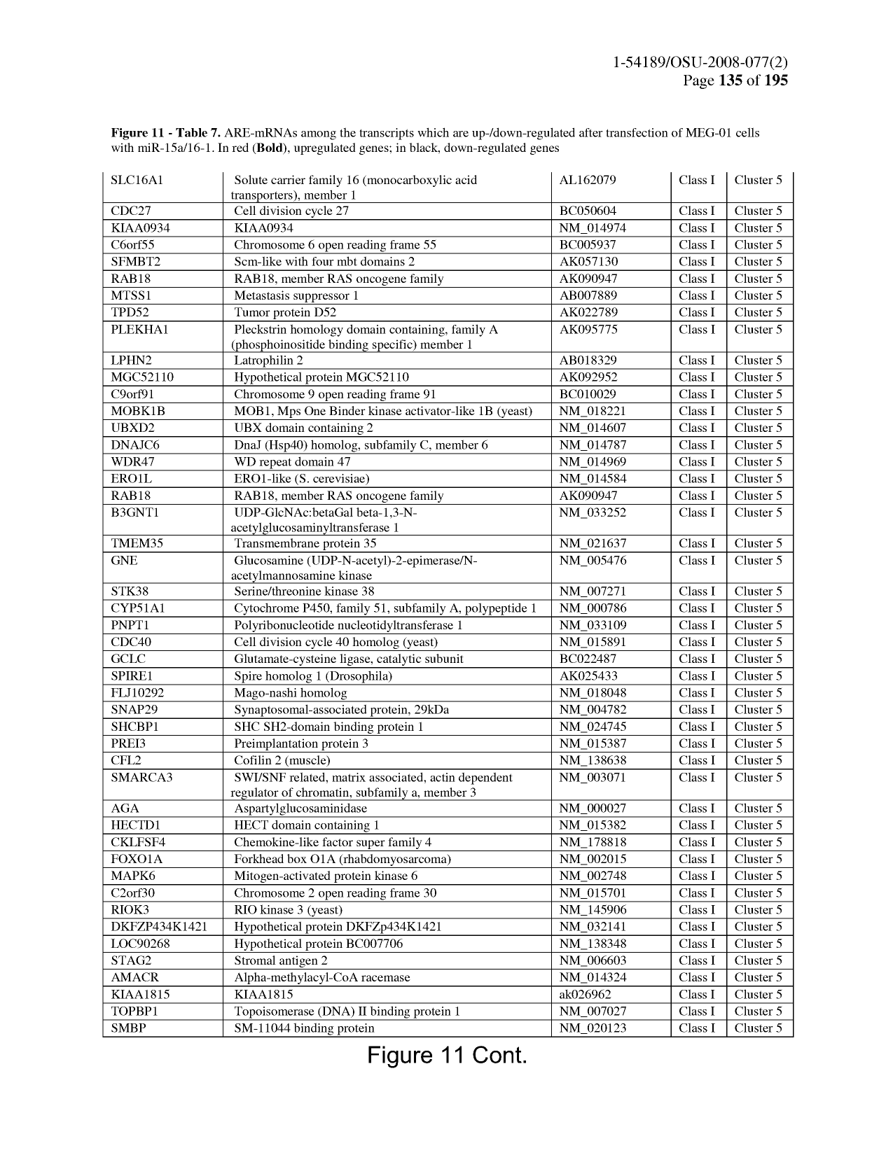

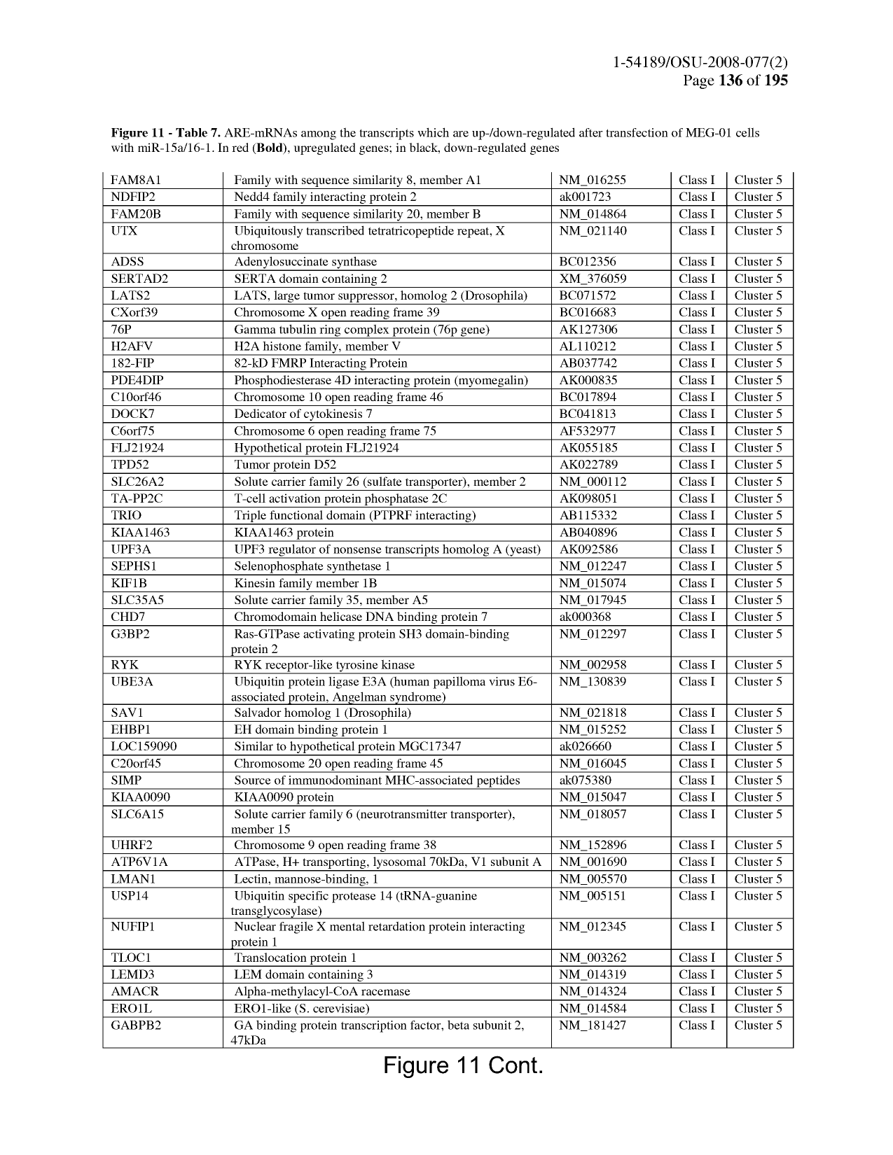

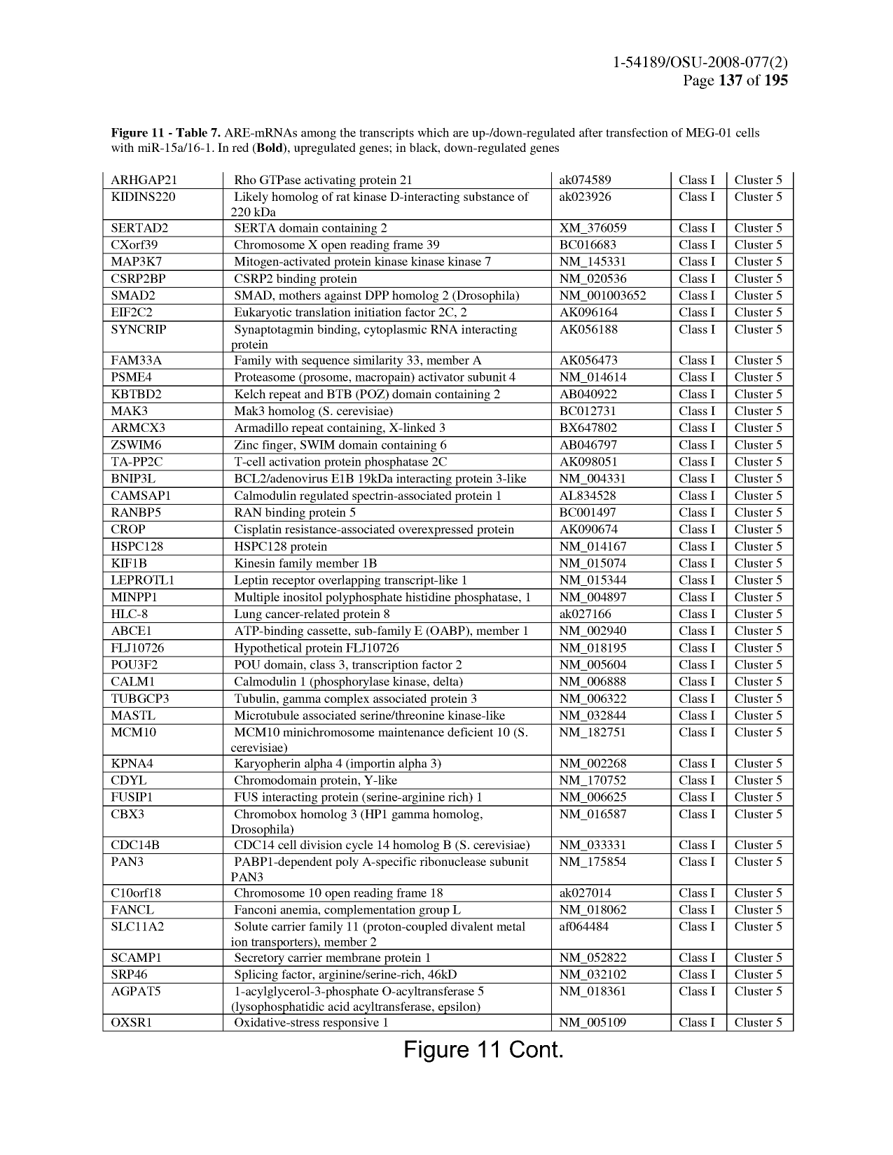

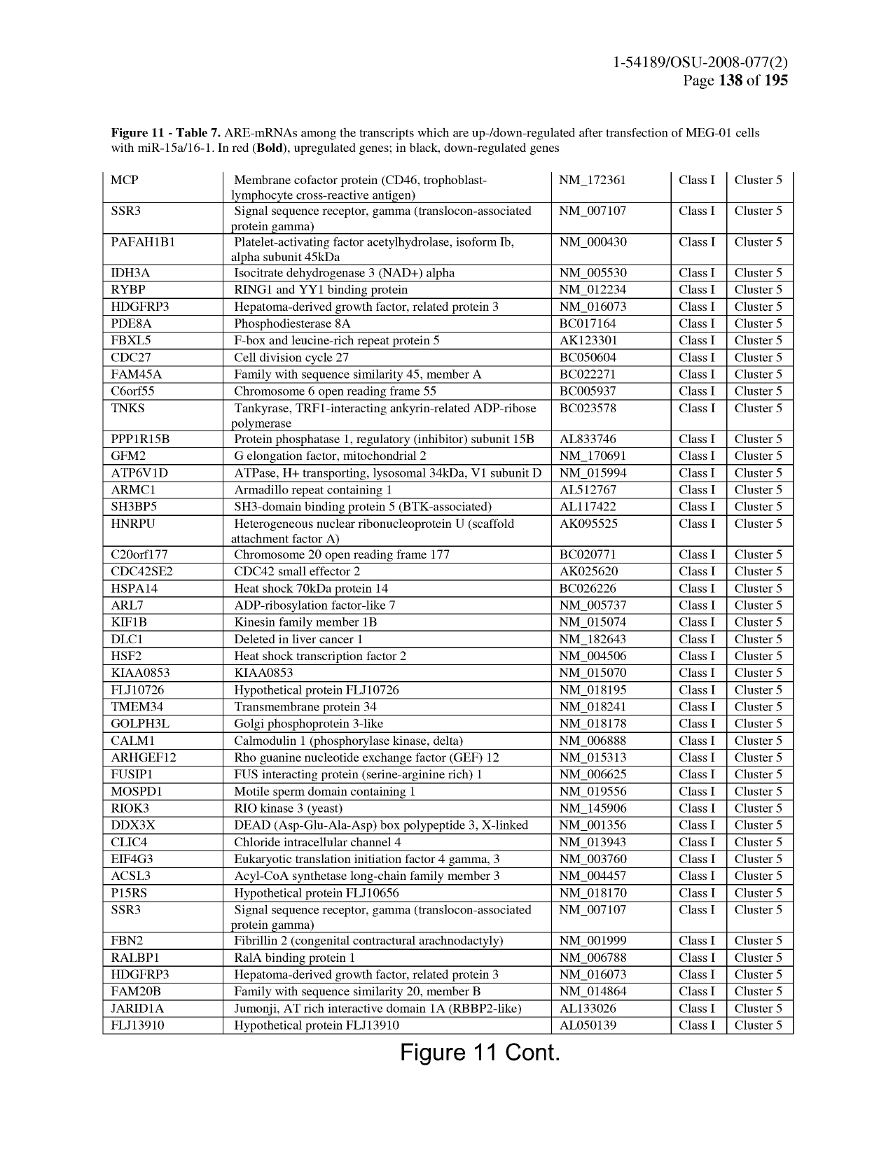

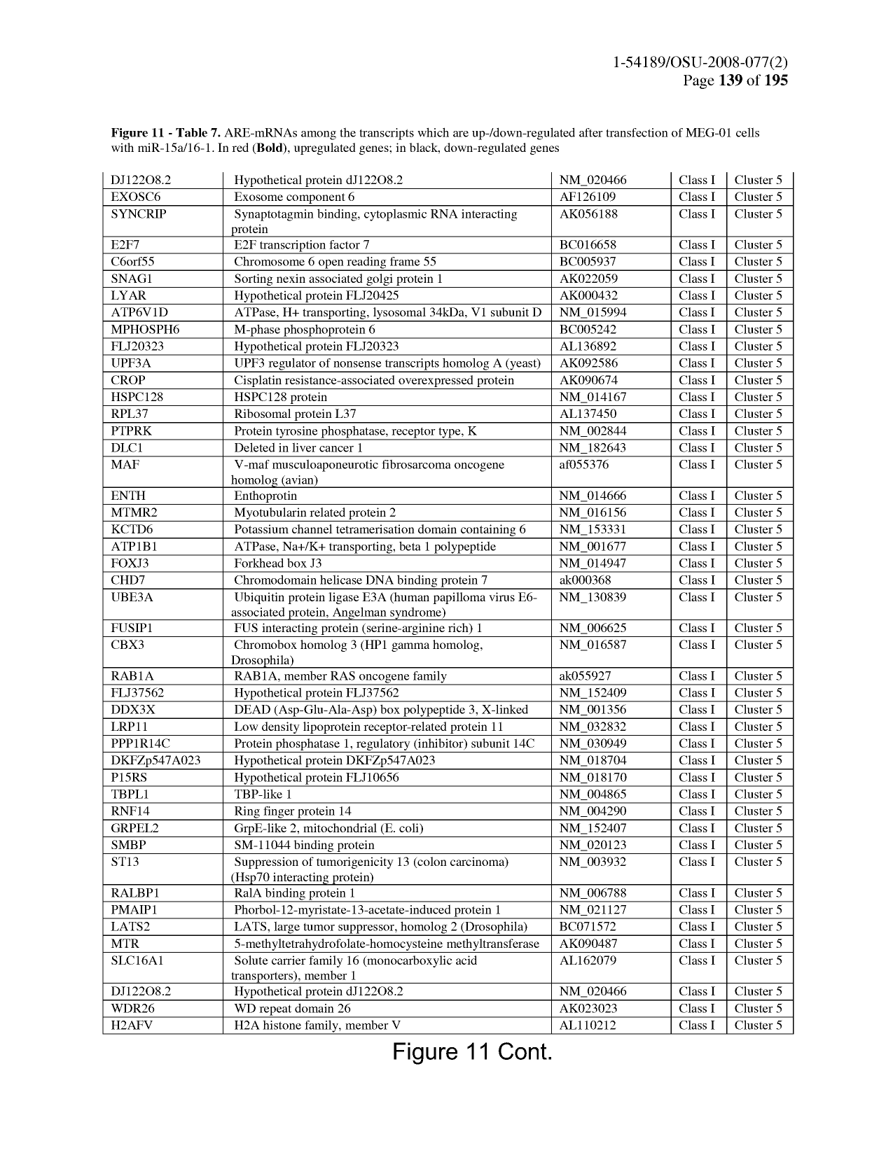

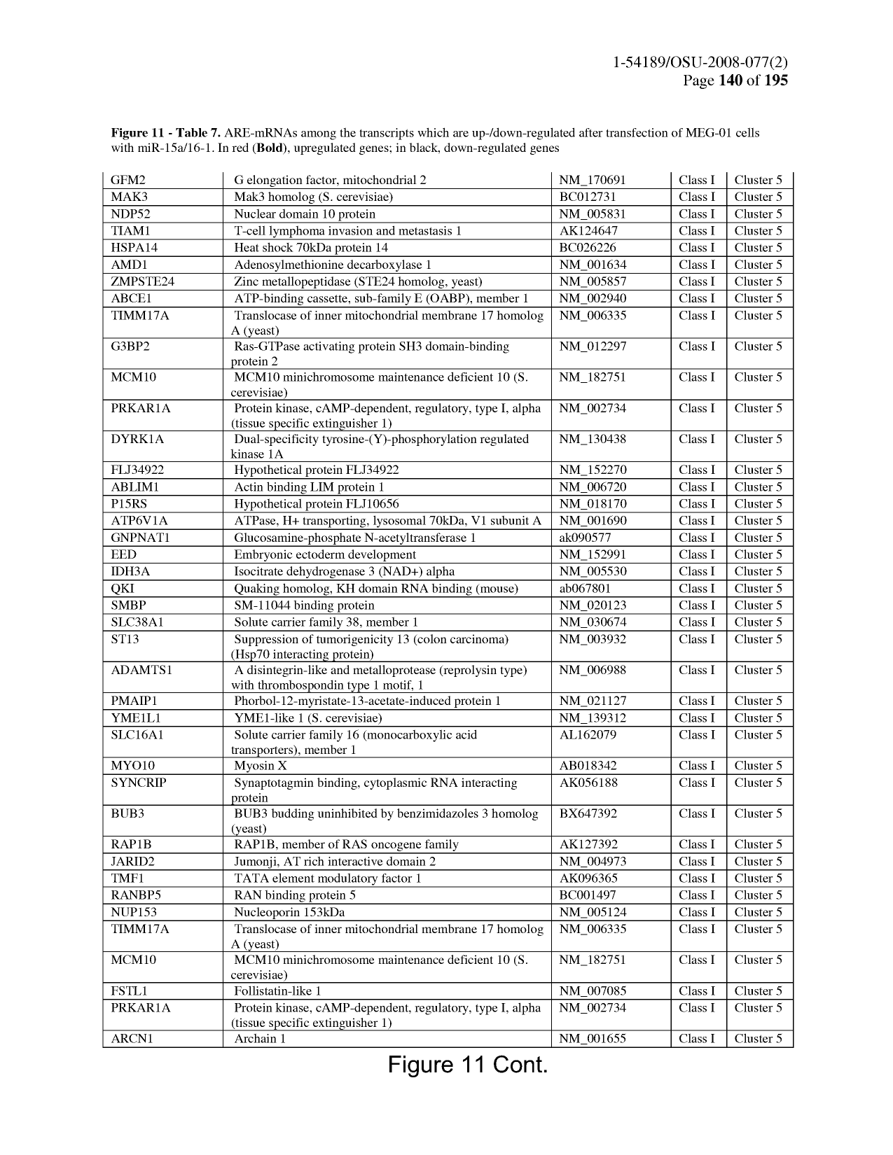

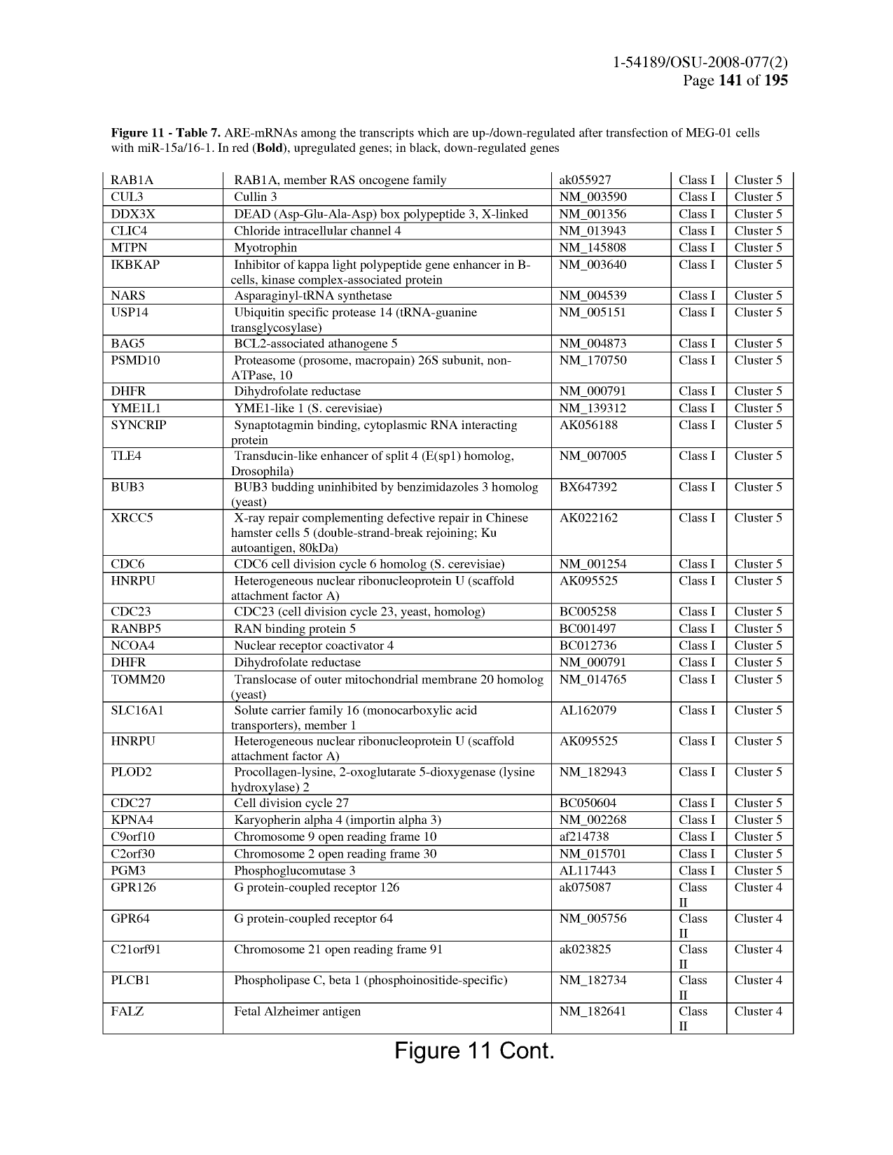

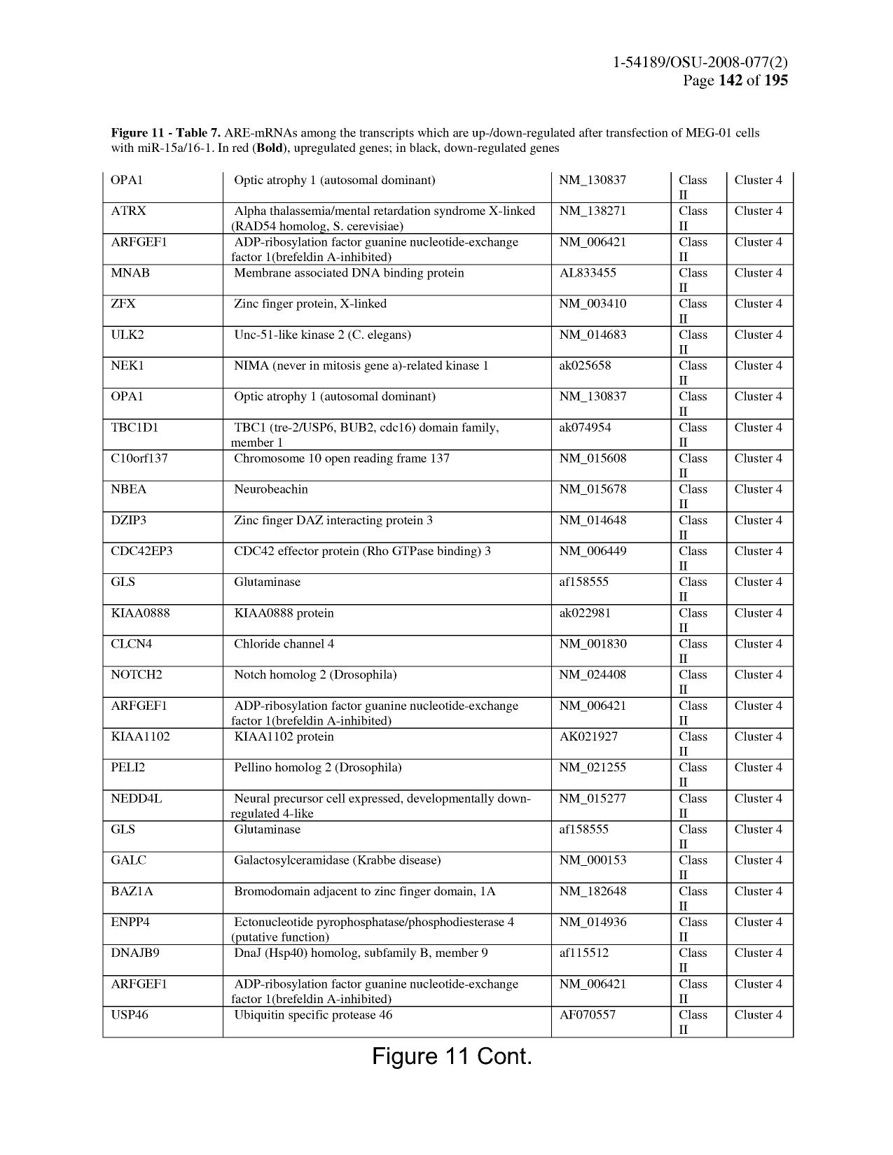

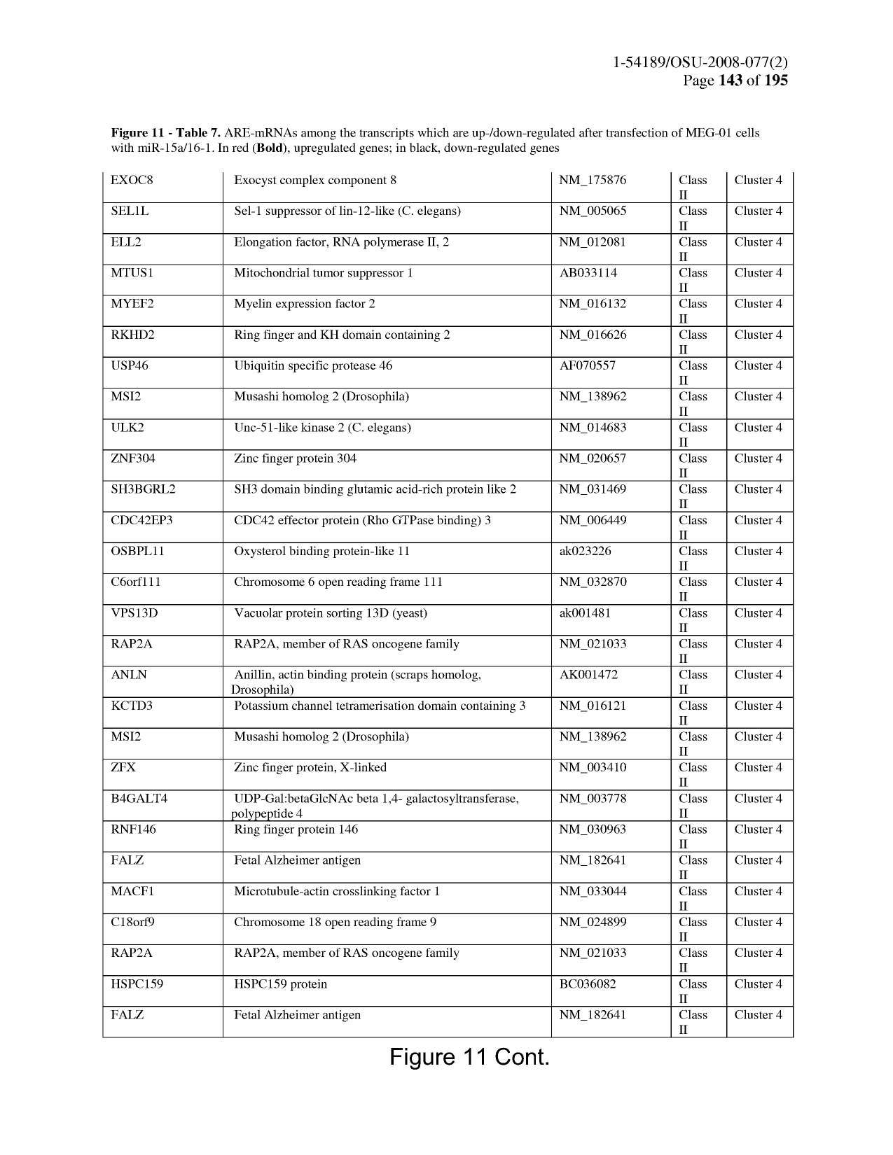

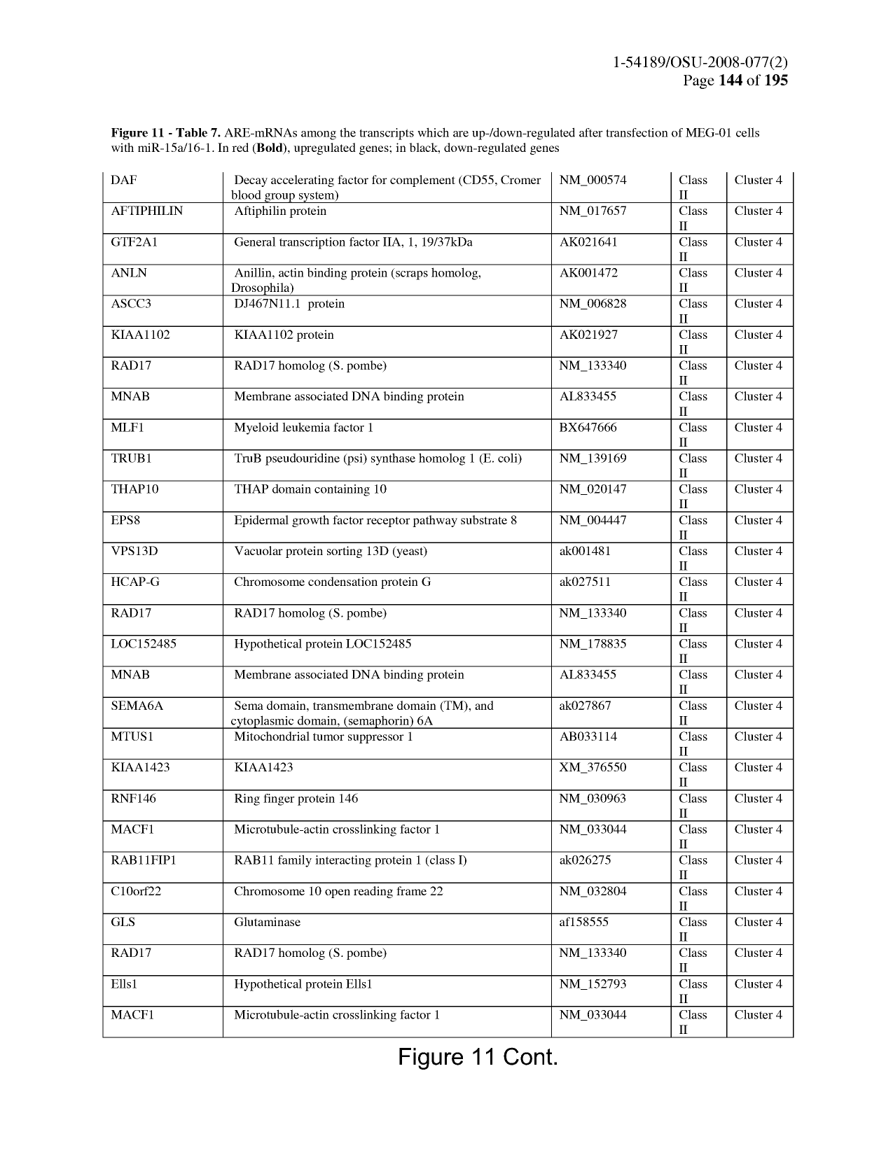

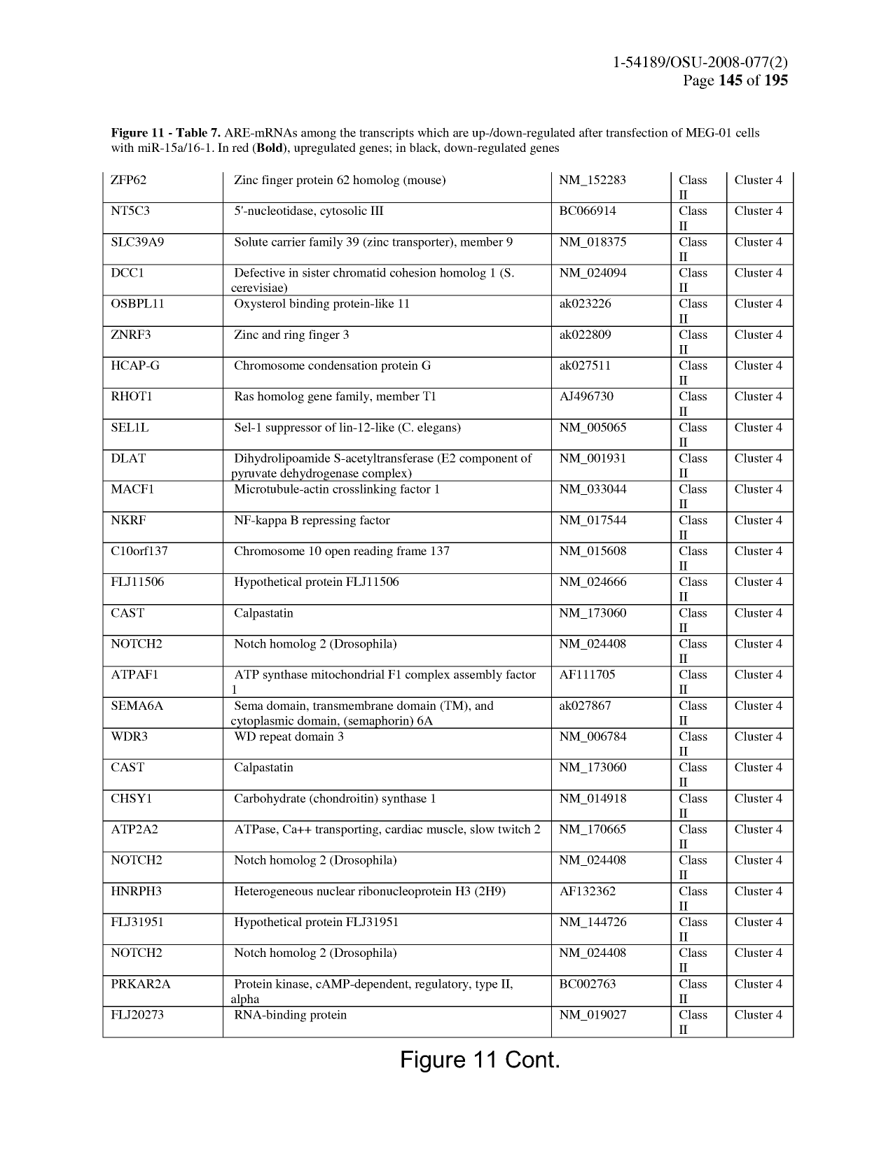

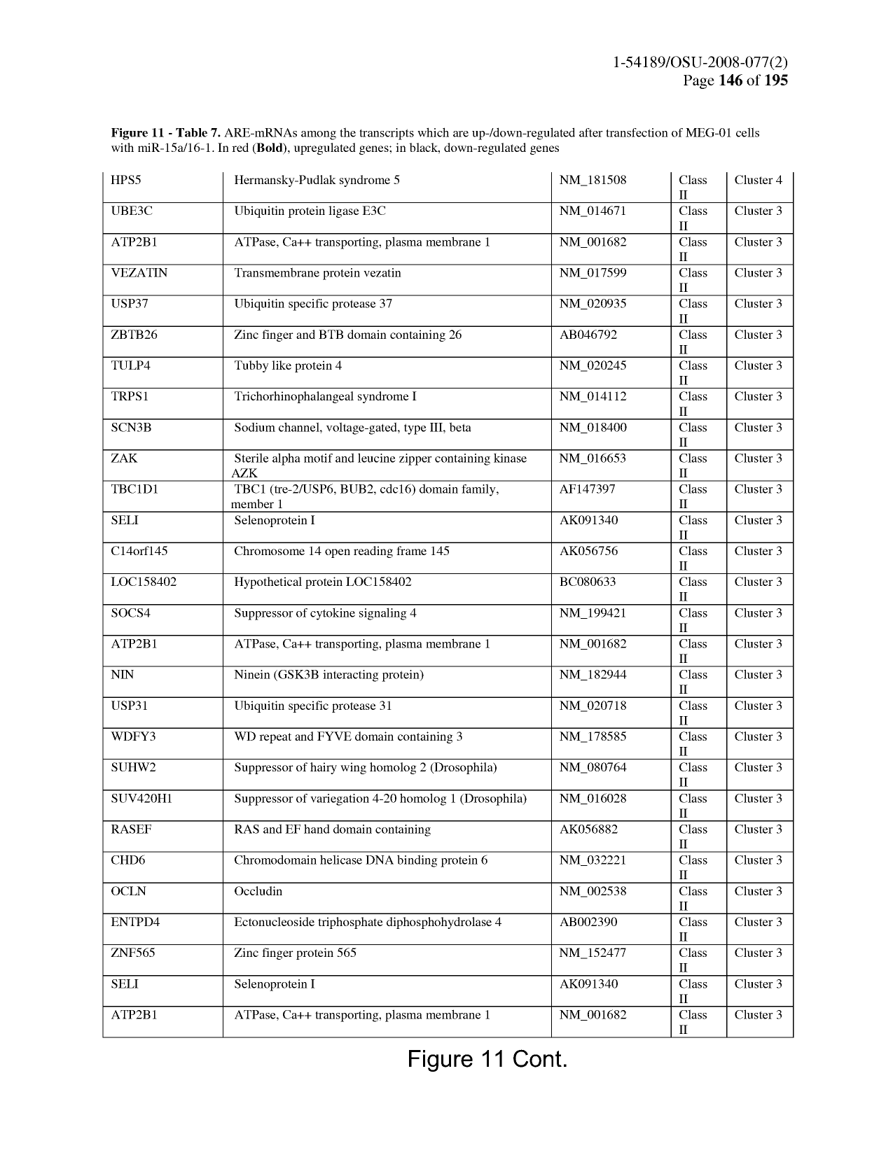

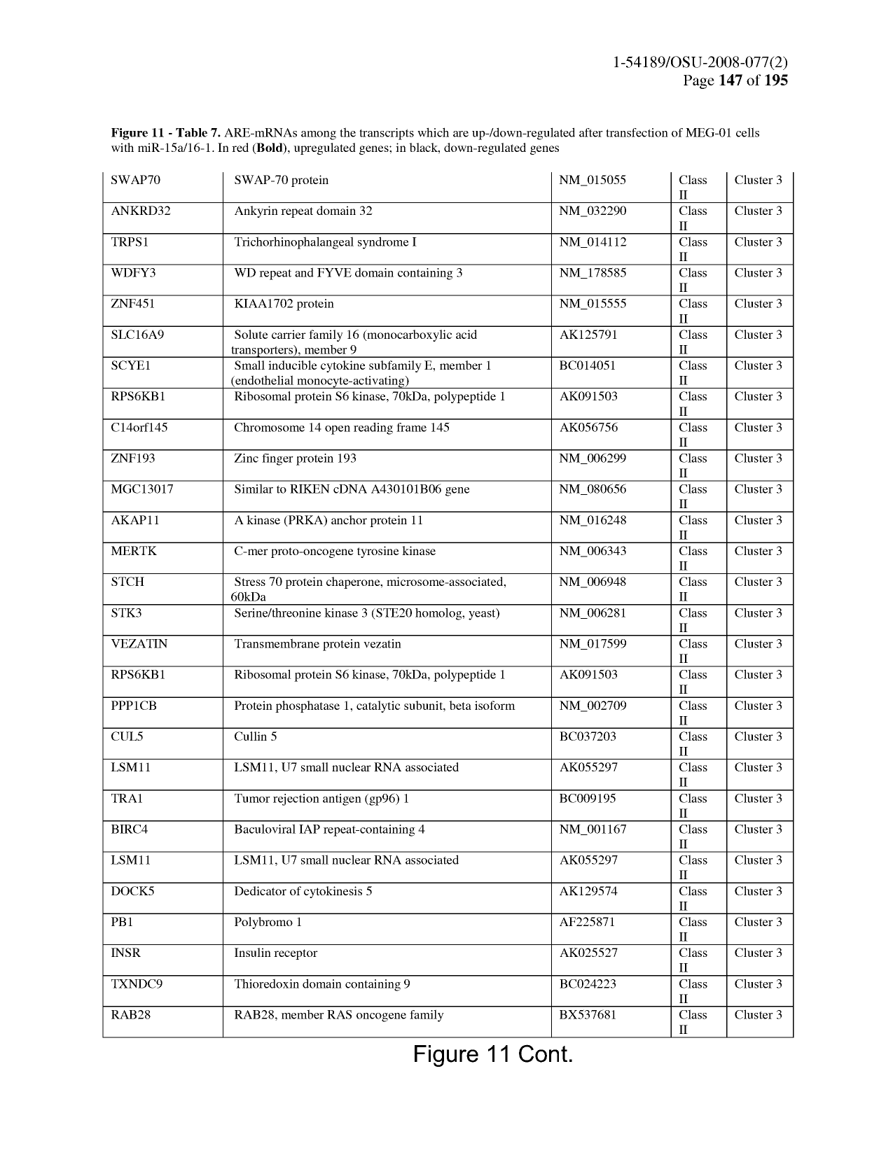

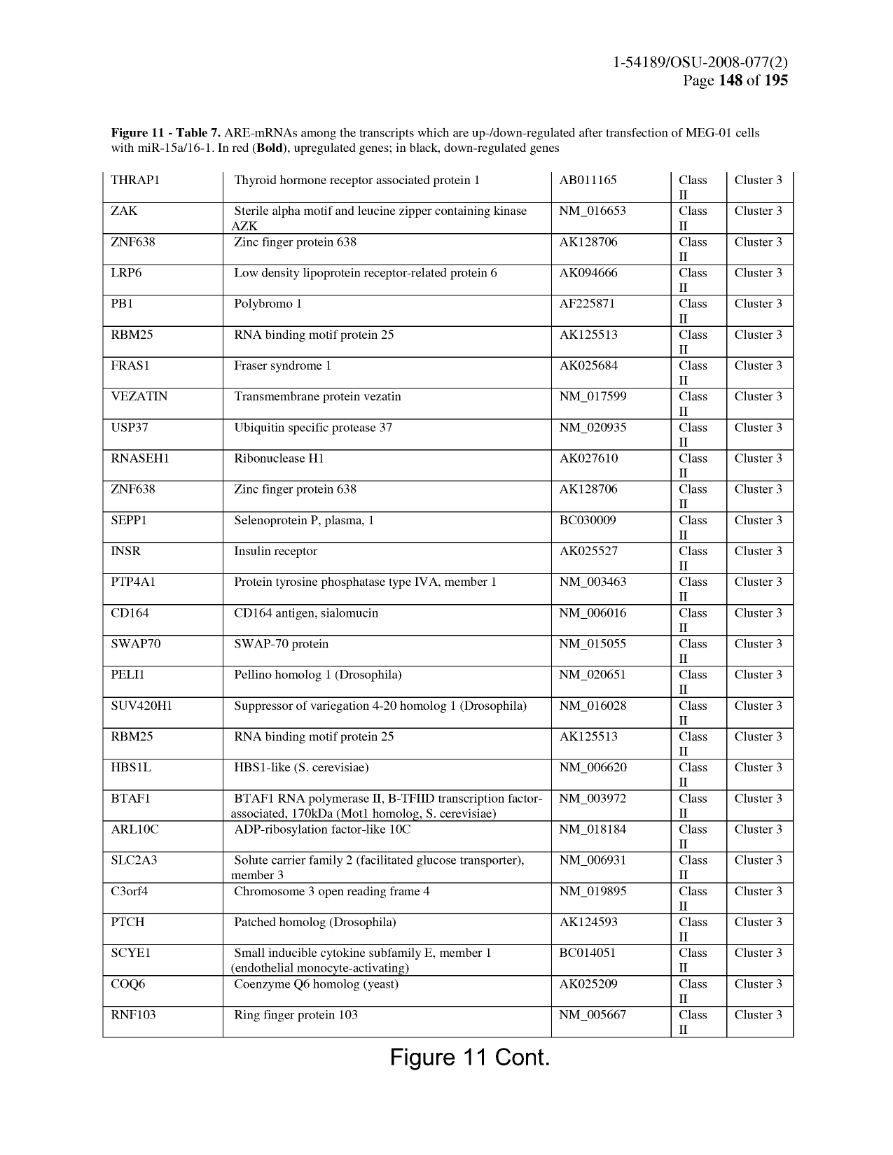

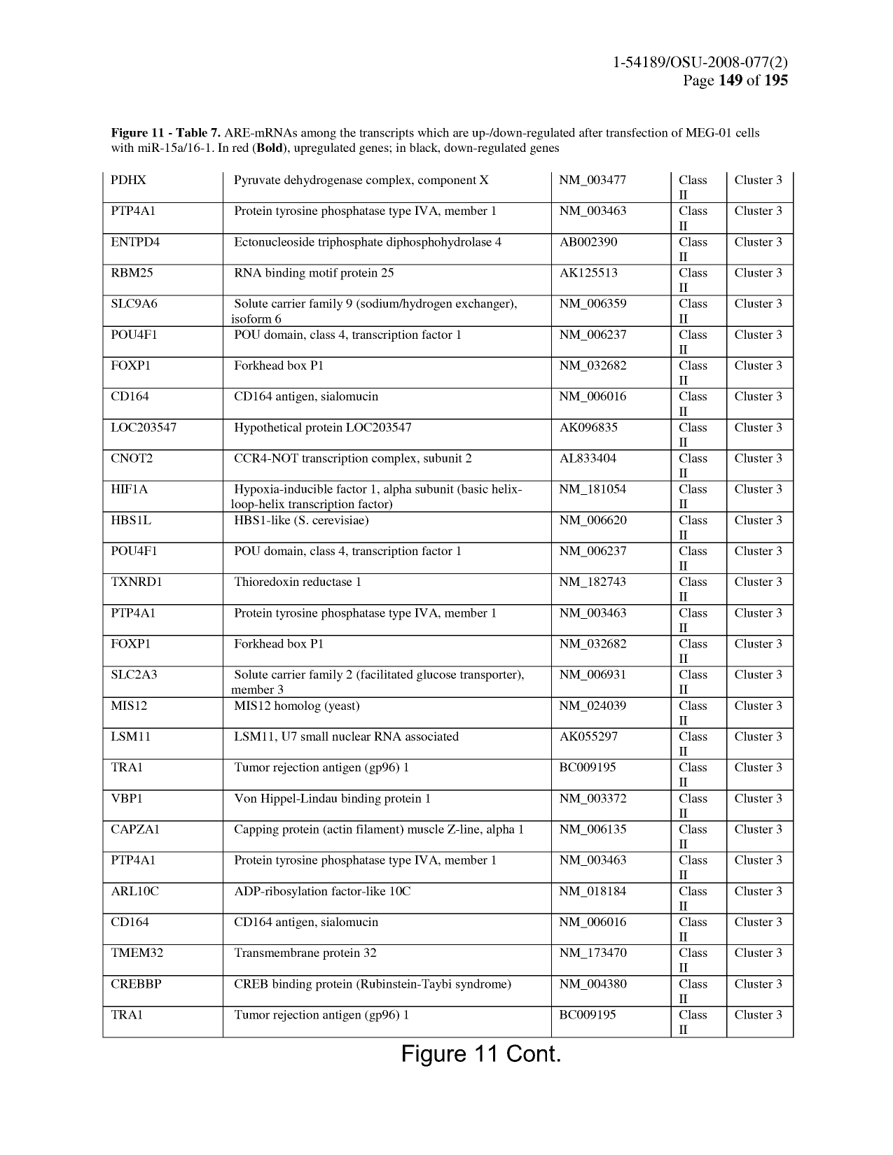

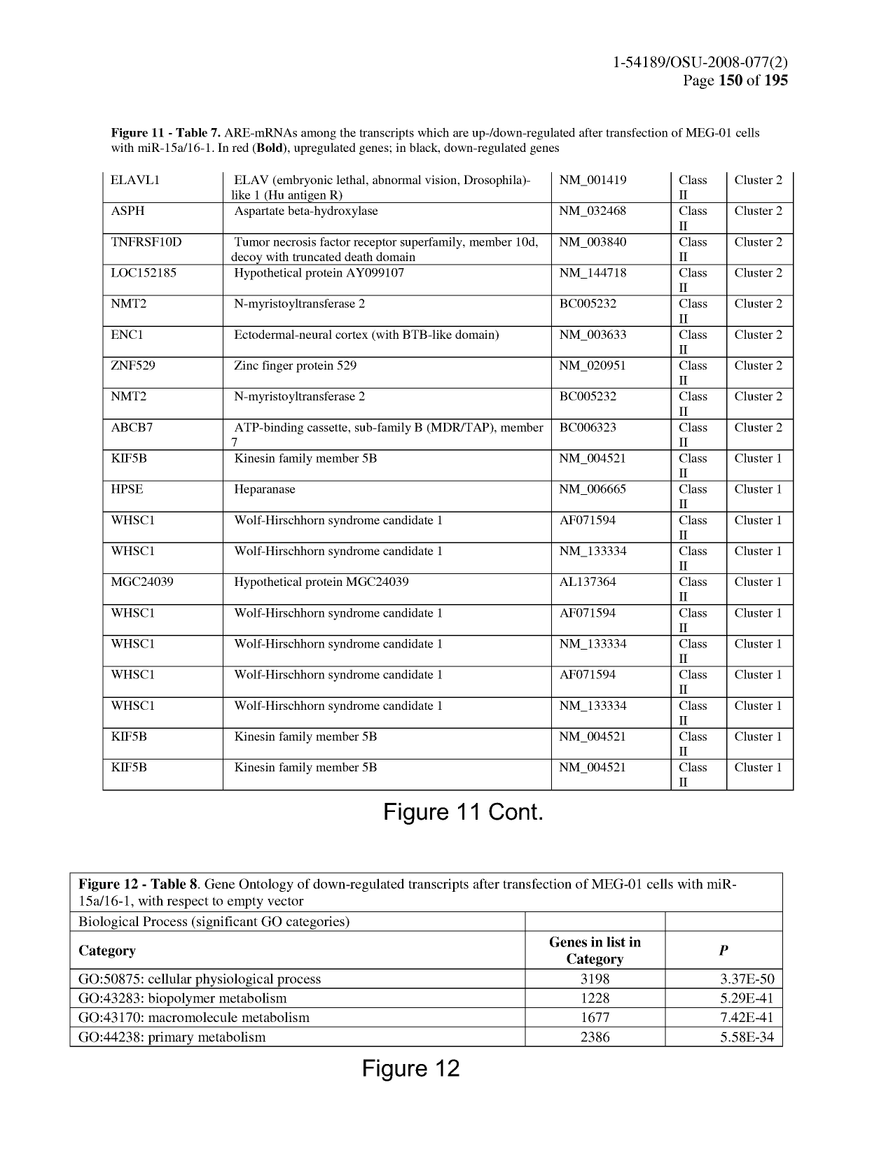

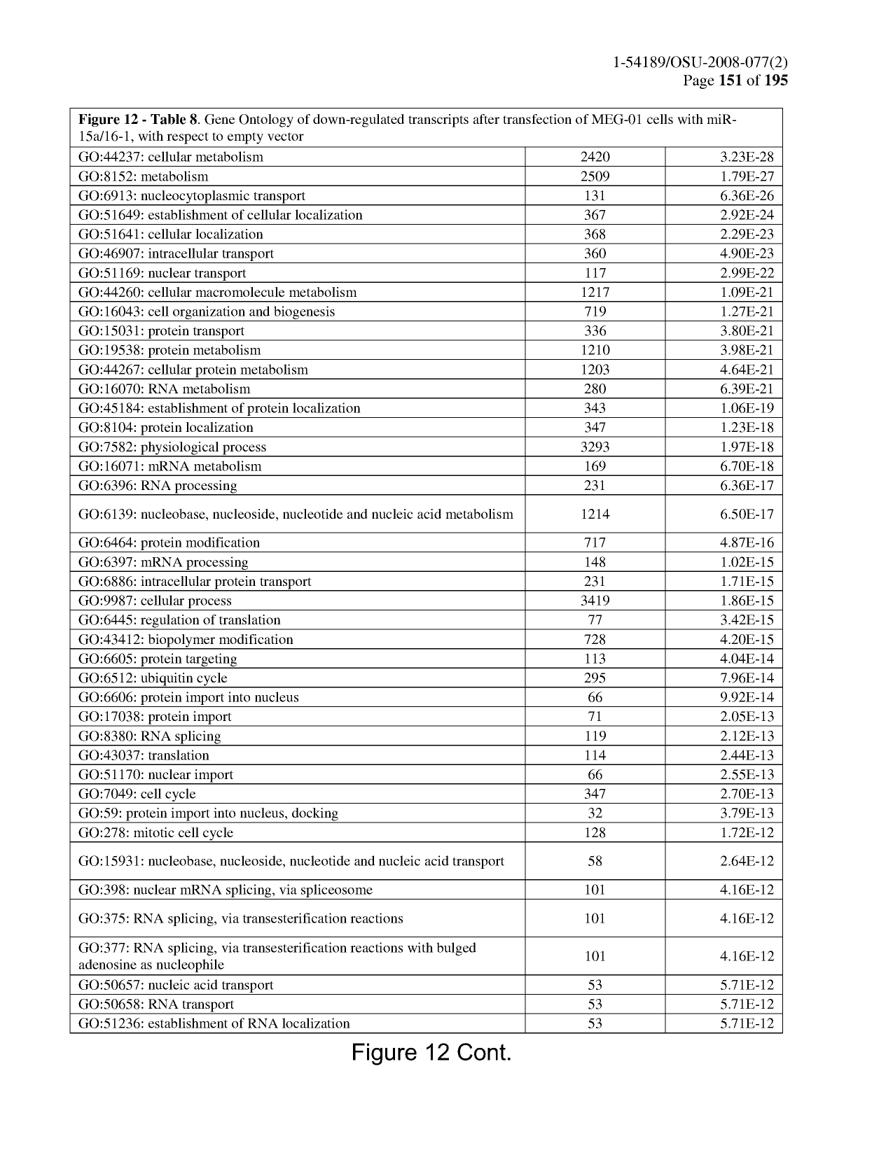

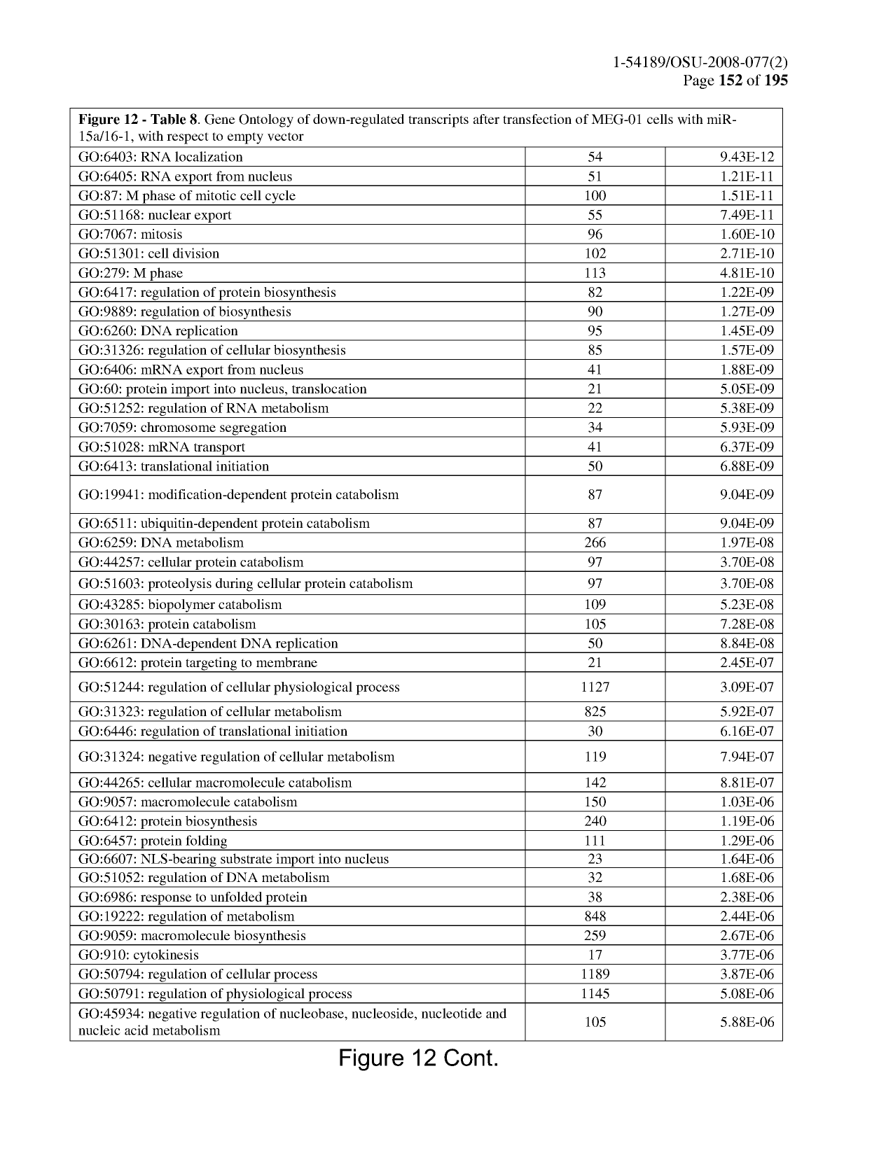

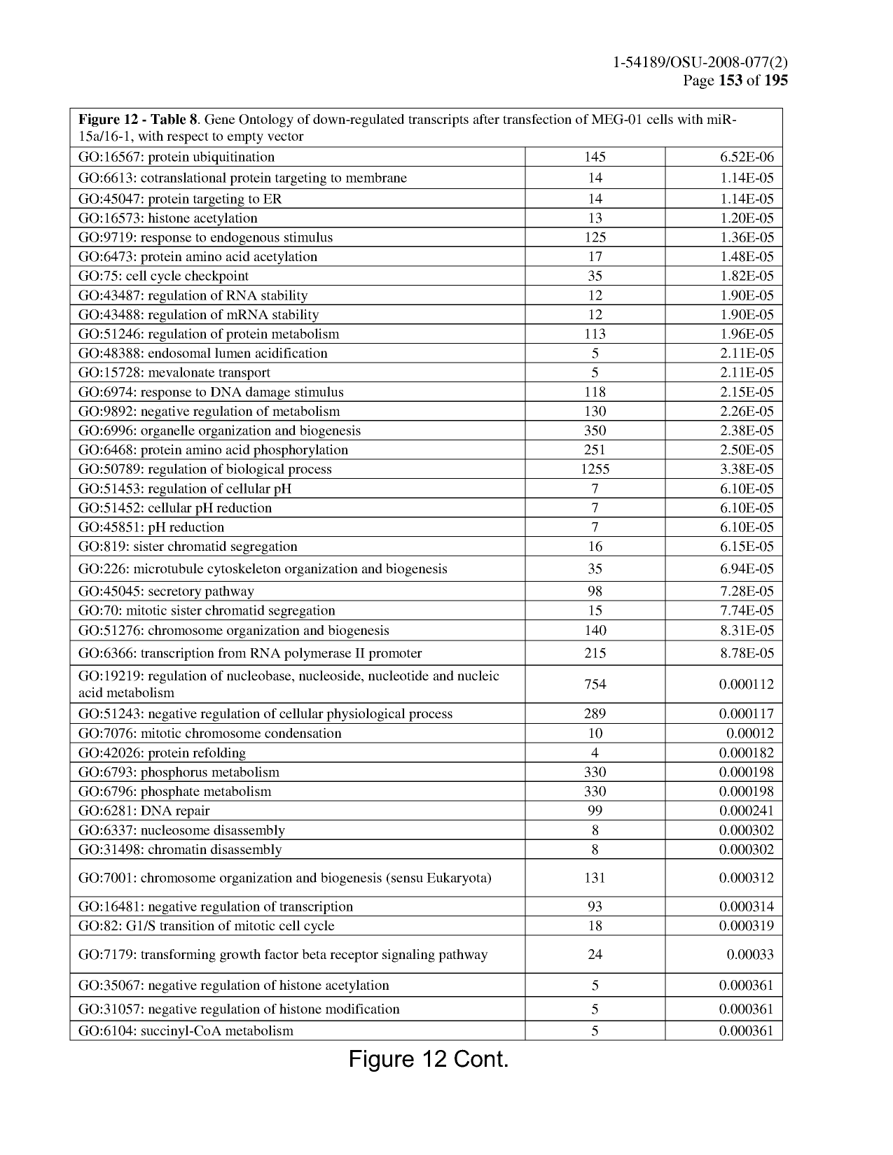

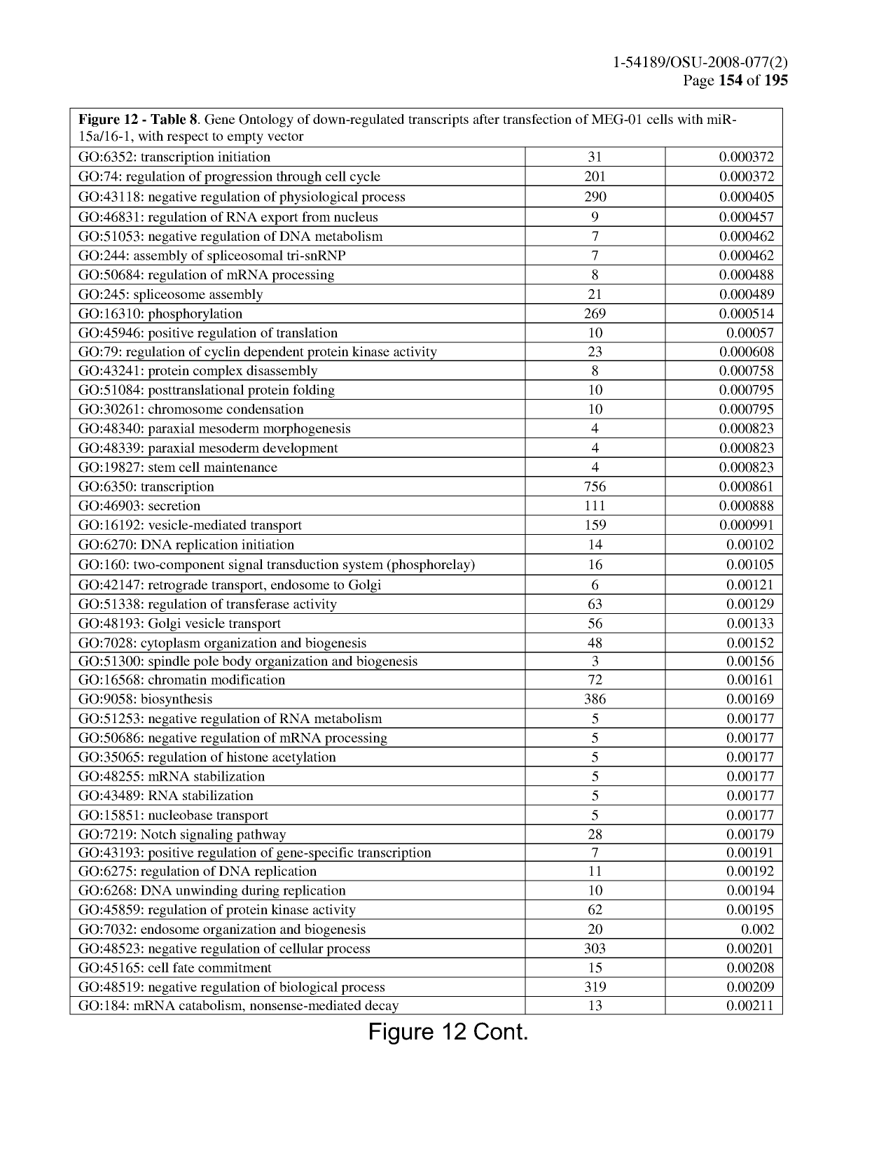

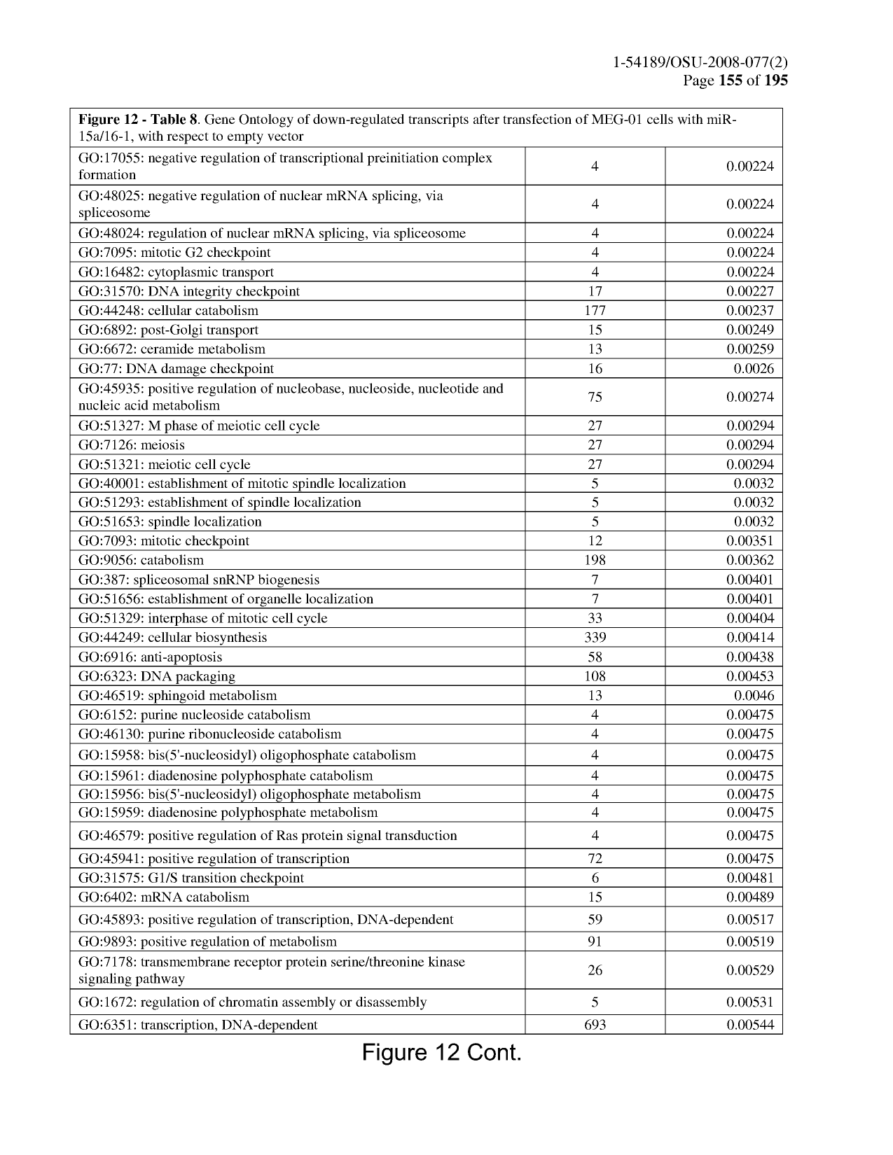

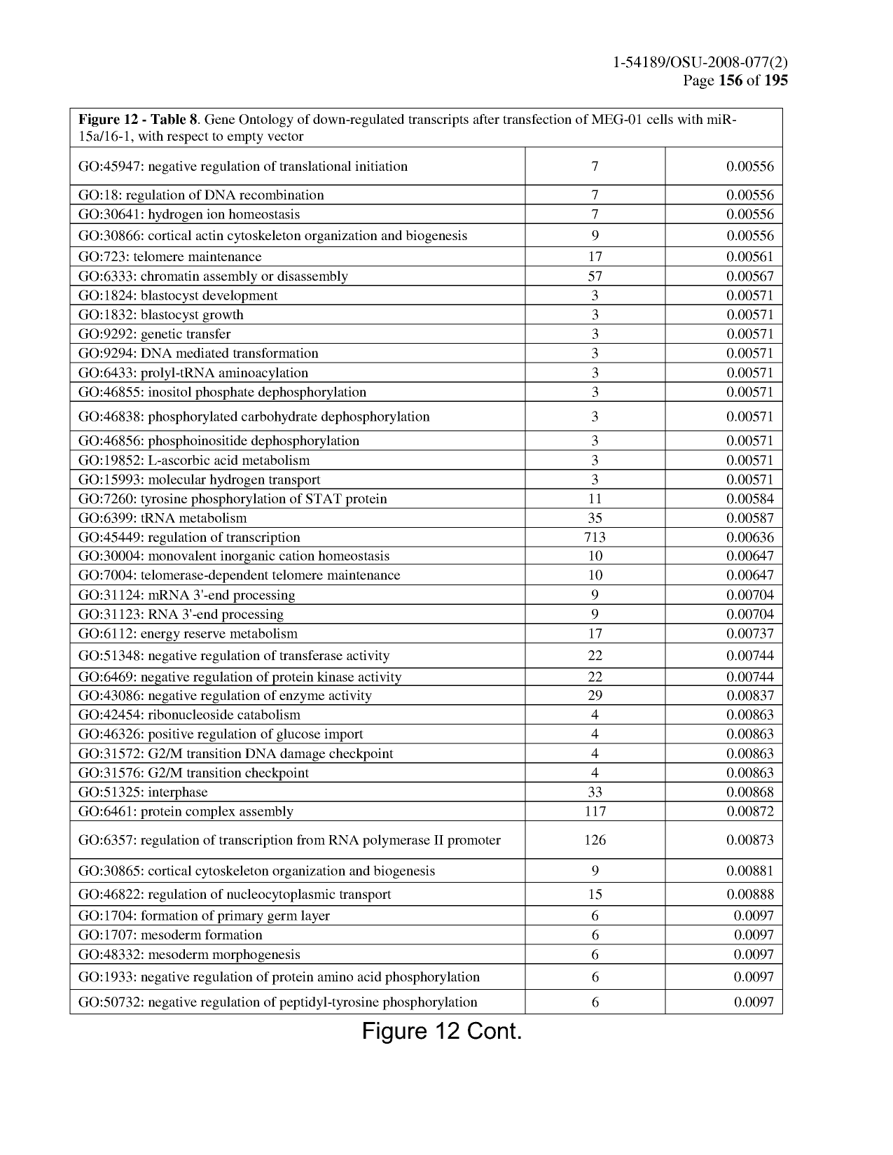

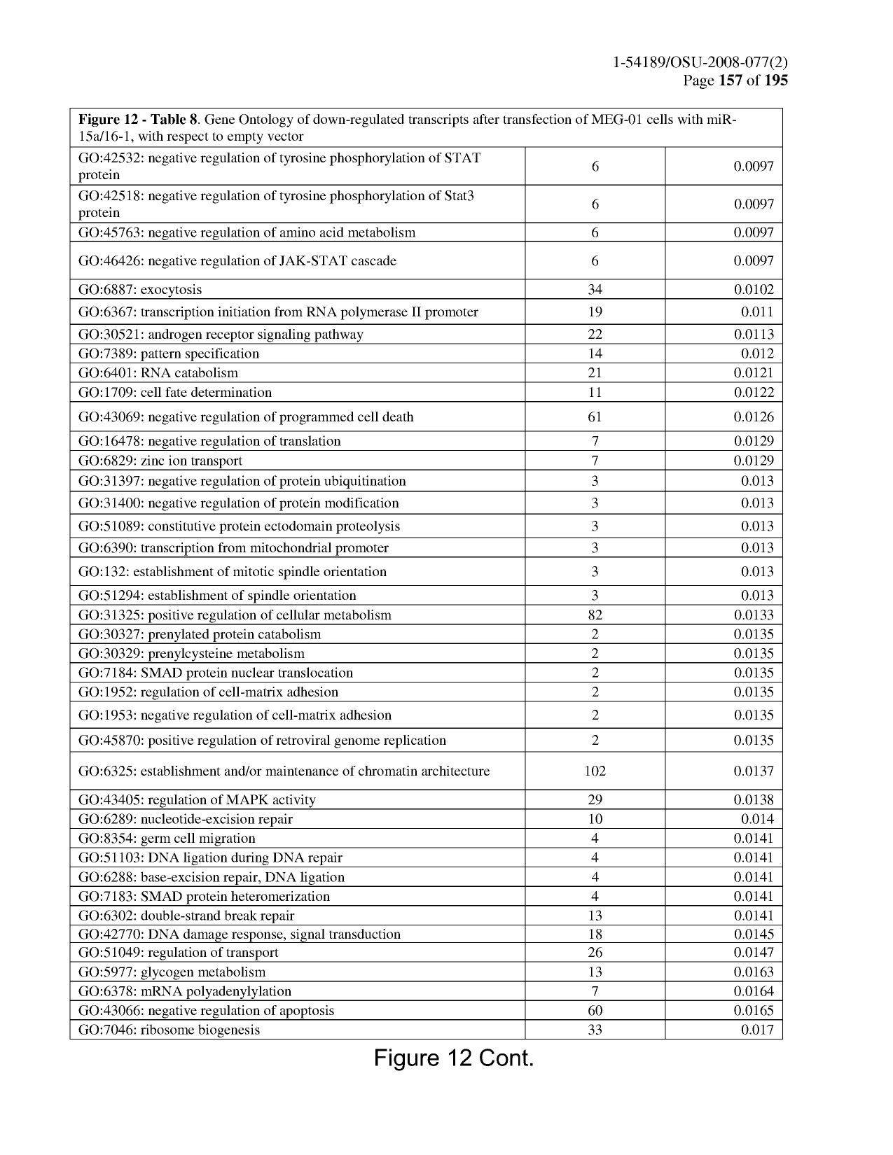

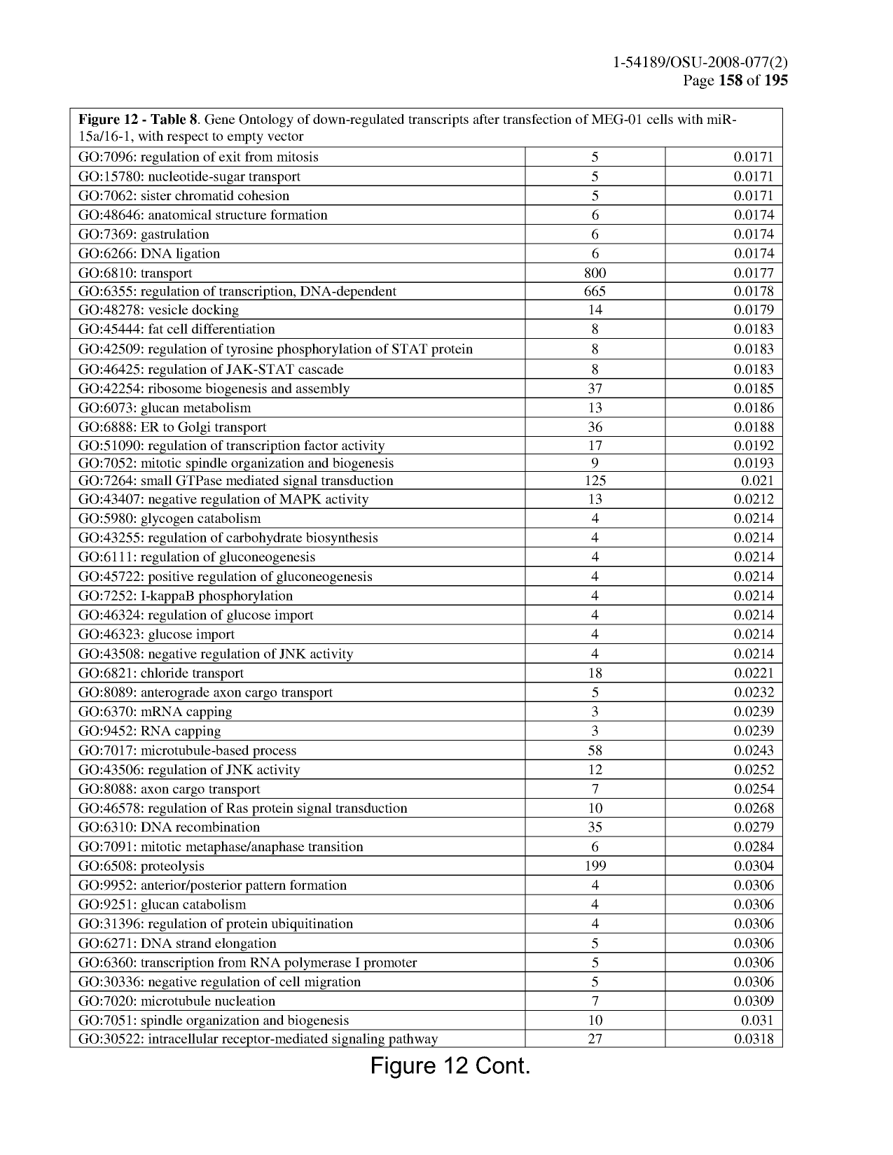

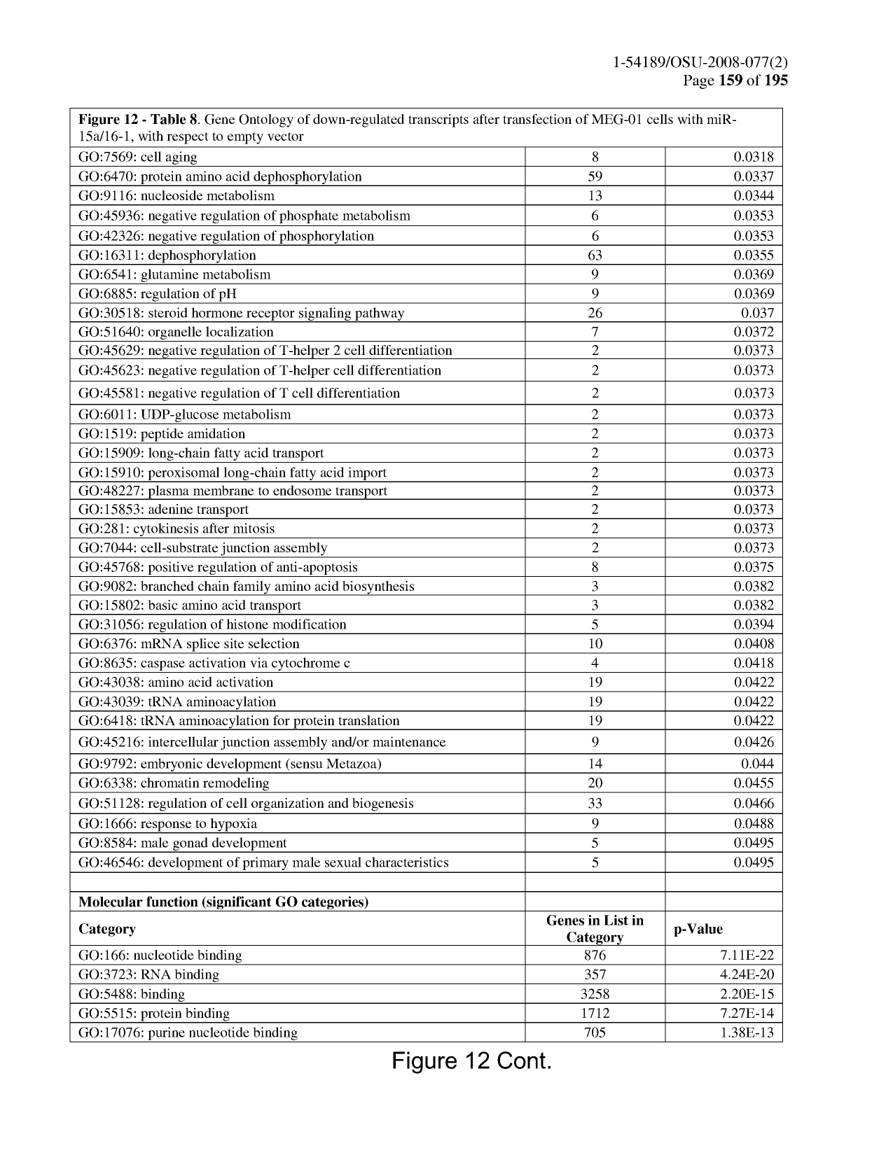

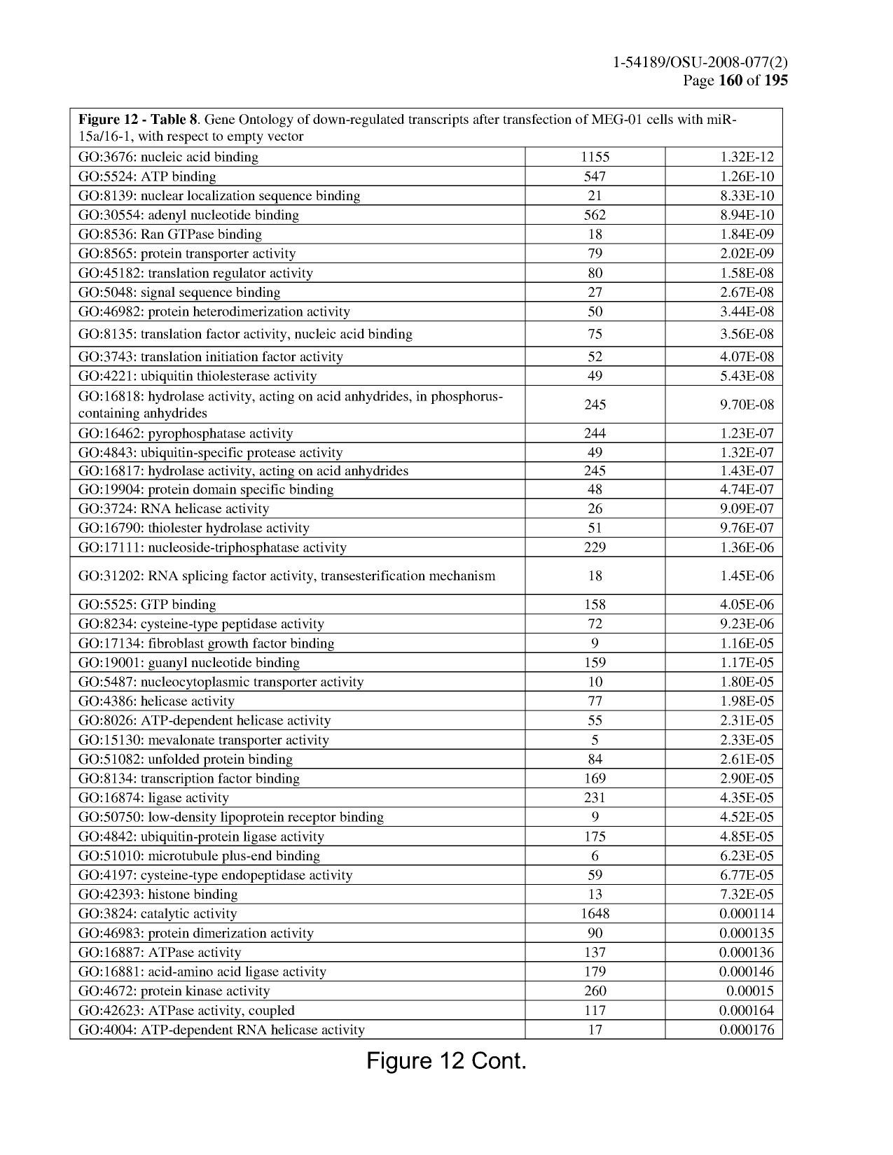

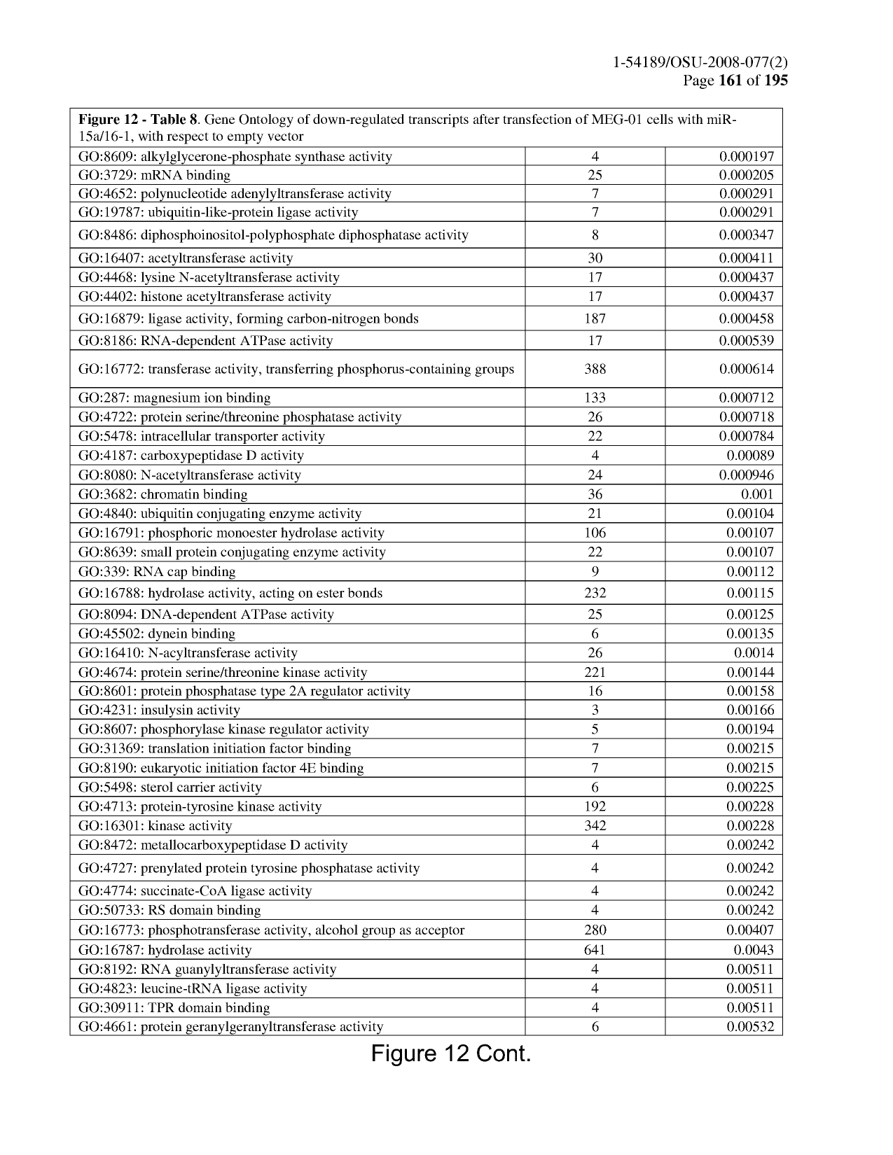

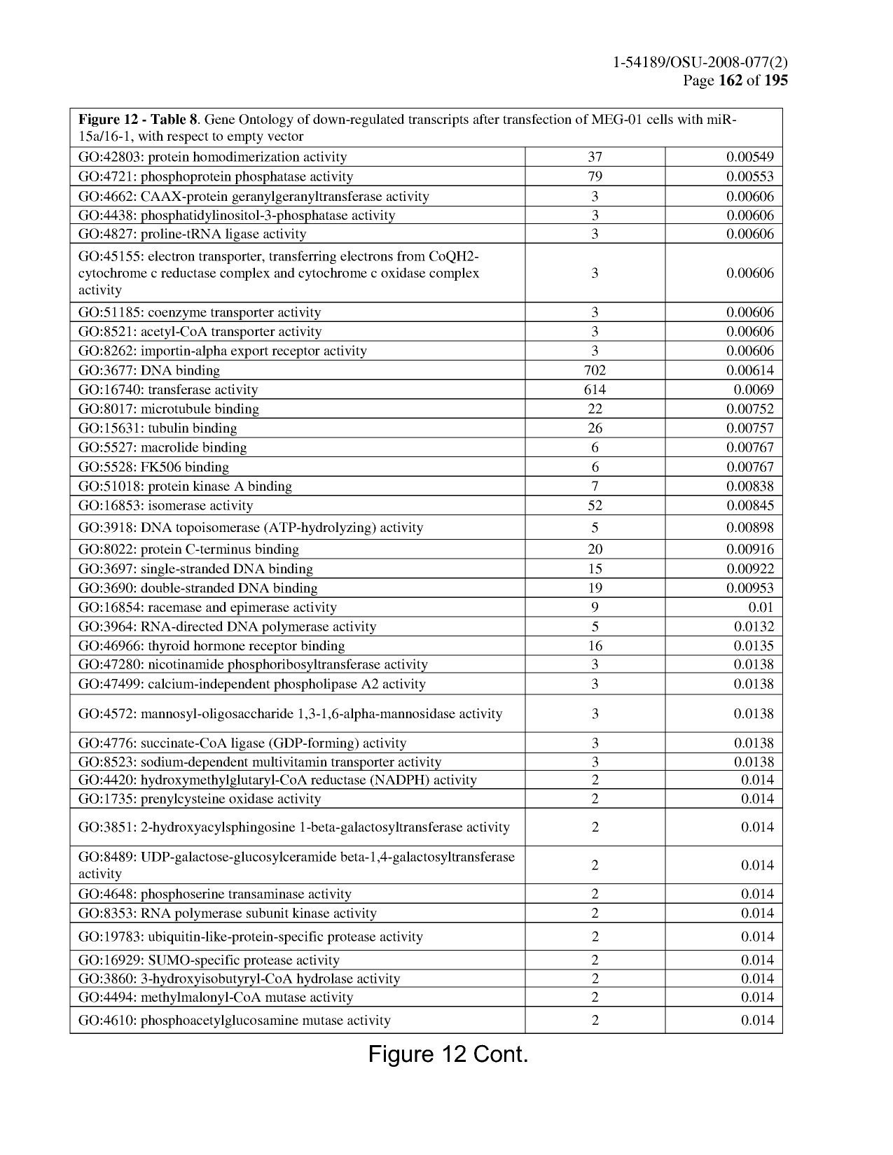

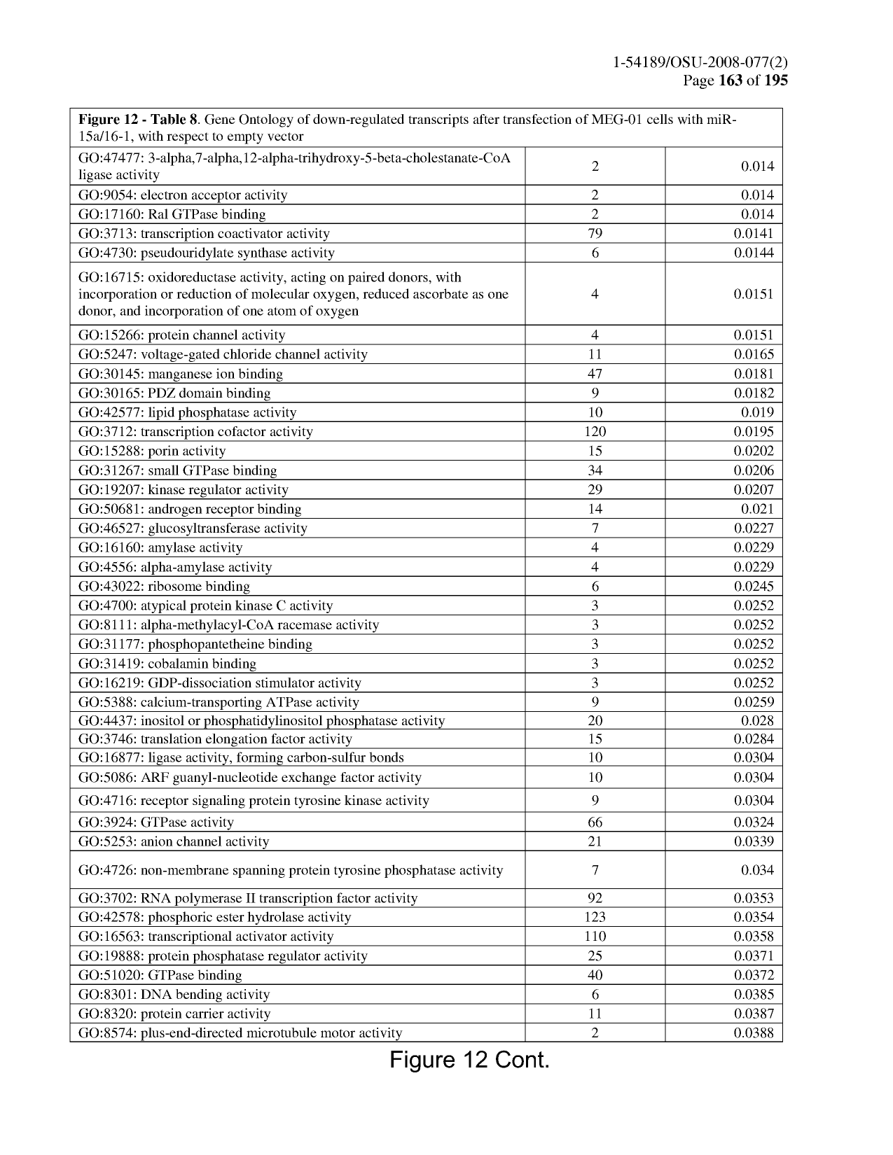

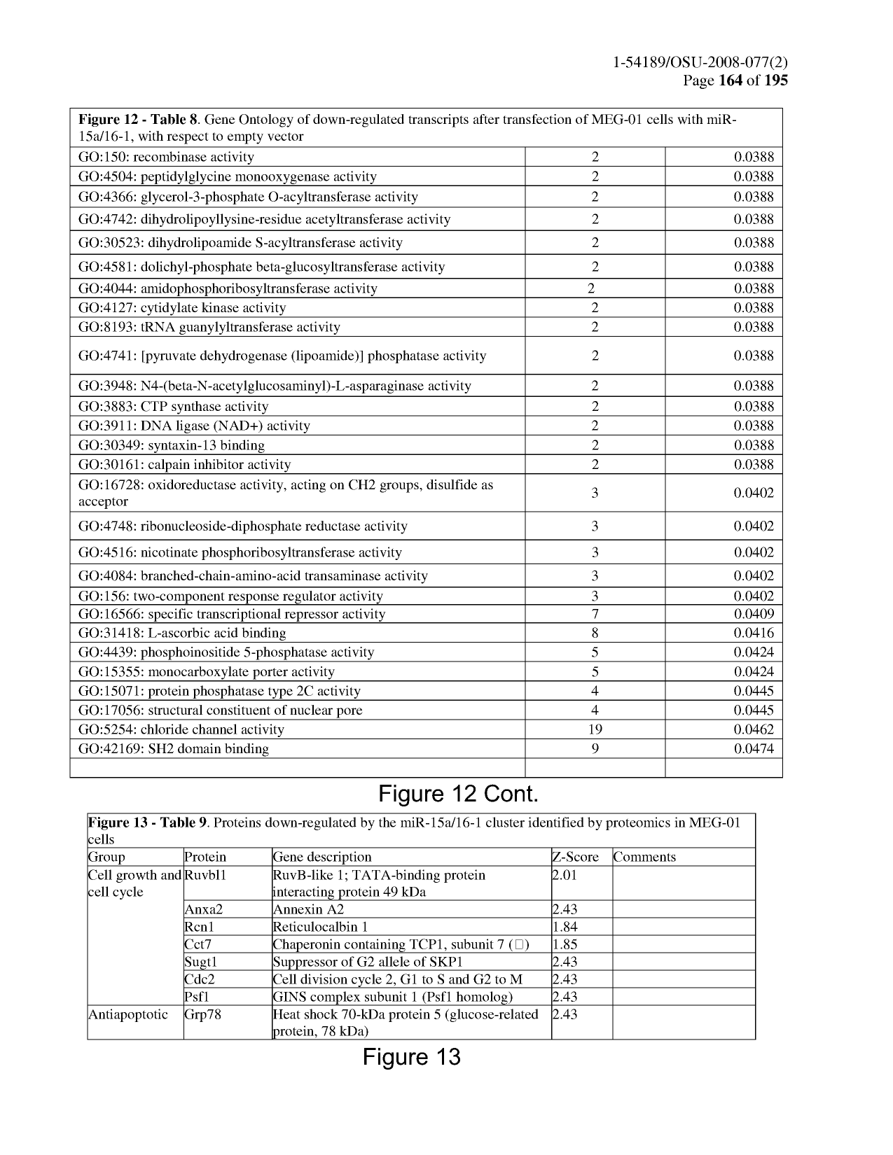

As shown in FIG. 11—Table 7, the number of genes containing AREs in their 3′ UTR was 36 of 265 (13.6%) up-regulated genes, and 666 of 3,307 (20.1%) among the down-regulated genes. This difference was statistically significant, with a _{X}² value of 6.674 (P=0.0098). Among the 85 genes that are predicted targets of miR-15α/16-1, 28 (32.9%) contain AREs, whereas among the remaining 3,222 down-regulated genes that are not commonly predicted targets, 638 (19.8%) mRNAs contain AREs (χ² value=8.89, P=0.003). According to the number of motifs in the ARE stretch, the ARE-mRNAs can be clustered into five groups, containing five (cluster I), four (cluster II), three (cluster III), and two (cluster IV) pentameric repeats, whereas cluster V contains only one pentamer within the 13-bp ARE pattern as described (25).

The ARE-cluster distribution of the Mir-15α/16-1 deregulated genes is shown in FIG. 4—Table 1. These results indicate that AREs are more frequently found among down-regulated targets of Mir-15α/16-1, especially the commonly predicted targets, further confirming the influence of AREs in miR-16 targeting.

Gene Ontology (GO) of Genes Deregulated by Mir-15α/16-1 Cluster

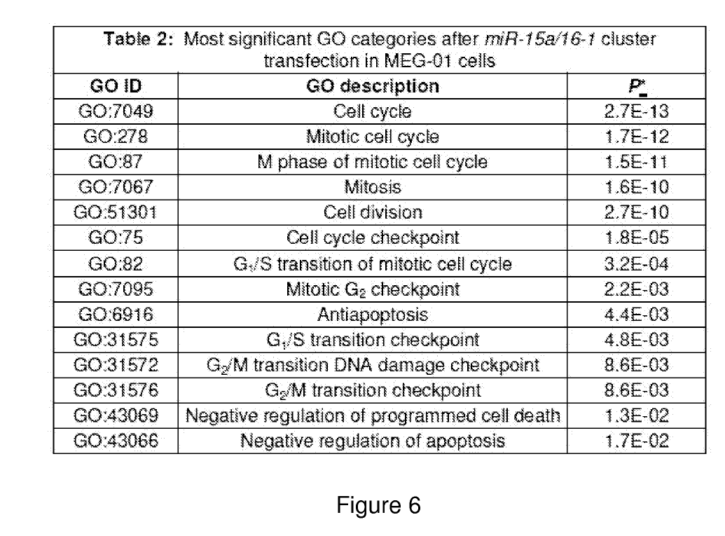

Genes found to be differentially expressed in MEG-01 cells after transfection with pRS15/16 versus pRS-E were analyzed with the GeneSpring Gene Ontology browser tool to identify the Gene Ontology categories most represented in down-regulated genes (FIG. 6—Table 2, FIG. 4—Table 8). These results show that the Mir-15α/16-1 cluster directly or indirectly affects the expression of many cell cycle-related genes.

In particular, many genes involved in the different transition checkpoints of the cell cycle are targeted by the miRNAs. Consistent with our previous finding that BCL2 is a target of miR-15a/16-1, in this GO ontology analysis, the category “antiapoptosis” (GO:6916) is significantly represented among the down-regulated transcripts.

Effect of miR-15a and miR-16-1 on MEG-01 Proteome

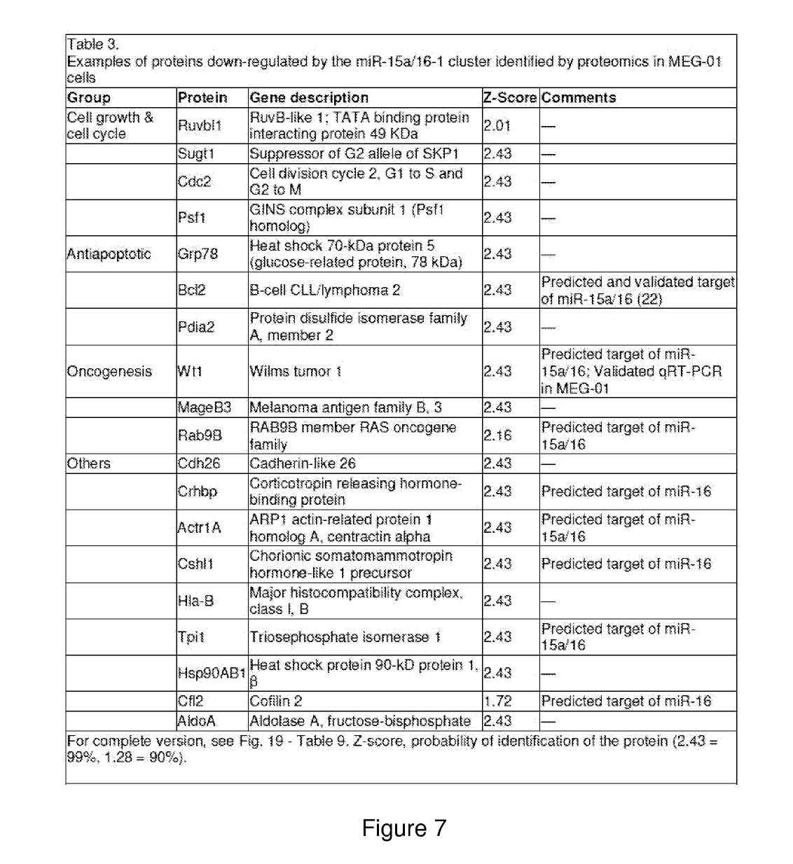

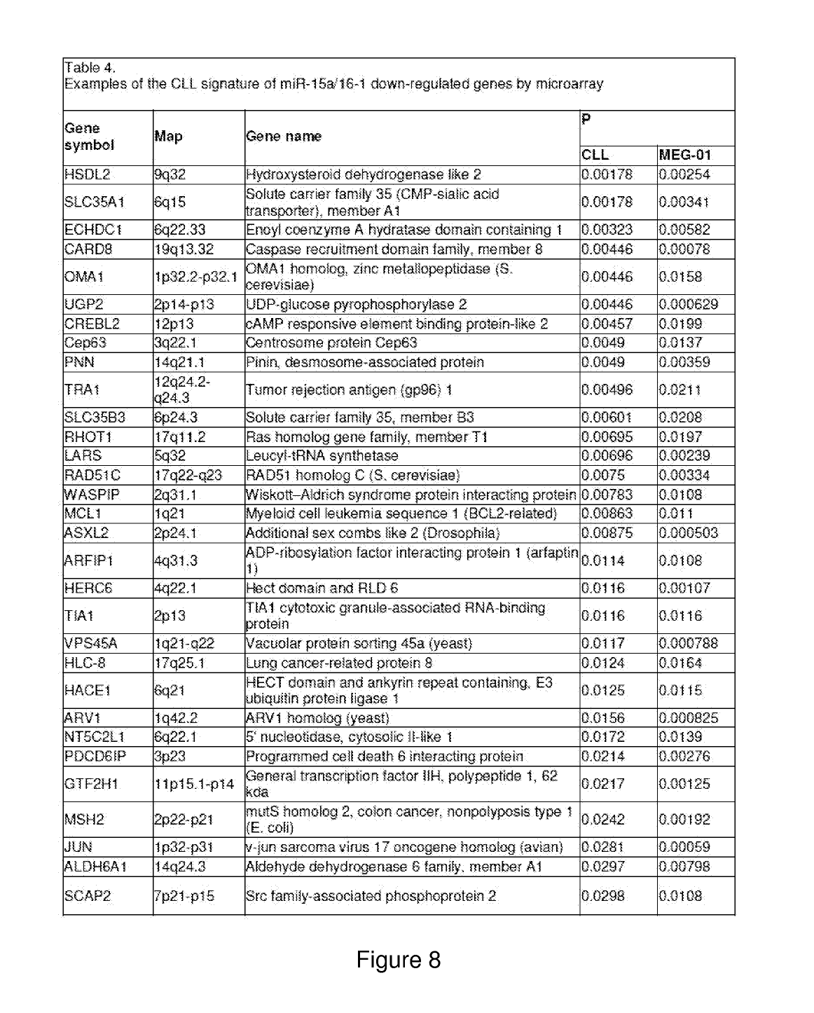

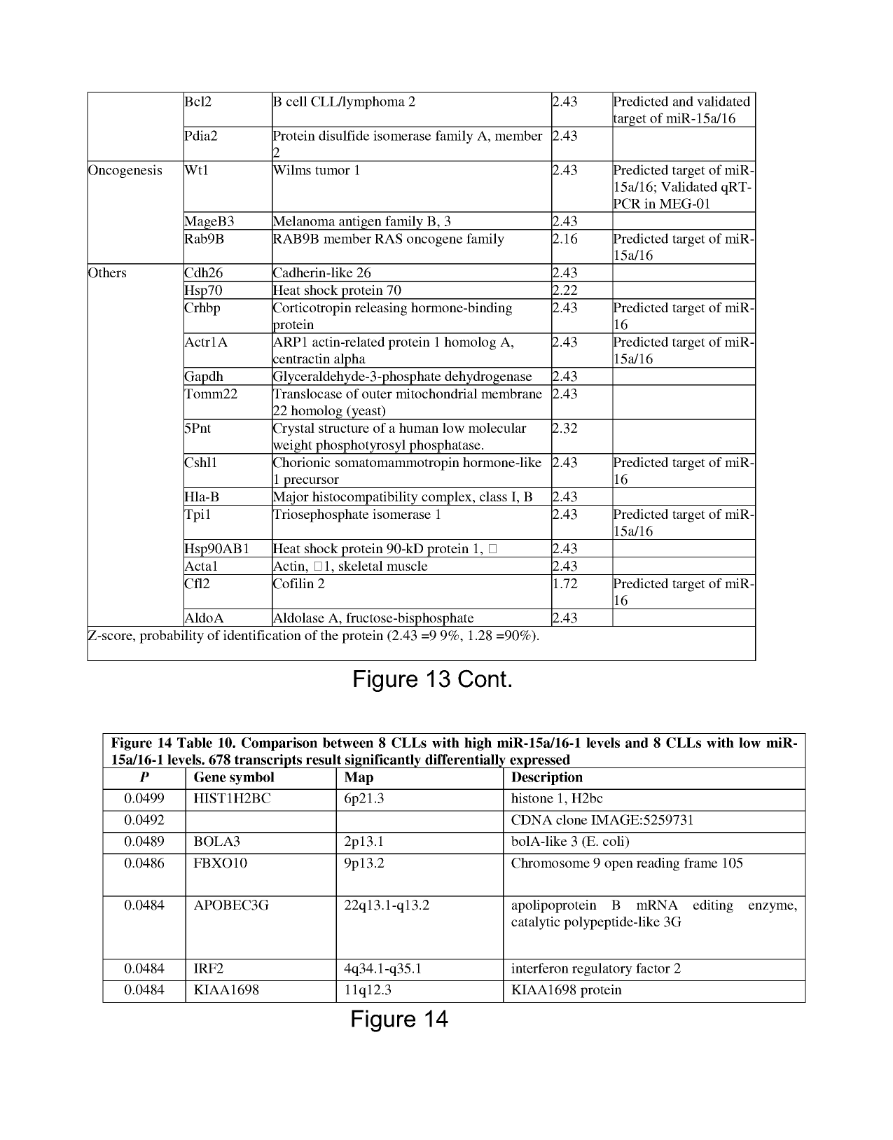

Because both transcriptional and translational levels of miRNA-dependent gene regulation have been described (26), to investigate the effects of Mir-15α/16-1 on MEG-01 cells at the protein level, we analyzed the proteins differentially expressed between MEG-01 cells transfected with pSR15/16 or pRS-E vector 48 h after transfection. By proteomics analysis, we identified proteins whose intensity was reduced 4-fold or more in the pRS15/16 group with respect to the pRS-E group. We isolated 27 different proteins (FIG. 7 - Table 3, FIG. 13 - Table 9).

Interestingly, BCL2, which we had already shown as a target of Mir-15α/16-1 (22), and WTI , another predicted target of these miRNAs, were identified. The targeted proteins have a variety of biological functions and can be grouped into four groups.

The first group includes proteins that play a role in regulation of cell growth and cell cycle (Ruvb11, Anxa2, Rcn1, Cct7, Sugt1, Cdc2, Psf1), another category is formed by antiapoptotic proteins (Grp78, Bc12, Pdia2), and proteins involved in human tumorigenesis, either as oncogenes, or as tumor-suppressor genes (Wt1, MageB3, Rab9B). The remaining 14 proteins have different biological functions, and we identified them as “others.” Among the 27 experimentally identified down-regulated proteins, 8 (29.6%) are predicted targets of Mir-15α/116 by at least one of the prediction algorithms Finally, among this group of eight proteins, two (Bc12, and Cf12) were present also in the group of down-regulated mRNAs.

Validation of the Results in the MEG-01 Cell Line

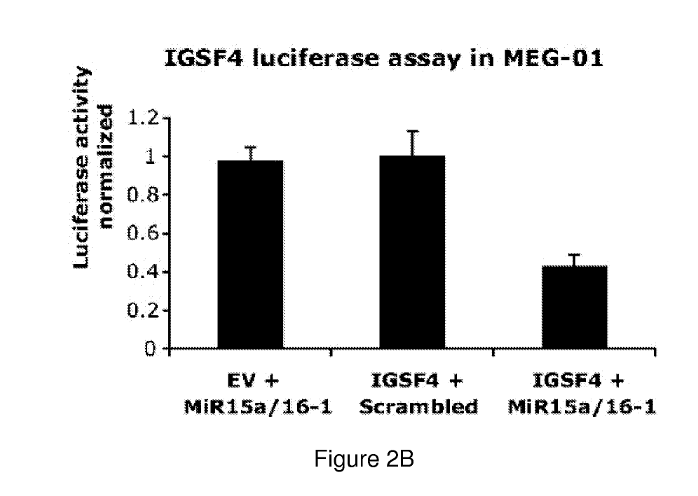

To validate the results obtained by transcriptomic or proteomic analyses, we assayed the expression of nine genes (four identified by the EST microarray, two by proteomics, and three identified by neither of the techniques and therefore considered as negative controls), by qRT-PCR in MEG-01 cells transfected with pRS15/16 or pRS-E (control). As shown in FIG. 2A, the transfection with Mir-15α/16-1 reduces the expression of both microarray identified mRNAs (PDCD4, RAB21, IGSF4, SCAP2) and proteomics identified proteins (Bc12, Wt1). MiR-15a/16-1 transfection does not affect the expression of any of the control genes (IGF1, ACE, and ERBB2).

We also performed the luciferase assay on one of the validated genes (IGSF4) and demonstrated that the Mir-15α/16-1 cluster directly targets IGSF4 (FIG. 2B).

The direct interactions with BCL2 and DMTF1 were proved by us and others (7, 22). Therefore, we were able to consistently confirm the MEG-01 profile of down-regulated genes and identified another direct target of Mir-15α/16-1 in this leukemic model.

Variation of Expression of miR-15α/miR 16-1 Targets in Primary CLLs

Because MEG-01 is a leukemia cell model with abnormal 13q14 and loss of the miR15α/16-1 cluster (similar to CLL), but is a megakaryocytic established leukemic cell line, we investigated the effects of the different expression of Mir-15α/16-1 cluster also in primary CLLs.

Therefore, to verify whether some of the targets of Mir-15α/16-1 identified in MEG-01 cells were inversely correlated to the expression of these two miRNAs in CLL patients, we selected a group of 16 CLL samples in whom the expression of Mir-15α/16-1 had already been determined by miRNA microarray analysis in our previous studies (18, 27).

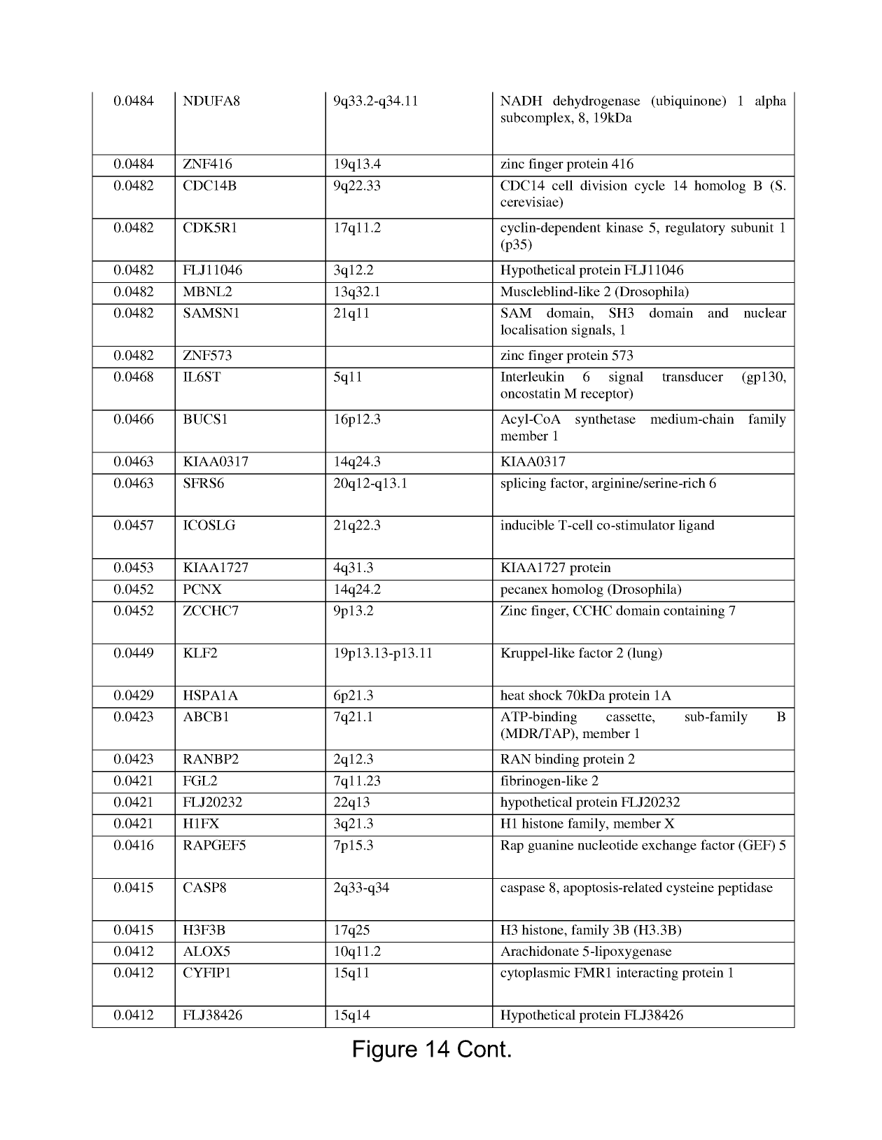

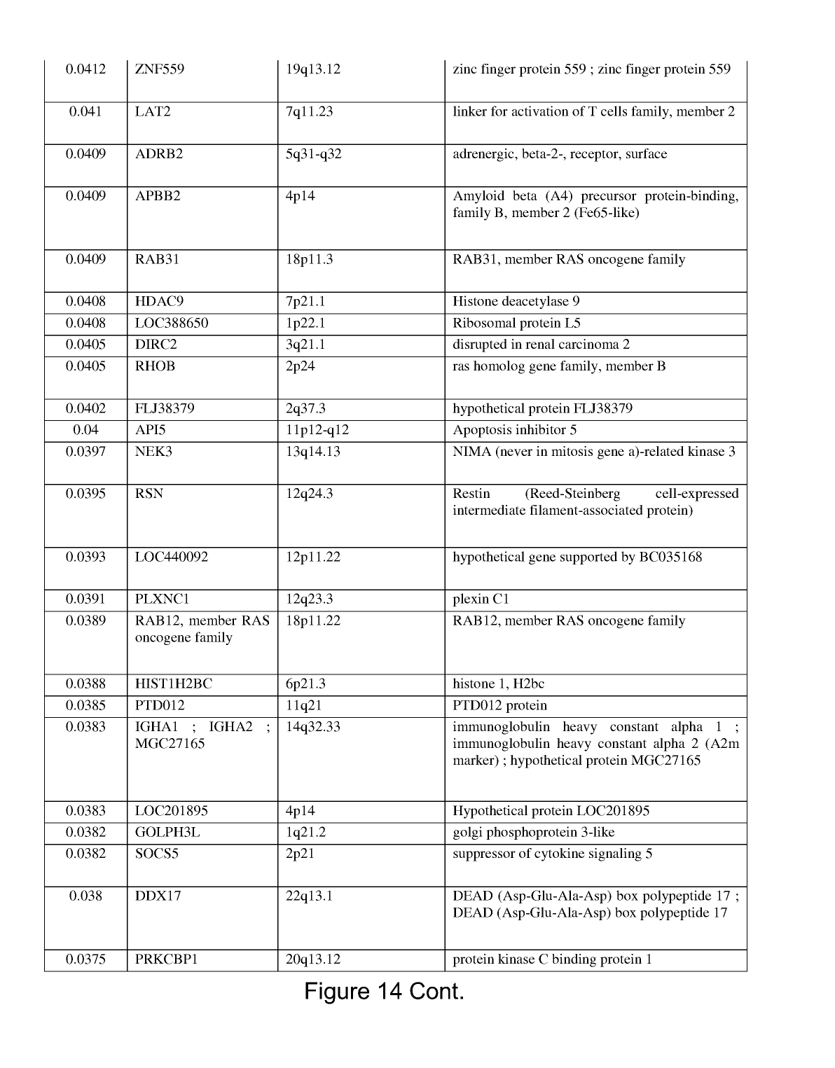

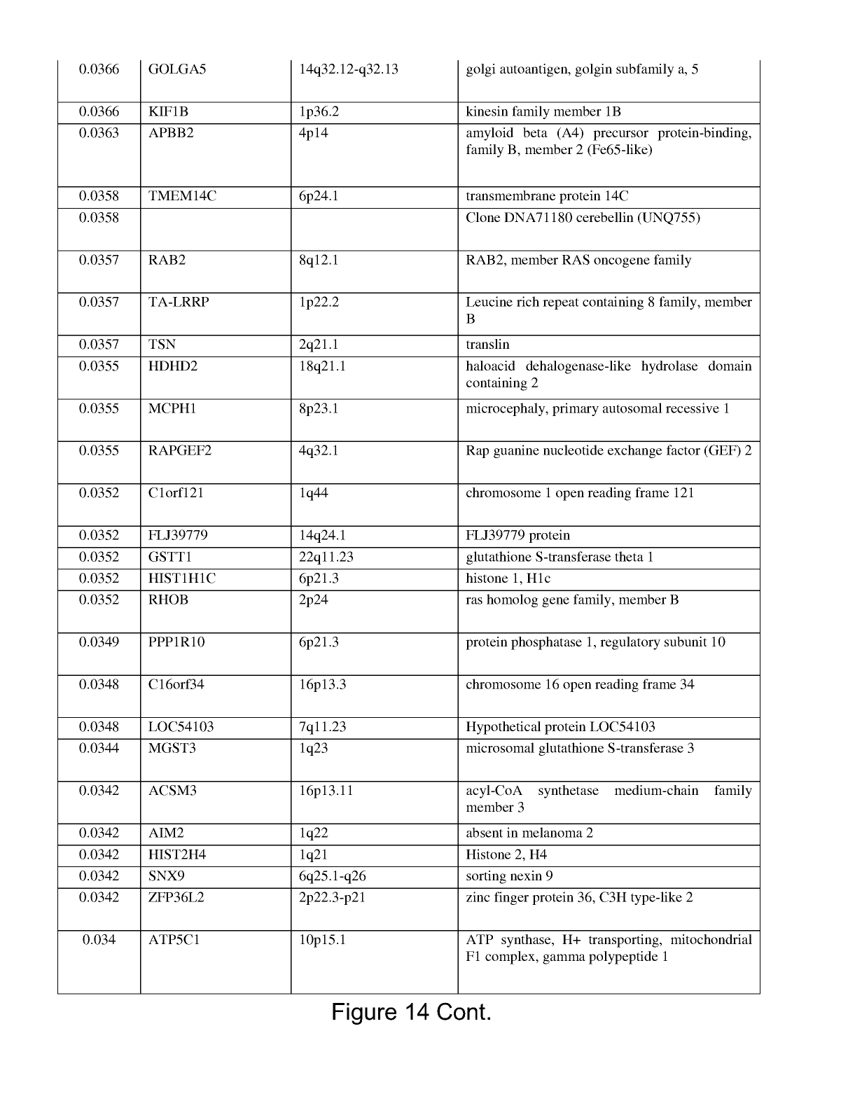

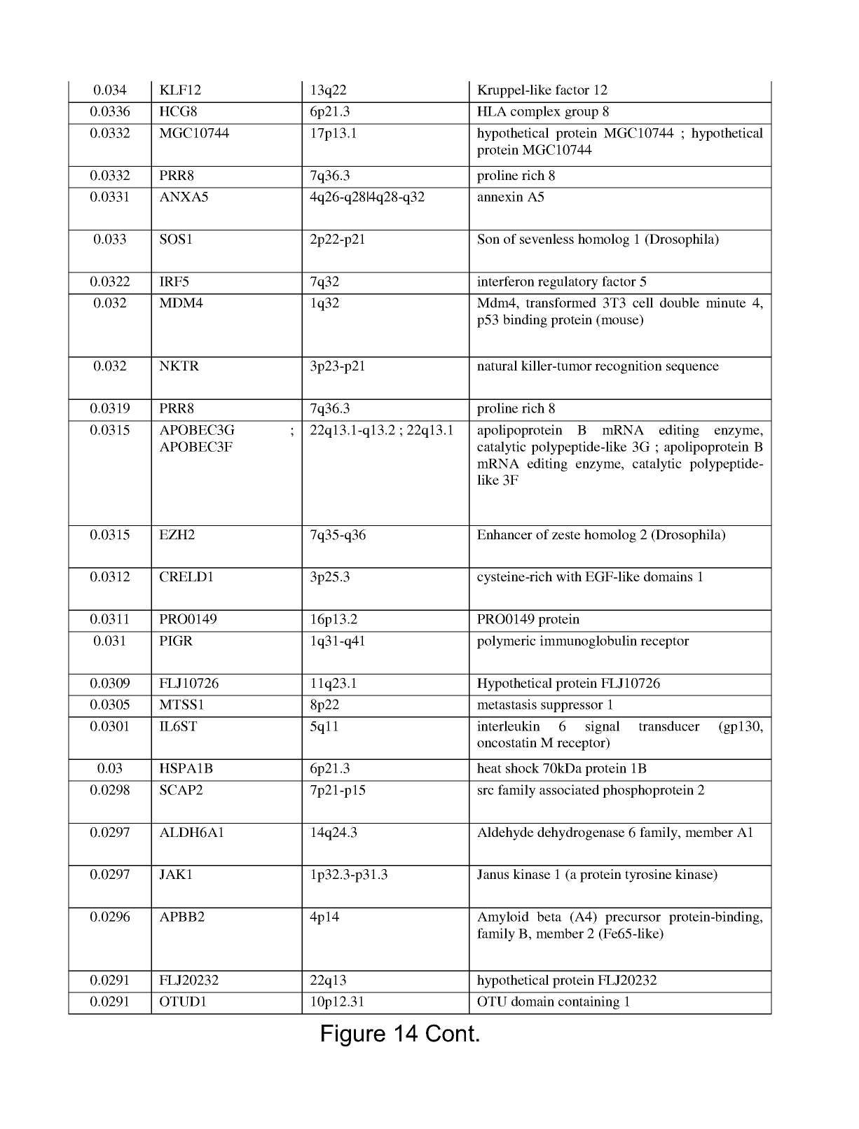

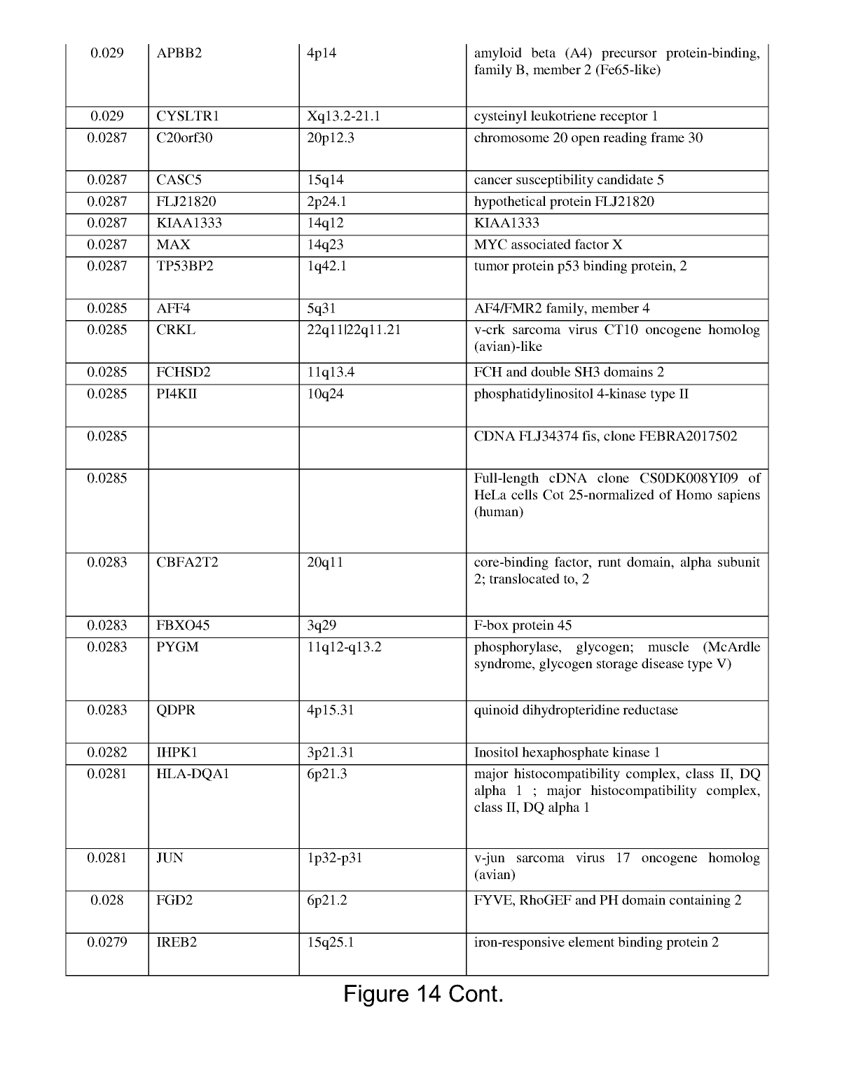

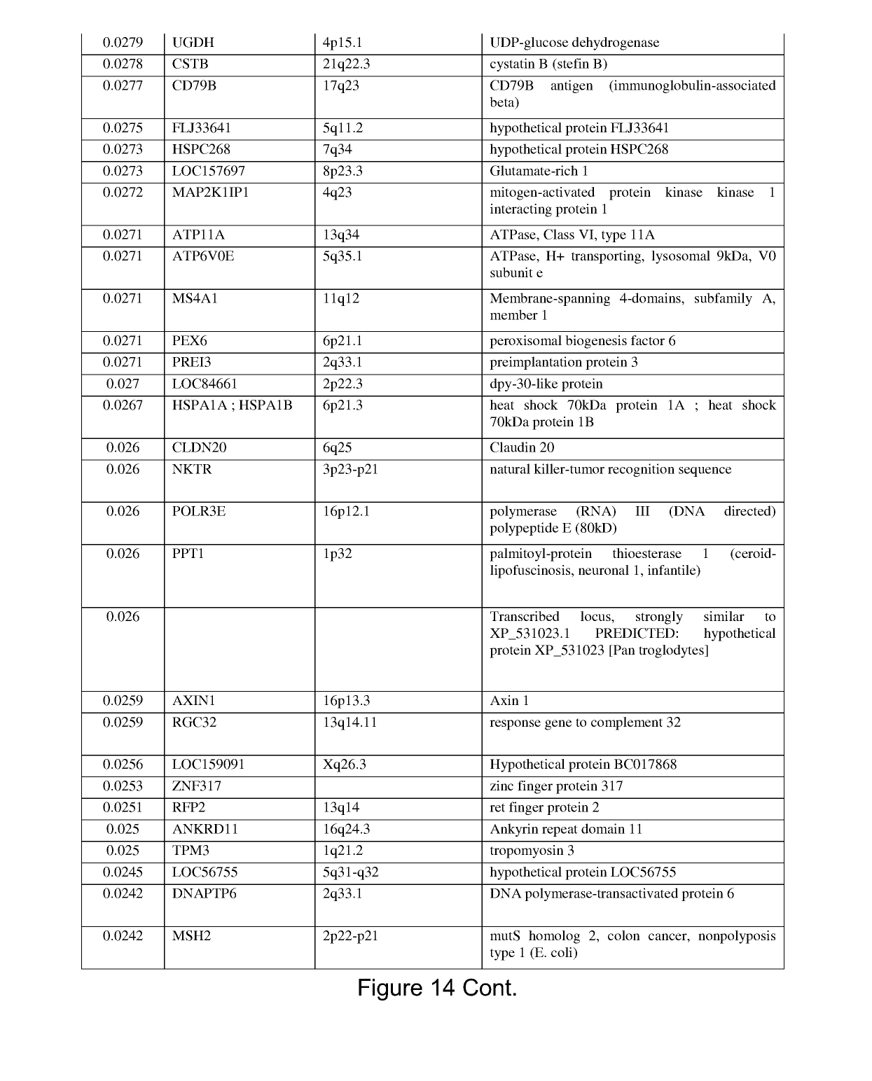

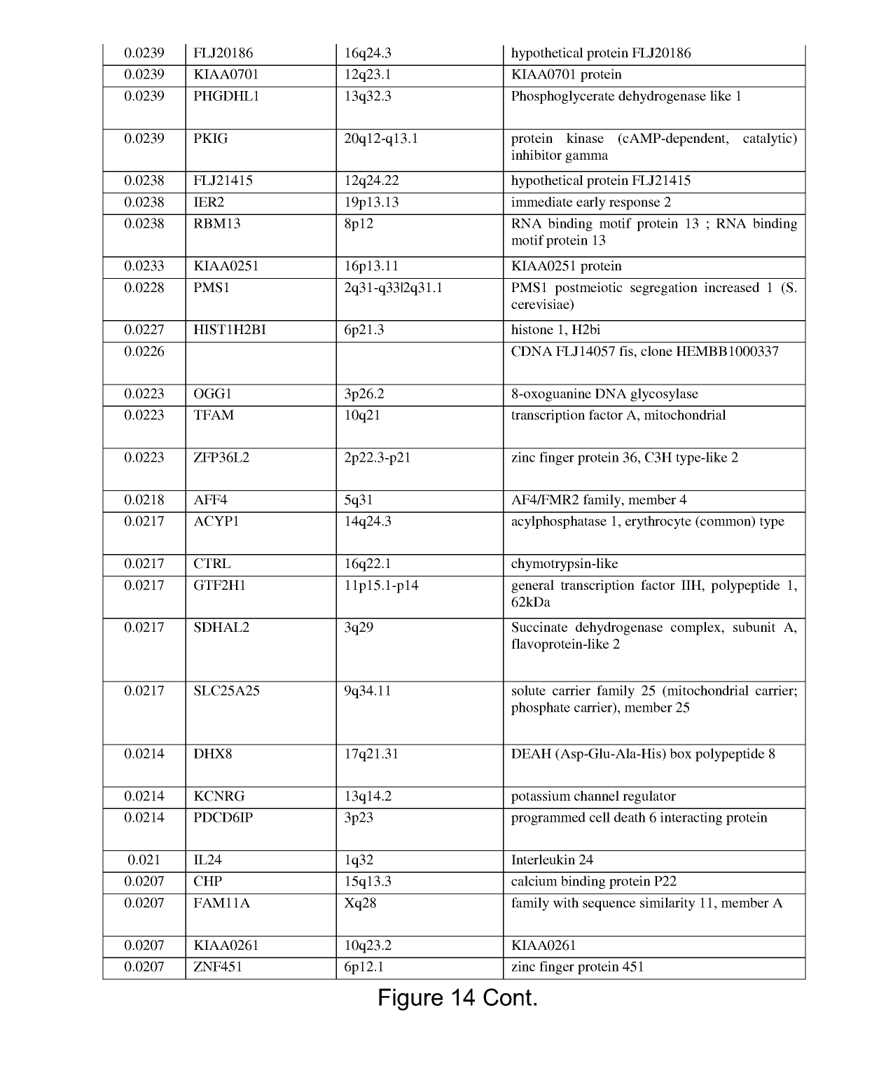

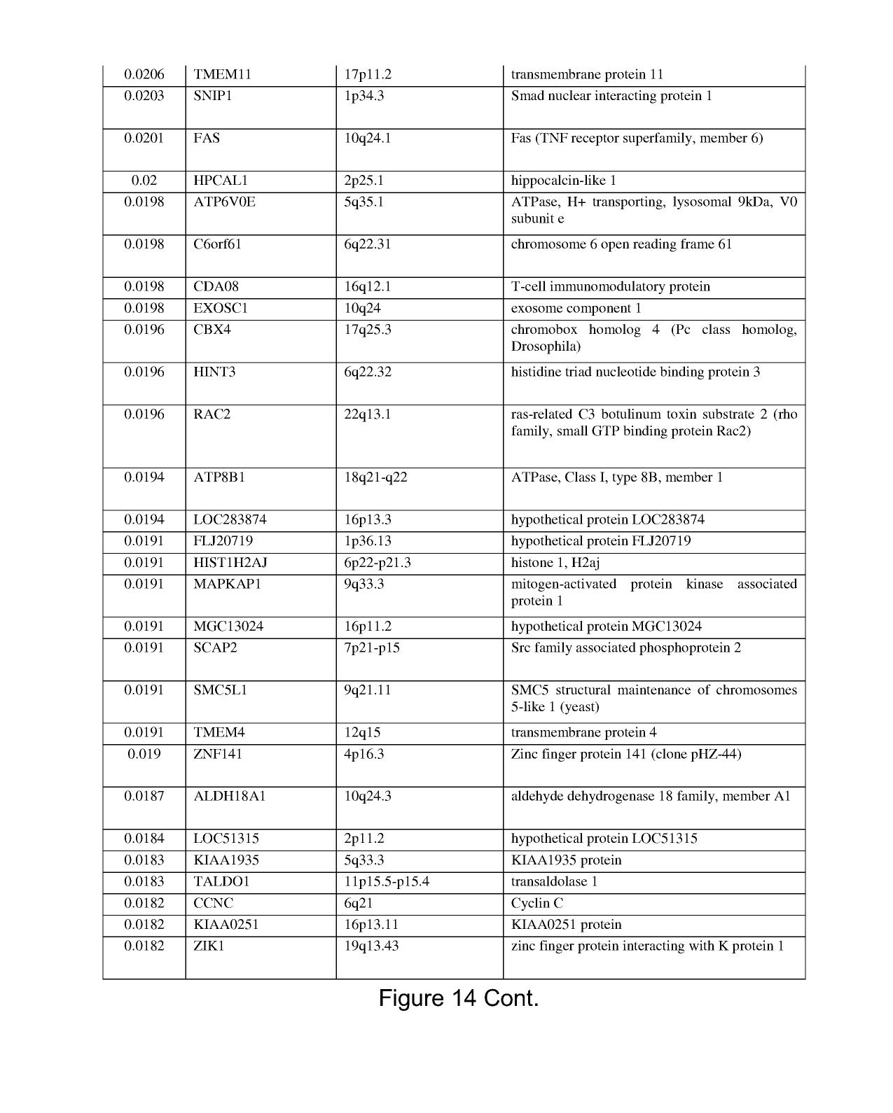

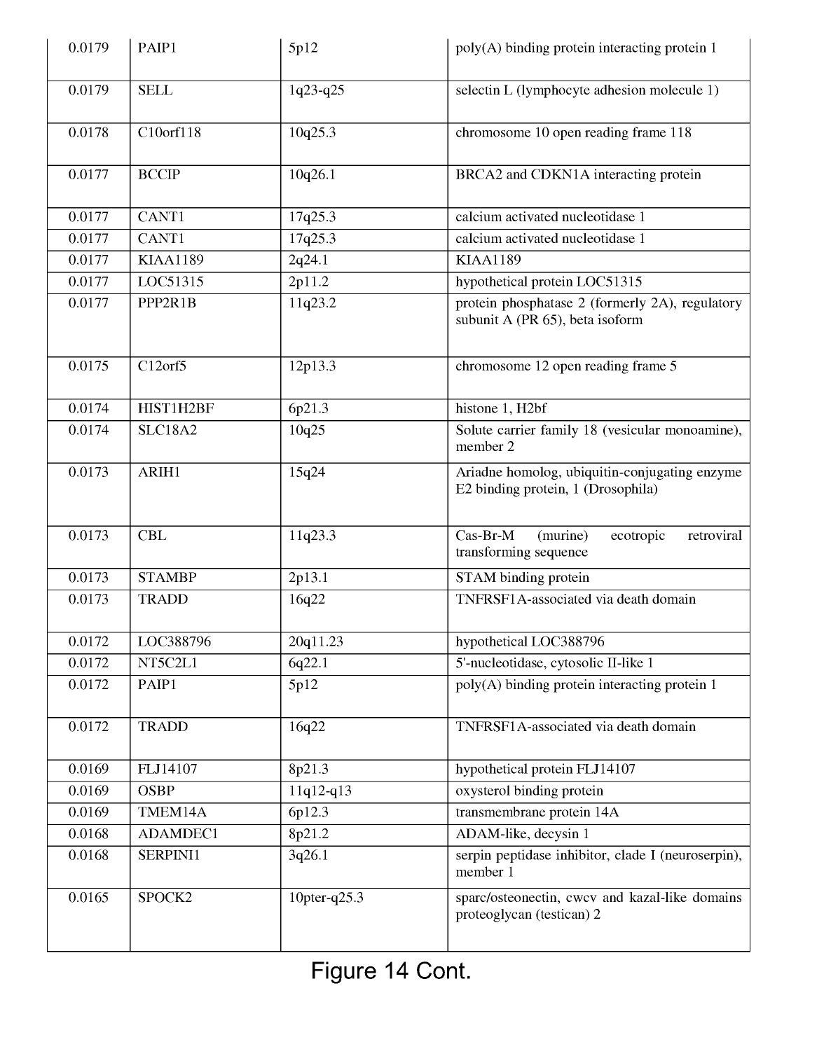

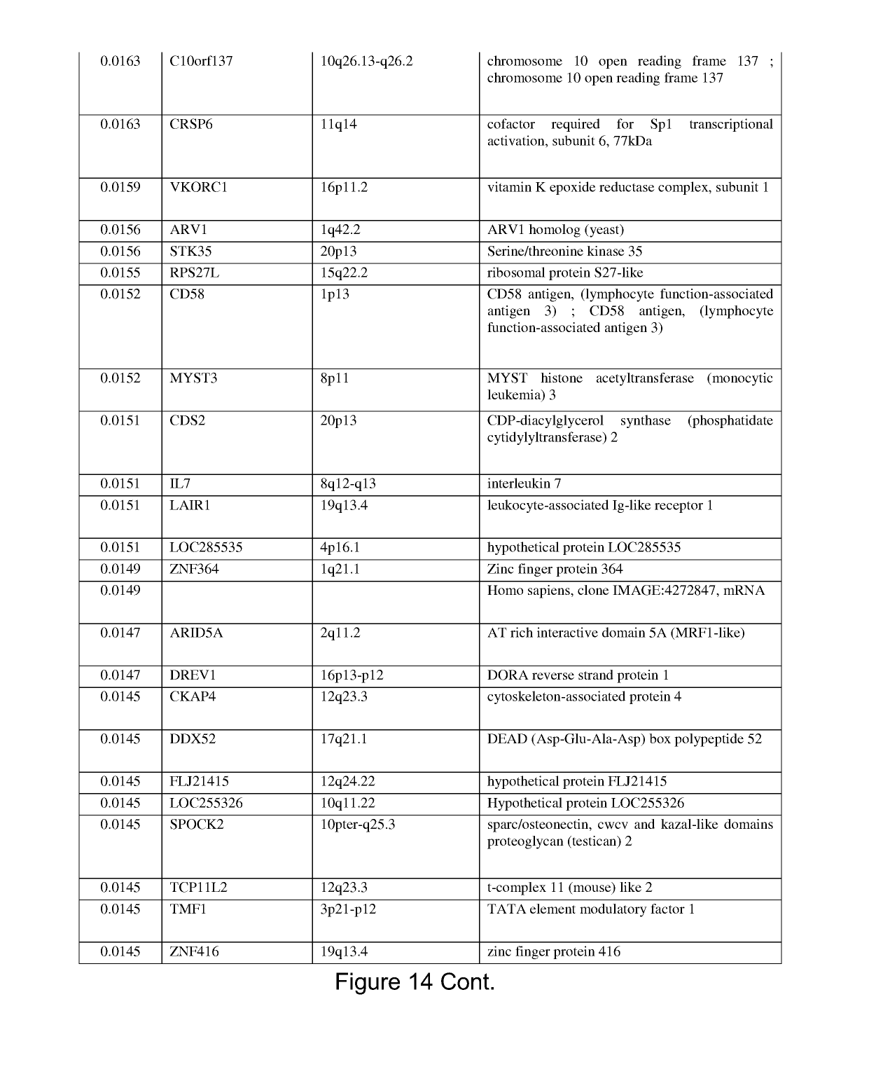

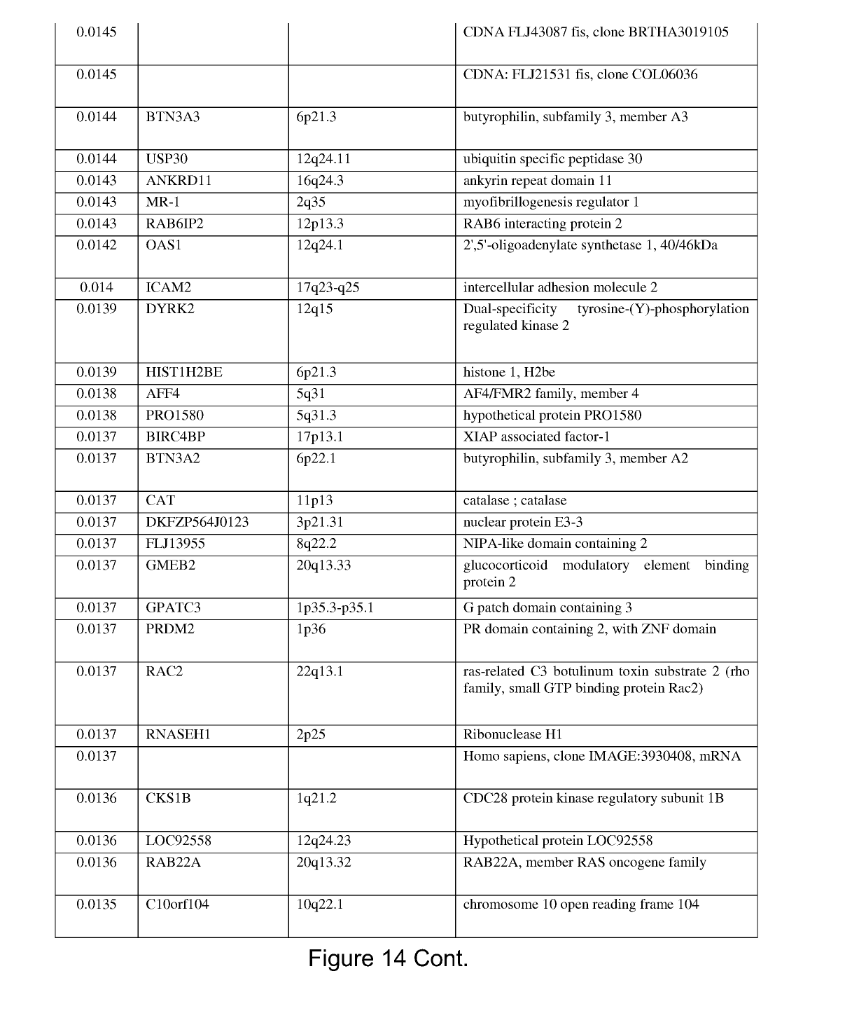

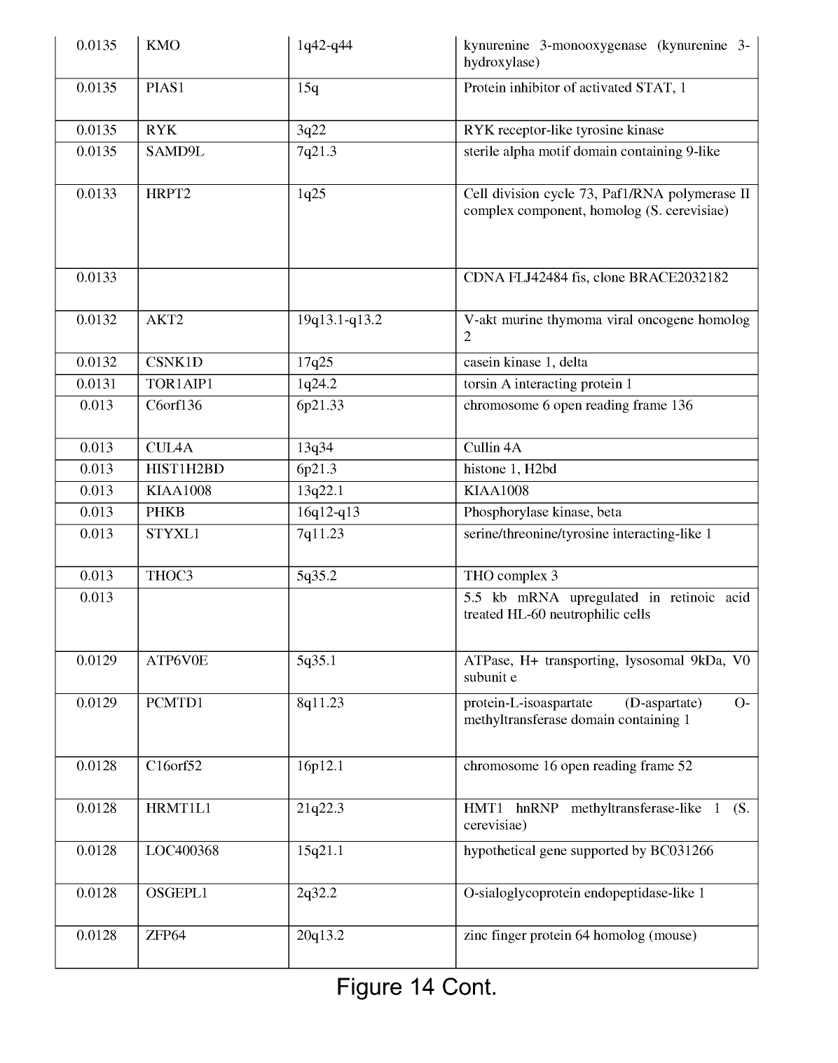

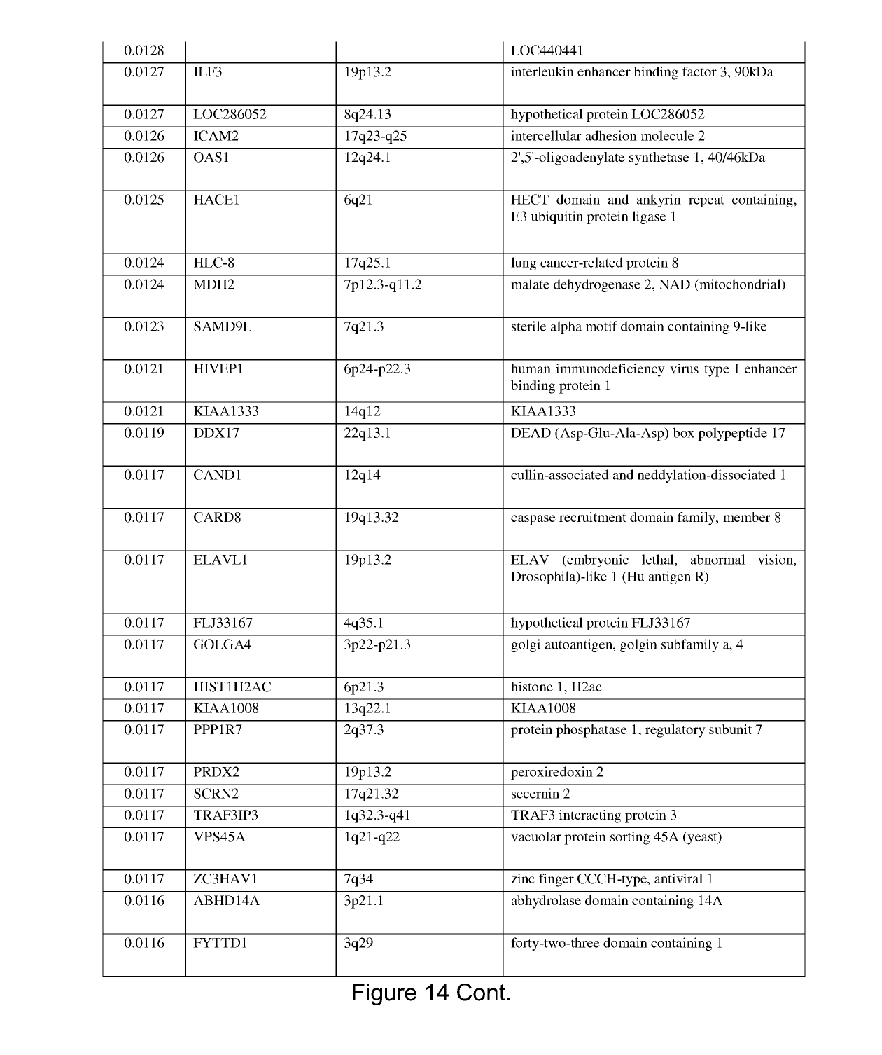

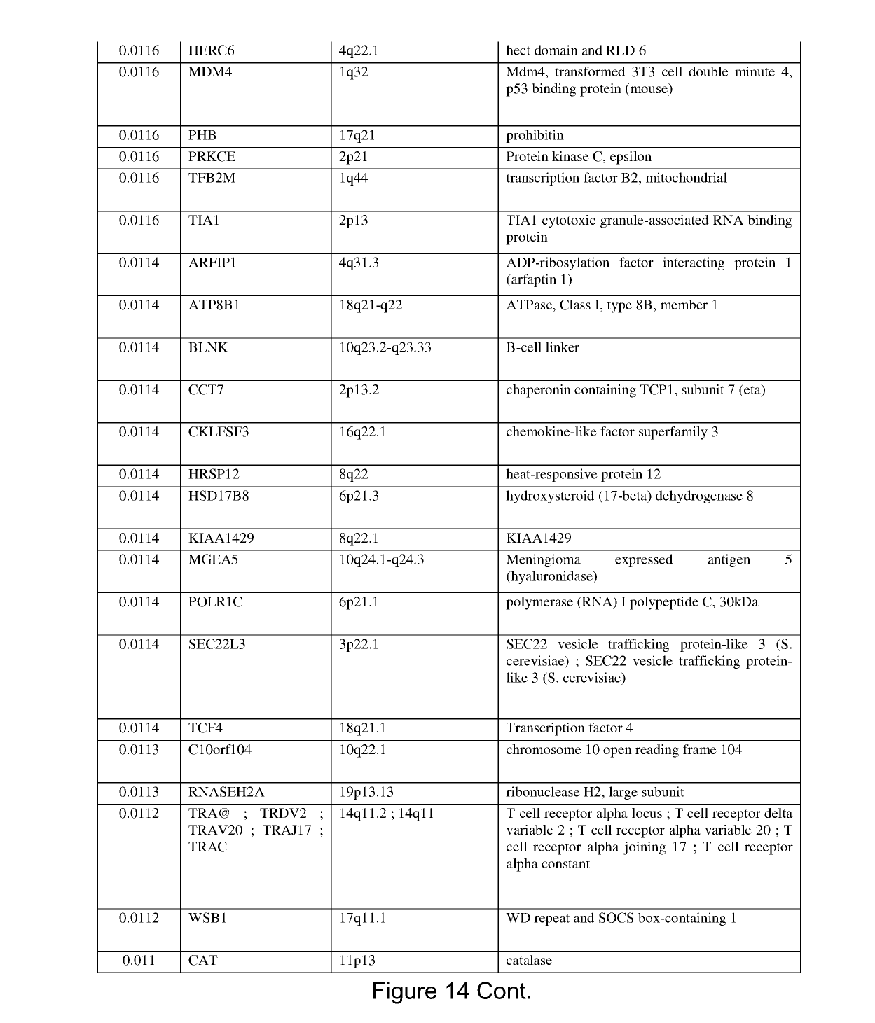

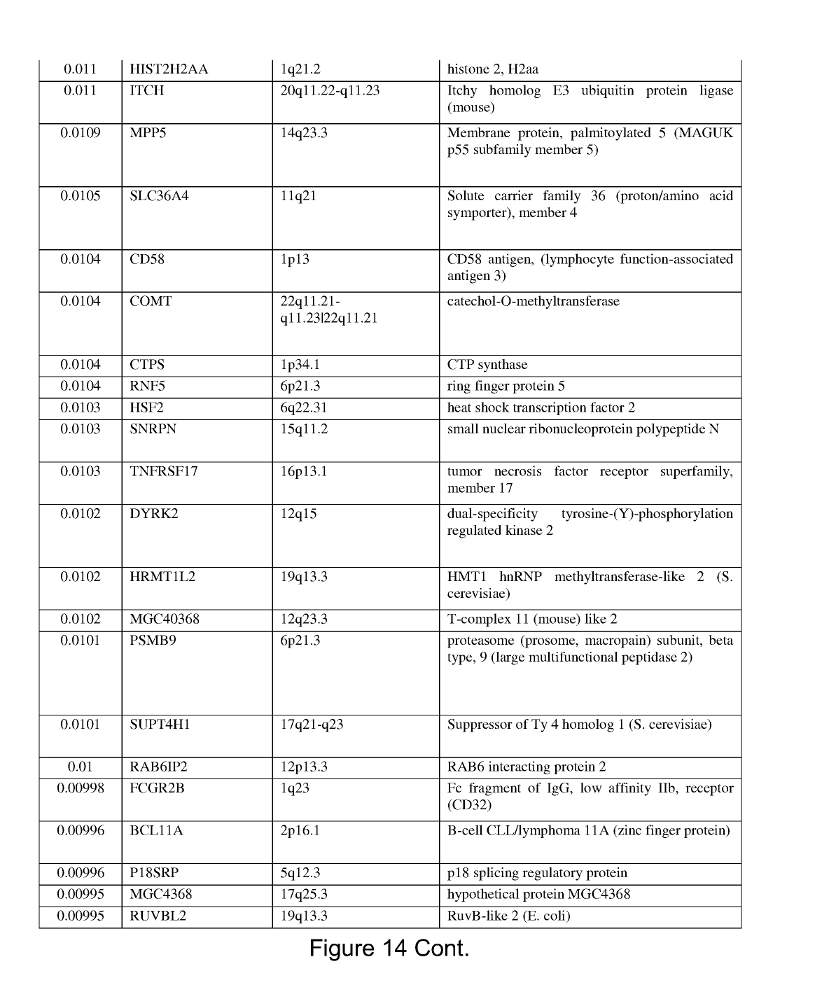

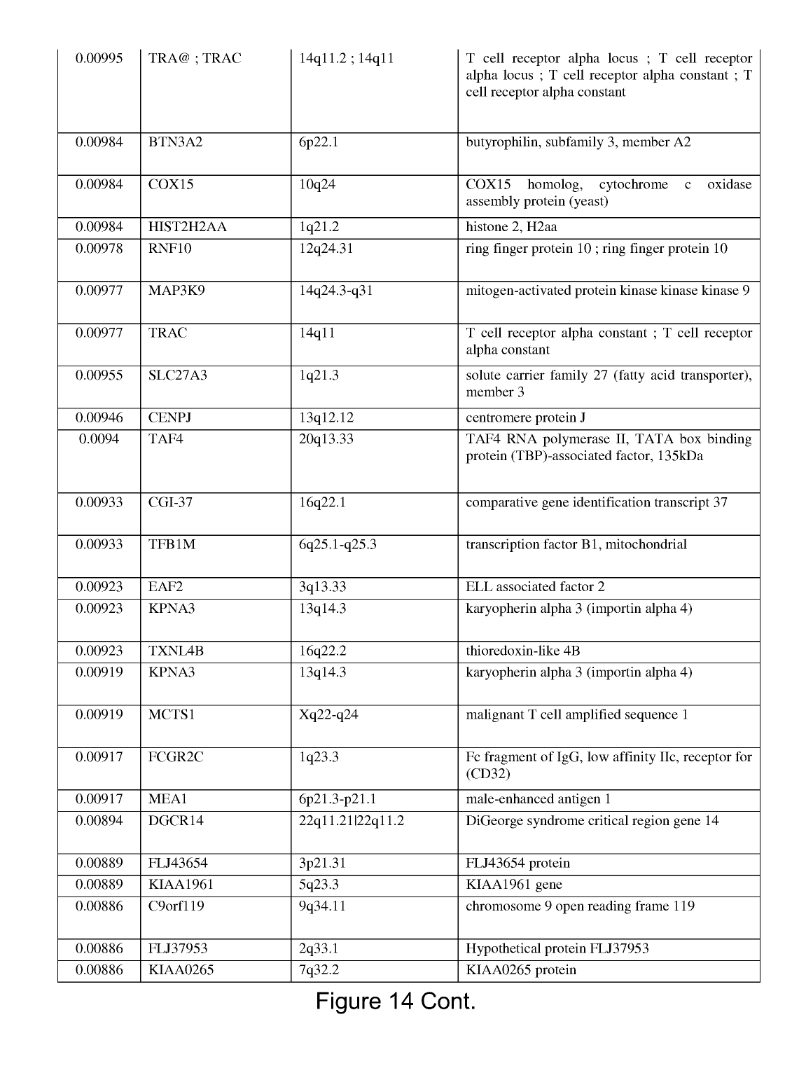

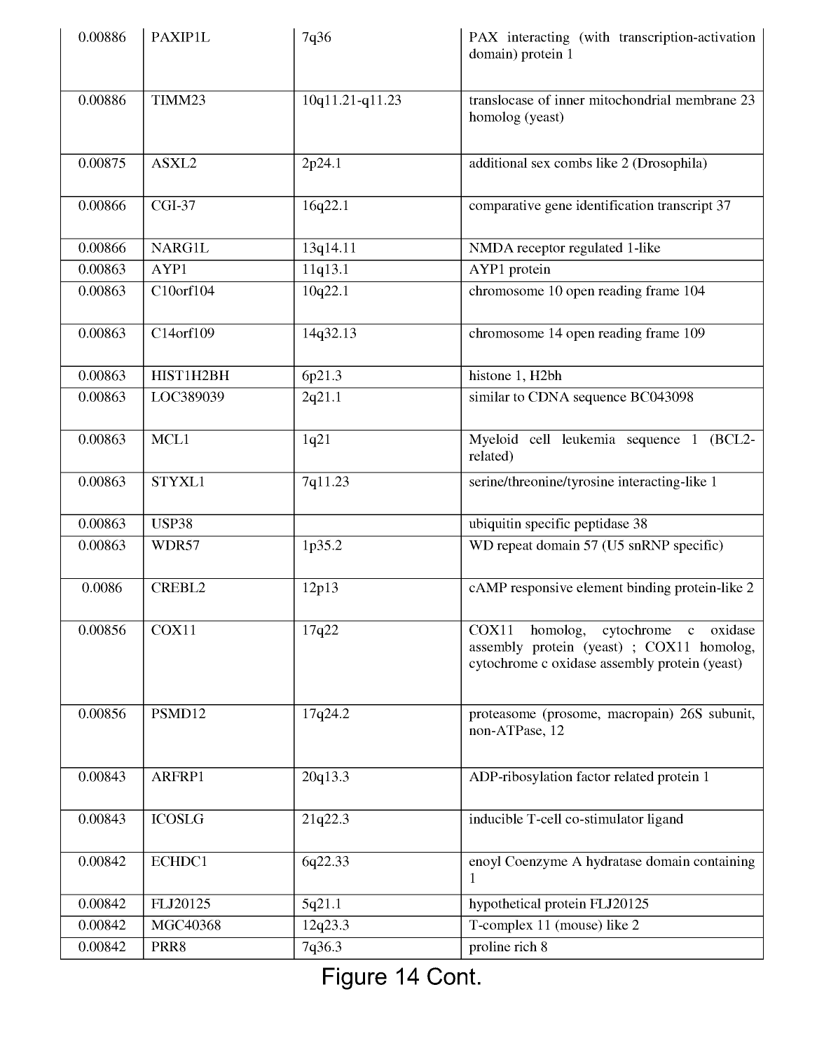

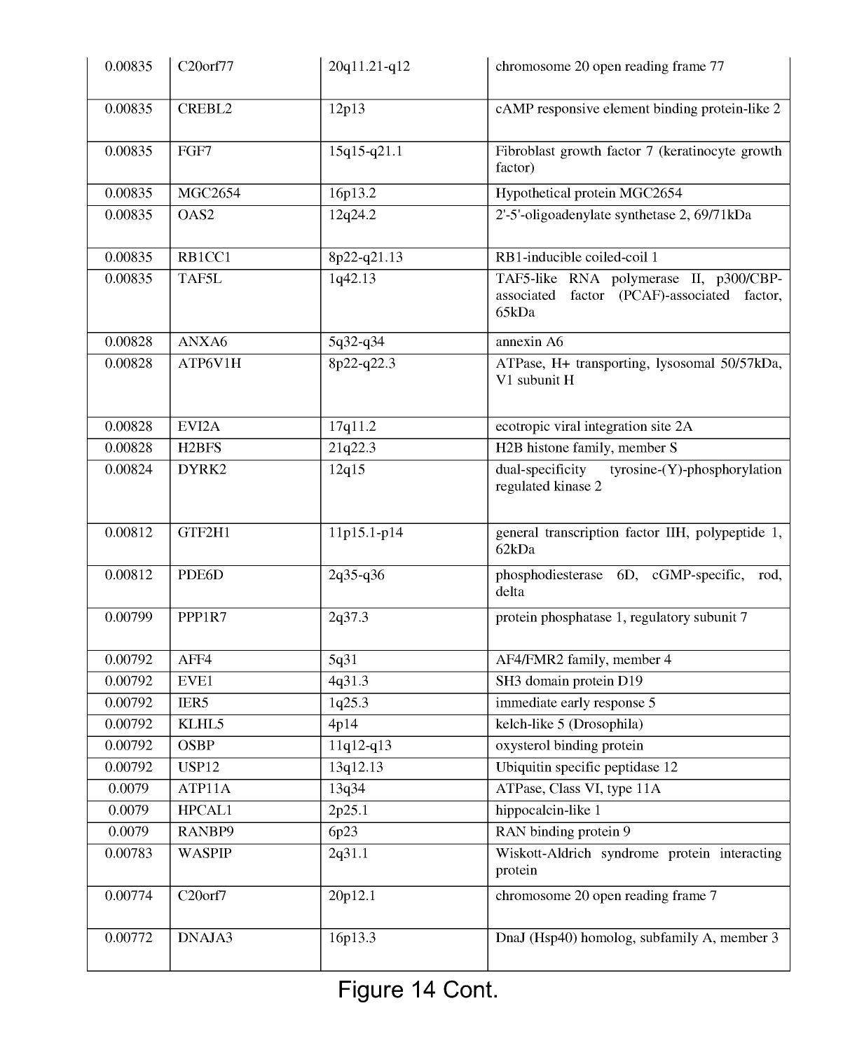

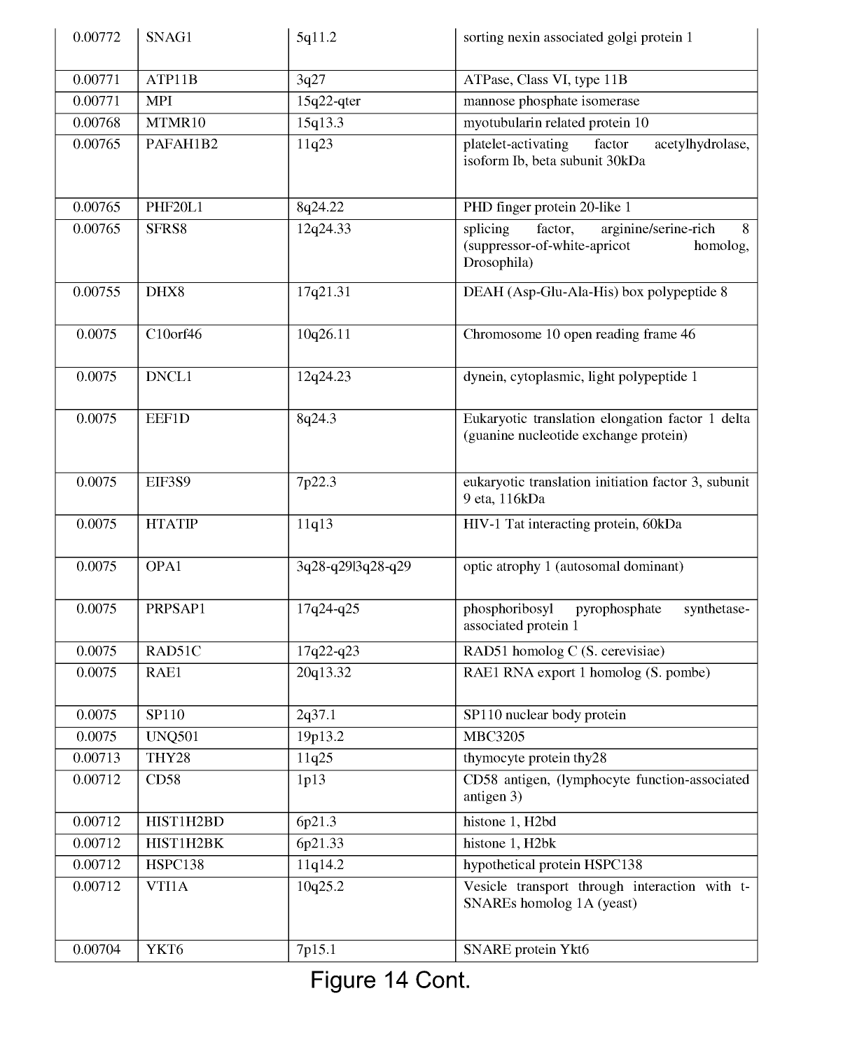

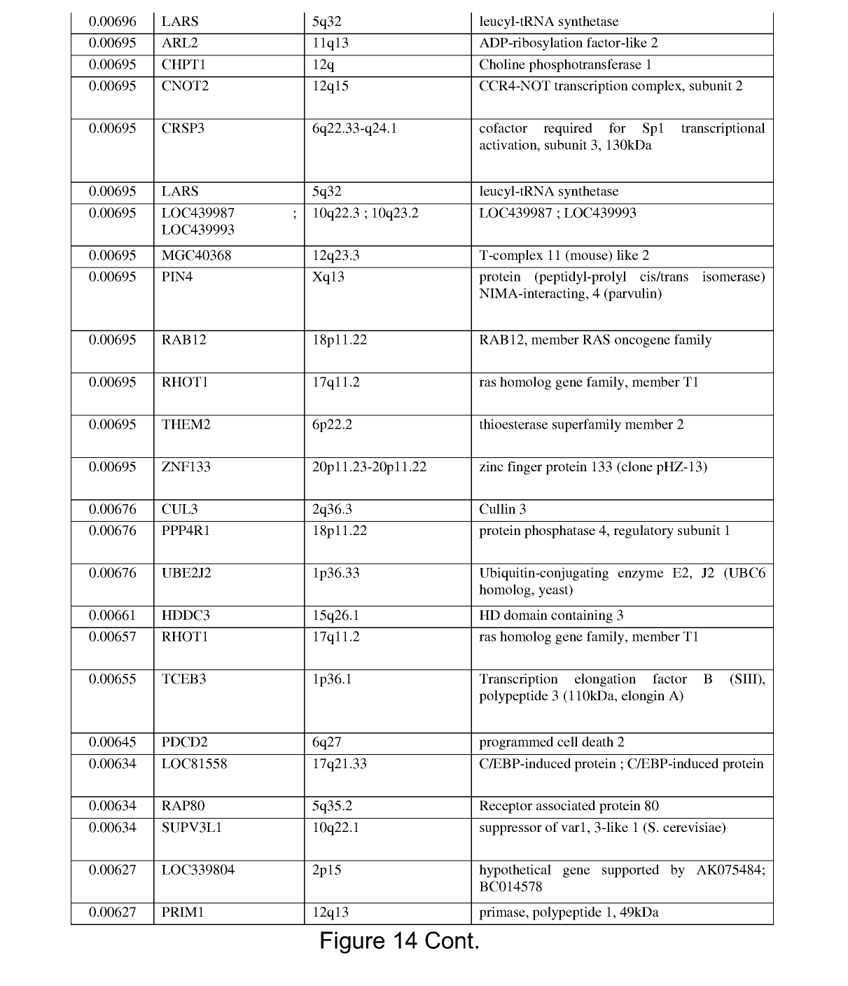

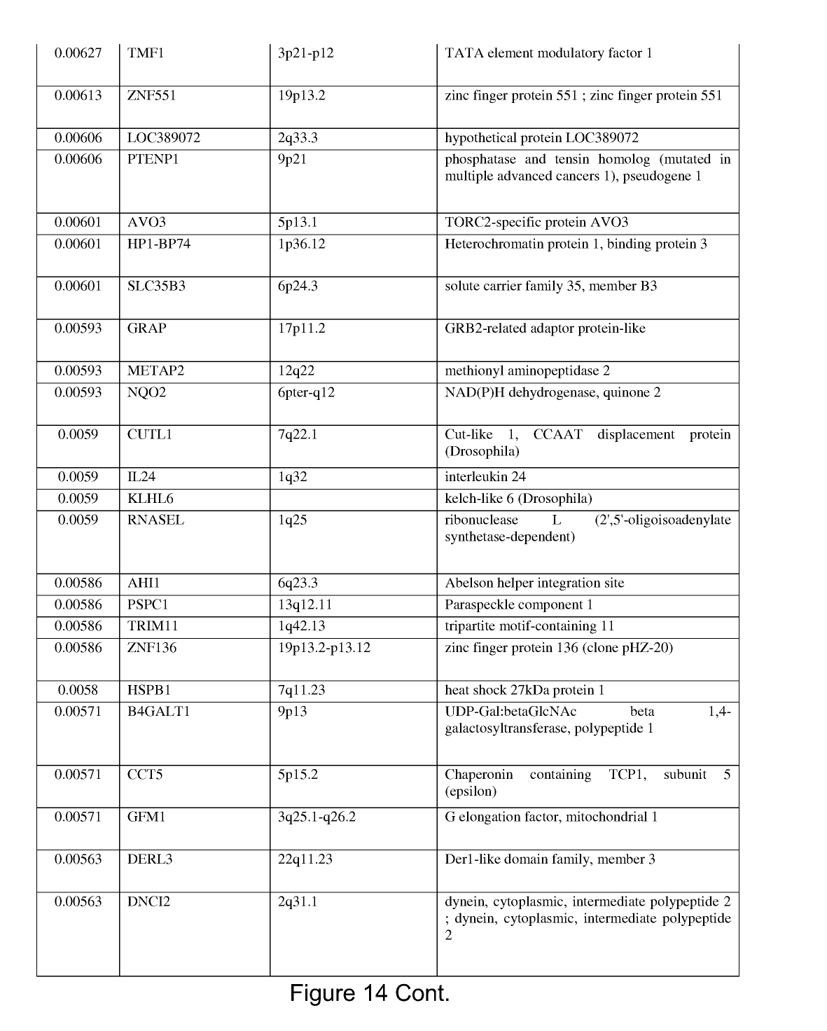

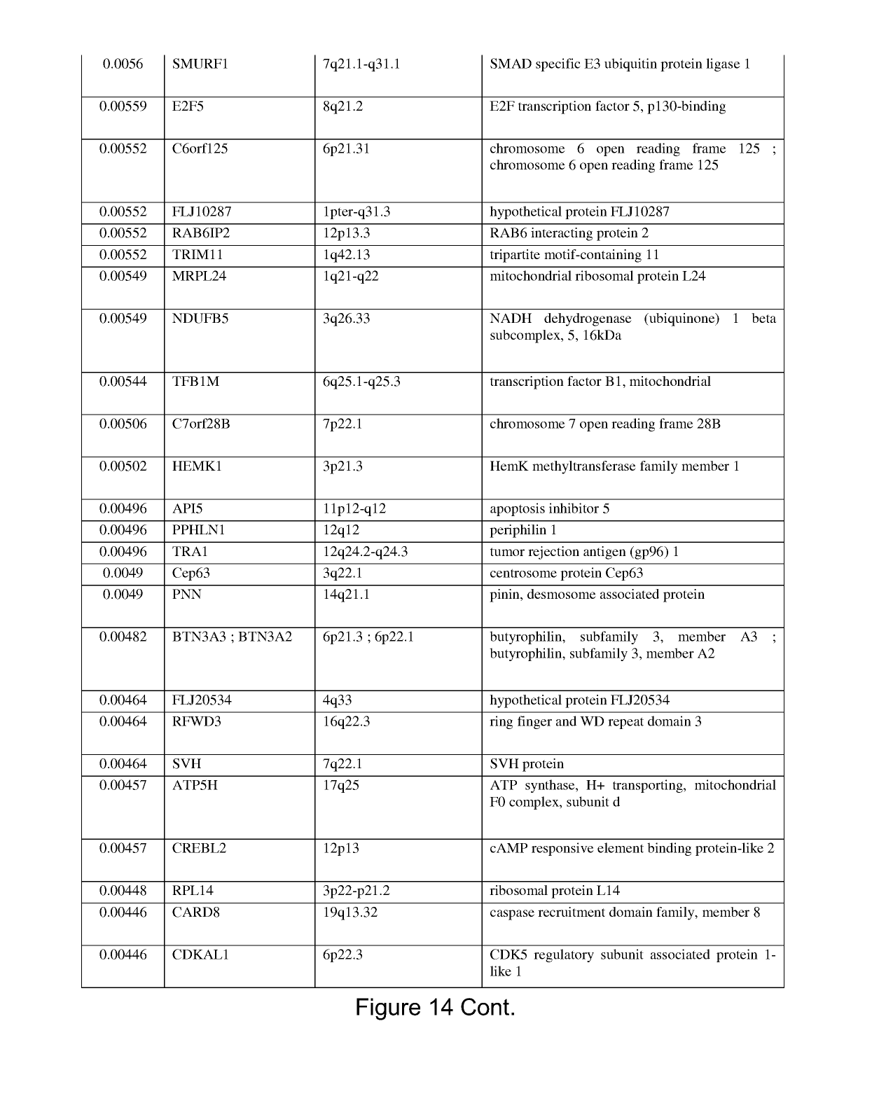

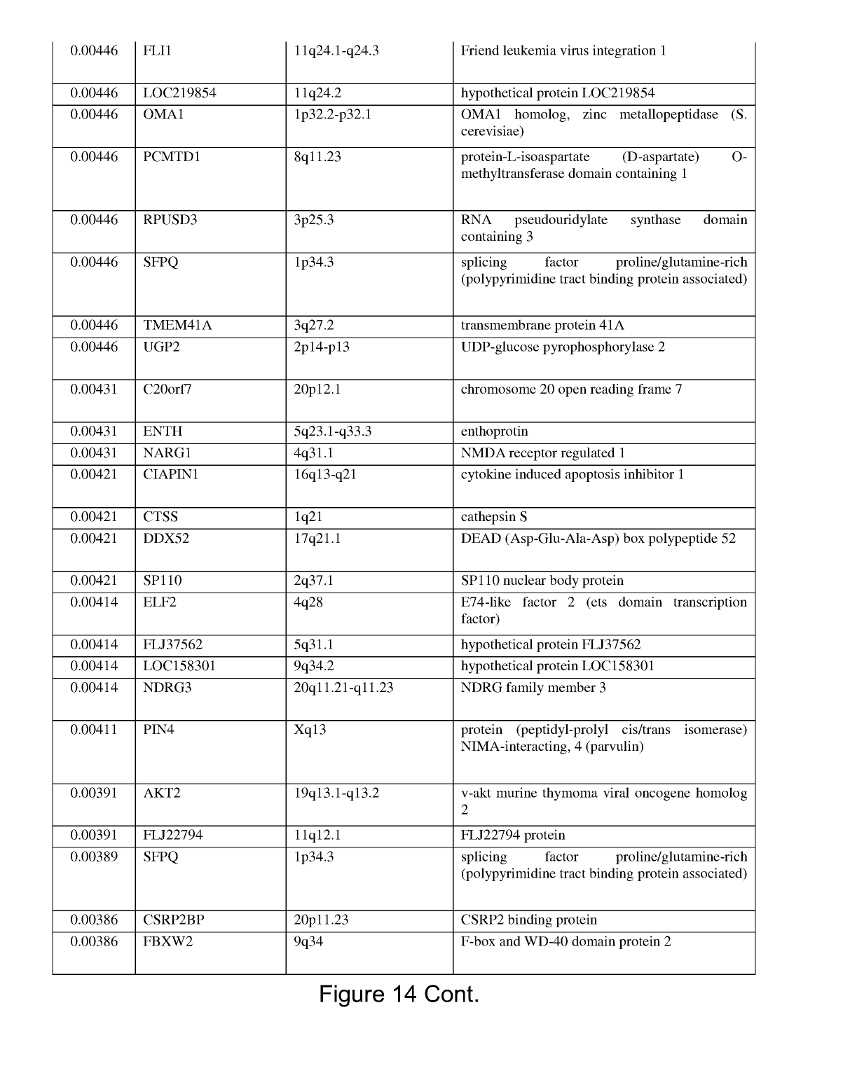

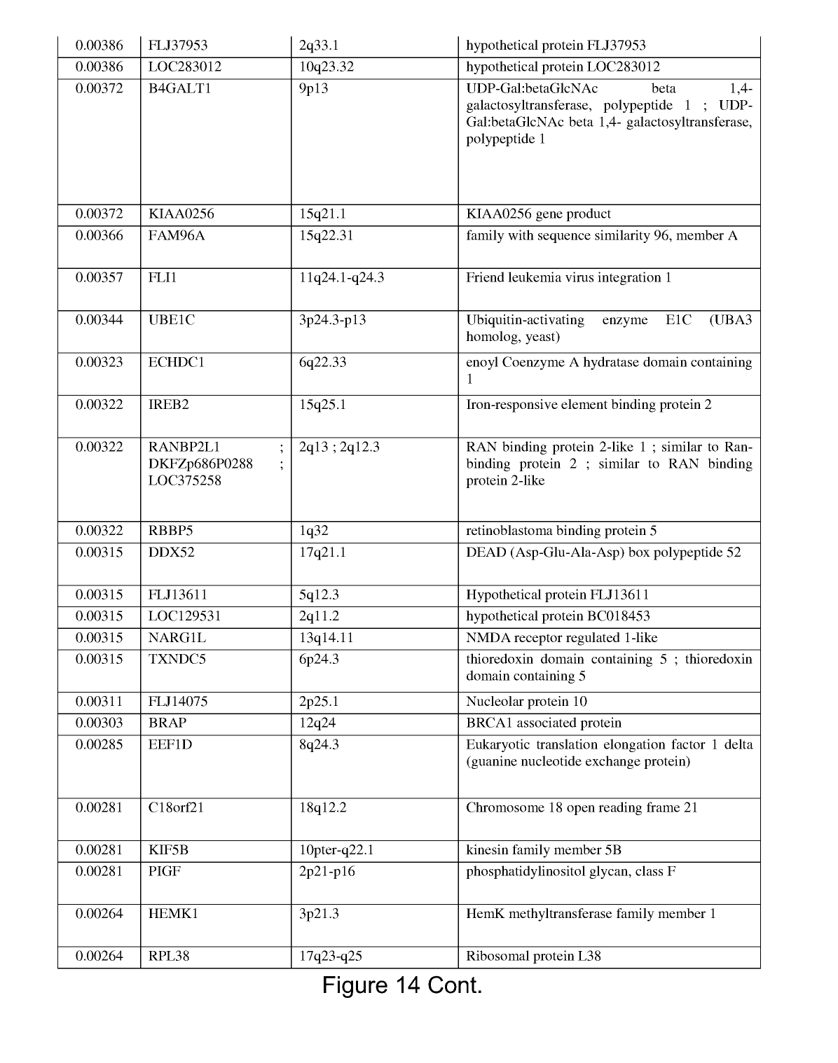

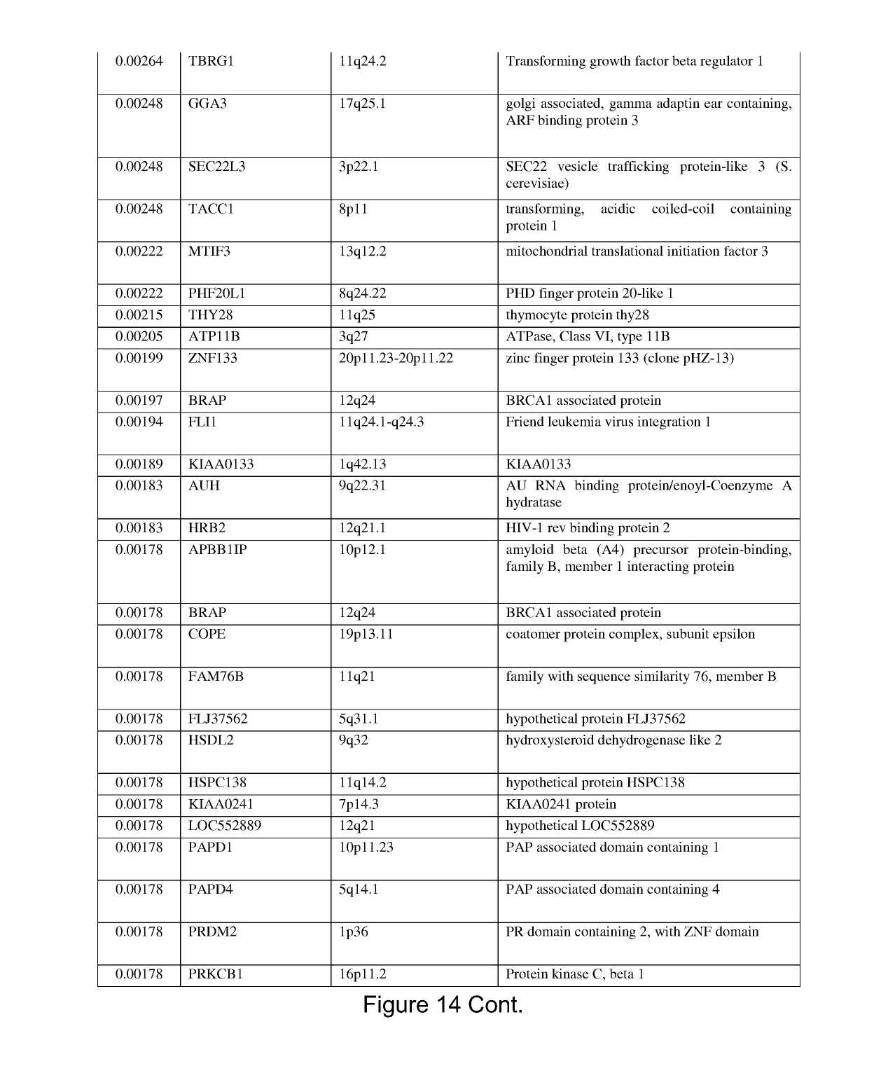

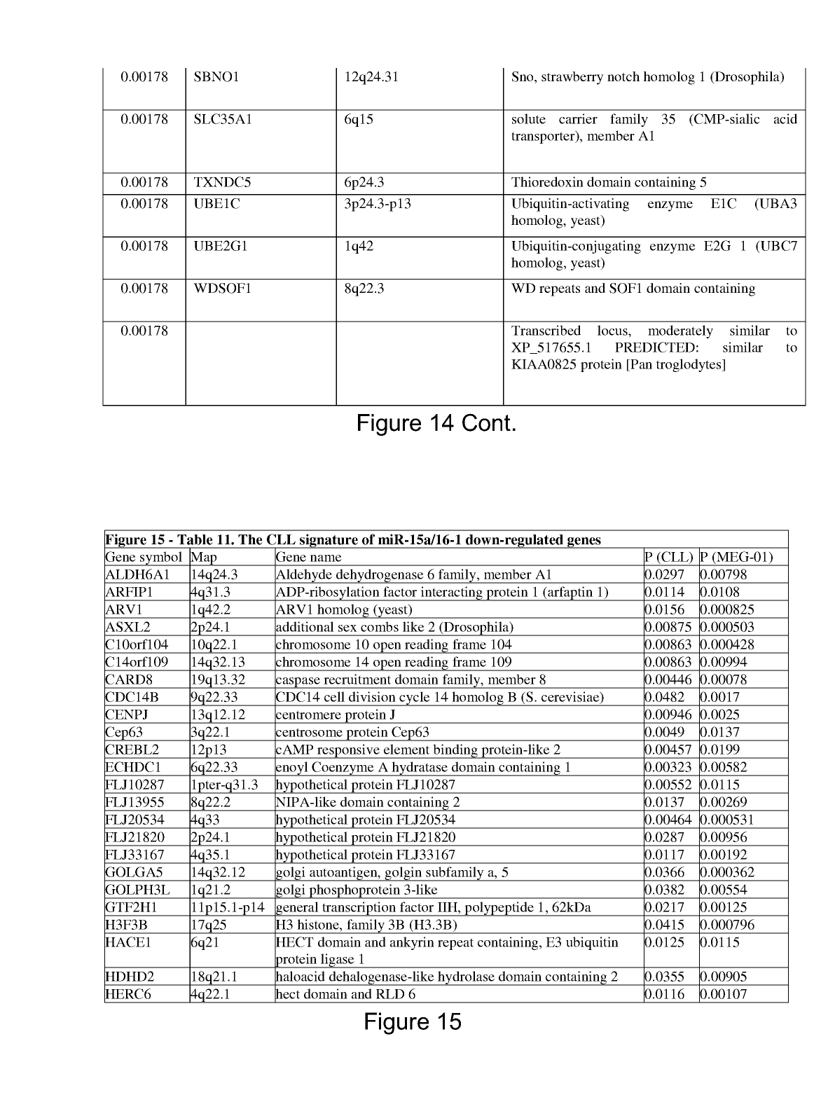

We have shown that a signature of 13 miRNAs distinguished between indolent and aggressive CLL and that loss of the Mir-15α/16-1 cluster is a characteristic of indolent CLLs (18). First, we validated the expression of Mir-15α/16-1 by qRT-PCR and confirmed the microarray data by qRT-PCR (data not shown). Among the considered 16 patients, 8 have higher expression of miR-15a/16-1, with respect to the other 8 patients (P =7.7×10⁻⁶ at microarray analysis, P=0.019 at qRT-PCR analysis). The comparison between eight CLLs with high and low Mir-15α/16-1 expression by EST oligonucleotide microarray analysis showed 678 Affymetrix probes (539 genes) significantly differentially expressed among the two groups (FIG. 14—Table 10). Overall, 82 of 539 genes (15.2%) are ARE mRNAs, and 4 are predicted as targets by all three bioinformatics algorithms.

A Signature of Mir-15α/16-1 Down-Regulated Transcripts

We selected genes that were low in miR-15/16 high-expressor CLLs and high in miR-15/16 low-expressor CLLs, which were intersected with genes down-regulated in MEG-01 cells after transfection with pRS15/16.

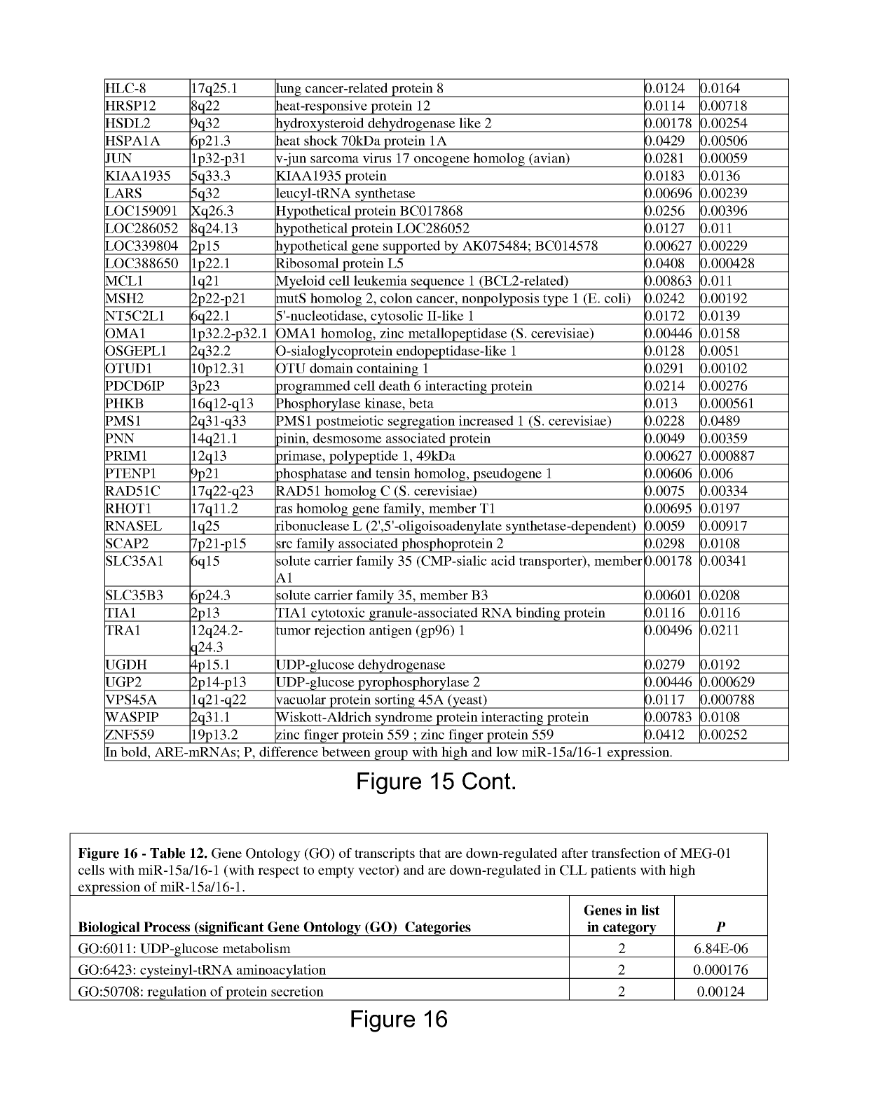

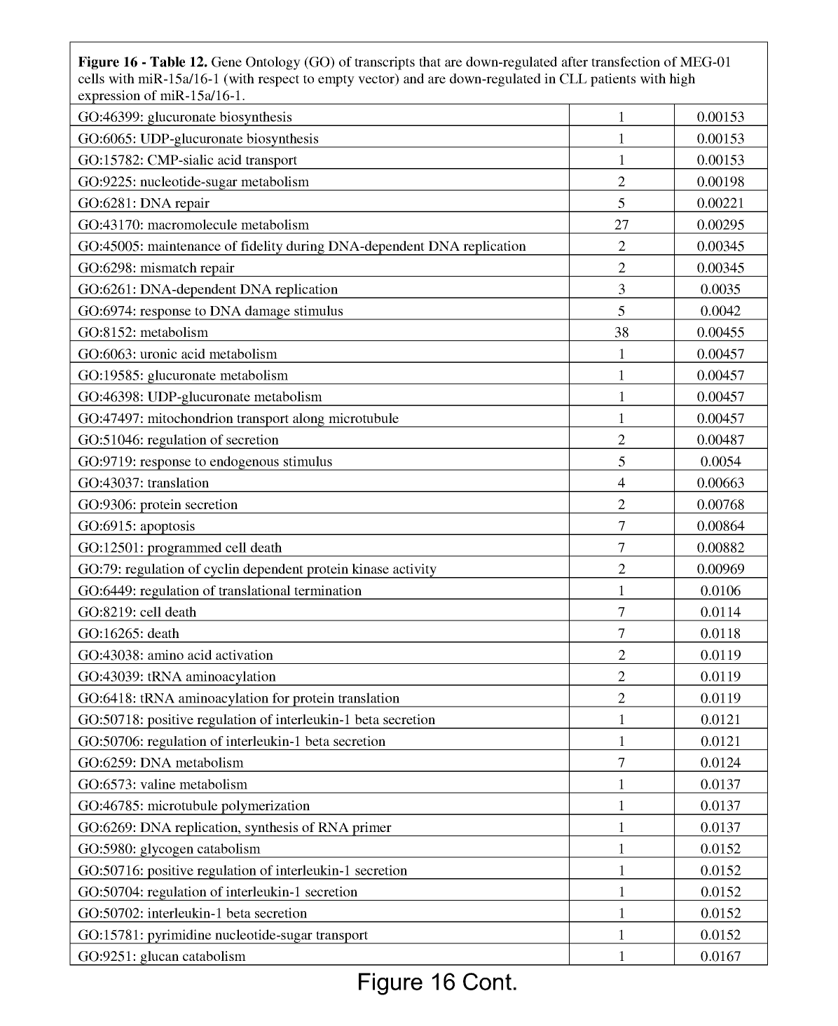

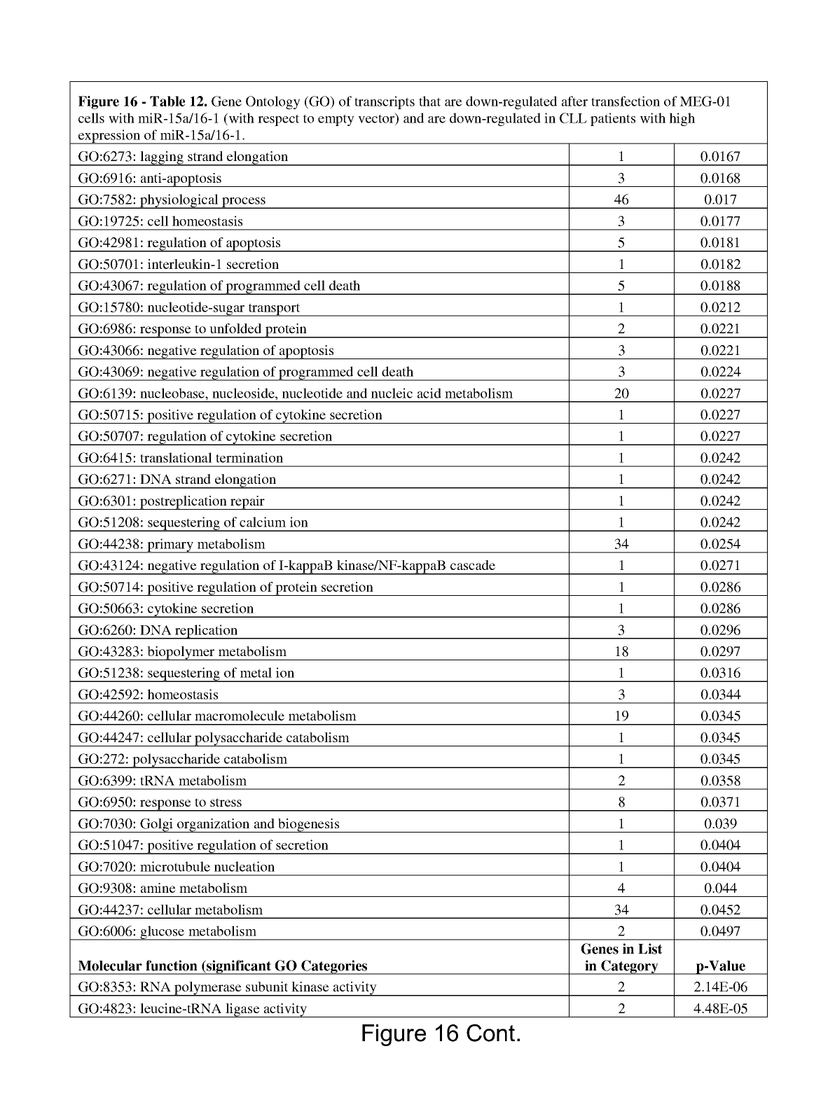

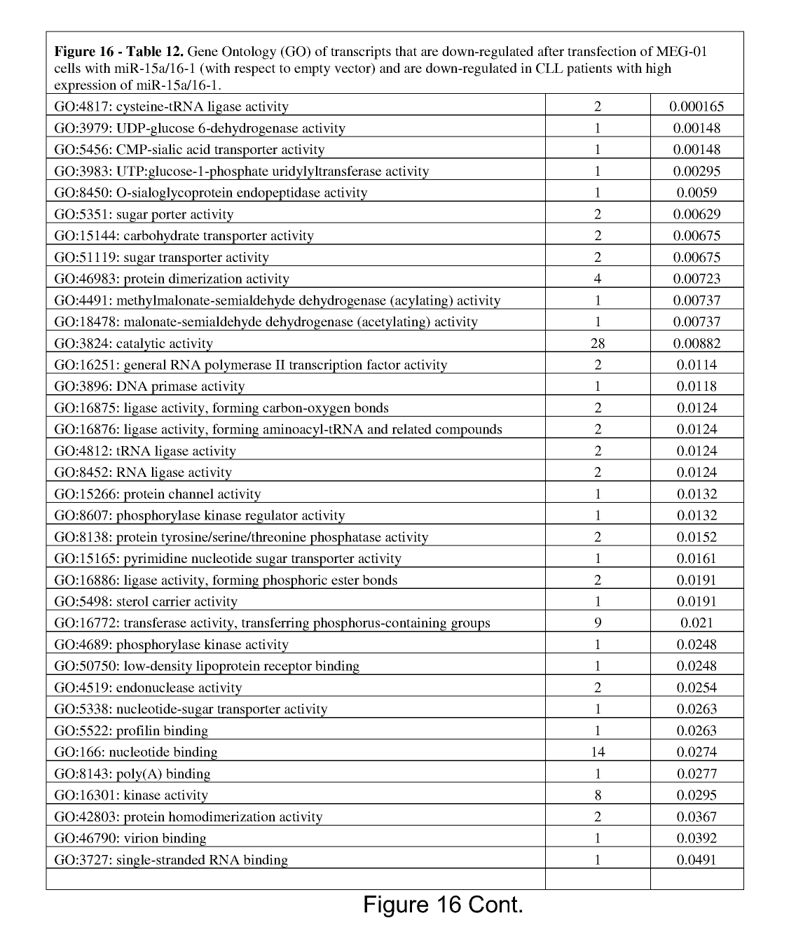

A signature of 60 genes (70 probes) emerged (FIG. 8—Table 4, FIG. 15—Table 11). Thirteen of these genes (21.7%) are ARE-mRNAs, distributed in cluster III (7.8%), IV (7.8%), and V (84.6%). No statistically significant enrichment in ARE-mRNAs was observed in this signature with respect to both the total of down-regulated mRNAs in MEG-01 (P=0.76) and the total of repressed transcripts in patients with high expression of Mir-15α/16-1 (P=0.14). We performed the GO analysis of these 70 transcripts and found, among the significantly represented categories, some of those previously identified in transfected MEG-01 and involved in regulation of cell cycle and apoptosis, such as “antiapoptosis” (GO:6916), “negative regulation of apoptosis” (GO:43066), and “negative regulation of programmed cell death” (G0:43069) (FIG. 16 - Table 12). The consistency of the results in MEG-01 and in CLL patients confirms the validity of our in vitro model and identifies GO categories and a panel of protein coding genes, whose expression is consistently controlled by the cluster.

DISCUSSION

We show that Mir-15α/16-1 exert a tumor suppressor function in vivo by inhibiting the growth of tumor engraftments of leukemic cells in nude mice.

To investigate the molecular bases of Mir-15α/16-1 tumor-suppressor function, we performed an extensive microarray analysis of the deregulated genes after transfection of MEG-01 cells with pRS15/16, a vector expressing Mir-15α/16-1, and using the same empty vector (pRS-E) as a control.

We confirmed some of the targets observed by other groups in different models, such as CDK6, CDC27, and RAB11FIP2 (28) in solid tumor cell lines and ACVR2A in Xenopus laevis (29). We matched our experimentally identified down-regulated genes with the targets of Mir-15α/16-1 commonly predicted by three of the most widely used algorithms for the identification of miRNA-targets (PicTar, TargetScan, MiRanda), and found 85 genes (2.6%) in common

Interestingly, by matching our results with a computational method that identifies miRNA targets by predicting miRNA regulatory modules (MRMs) or groups of miRNAs and target genes that are believed to participate cooperatively in posttranscriptional gene regulation (30), we found 5 of 13 (38.5%) miR-15/16 MRM predicted genes (ATP2B1, FBXW7, PPM1D, SON, and WTI) among our differentially expressed genes. This percentage represents the highest among all of the considered prediction algorithms

Among the 265 experimentally up-regulated mRNAs, none is predicted as a target of miR-15α/16. This finding can be explained by indirect effects, for example by the regulation of transcription factor(s) targeted by these two miRNAs. The effects of the exogenous expression of Mir-15α/16-1 in MEG-01 cells was also investigated by proteomics 48 h after the transfection. We also studied different time-from-transfection intervals to analyze the effects of Mir-15α/16-1 at a transcriptional (24 h) or translational (48 h) level, because after 24 h, mRNA silencing is maximal, but secondary transcriptional effects due to protein depletion are minimal (31).

Our proteomic approach was able to detect 27 targets of Mir-15α/16-1, approximately one-third of which are also predicted targets. Interestingly, 25% (two of eight) of the predicted targets were down-regulated both in the transcriptome and in the proteome. Among the Mir-15α/16-1 down-regulated genes, we demonstrated that IGSF4 is a direct target of the cluster.

IGSF4 was originally identified as a tumor-suppressor gene in lung cancer and is involved in cell adhesion (32, 33). Sasaki et al. (34) have demonstrated that TSLC1/IGSF4 acts as an oncoprotein involved in the development and progression of adult T cell leukemia (ATL). The inventors herein now believe that, by directly silencing IGSF4, Mir-15α/16-1 is useful in exerting a more general antileukemic effect.

We also studied by microarray the down-regulated mRNAs in eight CLL patients with high levels of Mir-15α/16-1 with respect to eight CLL patients with low levels of these two miRNAs and identified a signature of 60 genes in common between CLLs and MEG-01 transfected with miR-15α/16-1.

This signature (which includes ≈2% of the down-regulated genes in MEG-01 and ≈11% of those repressed in patients) contains oncogenes such as MCL1, JUN, SCAP2, TRA1, PDCD6IP, RAD51C, and HSPA1A/1B, which can be used to explain the oncosuppressor effect of Mir-15α/16-1 observed in MEG-01 both in vitro (22), and in vivo, as now shown herein.

MCL1 is an antiapoptotic BCL-2 family member that contributes to B cell survival in CLL and has been associated with resistance to chemotherapy (35, 36). Despite the fact that MCL-1 expression is not different in ZAP 70-positive (aggressive) vs. ZAP 70-negative (indolent) B-CLL cells (37), it represents a relevant therapeutic target in both acute and chronic lymphoid malignancies, because its silencing is sufficient to promote apoptosis in ALL and CLL cells and increase sensitivity to rituximab-mediated apoptosis (38). Interestingly, miR-29b has also been identified to target Mc11 in a cholangiocarcinoma model (39), and many pieces of evidence converge in defining a role of the miR-29 family as TSGs in both solid (40) and hematologic malignancies (41).

These findings provide a rationale to an association of Mir-15α/16-1 and miR-29s in the treatment of CLL.

Moreover, a sustained signaling through the B cell receptor promotes survival of B-CLL cells both by induction of MCL1 and, to a lesser extent, by activation of c-JUN NH₂-terminal kinase (JNK) (42).

Therefore, by targeting both MCL1, and c-JUN transcripts, the impact of the Mir-15α/16-1 cluster on the survival of B-CLL cells could be even more robust. The presence of BCL2 in the proteomics list confirms our previous statement of a posttranscriptional regulation of this target (22).

Moreover the repression of LARS (leucyl-tRNA synthetase), involved in the same pathway of RARS (arginyl-tRNA synthetase), and the presence of RARS among the down-regulated genes in MEG-01 confirms our previous hypothesis that this pathway could be targeted by Mir-15α/16-1 (16).

Interestingly, the signature includes also many important tumor-suppressor genes (RNASEL, HACE1, CEP63, CREBL2, MSH2, TIA1, and PMS1) and reveals an explanation for the link between Mir-15α/16-1 expression and CLL prognosis.

We described that in CLL patients with unmutated IgV_{H}, and high expression of ZAP-70 (poor prognosis), the levels of Mir-15α/16-1 are higher than in CLL patients with a better prognosis (18). The observed coexistence of oncogenes and TSGs in Mir-15α/16-1 CLL signature give a molecular explanation as to why high levels of these two miRNAs are associated with CLLs with a worse prognosis (18). High Mir-15α/16-1 levels could down-regulate many TSGs and consequently negatively affect many oncosuppressor pathways, therefore leading to a more oncogenic phenotype.

Recently, it has been demonstrated that miR-16 is critically involved in ARE-mediated mRNA instability (24). In MEG-01 cells, we found that ARE-mRNAs are significantly more represented among the down-regulated genes (20.1%) than among the up-regulated (13.6%, P=0.0098). Although the identified signature is not enriched with ARE-mRNAs, it shows a predominance (84.6%) of cluster V ARE-mRNAs (which reflects the higher number of members of this cluster in both MEG-01 and patients), indicating that a higher number of pentameric AU-repeat does not correspond to a higher silencing effect by Mir-15α/16-1.

Finally the GO analysis of the deregulated genes indicates that Mir-15α/16-1 impacts strongly on metabolic pathways, on nucleic acid-binding pathways, and the activities of translation factors. In solid tumor cell lines miR-16-down-regulated transcripts are enriched with genes whose silencing causes an accumulation of cells in G₀/G₁ and that this function does not depend on AU-rich elements (28).

We now have found that some of the described miR-16 targets whose disruption triggered

G₀/G₁-cell accumulation were down-regulated also in our cell model (CDK6, CDC27, RAB11FIP2) and that some of the previously described GO categories [namely “mitotic cell cycle” (GO:278), and “cell cycle” (GO:7049)] are represented also in our data.

In contrast with the previous report, we found a statistically significantly higher number of ARE-mRNAs among the down-regulated targets with respect to the up-regulated. These differences may reflect cell-specific functions of Mir-15α/16-1, whereas the common finding that Mir-15α/16-1 targets “cell cycle”-involved genes, both in solid and in hematologic tumor models, suggests a more general and robust effect of the cluster on this group of genes.

We now show Mir-15α/16-1 deregulated genes in both a leukemic cell model and in primary CLLs, and identify a signature of common genes whose silencing characterizes the Mir-15α/16-1-induced phenotype in CLL.

These findings could have important significance for the development of therapeutic approaches for CLLs.

Materials and Methods

Cell Culture and Patient Samples

The human megakaryocytic MEG-01 cell line was purchased from the American Type Culture Collection and grown in 10% FBS RPMI medium 1640, supplemented with lx nonessential amino acids and 1 mmol of sodium pyruvate at 37° C. and 5% CO₂. For the patient study, we used 16 CLL samples obtained after informed consent from patients diagnosed with CLL at the CLL Research Consortium institutions. Briefly, blood was obtained from CLL patients and mononuclear cells were isolated through Ficoll/Hypaque gradient centrifugation (Amersham Pharmacia Biotech) and processed for RNA extraction according to the described protocols (18). For all of the samples, the microarray expression data were known as reported in ref. 18, and we further performed confirmation with qRT-PCR.

In Vivo Studies

Animal studies were performed according to institutional guidelines. MEG-01 cell lines were transfected in vitro with p-Retrosuper vector (43) expressing miR-15almiR-16-1 (pRS15/16). Untransfected (mock) or cells transfected with the same empty plasmid (pRS-E) served as tumorigenic controls. At 24 h after the transfection, 10⁷ viable cells were injected s.c. into the left flanks of 5-week-old female nude mice (Charles River Breeding Laboratories), five mice per transfected or control cell line. Tumor diameters were measured on days 7, 15, 21, and 28. After 28 days, the mice were killed, necropsies were performed, and tumors were weighed. Tumor volumes were calculated by using the equation V (in mm³) =A x B ²/2, where A is the largest diameter, and B is the perpendicular diameter.

In Vitro Transfection

MEG-01 cells were transiently transfected with 1 μg/ml (final concentration) pRS-15/16 or pRS-E vector by using Lipofectamine 2000 reagent (Invitrogen) according to the manufacturer's instructions. After 24 h, total RNA was extracted by using TRIzol reagent (Invitrogen) according to the manufacturer's instructions

Microarray Hybridization and Data Analysis

Two samples obtained from the MEGO1 cell line transfected with pRS-15/16 and pRS-E vector, each one in triplicate, and 16 CLL samples were analyzed by microarray using Human Genome U133A Plus 2.0 GeneChip arrays (Affymetrix). The CEL files generated by the GeneChip scanner were imported in GeneSpring GX 7.3 software (Agilent Technologies) and further processed. Details about the microarray experiment are described in EXAMPLE II herein

MiRNA Target Prediction

The analysis of miRNA predicted targets was determined by using the algorithms TargetScan (genes.mit.edu/targetscan/), PicTar (pictar.bio.nyu.edu/), and miRanda (cbio.mskcc.org/cgi-bin/mirnaviewer/mirnaviewer.p1).

Adenylate Uridylate-Rich Elements (ARE)-Containing Genes Identification

The ARE-mRNA database version 3.0 (ARED), as described (44), was used (see EXAMPLE II).

Two-Dimensional PAGE and Protein Identification by Matrix Assisted Laser Desorption/Ionization Time-of-Flight (MALDI-TOF) and Mass Spectrometry (MS).

MEG-01 cells were transiently transfected for 48 hr with 1 μg/ml (final concentration) pRS 15/16 or pRS-E vector by using Lipofectamine 2000 reagent (Invitrogen) according to the manufacturer's instructions and the details of the two-dimensional PAGE, and protein identification by MALDI-TOF and MS are described in EXAMPLE II.

qRT-PCR

qRT-PCR analysis for miRNAs was performed in triplicate with the TaqMan MicroRNA assays kit (Applied Biosystems) according to the manufacturer's instructions and as described (45). For normalization, 18S RNA was used; qRT-PCR analyses for other genes of interest were performed by reverse transcription of RNA to cDNA with gene-specific primers and IQ SYBR green Supermix (Bio-Rad) according to the manufacturer's instructions. β-Tubulin was used for normalization.

Luciferase Reporter Assay

For luciferase reporter experiments, a IGSF4 3′ UTR segment of 237 by was amplified by PCR from human cDNA and inserted into the pGL3-control vector with SV40 promoter (Promega) by using the XbaI site immediately downstream from the stop codon of luciferase. Details about the microarray experiment are described in EXAMPLE II. The experiments were performed in triplicate.

### EXAMPLE II

Microarray Hybridization

Two samples obtained from MEGO1 cell line transfected with pRS-15/16 and pRS-E vector, each one in triplicate, and 16 CLL samples were analyzed by microarray. The experiments were performed at the Ohio State University microarray facility. The amount of extracted RNA was quantified by using the NanoDrop spectrophotometer (NanoDrop Technologies) and the RNA quality was assessed by using an Agilent Bioanalyzer 2100 (Agilent Technologies). Total RNA (1.2 μg) was used to generate biotin-labeled cRNA by means of Enzo BioArray HighYield RNA Transcript Labeling kit (Affymetrix). After fragmentation, labeled cRNA was used for hybridization on Human Genome U133A Plus 2.0 GeneChip arrays (Affymetrix). Hybridizations, washing, and staining were performed according to manufacturer's instructions. Hybridized arrays were scanned with the Genechip 7G.

Microarray Data Analysis

The CEL files generated by the GeneChip scanner were imported in GeneSpring GX 7.3 software (Agilent Technologies). Raw data were normalized by using the GC Robust Multiarray Average (GCRMA) procedure followed by a data transformation, to set negative values to 0.01. Each measurement was then divided by the 50th percentile of all measurements in that sample, and each gene was divided by the median of its measurements in all samples. The genes differentially expressed in MEGO1 after miR-15/16 transfection and among the two CLL groups were selected as having a 2-fold difference between their geometrical mean expression in the compared groups and a statistically significant P-value (<0.05) by ANOVA, followed by the application of the Benjamini and Hoechberg correction for false-positive reduction. Differentially expressed genes were used for cluster analysis of samples, using standard correlation as a measure of similarity. The list of putative miR-15/16 targets was imported in GeneSpring using the gene symbols and the intersection with the lists of interest was performed by using the Venn Diagram GeneSpring tool. The Gene Ontology (GO) analysis on differentially expressed genes was performed with the GeneSpring software using a P <0.05 to find statistically enriched GO categories.

Adenylate Uridylate-Rich Elements (ARE)-Containing Genes Identification

All of the deregulated (up- and down-regulated) genes identified by the EST oligonucleotide microarray analysis, after transfection with pRS 15/16 were scrutinized for the presence of AREs in their 3′-UTR, by using the ARE-mRNA database version 3.0 (ARED), which contains >4,000 ARE-mRNAs computationally mapped to the human genome. The probability that more ARE-containing mRNAs are in the group of down-regulated with respect to the group of up-regulated genes in pRS15/16 vs. pRS-E-transfected MEG-01 cells, was calculated with the χ² test (α=0.05).

Two-Dimensional PAGE and Protein Identification by MALDI-TOF and MS

EG-01 cells were transiently transfected with 1 μg/ml (final concentration) of pRS15/16 or pRS-E vector by using Lipofectamine 2000 reagent (Invitrogen), according to the manufacturer's instructions. After 48 h from the transfection, cells were lysed in sample buffer containing 7 mol/liter urea, 2 mol/liter triourea, 4% CHAPS, 2 mmol/liter tributyl phosphine, and 0.2% BioLyte 3/10 ampholytes (Bio-Rad). The crude cell homogenate was sonicated and centrifuged at 10,000×for 10 min Immobilized pH gradient strips (11 cm) with pH range 3-10 were hydrated overnight in sample buffer containing 200 μg of total protein. After isoelectric focusing, using Protean Cell (Bio-Rad), proteins were separated in the second dimension by 8-16% gradient SDS-PAGE for 1 h at 200 V. All gels were run thrice, stained with colloidal Coomassie blue (Pierce), and scanned with Versadoc 3000 image system (Bio-Rad). Gel images were captured with an 800 GS scanner (Bio-Rad) and analyzed by using PDQuest software (Bio-Rad) by the total protein density in each of the gel images. Protein spots were quantified after normalization for total protein on the gel. For statistical analyses, the average results of the triplicates were calculated, and the resulting values were used as independent data points in statistical analyses (Student's t test).

MS was carried out in the Ohio State University Davis Heart and Lung Research Institute Proteomics Core Laboratory. We attempted to identify proteins only from spots that were consistently reduced or induced at least 4-fold in all comparative gels. The protein spots were transferred to the MassPrep station (PerkinElmer) for automated in-gel protein digestion following the protocol included with the WinPREP Multiprobe II software (PerkinElmer). Briefly, gel pieces were destained and then reduced with DTT. After incubation with iodoacetamide, gels were washed and dehydrated with acetonitrile. In-gel digestion of the extracted proteins was carried out with 6 μg/ml trypsin in 50 mmol/liter ammonium bicarbonate. The digested peptides were extracted with a mixture of 1% formic acid/2% acetonitrile and applied onto a stainless steel MALDI plate (Waters). MS of the resulting peptides was recorded on the MALDI-TOF spectrometer (Waters) in reflectron mode. Resulting peptides were matched with their corresponding proteins with ProFound by searching the NIH National Center for Biotechnology Information database.

Luciferase Reporter Assay

For luciferase reporter experiments a IGSF4 3′ UTR segment of 237 by was amplified by PCR from human cDNA and inserted into the pGL3-control vector with SV40 promoter (Promega), using the XbaI site immediately downstream from the stop codon of luciferase. The following sets of primers were used to generate specific fragments:

[SEQ ID NO: 1]
IGSF4-UTR Fw: 5′-GCTCTAGAAAAAGGAGAACCAGCACAGC-3′,
and
[SEQ ID NO: 2]
IGSF4-UTR Rv: 5′-GCTCTAGATGACACACCTCACTTGCAGA-3′.

The italicized nucleotides correspond to the endonuclease restriction site. MEG-01 cells were cotransfected in 12-well plates by using Lipofectamine 2000 reagent (Invitrogen), according to the manufacturer's protocol, with 0.4 μg of the firefly luciferase report vector and 0.08 μg of the control vector containing Renilla luciferase pRL-TK vector (Promega). For each well, 1 μg/ml (final concentration) of pRS 15/16 or pRS-E vector were used. Firefly and Renilla luciferase activities were measured consecutively by using dual-luciferase assays (Promega), 24 h after the transfection. The experiments were performed in triplicate.

### EXAMPLES of USES and DEFINITIONS THEREOF

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

As such, the definitions herein are provided for further explanation and are not to be construed as limiting.

The articles “a” and “an” are used herein to refer to one or to more than one (i.e., to at least one) of the grammatical object of the article. By way of example, “an element” means one element or more than one element.

A “marker” and “biomarker” is a gene and/or protein and/or functional variants thereof whose altered level of expression in a tissue or cell from its expression level in normal or healthy tissue or cell is associated with a disorder and/or disease state.

The “normal” level of expression of a marker is the level of expression of the marker in cells of a human subject or patient not afflicted with a disorder and/or disease state.

An “over-expression” or “significantly higher level of expression” of a marker refers to an expression level in a test sample that is greater than the standard error of the assay employed to assess expression, and in certain embodiments, at least twice, and in other embodiments, three, four, five or ten times the expression level of the marker in a control sample (e.g., sample from a healthy subject not having the marker associated disorder and/or disease state) and in certain embodiments, the average expression level of the marker in several control samples.

A “significantly lower level of expression” of a marker refers to an expression level in a test sample that is at least twice, and in certain embodiments, three, four, five or ten times lower than the expression level of the marker in a control sample (e.g., sample from a healthy subject not having the marker associated disorder and/or disease state) and in certain embodiments, the average expression level of the marker in several control samples.

A kit is any manufacture (e.g. a package or container) comprising at least one reagent, e.g., a probe, for specifically detecting the expression of a marker. The kit may be promoted, distributed or sold as a unit for performing the methods of the present invention.

“Proteins” encompass marker proteins and their fragments; variant marker proteins and their fragments; peptides and polypeptides comprising an at least 15 amino acid segment of a marker or variant marker protein; and fusion proteins comprising a marker or variant marker protein, or an at least 15 amino acid segment of a marker or variant marker protein.

The compositions, kits and methods described herein have the following non-limiting uses, among others:
assessing whether a subject is afflicted with a disorder and/or disease state;
assessing the stage of a disorder and/or disease state in a subject;
assessing the grade of a disorder and/or disease state in a subject;
assessing the nature of a disorder and/or disease state in a subject;
assessing the potential to develop a disorder and/or disease state in a subject;
assessing the histological type of cells associated with a disorder and/or disease state in a subject;
making antibodies, antibody fragments or antibody derivatives that are useful for treating a disorder and/or disease state in a subject;
assessing the presence of a disorder and/or disease state in a subject's cells;
assessing the efficacy of one or more test compounds for inhibiting a disorder and/or disease state in a subject;
assessing the efficacy of a therapy for inhibiting a disorder and/or disease state in a subject; monitoring the progression of a disorder and/or disease state in a subject;
selecting a composition or therapy for inhibiting a disorder and/or disease state in a subject;
treating a subject afflicted with a disorder and/or disease state;
inhibiting a disorder and/or disease state in a subject;
assessing the harmful potential of a test compound; and
preventing the onset of a disorder and/or disease state in a subject at risk therefor.

Screening Methods

Animal models can be created to enable screening of therapeutic agents useful for treating or preventing a disorder and/or disease state in a subject. Accordingly, the methods are useful for identifying therapeutic agents for treating or preventing a disorder and/or disease state in a subject. The methods comprise administering a candidate agent to an animal model made by the methods described herein, and assessing at least one response in the animal model as compared to a control animal model to which the candidate agent has not been administered. If at least one response is reduced in symptoms or delayed in onset, the candidate agent is an agent for treating or preventing the disease.

The candidate agents may be pharmacologic agents already known in the art or may be agents previously unknown to have any pharmacological activity. The agents may be naturally arising or designed in the laboratory. They may be isolated from microorganisms, animals or plants, or may be produced recombinantly, or synthesized by any suitable chemical method. They may be small molecules, nucleic acids, proteins, peptides or peptidomimetics. In certain embodiments, candidate agents are small organic compounds having a molecular weight of more than 50 and less than about 2,500 daltons. Candidate agents comprise functional groups necessary for structural interaction with proteins. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. There are, for example, numerous means available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. In certain embodiments, the candidate agents can be obtained using any of the numerous approaches in combinatorial library methods art, including, by non-limiting example: biological libraries; spatially addressable parallel solid phase or solution phase libraries; synthetic library methods requiring deconvolution; the “one-bead one-compound” library method; and synthetic library methods using affinity chromatography selection.

In certain further embodiments, certain pharmacological agents may be subjected to directed or random chemical modifications, such as acylation, alkylation, esterification, amidification, etc. to produce structural analogs.

The same methods for identifying therapeutic agents for treating a disorder and/or disease state in a subject can also be used to validate lead compounds/agents generated from in vitro studies.

The candidate agent may be an agent that up- or down-regulates one or more of a disorder and/or disease state in a subject response pathway. In certain embodiments, the candidate agent may be an antagonist that affects such pathway.

Methods for Treating a Disorder and/or Disease State

There is provided herein methods for treating, inhibiting, relieving or reversing a disorder and/or disease state response. In the methods described herein, an agent that interferes with a signaling cascade is administered to an individual in need thereof, such as, but not limited to, subjects in whom such complications are not yet evident and those who already have at least one such response.

In the former instance, such treatment is useful to prevent the occurrence of such response and/or reduce the extent to which they occur. In the latter instance, such treatment is useful to reduce the extent to which such response occurs, prevent their further development or reverse the response.

In certain embodiments, the agent that interferes with the response cascade may be an antibody specific for such response.

Expression of Biomarker(s)

Expression of a marker can be inhibited in a number of ways, including, by way of a non-limiting example, an antisense oligonucleotide can be provided to the disease cells in order to inhibit transcription, translation, or both, of the marker(s). Alternately, a polynucleotide encoding an antibody, an antibody derivative, or an antibody fragment which specifically binds a marker protein, and operably linked with an appropriate promoter/regulator region, can be provided to the cell in order to generate intracellular antibodies which will inhibit the function or activity of the protein. The expression and/or function of a marker may also be inhibited by treating the disease cell with an antibody, antibody derivative or antibody fragment that specifically binds a marker protein. Using the methods described herein, a variety of molecules, particularly including molecules sufficiently small that they are able to cross the cell membrane, can be screened in order to identify molecules which inhibit expression of a marker or inhibit the function of a marker protein. The compound so identified can be provided to the subject in order to inhibit disease cells of the subject.

Any marker or combination of markers, as well as any certain markers in combination with the markers, may be used in the compositions, kits and methods described herein. In general, it is desirable to use markers for which the difference between the level of expression of the marker in disease cells and the level of expression of the same marker in normal colon system cells is as great as possible. Although this difference can be as small as the limit of detection of the method for assessing expression of the marker, it is desirable that the difference be at least greater than the standard error of the assessment method, and, in certain embodiments, a difference of at least 2-, 3-, 4-, 5-, 6-, 7-, 8-, 9-, 10-, 15-, 20-, 100-, 500-, 1000-fold or greater than the level of expression of the same marker in normal tissue.

It is recognized that certain marker proteins are secreted to the extracellular space surrounding the cells. These markers are used in certain embodiments of the compositions, kits and methods, owing to the fact that such marker proteins can be detected in a body fluid sample, which may be more easily collected from a human subject than a tissue biopsy sample. In addition, in vivo techniques for detection of a marker protein include introducing into a subject a labeled antibody directed against the protein. For example, the antibody can be labeled with a radioactive marker whose presence and location in a subject can be detected by standard imaging techniques.

In order to determine whether any particular marker protein is a secreted protein, the marker protein is expressed in, for example, a mammalian cell, such as a human cell line, extracellular fluid is collected, and the presence or absence of the protein in the extracellular fluid is assessed (e.g. using a labeled antibody which binds specifically with the protein).

It will be appreciated that subject samples containing such cells may be used in the methods described herein. In these embodiments, the level of expression of the marker can be assessed by assessing the amount (e.g., absolute amount or concentration) of the marker in a sample. The cell sample can, of course, be subjected to a variety of post-collection preparative and storage techniques (e.g., nucleic acid and/or protein extraction, fixation, storage, freezing, ultrafiltration, concentration, evaporation, centrifugation, etc.) prior to assessing the amount of the marker in the sample.

It will also be appreciated that the markers may be shed from the cells into, for example, the respiratory system, digestive system, the blood stream and/or interstitial spaces. The shed markers can be tested, for example, by examining the sputum, BAL, serum, plasma, urine, stool, etc.

The compositions, kits and methods can be used to detect expression of marker proteins having at least one portion which is displayed on the surface of cells which express it. For example, immunological methods may be used to detect such proteins on whole cells, or computer-based sequence analysis methods may be used to predict the presence of at least one extracellular domain (i.e., including both secreted proteins and proteins having at least one cell-surface domain) Expression of a marker protein having at least one portion which is displayed on the surface of a cell which expresses it may be detected without necessarily lysing the cell (e.g., using a labeled antibody which binds specifically with a cell-surface domain of the protein).

Expression of a marker may be assessed by any of a wide variety of methods for detecting expression of a transcribed nucleic acid or protein. Non-limiting examples of such methods include immunological methods for detection of secreted, cell-surface, cytoplasmic or nuclear proteins, protein purification methods, protein function or activity assays, nucleic acid hybridization methods, nucleic acid reverse transcription methods and nucleic acid amplification methods.

In a particular embodiment, expression of a marker is assessed using an antibody (e.g., a radio-labeled, chromophore-labeled, fluorophore-labeled or enzyme-labeled antibody), an antibody derivative (e.g., an antibody conjugated with a substrate or with the protein or ligand of a protein-ligand pair), or an antibody fragment (e.g., a single-chain antibody, an isolated antibody hypervariable domain, etc.) which binds specifically with a marker protein or fragment thereof, including a marker protein which has undergone all or a portion of its normal post-translational modification.

In another particular embodiment, expression of a marker is assessed by preparing mRNA/cDNA (i.e., a transcribed polynucleotide) from cells in a subject sample, and by hybridizing the mRNA/cDNA with a reference polynucleotide which is a complement of a marker nucleic acid, or a fragment thereof. cDNA can, optionally, be amplified using any of a variety of polymerase chain reaction methods prior to hybridization with the reference polynucleotide; preferably, it is not amplified. Expression of one or more markers can likewise be detected using quantitative PCR to assess the level of expression of the marker(s). Alternatively, any of the many methods of detecting mutations or variants (e.g., single nucleotide polymorphisms, deletions, etc.) of a marker may be used to detect occurrence of a marker in a subject.

In a related embodiment, a mixture of transcribed polynucleotides obtained from the sample is contacted with a substrate having fixed thereto a polynucleotide complementary to or homologous with at least a portion (e.g., at least 7, 10, 15, 20, 25, 30, 40, 50, 100, 500, or more nucleotide residues) of a marker nucleic acid. If polynucleotides complementary to or homologous with are differentially detectable on the substrate (e.g., detectable using different chromophores or fluorophores, or fixed to different selected positions), then the levels of expression of a plurality of markers can be assessed simultaneously using a single substrate (e.g., a “gene chip” microarray of polynucleotides fixed at selected positions). When a method of assessing marker expression is used which involves hybridization of one nucleic acid with another, it is desired that the hybridization be performed under stringent hybridization conditions.

In certain embodiments, the biomarker assays can be performed using mass spectrometry or surface plasmon resonance. In various embodiments, the method of identifying an agent active against a disorder and/or disease state in a subject can include one or more of: a) providing a sample of cells containing one or more markers or derivative thereof; b) preparing an extract from such cells; c) mixing the extract with a labeled nucleic acid probe containing a marker binding site; and, d) determining the formation of a complex between the marker and the nucleic acid probe in the presence or absence of the test agent. The determining step can include subjecting said extract/nucleic acid probe mixture to an electrophoretic mobility shift assay.

In certain embodiments, the determining step comprises an assay selected from an enzyme linked immunoabsorption assay (ELISA), fluorescence based assays and ultra high throughput assays, for example surface plasmon resonance (SPR) or fluorescence correlation spectroscopy (FCS) assays. In such embodiments, the SPR sensor is useful for direct real-time observation of biomolecular interactions since SPR is sensitive to minute refractive index changes at a metal-dielectric surface. SPR is a surface technique that is sensitive to changes of 10⁵ to 10⁻⁶ refractive index (RI) units within approximately 200 nm of the SPR sensor/sample interface. Thus, SPR spectroscopy is useful for monitoring the growth of thin organic films deposited on the sensing layer.

Because the compositions, kits, and methods rely on detection of a difference in expression levels of one or more markers, it is desired that the level of expression of the marker is significantly greater than the minimum detection limit of the method used to assess expression in at least one of normal cells and colon cancer-affected cells.

It is understood that by routine screening of additional subject samples using one or more of the markers, it will be realized that certain of the markers are over-expressed in cells of various types, including a specific disorder and/or disease state in a subject.

In addition, as a greater number of subject samples are assessed for expression of the markers and the outcomes of the individual subjects from whom the samples were obtained are correlated, it will also be confirmed that altered expression of certain of the markers are strongly correlated with a disorder and/or disease state in a subject and that altered expression of other markers are strongly correlated with other diseases. The compositions, kits, and methods are thus useful for characterizing one or more of the stage, grade, histological type, and nature of a disorder and/or disease state in a subject.

When the compositions, kits, and methods are used for characterizing one or more of the stage, grade, histological type, and nature of a disorder and/or disease state in a subject, it is desired that the marker or panel of markers is selected such that a positive result is obtained in at least about 20%, and in certain embodiments, at least about 40%, 60%, or 80%, and in substantially all subjects afflicted with a disorder and/or disease state of the corresponding stage, grade, histological type, or nature. The marker or panel of markers invention can be selected such that a positive predictive value of greater than about 10% is obtained for the general population (in a non-limiting example, coupled with an assay specificity greater than 80%).

When a plurality of markers are used in the compositions, kits, and methods, the level of expression of each marker in a subject sample can be compared with the normal level of expression of each of the plurality of markers in non-disorder and/or non-disease samples of the same type, either in a single reaction mixture (i.e. using reagents, such as different fluorescent probes, for each marker) or in individual reaction mixtures corresponding to one or more of the markers. In one embodiment, a significantly increased level of expression of more than one of the plurality of markers in the sample, relative to the corresponding normal levels, is an indication that the subject is afflicted with a disorder and/or disease state. When a plurality of markers is used, 2, 3, 4, 5, 8, 10, 12, 15, 20, 30, or 50 or more individual markers can be used; in certain embodiments, the use of fewer markers may be desired.

In order to maximize the sensitivity of the compositions, kits, and methods (i.e. by interference attributable to cells of system origin in a subject sample), it is desirable that the marker used therein be a marker which has a restricted tissue distribution, e.g., normally not expressed in a non-system tissue.

It is recognized that the compositions, kits, and methods will be of particular utility to subjects having an enhanced risk of developing a disorder and/or disease state in a subject and their medical advisors. Subjects recognized as having an enhanced risk of developing a disorder and/or disease include, for example, subjects having a familial history of such disorder or disease.

The level of expression of a marker in normal human system tissue can be assessed in a variety of ways. In one embodiment, this normal level of expression is assessed by assessing the level of expression of the marker in a portion of system cells which appear to be normal and by comparing this normal level of expression with the level of expression in a portion of the system cells which is suspected of being abnormal. Alternately, and particularly as further information becomes available as a result of routine performance of the methods described herein, population-average values for normal expression of the markers may be used. In other embodiments, the ‘normal’ level of expression of a marker may be determined by assessing expression of the marker in a subject sample obtained from a non-afflicted subject, from a subject sample obtained from a subject before the suspected onset of a disorder and/or disease state in the subject, from archived subject samples, and the like.

There is also provided herein compositions, kits, and methods for assessing the presence of disorder and/or disease state cells in a sample (e.g. an archived tissue sample or a sample obtained from a subject). These compositions, kits, and methods are substantially the same as those described above, except that, where necessary, the compositions, kits, and methods are adapted for use with samples other than subject samples. For example, when the sample to be used is a parafinized, archived human tissue sample, it can be necessary to adjust the ratio of compounds in the compositions, in the kits, or the methods used to assess levels of marker expression in the sample.

Kits and Reagents

The kits are useful for assessing the presence of disease cells (e.g. in a sample such as a subject sample). The kit comprises a plurality of reagents, each of which is capable of binding specifically with a marker nucleic acid or protein. Suitable reagents for binding with a marker protein include antibodies, antibody derivatives, antibody fragments, and the like. Suitable reagents for binding with a marker nucleic acid (e.g. a genomic DNA, an MRNA, a spliced MRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The kits may optionally comprise additional components useful for performing the methods described herein. By way of example, the kit may comprise fluids (e.g. SSC buffer) suitable for annealing complementary nucleic acids or for binding an antibody with a protein with which it specifically binds, one or more sample compartments, an instructional material which describes performance of the method, a sample of normal colon system cells, a sample of colon cancer-related disease cells, and the like.

Methods of Producing Antibodies

There is also provided herein a method of making an isolated hybridoma which produces an antibody useful for assessing whether a subject is afflicted with a disorder and/or disease state. In this method, a protein or peptide comprising the entirety or a segment of a marker protein is synthesized or isolated (e.g. by purification from a cell in which it is expressed or by transcription and translation of a nucleic acid encoding the protein or peptide in vivo or in vitro). A vertebrate, for example, a mammal such as a mouse, rat, rabbit, or sheep, is immunized using the protein or peptide. The vertebrate may optionally (and preferably) be immunized at least one additional time with the protein or peptide, so that the vertebrate exhibits a robust immune response to the protein or peptide. Splenocytes are isolated from the immunized vertebrate and fused with an immortalized cell line to form hybridomas, using any of a variety of methods. Hybridomas formed in this manner are then screened using standard methods to identify one or more hybridomas which produce an antibody which specifically binds with the marker protein or a fragment thereof. There is also provided herein hybridomas made by this method and antibodies made using such hybridomas.

Methods of Assessing Efficacy

There is also provided herein a method of assessing the efficacy of a test compound for inhibiting disease cells. As described above, differences in the level of expression of the markers correlate with the abnormal state of the subject's cells. Although it is recognized that changes in the levels of expression of certain of the markers likely result from the abnormal state of such cells, it is likewise recognized that changes in the levels of expression of other of the markers induce, maintain, and promote the abnormal state of those cells. Thus, compounds which inhibit a disorder and/or disease state in a subject will cause the level of expression of one or more of the markers to change to a level nearer the normal level of expression for that marker (i.e., the level of expression for the marker in normal cells).

This method thus comprises comparing expression of a marker in a first cell sample and maintained in the presence of the test compound and expression of the marker in a second colon cell sample and maintained in the absence of the test compound. A significantly reduced expression of a marker in the presence of the test compound is an indication that the test compound inhibits a related disease. The cell samples may, for example, be aliquots of a single sample of normal cells obtained from a subject, pooled samples of normal cells obtained from a subject, cells of a normal cell line, aliquots of a single sample of related disease cells obtained from a subject, pooled samples of related disease cells obtained from a subject, cells of a related disease cell line, or the like.

In one embodiment, the samples are cancer-related disease cells obtained from a subject and a plurality of compounds believed to be effective for inhibiting various cancer-related diseases are tested in order to identify the compound which is likely to best inhibit the cancer-related disease in the subject.

This method may likewise be used to assess the efficacy of a therapy for inhibiting a related disease in a subject. In this method, the level of expression of one or more markers in a pair of samples (one subjected to the therapy, the other not subjected to the therapy) is assessed. As with the method of assessing the efficacy of test compounds, if the therapy induces a significantly lower level of expression of a marker then the therapy is efficacious for inhibiting a cancer-related disease. As above, if samples from a selected subject are used in this method, then alternative therapies can be assessed in vitro in order to select a therapy most likely to be efficacious for inhibiting a cancer-related disease in the subject.

As described herein, the abnormal state of human cells is correlated with changes in the levels of expression of the markers. There is also provided a method for assessing the harmful potential of a test compound. This method comprises maintaining separate aliquots of human cells in the presence and absence of the test compound. Expression of a marker in each of the aliquots is compared. A significantly higher level of expression of a marker in the aliquot maintained in the presence of the test compound (relative to the aliquot maintained in the absence of the test compound) is an indication that the test compound possesses a harmful potential. The relative harmful potential of various test compounds can be assessed by comparing the degree of enhancement or inhibition of the level of expression of the relevant markers, by comparing the number of markers for which the level of expression is enhanced or inhibited, or by comparing both. Various aspects are described in further detail in the following subsections.

Isolated Proteins and Antibodies

One aspect pertains to isolated marker proteins and biologically active portions thereof, as well as polypeptide fragments suitable for use as immunogens to raise antibodies directed against a marker protein or a fragment thereof. In one embodiment, the native marker protein can be isolated from cells or tissue sources by an appropriate purification scheme using standard protein purification techniques. In another embodiment, a protein or peptide comprising the whole or a segment of the marker protein is produced by recombinant DNA techniques. Alternative to recombinant expression, such protein or peptide can be synthesized chemically using standard peptide synthesis techniques.

“isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”).

When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a marker protein include polypeptides comprising amino acid sequences sufficiently identical to or derived from the amino acid sequence of the marker protein, which include fewer amino acids than the full length protein, and exhibit at least one activity of the corresponding full-length protein. Typically, biologically active portions comprise a domain or motif with at least one activity of the corresponding full-length protein. A biologically active portion of a marker protein can be a polypeptide which is, for example, 10, 25, 50, 100 or more amino acids in length. Moreover, other biologically active portions, in which other regions of the marker protein are deleted, can be prepared by recombinant techniques and evaluated for one or more of the functional activities of the native form of the marker protein. In certain embodiments, useful proteins are substantially identical (e.g., at least about 40%, and in certain embodiments, 50%, 60%, 70%, 80%, 90%, 95%, or 99%) to one of these sequences and retain the functional activity of the corresponding naturally-occurring marker protein yet differ in amino acid sequence due to natural allelic variation or mutagenesis.

In addition, libraries of segments of a marker protein can be used to generate a variegated population of polypeptides for screening and subsequent selection of variant marker proteins or segments thereof.

Predictive Medicine

There is also provided herein uses of the animal models and markers in the field of predictive medicine in which diagnostic assays, prognostic assays, pharmacogenomics, and monitoring clinical trials are used for prognostic (predictive) purposes to thereby treat an individual prophylactically. Accordingly, there is also provided herein diagnostic assays for determining the level of expression of one or more marker proteins or nucleic acids, in order to determine whether an individual is at risk of developing a particular disorder and/or disease. Such assays can be used for prognostic or predictive purposes to thereby prophylactically treat an individual prior to the onset of the disorder and/or disease.

In another aspect, the methods are useful for at least periodic screening of the same individual to see if that individual has been exposed to chemicals or toxins that change his/her expression patterns.

Yet another aspect pertains to monitoring the influence of agents (e.g., drugs or other compounds) administered either to inhibit a disorder and/or disease or to treat or prevent any other disorder (e.g., in order to understand any system effects that such treatment may have) on the expression or activity of a marker in clinical trials.

Pharmaceutical Compositions

The compounds may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles or microspheres formed of biodegradable or non-biodegradable polymers or proteins or liposomes for targeting to cells. Such systems are well known to those skilled in the art and may be optimized for use with the appropriate nucleic acid.

Various methods for nucleic acid delivery are described, for example in Sambrook et al., 1989, Molecular Cloning: A Laboratory Manual, Cold Spring Harbor Laboratory, New York; and Ausubel et al., 1994, Current Protocols in Molecular Biology, John Wiley & Sons, New York. Such nucleic acid delivery systems comprise the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process.

Formulations for topical administration may include ointments, lotions, creams, gels, drops, suppositories, sprays, liquids and powders. Conventional pharmaceutical carriers, aqueous, powder or oily bases, or thickeners can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, with an added preservative. Those of skill in the art can readily determine the various parameters for preparing and formulating the compositions without resort to undue experimentation. The compound can be used alone or in combination with other suitable components.

In general, methods of administering compounds, including nucleic acids, are well known in the art. In particular, the routes of administration already in use for nucleic acid therapeutics, along with formulations in current use, provide preferred routes of administration and formulation for the nucleic acids selected will depend of course, upon factors such as the particular formulation, the severity of the state of the subject being treated, and the dosage required for therapeutic efficacy. As generally used herein, an “effective amount” is that amount which is able to treat one or more symptoms of the disorder, reverse the progression of one or more symptoms of the disorder, halt the progression of one or more symptoms of the disorder, or prevent the occurrence of one or more symptoms of the disorder in a subject to whom the formulation is administered, as compared to a matched subject not receiving the compound. The actual effective amounts of compound can vary according to the specific compound or combination thereof being utilized, the particular composition formulated, the mode of administration, and the age, weight, condition of the individual, and severity of the symptoms or condition being treated.

Any acceptable method known to one of ordinary skill in the art may be used to administer a formulation to the subject. The administration may be localized (i.e., to a particular region, physiological system, tissue, organ, or cell type) or systemic, depending on the condition being treated.

Pharmacogenomics

The markers are also useful as pharmacogenomic markers. As used herein, a “pharmacogenomic marker” is an objective biochemical marker whose expression level correlates with a specific clinical drug response or susceptibility in a subject. The presence or quantity of the pharmacogenomic marker expression is related to the predicted response of the subject and more particularly the subject's tumor to therapy with a specific drug or class of drugs. By assessing the presence or quantity of the expression of one or more pharmacogenomic markers in a subject, a drug therapy which is most appropriate for the subject, or which is predicted to have a greater degree of success, may be selected.

Monitoring Clinical Trials

Monitoring the influence of agents (e.g., drug compounds) on the level of expression of a marker can be applied not only in basic drug screening, but also in clinical trials. For example, the effectiveness of an agent to affect marker expression can be monitored in clinical trials of subjects receiving treatment for a colon cancer-related disease.

In one non-limiting embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of:

obtaining a pre-administration sample from a subject prior to administration of the agent;

detecting the level of expression of one or more selected markers in the pre-administration sample;

obtaining one or more post-administration samples from the subject;

detecting the level of expression of the marker(s) in the post-administration samples;

comparing the level of expression of the marker(s) in the pre-administration sample with the level of expression of the marker(s) in the post-administration sample or samples; and

altering the administration of the agent to the subject accordingly.

For example, increased expression of the marker gene(s) during the course of treatment may indicate ineffective dosage and the desirability of increasing the dosage. Conversely, decreased expression of the marker gene(s) may indicate efficacious treatment and no need to change dosage.

Electronic Apparatus Readable Media, Systems, Arrays and Methods of Using Same

As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; and general hard disks and hybrids of these categories such as magnetic/optical storage media. The medium is adapted or configured for having recorded thereon a marker as described herein.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as personal digital assistants (PDAs), cellular phone, pager and the like; and local and distributed processing systems.

As used herein, “recorded” refers to a process for storing or encoding information on the electronic apparatus readable medium. Those skilled in the art can readily adopt any method for recording information on media to generate materials comprising the markers described herein.

A variety of software programs and formats can be used to store the marker information of the present invention on the electronic apparatus readable medium. Any number of data processor structuring formats (e.g., text file or database) may be employed in order to obtain or create a medium having recorded thereon the markers. By providing the markers in readable form, one can routinely access the marker sequence information for a variety of purposes. For example, one skilled in the art can use the nucleotide or amino acid sequences in readable form to compare a target sequence or target structural motif with the sequence information stored within the data storage means. Search means are used to identify fragments or regions of the sequences which match a particular target sequence or target motif.

Thus, there is also provided herein a medium for holding instructions for performing a method for determining whether a subject has a cancer-related disease or a pre-disposition to a cancer-related disease, wherein the method comprises the steps of determining the presence or absence of a marker and based on the presence or absence of the marker, determining whether the subject has a cancer-related disease or a pre-disposition to a cancer-related disease and/or recommending a particular treatment for a cancer-related disease or pre-cancer-related disease condition.

There is also provided herein an electronic system and/or in a network, a method for determining whether a subject has a cancer-related disease or a pre-disposition to a cancer-related disease associated with a marker wherein the method comprises the steps of determining the presence or absence of the marker, and based on the presence or absence of the marker, determining whether the subject has a particular disorder and/or disease or a pre-disposition to such disorder and/or disease, and/or recommending a particular treatment for such disease or disorder and/or such pre-cancer-related disease condition. The method may further comprise the step of receiving phenotypic information associated with the subject and/or acquiring from a network phenotypic information associated with the subject.

Also provided herein is a network, a method for determining whether a subject has a disorder and/or disease or a pre-disposition to a disorder and/or disease associated with a marker, the method comprising the steps of receiving information associated with the marker, receiving phenotypic information associated with the subject, acquiring information from the network corresponding to the marker and/or disorder and/or disease, and based on one or more of the phenotypic information, the marker, and the acquired information, determining whether the subject has a disorder and/or disease or a pre-disposition thereto. The method may further comprise the step of recommending a particular treatment for the disorder and/or disease or pre-disposition thereto.

There is also provided herein a business method for determining whether a subject has a disorder and/or disease or a pre-disposition thereto, the method comprising the steps of receiving information associated with the marker, receiving phenotypic information associated with the subject, acquiring information from the network corresponding to the marker and/or a disorder and/or disease, and based on one or more of the phenotypic information, the marker, and the acquired information, determining whether the subject has a disorder and/or disease or a pre-disposition thereto. The method may further comprise the step of recommending a particular treatment therefor.

There is also provided herein an array that can be used to assay expression of one or more genes in the array. In one embodiment, the array can be used to assay gene expression in a tissue to ascertain tissue specificity of genes in the array. In this manner, up to about 7000 or more genes can be simultaneously assayed for expression. This allows a profile to be developed showing a battery of genes specifically expressed in one or more tissues.

In addition to such qualitative determination, there is provided herein the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined In this context, the effect of one cell type on another cell type in response to a biological stimulus can be determined

Such a determination is useful, for example, to know the effect of cell-cell interaction at the level of gene expression. If an agent is administered therapeutically to treat one cell type but has an undesirable effect on another cell type, the method provides an assay to determine the molecular basis of the undesirable effect and thus provides the opportunity to co-administer a counteracting agent or otherwise treat the undesired effect Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a disorder and/or disease, progression thereof, and processes, such as cellular transformation associated therewith.

The array is also useful for ascertaining the effect of the expression of a gene or the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes that could serve as a molecular target for diagnosis or therapeutic intervention.

Surrogate Markers

The markers may serve as surrogate markers for one or more disorders or disease states or for conditions leading up thereto. As used herein, a “surrogate marker” is an objective biochemical marker which correlates with the absence or presence of a disease or disorder, or with the progression of a disease or disorder. The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies, or when an assessment of disease progression is desired before a potentially dangerous clinical endpoint is reached.

The markers are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker Similarly, the presence or quantity of the pharmacodynamic marker may be related to the presence or quantity of the metabolic product of a drug, such that the presence or quantity of the marker is indicative of the relative breakdown rate of the drug in vivo.

Pharmacodynamic markers are of particular use in increasing the sensitivity of detection of drug effects, particularly when the drug is administered in low doses. Since even a small amount of a drug may be sufficient to activate multiple rounds of marker transcription or expression, the amplified marker may be in a quantity which is more readily detectable than the drug itself. Also, the marker may be more easily detected due to the nature of the marker itself; for example, using the methods described herein, antibodies may be employed in an immune-based detection system for a protein marker, or marker-specific radiolabeled probes may be used to detect a mRNA marker. Furthermore, the use of a pharmacodynamic marker may offer mechanism-based prediction of risk due to drug treatment beyond the range of possible direct observations.

Protocols for Testing

The method of testing for a disorder and/or disease may comprise, for example measuring the expression level of each marker gene in a biological sample from a subject over time and comparing the level with that of the marker gene in a control biological sample.

When the marker gene is one of the genes described herein and the expression level is differentially expressed (for examples, higher or lower than that in the control), the subject is judged to be affected with a disorder and/or disease. When the expression level of the marker gene falls within the permissible range, the subject is unlikely to be affected therewith.

The standard value for the control may be pre-determined by measuring the expression level of the marker gene in the control, in order to compare the expression levels. For example, the standard value can be determined based on the expression level of the above-mentioned marker gene in the control. For example, in certain embodiments, the permissible range is taken as ±2S.D. based on the standard value. Once the standard value is determined, the testing method may be performed by measuring only the expression level in a biological sample from a subject and comparing the value with the determined standard value for the control.

Expression levels of marker genes include transcription of the marker genes to mRNA, and translation into proteins. Therefore, one method of testing for a disorder and/or disease is performed based on a comparison of the intensity of expression of mRNA corresponding to the marker genes, or the expression level of proteins encoded by the marker genes.

The measurement of the expression levels of marker genes in the testing for a disorder and/or disease can be carried out according to various gene analysis methods. Specifically, one can use, for example, a hybridization technique using nucleic acids that hybridize to these genes as probes, or a gene amplification technique using DNA that hybridize to the marker genes as primers.

The probes or primers used for the testing can be designed based on the nucleotide sequences of the marker genes. The identification numbers for the nucleotide sequences of the respective marker genes are described herein.

Further, it is to be understood that genes of higher animals generally accompany polymorphism in a high frequency. There are also many molecules that produce isoforms comprising mutually different amino acid sequences during the splicing process. Any gene associated with a colon cancer-related disease that has an activity similar to that of a marker gene is included in the marker genes, even if it has nucleotide sequence differences due to polymorphism or being an isoform.

It is also to be understood that the marker genes can include homologs of other species in addition to humans. Thus, unless otherwise specified, the expression “marker gene” refers to a homolog of the marker gene unique to the species or a foreign marker gene which has been introduced into an individual.

Also, it is to be understood that a “homolog of a marker gene” refers to a gene derived from a species other than a human, which can hybridize to the human marker gene as a probe under stringent conditions. Such stringent conditions are known to one skilled in the art who can select an appropriate condition to produce an equal stringency experimentally or empirically.

A polynucleotide comprising the nucleotide sequence of a marker gene or a nucleotide sequence that is complementary to the complementary strand of the nucleotide sequence of a marker gene and has at least 15 nucleotides, can be used as a primer or probe. Thus, a “complementary strand” means one strand of a double stranded DNA with respect to the other strand and which is composed of A:T (U for RNA) and G:C base pairs.

In addition, “complementary” means not only those that are completely complementary to a region of at least 15 continuous nucleotides, but also those that have a nucleotide sequence homology of at least 40% in certain instances, 50% in certain instances, 60% in certain instances, 70% in certain instances, 80% in certain instances, 90% in certain instances, and 95% in certain instances, or higher. The degree of homology between nucleotide sequences can be determined by an algorithm, BLAST, etc.

Such polynucleotides are useful as a probe to detect a marker gene, or as a primer to amplify a marker gene. When used as a primer, the polynucleotide comprises usually 15 by to 100 bp, and in certain embodiments 15 by to 35 by of nucleotides. When used as a probe, a DNA comprises the whole nucleotide sequence of the marker gene (or the complementary strand thereof), or a partial sequence thereof that has at least 15 by nucleotides. When used as a primer, the 3′ region must be complementary to the marker gene, while the 5′ region can be linked to a restriction enzyme-recognition sequence or a tag.

“Polynucleotides” may be either DNA or RNA. These polynucleotides may be either synthetic or naturally-occurring. Also, DNA used as a probe for hybridization is usually labeled. Those skilled in the art readily understand such labeling methods. Herein, the term “oligonucleotide” means a polynucleotide with a relatively low degree of polymerization. Oligonucleotides are included in polynucleotides.

Tests for a disorder and/or disease using hybridization techniques can be performed using, for example, Northern hybridization, dot blot hybridization, or the DNA micro array technique. Furthermore, gene amplification techniques, such as the RT-PCR method may be used. By using the PCR amplification monitoring method during the gene amplification step in RT-PCR, one can achieve a more quantitative analysis of the expression of a marker gene.

In the PCR gene amplification monitoring method, the detection target (DNA or reverse transcript of RNA) is hybridized to probes that are labeled with a fluorescent dye and a quencher which absorbs the fluorescence. When the PCR proceeds and Taq polymerase degrades the probe with its 5′-3′ exonuclease activity, the fluorescent dye and the quencher draw away from each other and the fluorescence is detected. The fluorescence is detected in real time. By simultaneously measuring a standard sample in which the copy number of a target is known, it is possible to determine the copy number of the target in the subject sample with the cycle number where PCR amplification is linear. Also, one skilled in the art recognizes that the PCR amplification monitoring method can be carried out using any suitable method.

The method of testing for a colon cancer-related disease can be also carried out by detecting a protein encoded by a marker gene. Hereinafter, a protein encoded by a marker gene is described as a “marker protein.” For such test methods, for example, the Western blotting method, the immunoprecipitation method, and the ELISA method may be employed using an antibody that binds to each marker protein.

Antibodies used in the detection that bind to the marker protein may be produced by any suitable technique. Also, in order to detect a marker protein, such an antibody may be appropriately labeled. Alternatively, instead of labeling the antibody, a substance that specifically binds to the antibody, for example, protein A or protein G, may be labeled to detect the marker protein indirectly. More specifically, such a detection method can include the ELISA method.

A protein or a partial peptide thereof used as an antigen may be obtained, for example, by inserting a marker gene or a portion thereof into an expression vector, introducing the construct into an appropriate host cell to produce a transformant, culturing the transformant to express the recombinant protein, and purifying the expressed recombinant protein from the culture or the culture supernatant. Alternatively, the amino acid sequence encoded by a gene or an oligopeptide comprising a portion of the amino acid sequence encoded by a full-length cDNA are chemically synthesized to be used as an immunogen.

Furthermore, a test for a colon cancer-related disease can be performed using as an index not only the expression level of a marker gene but also the activity of a marker protein in a biological sample. Activity of a marker protein means the biological activity intrinsic to the protein. Various methods can be used for measuring the activity of each protein.

Even if a subject is not diagnosed as being affected with a disorder and/or disease in a routine test in spite of symptoms suggesting these diseases, whether or not such a subject is suffering from a disorder and/or disease can be easily determined by performing a test according to the methods described herein.

More specifically, in certain embodiments, when the marker gene is one of the genes described herein, an increase or decrease in the expression level of the marker gene in a subject whose symptoms suggest at least a susceptibility to a disorder and/or disease indicates that the symptoms are primarily caused thereby.

In addition, the tests are useful to determine whether a disorder and/or disease is improving in a subject. In other words, the methods described herein can be used to judge the therapeutic effect of a treatment therefor. Furthermore, when the marker gene is one of the genes described herein, an increase or decrease in the expression level of the marker gene in a subject, who has been diagnosed as being affected thereby, implies that the disease has progressed more.

The severity and/or susceptibility to a disorder and/or disease may also be determined based on the difference in expression levels. For example, when the marker gene is one of the genes described herein, the degree of increase in the expression level of the marker gene is correlated with the presence and/or severity of a disorder and/or disease.

Animal Models

Animal models for a disorder and/or disease where the expression level of one or more marker genes or a gene functionally equivalent to the marker gene has been elevated in the animal model can also be made. A “functionally equivalent gene” as used herein generally is a gene that encodes a protein having an activity similar to a known activity of a protein encoded by the marker gene. A representative example of a functionally equivalent gene includes a counterpart of a marker gene of a subject animal, which is intrinsic to the animal.

The animal model is useful for detecting physiological changes due to a disorder and/or disease. In certain embodiments, the animal model is useful to reveal additional functions of marker genes and to evaluate drugs whose targets are the marker genes.

An animal model can be created by controlling the expression level of a counterpart gene or administering a counterpart gene. The method can include creating an animal model by controlling the expression level of a gene selected from the group of genes described herein. In another embodiment, the method can include creating an animal model by administering the protein encoded by a gene described herein, or administering an antibody against the protein. It is to be also understood, that in certain other embodiments, the marker can be over-expressed such that the marker can then be measured using appropriate methods. In another embodiment, an animal model can be created by introducing a gene selected from such groups of genes, or by administering a protein encoded by such a gene. In another embodiment, a disorder and/or disease can be induced by suppressing the expression of a gene selected from such groups of genes or the activity of a protein encoded by such a gene. An antisense nucleic acid, a ribozyme, or an RNAi can be used to suppress the expression. The activity of a protein can be controlled effectively by administering a substance that inhibits the activity, such as an antibody.

The animal model is useful to elucidate the mechanism underlying a disorder and/or disease and also to test the safety of compounds obtained by screening. For example, when an animal model develops the symptoms of a particular disorder and/or disease, or when a measured value involved in a certain disorder and/or disease alters in the animal, a screening system can be constructed to explore compounds having activity to alleviate the disease.

As used herein, the expression “an increase in the expression level” refers to any one of the following: where a marker gene introduced as a foreign gene is expressed artificially; where the transcription of a marker gene intrinsic to the subject animal and the translation thereof into the protein are enhanced; or where the hydrolysis of the protein, which is the translation product, is suppressed.

As used herein, the expression “a decrease in the expression level” refers to either the state in which the transcription of a marker gene of the subject animal and the translation thereof into the protein are inhibited, or the state in which the hydrolysis of the protein, which is the translation product, is enhanced. The expression level of a gene can be determined, for example, by a difference in signal intensity on a DNA chip. Furthermore, the activity of the translation product—the protein—can be determined by comparing with that in the normal state.

It is also within the contemplated scope that the animal model can include transgenic animals, including, for example animals where a marker gene has been introduced and expressed artificially; marker gene knockout animals; and knock-in animals in which another gene has been substituted for a marker gene. A transgenic animal, into which an antisense nucleic acid of a marker gene, a ribozyme, a polynucleotide having an RNAi effect, or a DNA functioning as a decoy nucleic acid or such has been introduced, can be used as the transgenic animal. Such transgenic animals also include, for example, animals in which the activity of a marker protein has been enhanced or suppressed by introducing a mutation(s) into the coding region of the gene, or the amino acid sequence has been modified to become resistant or susceptible to hydrolysis. Mutations in an amino acid sequence include substitutions, deletions, insertions, and additions.

Examples of Expression

In addition, the expression itself of a marker gene can be controlled by introducing a mutation(s) into the transcriptional regulatory region of the gene. Those skilled in the art understand such amino acid substitutions. Also, the number of amino acids that are mutated is not particularly restricted, as long as the activity is maintained Normally, it is within 50 amino acids, in certain non-limiting embodiments, within 30 amino acids, within 10 amino acids, or within 3 amino acids. The site of mutation may be any site, as long as the activity is maintained

In yet another aspect, there is provided herein screening methods for candidate compounds for therapeutic agents to treat a particular disorder and/or disease. One or more marker genes are selected from the group of genes described herein. A therapeutic agent for a colon cancer-related disease can be obtained by selecting a compound capable of increasing or decreasing the expression level of the marker gene(s).

It is to be understood that the expression “a compound that increases the expression level of a gene” refers to a compound that promotes any one of the steps of gene transcription, gene translation, or expression of a protein activity. On the other hand, the expression “a compound that decreases the expression level of a gene”, as used herein, refers to a compound that inhibits any one of these steps.

In particular aspects, the method of screening for a therapeutic agent for a disorder and/or disease can be carried out either in vivo or in vitro. This screening method can be performed, for example, by:

administering a candidate compound to an animal subject;

measuring the expression level of a marker gene(s) in a biological sample from the animal subject; or

selecting a compound that increases or decreases the expression level of a marker gene(s) as compared to that in a control with which the candidate compound has not been contacted.

In still another aspect, there is provided herein a method to assess the efficacy of a candidate compound for a pharmaceutical agent on the expression level of a marker gene(s) by contacting an animal subject with the candidate compound and monitoring the effect of the compound on the expression level of the marker gene(s) in a biological sample derived from the animal subject. The variation in the expression level of the marker gene(s) in a biological sample derived from the animal subject can be monitored using the same technique as used in the testing method described above. Furthermore, based on the evaluation, a candidate compound for a pharmaceutical agent can be selected by screening.

All patents, patent applications and references cited herein are incorporated in their entirety by reference. While the invention has been described and exemplified in sufficient detail for those skilled in this art to make and use it, various alternatives, modifications and improvements should be apparent without departing from the spirit and scope of the invention. One skilled in the art readily appreciates that the present invention is well adapted to carry out the objects and obtain the ends and advantages mentioned, as well as those inherent therein.

Certain Nucleobase Sequences

Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compounds that may encompass such modified oligonucleotides may be complementary to any nucleobase sequence version of the miRNAs described herein.

It is understood that any nucleobase sequence set forth herein is independent of any modification to a sugar moiety, an internucleoside linkage, or a nucleobase. It is further understood that a nucleobase sequence comprising U's also encompasses the same nucleobase sequence wherein ‘U’ is replaced by ‘T’ at one or more positions having ‘U’. Conversely, it is understood that a nucleobase sequence comprising T's also encompasses the same nucleobase sequence wherein ‘T’ is replaced by ‘U’ at one or more positions having ‘T’.

In certain embodiments, a modified oligonucleotide has a nucleobase sequence that is complementary to a miRNA or a precursor thereof, meaning that the nucleobase sequence of a modified oligonucleotide is a least 60%, 65%, 70%, 75%, 80%, 85%, 90%, 95%, 97%, 98% or 99% identical to the complement of a miRNA or precursor thereof over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the two sequences hybridize under stringent hybridization conditions. Accordingly, in certain embodiments the nucleobase sequence of a modified oligonucleotide may have one or more mismatched basepairs with respect to its target miRNA or target miRNA precursor sequence, and is capable of hybridizing to its target sequence. In certain embodiments, a modified oligonucleotide has a nucleobase sequence that is 100% complementary to a miRNA or a precursor thereof. In certain embodiments, the nucleobase sequence of a modified oligonucleotide has full-length complementary to a miRNA.

miRNA (miR) Therapies

In some embodiments, the present invention provides microRNAs that inhibit the expression of one or more genes in a subject. MicroRNA expression profiles can serve as a new class of cancer biomarkers.

Included herein are methods of inhibiting gene expression and/or activity using one or more MiRs. In some embodiments, the miR(s) inhibit the expression of a protein. In other embodiments, the miRNA(s) inhibits gene activity (e.g., cell invasion activity).

The miRNA can be isolated from cells or tissues, recombinantly produced, or synthesized in vitro by a variety of techniques well known to one of ordinary skill in the art. In one embodiment, miRNA is isolated from cells or tissues. Techniques for isolating miRNA from cells or tissues are well known to one of ordinary skill in the art. For example, miRNA can be isolated from total RNA using the mirVana miRNA isolation kit from Ambion, Inc. Another technique utilizes the flashIPAGE™ Fractionator System (Ambion, Inc.) for PAGE purification of small nucleic acids.

For the use of miRNA therapeutics, it is understood by one of ordinary skill in the art that nucleic acids administered in vivo are taken up and distributed to cells and tissues.

The nucleic acid may be delivered in a suitable manner which enables tissue-specific uptake of the agent and/or nucleic acid delivery system. The formulations described herein can supplement treatment conditions by any known conventional therapy, including, but not limited to, antibody administration, vaccine administration, administration of cytotoxic agents, natural amino acid polypeptides, nucleic acids, nucleotide analogues, and biologic response modifiers. Two or more combined compounds may be used together or sequentially.

Certain embodiments of the invention provide pharmaceutical compositions containing (a) one or more nucleic acid or small molecule compounds and (b) one or more other chemotherapeutic agents.

Additional Useful Definitions

“Subject” means a human or non-human animal selected for treatment or therapy. “Subject suspected of having” means a subject exhibiting one or more clinical indicators of a disorder, disease or condition.

“Preventing” or “prevention” refers to delaying or forestalling the onset, development or progression of a condition or disease for a period of time, including weeks, months, or years. “Treatment” or “treat” means the application of one or more specific procedures used for the cure or amelioration of a disorder and/or disease. In certain embodiments, the specific procedure is the administration of one or more pharmaceutical agents.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Subject in need thereof” means a subject identified as in need of a therapy or treatment.

“Administering” means providing a pharmaceutical agent or composition to a subject, and includes, but is not limited to, administering by a medical professional and self-administering.

“Parenteral administration” means administration through injection or infusion. Parenteral administration includes, but is not limited to, subcutaneous administration, intravenous administration, intramuscular administration, intraarterial administration, and intracranial administration. “Subcutaneous administration” means administration just below the skin.

“Improves function” means the changes function toward normal parameters. In certain embodiments, function is assessed by measuring molecules found in a subject's bodily fluids. “Pharmaceutical composition” means a mixture of substances suitable for administering to an individual that includes a pharmaceutical agent. For example, a pharmaceutical composition may comprise a modified oligonucleotide and a sterile aqueous solution.

“Target nucleic acid,” “target RNA,” “target RNA transcript” and “nucleic acid target” all mean a nucleic acid capable of being targeted by antisense compounds. “Targeting” means the process of design and selection of nucleobase sequence that will hybridize to a target nucleic acid and induce a desired effect. “Targeted to” means having a nucleobase sequence that will allow hybridization to a target nucleic acid to induce a desired effect. In certain embodiments, a desired effect is reduction of a target nucleic acid.

“Modulation” means a perturbation of function or activity. In certain embodiments, modulation means an increase in gene expression. In certain embodiments, modulation means a decrease in gene expression.

“Expression” means any functions and steps by which a gene's coded information is converted into structures present and operating in a cell.

“Region” means a portion of linked nucleosides within a nucleic acid. In certain embodiments, a modified oligonucleotide has a nucleobase sequence that is complementary to a region of a target nucleic acid. For example, in certain such embodiments a modified oligonucleotide is complementary to a region of a miRNA stem-loop sequence. In certain such embodiments, a modified oligonucleotide is 100% identical to a region of a miRNA sequence.

“Segment” means a smaller or sub-portion of a region.

“Nucleobase sequence” means the order of contiguous nucleobases, in a 5′ to 3′ orientation, independent of any sugar, linkage, and/or nucleobase modification.

“Contiguous nucleobases” means nucleobases immediately adjacent to each other in a nucleic acid.

“Nucleobase complementarity” means the ability of two nucleobases to pair non-covalently via hydrogen bonding. “Complementary” means a first nucleobase sequence is at least 60%, 65%, 70%, 75%, 80%, 85%, 90%, 95%, 97%, 98% or 99% identical, or is 100% identical, to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the two sequences hybridize under stringent hybridization conditions. In certain embodiments a modified oligonucleotide that has a nucleobase sequence which is 100% complementary to a miRNA, or precursor thereof, may not be 100% complementary to the miRNA, or precursor thereof, over the entire length of the modified oligonucleotide.

“Complementarity” means the nucleobase pairing ability between a first nucleic acid and a second nucleic acid. “Full-length complementarity” means each nucleobase of a first nucleic acid is capable of pairing with each nucleobase at a corresponding position in a second nucleic acid. For example, in certain embodiments, a modified oligonucleotide wherein each nucleobase has complementarity to a nucleobase in an miRNA has full-length complementarity to the miRNA.

“Percent complementary” means the number of complementary nucleobases in a nucleic acid divided by the length of the nucleic acid. In certain embodiments, percent complementarity of a modified oligonucleotide means the number of nucleobases that are complementary to the target nucleic acid, divided by the number of nucleobases of the modified oligonucleotide. In certain embodiments, percent complementarity of a modified oligonucleotide means the number of nucleobases that are complementary to a miRNA, divided by the number of nucleobases of the modified oligonucleotide.

“Percent region bound” means the percent of a region complementary to an oligonucleotide region. Percent region bound is calculated by dividing the number of nucleobases of the target region that are complementary to the oligonucleotide by the length of the target region. In certain embodiments, percent region bound is at least 80%, at least 85%, at least 90%, at least 95%, at least 96%, at least 97%, at least 98%, at least 99%, or 100%.

“Percent identity” means the number of nucleobases in first nucleic acid that are identical to nucleobases at corresponding positions in a second nucleic acid, divided by the total number of nucleobases in the first nucleic acid.

“Substantially identical” used herein may mean that a first and second nucleobase sequence are at least 60%, 65%, 70%, 75%, 80%, 85%, 90%, 95%, 97%, 98% or 99% identical, or 100% identical, over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases.

“Hybridize” means the annealing of complementary nucleic acids that occurs through nucleobase complementarity.

“Mismatch” means a nucleobase of a first nucleic acid that is not capable of pairing with a nucleobase at a corresponding position of a second nucleic acid.

“Non-complementary nucleobase” means two nucleobases that are not capable of pairing through hydrogen bonding.

“Identical” means having the same nucleobase sequence.

“miRNA” or “miR” means a non-coding RNA between 18 and 25 nucleobases in length which hybridizes to and regulates the expression of a coding RNA. In certain embodiments, a miRNA is the product of cleavage of a pre-miRNA by the enzyme Dicer. Examples of miRNAs are found in the miRNA database known as miRBase (http://microma.sanger.ac.uk).

“Pre-miRNA” or “pre-miR” means a non-coding RNA having a hairpin structure, which contains a miRNA. In certain embodiments, a pre-miRNA is the product of cleavage of a pri-miR by the double-stranded RNA-specific ribonuclease known as Drosha.

“Stem-loop sequence” means an RNA having a hairpin structure and containing a mature miRNA sequence. Pre-miRNA sequences and stem-loop sequences may overlap. Examples of stem-loop sequences are found in the miRNA database known as miRBase (microma.sanger.ac.uk/.

“miRNA precursor” means a transcript that originates from a genomic DNA and that comprises a non-coding, structured RNA comprising one or more miRNA sequences. For example, in certain embodiments a miRNA precursor is a pre-miRNA. In certain embodiments, a miRNA precursor is a pri-miRNA.

“Antisense compound” means a compound having a nucleobase sequence that will allow hybridization to a target nucleic acid. In certain embodiments, an antisense compound is an oligonucleotide having a nucleobase sequence complementary to a target nucleic acid.

“Oligonucleotide” means a polymer of linked nucleosides, each of which can be modified or unmodified, independent from one another. “Naturally occurring internucleoside linkage” means a 3′ to 5′ phosphodiester linkage between nucleosides. “Natural nucleobase” means a nucleobase that is unmodified relative to its naturally occurring form. “miR antagonist”+means an agent designed to interfere with or inhibit the activity of a miRNA. In certain embodiments, a miR antagonist comprises an antisense compound targeted to a miRNA. In certain embodiments, a miR antagonist comprises a modified oligonucleotide having a nucleobase sequence that is complementary to the nucleobase sequence of a miRNA, or a precursor thereof. In certain embodiments, an miR antagonist comprises a small molecule, or the like that interferes with or inhibits the activity of an miRNA.

The methods and reagents described herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims. It will also be readily apparent to a person skilled in the art that varying substitutions and modifications may be made to the invention disclosed herein without departing from the scope and spirit of the invention.

It should be understood that although the present invention has been specifically disclosed by preferred embodiments and optional features, modifications and variations of the concepts herein disclosed may be resorted to by those skilled in the art, and that such modifications and variations are considered to be within the scope of this invention as defined by the appended claims.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

What is claimed is:

1. A method for inhibiting the proliferation of leukemic cells comprising:
administering at least one miRNA in the miR-15a16-1 cluster; and
inhibiting the growth of leukemic cells.

2. The method of claim 1, further comprising:
inducing apoptosis in leukemic cells.

3. The method of claim 1, wherein the leukemic cells are in vivo tumor engraftments and wherein exposing the cells to one or more miRs in the miR-15a16-1 cluster exerts a tumor suppressor function on such cells.

4. The method of claim 1, wherein the administration of at least one miRNA in the miR-15a16-1 cluster directly silences IGSF4.

5. The method of claim 1, comprising: contacting a cell expressing IGSF4 with one or more miRs in the miR-15a16-1 cluster, under conditions such that the expression of IGSF4 in the cell is inhibited.

6. The method of claim 5, wherein the cell is a cancer cell.

7. The method of claim 5, wherein the cell is a chronic lymphocytic leukemia cell.

8. The method of claim 5, wherein the cell is in an organism.

9. The method of claim 8, wherein the organism is an animal.

10. The method of claim 8, wherein the organism has been diagnosed with cancer.

11. A method for reducing expression of one or more proteins and/or reducing expression of one or more messenger RNA (mRNA) selected from: PDCD4, RAB21, IGSF4, SCAP2, comprising:
transfecting cells in need thereof with one or more miRs in the Mir-15α/16-1 cluster; and
reducing expression of the one or more proteins and/or reducing expression of one or more mRNA.

12. The method of claim 12 wherein the protein is selected from one or more of: Ruvb11, Anxa2, Rcn1, Cct7, Sugt1, Cdc2, Psf1, Grp78, Bc12, Pdia2, Wt1, MageB3, Rab9B, Cdh26, Hsp70, Crhbp, Actr1A, Gapdh, Tomm22, SPnt, Csh11, Hla-B, Tpi1, Hsp90AB1, Acta1, Cfl2, and AldoA.

13. A method of treating leukemia in a subject, comprising:
administering to the subject an effective amount of at least one miR selected from one or more of the miRs of the miR15a/16-1 cluster; or
administering to the subject an effective amount of at least one compound for inducing expression of the at least one miR; and
treating leukemia in the subject.

14. A method of treating, preventing, reversing or limiting the severity of a leukemia-associated disease complication in an individual in need thereof, comprising:
administering to the individual an agent that interferes with at least a leukemia associated disease response cascade, wherein the agent comprises at least one miR,
wherein the miR is selected from one or more of the miRs of the miR15a/16-1 cluster; and
treating, preventing, reversing or limiting the severity of the leukemia-associated disease complication.

15. A method to affect leukemia cancer cells comprising:
introducing a sense or antisense miR-15a and sense or antisense miR-16-1 to the leukemia cancer cells; and
affecting leukemia cancer cells.

16. The method of claim 15, for ameliorating leukemia in a human in need of such amelioration, comprising:
administering sense miR-15a and sense miR-16-1; and
ameliorating the leukemia.

17. The method of claim 16 wherein the leukemia is chronic lymphocytic leukemia (CLL).


SEQUENCE LISTING (4 sequences)

SEQ ID NO: 1 - DNA, 28 bp, Artificial Sequence
  Description of Artificial Sequence Synthetic primer
gctctagaaaaaggagaaccagcacagc

SEQ ID NO: 2 - DNA, 28 bp, Artificial Sequence
  Description of Artificial Sequence Synthetic primer
gctctagatgacacacctcacttgcaga

SEQ ID NO: 3 - protein, 4 aa, Artificial Sequence
  Description of Artificial Sequence Synthetic peptide
DEAD

SEQ ID NO: 4 - protein, 4 aa, Artificial Sequence
  Description of Artificial Sequence Synthetic peptide
DEAH